SEQUENCE LISTING (651 sequences)

SEQ ID NO: 1 - protein, 102 aa, Artificial Sequence
  Recombinant Polypeptide KERE-class Nanobody
EVQLVESGGGLVQPGGSLRLSCAASGIPFSXXXXXWFRQAPGKQRDSVAXXXXXRFTISRDNAKNTVYLQMNSLKPEDTAVYRCYFXXXXXWGQGTQVTVSS

SEQ ID NO: 2 - protein, 102 aa, Artificial Sequence
  Recombinant Polypeptide KERE-class Nanobody
QVKLEESGGGLVQAGGSLRLSCVGSGRTFSXXXXXWFRLAPGKEREFVAXXXXXRFTISRDTASNRGYLHMNNLTPEDTAVYYCAAXXXXXWGQGTQVTVSS

SEQ ID NO: 3 - protein, 102 aa, Artificial Sequence
  Recombinant Polypeptide KERE-class Nanobody
AVQLVDSGGGLVQAGDSLKLSCALTGGAFTXXXXXWFRQTPGREREFVAXXXXXRFTISRDNAKNMVYLRMNSLIPEDAAVYSCAAXXXXXWGQGTLVTVSS

SEQ ID NO: 4 - protein, 102 aa, Artificial Sequence
  Recombinant Polypeptide KERE-class Nanobody
QVQLVESGGGLVEAGGSLRLSCTASESPFRXXXXXWFRQTSGQEREFVAXXXXXRFTISRDDAKNTVWLHGSTLKPEDTAVYYCAAXXXXXWGQGTQVTVSS

SEQ ID NO: 5 - protein, 102 aa, Artificial Sequence
  Recombinant Polypeptide KERE-class Nanobody
AVQLVESGGGLVQGGGSLRLACAASERIFDXXXXXWYRQGPGNERELVAXXXXXRFTISMDYTKQTVYLHMNSLRPEDTGLYYCKIXXXXXWGQGTQVTVSS

SEQ ID NO: 6 - protein, 102 aa, Artificial Sequence
  Recombinant Polypeptide KERE-class Nanobody
DVKFVESGGGLVQAGGSLRLSCVASGFNFDXXXXXWFRQAPGKEREEVAXXXXXRFTISSEKDKNSVYLQMNSLKPEDTALYICAGXXXXXWGRGTQVTVSS

SEQ ID NO: 7 - protein, 102 aa, Artificial Sequence
  Recombinant Polypeptide KERE-class Nanobody
QVRLAESGGGLVQSGGSLRLSCVASGSTYTXXXXXWYRQYPGKQRALVAXXXXXRFTIARDSTKDTFCLQMNNLKPEDTAVYYCYAXXXXXWGQGTQVTVSS

SEQ ID NO: 8 - protein, 102 aa, Artificial Sequence
  Recombinant Polypeptide KERE-class Nanobody
EVQLVESGGGLVQAGGSLRLSCAASGFTSDXXXXXWFRQAPGKPREGVSXXXXXRFTISTDNAKNTVHLLMNRVNAEDTALYYCAVXXXXXWGRGTRVTVSS

SEQ ID NO: 9 - protein, 102 aa, Artificial Sequence
  Recombinant Polypeptide KERE-class Nanobody
QVQLVESGGGLVQPGGSLRLSCQASGDISTXXXXXWYRQVPGKLREFVAXXXXXRFTISGDNAKRAIYLQMNNLKPDDTAVYYCNRXXXXXWGQGTQVTVSP

SEQ ID NO: 10 - protein, 102 aa, Artificial Sequence
  Recombinant Polypeptide KERE-class Nanobody
QVPVVESGGGLVQAGDSLRLFCAVPSFTSTXXXXXWFRQAPGKEREFVAXXXXXRFTISRNATKNTLTLRMDSLKPEDTAVYYCAAXXXXXWGQGTQVTVSS

SEQ ID NO: 11 - protein, 102 aa, Artificial Sequence
  Recombinant Polypeptide KERE-class Nanobody
EVQLVESGGGLVQAGDSLRLFCTVSGGTASXXXXXWFRQAPGEKREFVAXXXXXRFTIARENAGNMVYLQMNNLKPDDTALYTCAAXXXXXWGRGTQVTVSS

SEQ ID NO: 12 - protein, 102 aa, Artificial Sequence
  Recombinant Polypeptide KERE-class Nanobody
AVQLVESGGDSVQPGDSQTLSCAASGRTNSXXXXXWFRQAPGKERVFLAXXXXXRFTISRDSAKNMMYLQMNNLKPQDTAVYYCAAXXXXXWGQGTQVTVSS

SEQ ID NO: 13 - protein, 102 aa, Artificial Sequence
  Recombinant Polypeptide KERE-class Nanobody
AVQLVESGGGLVQAGGSLRLSCVVSGLTSSXXXXXWFRQTPWQERDFVAXXXXXRFTISRDNYKDTVLLEMNFLKPEDTAIYYCAAXXXXXWGQGTQVTVSS

SEQ ID NO: 14 - protein, 102 aa, Artificial Sequence
  Recombinant Polypeptide KERE-class Nanobody
AVQLVESGGGLVQAGASLRLSCATSTRTLDXXXXXWFRQAPGRDREFVAXXXXXRFTVSRDSAENTVALQMNSLKPEDTAVYYCAAXXXXXWGQGTRVTVSS

SEQ ID NO: 15 - protein, 102 aa, Artificial Sequence
  Recombinant Polypeptide KERE-class Nanobody
QVQLVESGGGLVQPGGSLRLSCTVSRLTAHXXXXXWFRQAPGKEREAVSXXXXXRFTISRDYAGNTAFLQMDSLKPEDTGVYYCATXXXXXWGQGTQVTVSS

SEQ ID NO: 16 - protein, 102 aa, Artificial Sequence
  Recombinant Polypeptide KERE-class Nanobody
EVQLVESGGELVQAGGSLKLSCTASGRNFVXXXXXWFRRAPGKEREFVAXXXXXRFTVSRDNGKNTAYLRMNSLKPEDTADYYCAVXXXXXLGSGTQVTVSS

SEQ ID NO: 17 - protein, 102 aa, Artificial Sequence
  Recombinant Polypeptide GLEW-class Nanobody
AVQLVESGGGLVQPGGSLRLSCAASGFTFSXXXXXWVRQAPGKVLEWVSXXXXXRFTISRDNAKNTLYLQMNSLKPEDTAVYYCVKXXXXXGSQGTQVTVSS

SEQ ID NO: 18 - protein, 102 aa, Artificial Sequence
  Recombinant Polypeptide GLEW-class Nanobody
EVQLVESGGGLVQPGGSLRLSCVCVSSGCTXXXXXWVRQAPGKAEEWVSXXXXXRFKISRDNAKKTLYLQMNSLGPEDTAMYYCQRXXXXXRGQGTQVTVSS

SEQ ID NO: 19 - protein, 102 aa, Artificial Sequence
  Recombinant Polypeptide GLEW-class Nanobody
EVQLVESGGGLALPGGSLTLSCVFSGSTFSXXXXXWVRHTPGKAEEWVSXXXXXRFTISRDNAKNTLYLEMNSLSPEDTAMYYCGRXXXXXRSKGIQVTVSS

SEQ ID NO: 20 - protein, 102 aa, Artificial Sequence
  Recombinant Polypeptide P,R,S 103-class Nanobody
AVQLVESGGGLVQAGGSLRLSCAASGRTFSXXXXXWFRQAPGKEREFVAXXXXXRFTISRDNAKNTVYLQMNSLKPEDTAVYYCAAXXXXXRGQGTQVTVSS

SEQ ID NO: 21 - protein, 102 aa, Artificial Sequence
  Recombinant Polypeptide P,R,S 103-class Nanobody
DVQLVESGGDLVQPGGSLRLSCAASGFSFDXXXXXWLRQTPGKGLEWVGXXXXXRFTISRDNAKNMLYLHLNNLKSEDTAVYYCRRXXXXXLGQGTQVTVSS

SEQ ID NO: 22 - protein, 102 aa, Artificial Sequence; identical to SEQ ID NO: 18
  Recombinant Polypeptide P,R,S 103-class Nanobody

SEQ ID NO: 23 - protein, 30 aa, Artificial Sequence
  Recombinant Polypeptide KERE-class Nanobody FW1 sequence
QVQRVESGGGLVQAGGSLRLSCAASGRTSS

SEQ ID NO: 24 - protein, 30 aa, Artificial Sequence
  Recombinant Polypeptide KERE-class Nanobody FW1 sequence
QVQLVESGGGLVQTGDSLSLSCSASGRTFS

SEQ ID NO: 25 - protein, 30 aa, Artificial Sequence
  Recombinant Polypeptide KERE-class Nanobody FW1 sequence
QVKLEESGGGLVQAGDSLRLSCAATGRAFG

SEQ ID NO: 26 - protein, 30 aa, Artificial Sequence
  Recombinant Polypeptide KERE-class Nanobody FW1 sequence
AVQLVESGGGLVQPGESLGLSCVASGRDFV

SEQ ID NO: 27 - protein, 30 aa, Artificial Sequence
  Recombinant Polypeptide KERE-class Nanobody FW1 sequence
EVQLVESGGGLVQAGGSLRLSCEVLGRTAG

SEQ ID NO: 28 - protein, 30 aa, Artificial Sequence
  Recombinant Polypeptide KERE-class Nanobody FW1 sequence
QVQLVESGGGWVQPGGSLRLSCAASETILS

SEQ ID NO: 29 - protein, 30 aa, Artificial Sequence
  Recombinant Polypeptide KERE-class Nanobody FW1 sequence
QVQLVESGGGTVQPGGSLNLSCVASGNTFN

SEQ ID NO: 30 - protein, 30 aa, Artificial Sequence
  Recombinant Polypeptide KERE-class Nanobody FW1 sequence
EVQLVESGGGLAQPGGSLQLSCSAPGFTLD

SEQ ID NO: 31 - protein, 30 aa, Artificial Sequence
  Recombinant Polypeptide KERE-class Nanobody FW1 sequence
AQELEESGGGLVQAGGSLRLSCAASGRTFN

SEQ ID NO: 32 - protein, 22 aa, Artificial Sequence
  Recombinant Polypeptide KERE-class Nanobody FW1 sequence
VESGGGLVQPGGSLRLSCAASG

SEQ ID NO: 33 - protein, 22 aa, Artificial Sequence
  Recombinant Polypeptide KERE-class Nanobody FW1 sequence
VDSGGGLVQAGDSLKLSCALTG

SEQ ID NO: 34 - protein, 22 aa, Artificial Sequence
  Recombinant Polypeptide KERE-class Nanobody FW1 sequence
VDSGGGLVQAGDSLRLSCAASG

SEQ ID NO: 35 - protein, 22 aa, Artificial Sequence
  Recombinant Polypeptide KERE-class Nanobody FW1 sequence
VDSGGGLVEAGGSLRLSCQVSE

SEQ ID NO: 36 - protein, 22 aa, Artificial Sequence
  Recombinant Polypeptide KERE-class Nanobody FW1 sequence
QDSGGGSVQAGGSLKLSCAASG

SEQ ID NO: 37 - protein, 22 aa, Artificial Sequence
  Recombinant Polypeptide KERE-class Nanobody FW1 sequence
VQSGGRLVQAGDSLRLSCAASE

SEQ ID NO: 38 - protein, 22 aa, Artificial Sequence
  Recombinant Polypeptide KERE-class Nanobody FW1 sequence
VESGGTLVQSGDSLKLSCASST

SEQ ID NO: 39 - protein, 22 aa, Artificial Sequence
  Recombinant Polypeptide KERE-class Nanobody FW1 sequence
MESGGDSVQSGGSLTLSCVASG

SEQ ID NO: 40 - protein, 22 aa, Artificial Sequence
  Recombinant Polypeptide KERE-class Nanobody FW1 sequence
QASGGGLVQAGGSLRLSCSASV

SEQ ID NO: 41 - protein, 14 aa, Artificial Sequence
  Recombinant Polypeptide KERE-class Nanobody FW2 sequence
WFRQAPGKEREFVA

SEQ ID NO: 42 - protein, 14 aa, Artificial Sequence
  Recombinant Polypeptide KERE-class Nanobody FW2 sequence
WFRQTPGREREFVA

SEQ ID NO: 43 - protein, 14 aa, Artificial Sequence
  Recombinant Polypeptide KERE-class Nanobody FW2 sequence
WYRQAPGKQREMVA

SEQ ID NO: 44 - protein, 14 aa, Artificial Sequence
  Recombinant Polypeptide KERE-class Nanobody FW2 sequence
WYRQGPGKQRELVA

SEQ ID NO: 45 - protein, 14 aa, Artificial Sequence
  Recombinant Polypeptide KERE-class Nanobody FW2 sequence
WIRQAPGKEREGVS

SEQ ID NO: 46 - protein, 14 aa, Artificial Sequence
  Recombinant Polypeptide KERE-class Nanobody FW2 sequence
WFREAPGKEREGIS

SEQ ID NO: 47 - protein, 14 aa, Artificial Sequence
  Recombinant Polypeptide KERE-class Nanobody FW2 sequence
WYRQAPGKERDLVA

SEQ ID NO: 48 - protein, 14 aa, Artificial Sequence
  Recombinant Polypeptide KERE-class Nanobody FW2 sequence
WFRQAPGKQREEVS

SEQ ID NO: 49 - protein, 14 aa, Artificial Sequence
  Recombinant Polypeptide KERE-class Nanobody FW2 sequence
WFRQPPGKVREFVG

SEQ ID NO: 50 - protein, 32 aa, Artificial Sequence
  Recombinant Polypeptide KERE-class Nanobody FW3 sequence
RFTISRDNAKNTVYLQMNSLKPEDTAVYRCYF

SEQ ID NO: 51 - protein, 32 aa, Artificial Sequence
  Recombinant Polypeptide KERE-class Nanobody FW3 sequence
RFAISRDNNKNTGYLQMNSLEPEDTAVYYCAA

SEQ ID NO: 52 - protein, 32 aa, Artificial Sequence
  Recombinant Polypeptide KERE-class Nanobody FW3 sequence
RFTVARNNAKNTVNLEMNSLKPEDTAVYYCAA

SEQ ID NO: 53 - protein, 32 aa, Artificial Sequence
  Recombinant Polypeptide KERE-class Nanobody FW3 sequence
RFTISRDIAKNTVDLLMNNLEPEDTAVYYCAA

SEQ ID NO: 54 - protein, 32 aa, Artificial Sequence
  Recombinant Polypeptide KERE-class Nanobody FW3 sequence
RLTISRDNAVDTMYLQMNSLKPEDTAVYYCAA

SEQ ID NO: 55 - protein, 32 aa, Artificial Sequence
  Recombinant Polypeptide KERE-class Nanobody FW3 sequence
RFTISRDNAKNTVYLQMDNVKPEDTAIYYCAA

SEQ ID NO: 56 - protein, 32 aa, Artificial Sequence
  Recombinant Polypeptide KERE-class Nanobody FW3 sequence
RFTISKDSGKNTVYLQMTSLKPEDTAVYYCAT

SEQ ID NO: 57 - protein, 32 aa, Artificial Sequence
  Recombinant Polypeptide KERE-class Nanobody FW3 sequence
RFTISRDSAKNMMYLQMNNLKPQDTAVYYCAA

SEQ ID NO: 58 - protein, 32 aa, Artificial Sequence
  Recombinant Polypeptide KERE-class Nanobody FW3 sequence
RFTISRENDKSTVYLQLNSLKPEDTAVYYCAA

SEQ ID NO: 59 - protein, 32 aa, Artificial Sequence
  Recombinant Polypeptide KERE-class Nanobody FW3 sequence
RFTISRDYAGNTAYLQMNSLKPEDTGVYYCAT

SEQ ID NO: 60 - protein, 11 aa, Artificial Sequence
  Recombinant Polypeptide KERE-class Nanobody FW4 sequence
WGQGTQVTVSS

SEQ ID NO: 61 - protein, 11 aa, Artificial Sequence
  Recombinant Polypeptide KERE-class Nanobody FW4 sequence
WGKGTLVTVSS

SEQ ID NO: 62 - protein, 11 aa, Artificial Sequence
  Recombinant Polypeptide KERE-class Nanobody FW4 sequence
RGQGTRVTVSS

SEQ ID NO: 63 - protein, 11 aa, Artificial Sequence
  Recombinant Polypeptide KERE-class Nanobody FW4 sequence
WGLGTQVTISS

SEQ ID NO: 64 - protein, 30 aa, Artificial Sequence
  Recombinant Polypeptide GLEW-class Nanobody FW1 sequence
QVQLVESGGGLVQPGGSLRLSCAASGFTFS

SEQ ID NO: 65 - protein, 30 aa, Artificial Sequence
  Recombinant Polypeptide GLEW-class Nanobody FW1 sequence
EVHLVESGGGLVRPGGSLRLSCAAFGFIFK

SEQ ID NO: 66 - protein, 30 aa, Artificial Sequence
  Recombinant Polypeptide GLEW-class Nanobody FW1 sequence
QVKLEESGGGLAQPGGSLRLSCVASGFTFS

SEQ ID NO: 67 - protein, 30 aa, Artificial Sequence
  Recombinant Polypeptide GLEW-class Nanobody FW1 sequence
EVQLVESGGGLVQPGGSLRLSCVCVSSGCT

SEQ ID NO: 68 - protein, 30 aa, Artificial Sequence
  Recombinant Polypeptide GLEW-class Nanobody FW1 sequence
EVQLVESGGGLALPGGSLTLSCVFSGSTFS

SEQ ID NO: 69 - protein, 22 aa, Artificial Sequence
  Recombinant Polypeptide GLEW-class Nanobody FW1 sequence
VESGGGLVQPGGSLRLSCAASG

SEQ ID NO: 70 - protein, 22 aa, Artificial Sequence
  Recombinant Polypeptide GLEW-class Nanobody FW1 sequence
EESGGGLAQPGGSLRLSCVASG

SEQ ID NO: 71 - protein, 22 aa, Artificial Sequence
  Recombinant Polypeptide GLEW-class Nanobody FW1 sequence
VESGGGLALPGGSLTLSCVFSG

SEQ ID NO: 72 - protein, 14 aa, Artificial Sequence
  Recombinant Polypeptide GLEW-class Nanobody FW2 sequence
WVRQAPGKVLEWVS

SEQ ID NO: 73 - protein, 14 aa, Artificial Sequence
  Recombinant Polypeptide GLEW-class Nanobody FW2 sequence
WVRRPPGKGLEWVS

SEQ ID NO: 74 - protein, 14 aa, Artificial Sequence
  Recombinant Polypeptide GLEW-class Nanobody FW2 sequence
WVRQAPGMGLEWVS

SEQ ID NO: 75 - protein, 14 aa, Artificial Sequence
  Recombinant Polypeptide GLEW-class Nanobody FW2 sequence
WVRQAPGKEPEWVS

SEQ ID NO: 76 - protein, 14 aa, Artificial Sequence
  Recombinant Polypeptide GLEW-class Nanobody FW2 sequence
WVRQAPGKDQEWVS

SEQ ID NO: 77 - protein, 14 aa, Artificial Sequence
  Recombinant Polypeptide GLEW-class Nanobody FW2 sequence
WVRQAPGKAEEWVS

SEQ ID NO: 78 - protein, 14 aa, Artificial Sequence
  Recombinant Polypeptide GLEW-class Nanobody FW2 sequence
WVRQAPGKGLEWVA

SEQ ID NO: 79 - protein, 14 aa, Artificial Sequence
  Recombinant Polypeptide GLEW-class Nanobody FW2 sequence
WVRQAPGRATEWVS

SEQ ID NO: 80 - protein, 32 aa, Artificial Sequence
  Recombinant Polypeptide GLEW-class Nanobody FW3 sequence
RFTISRDNAKNTLYLQMNSLKPEDTAVYYCVK

SEQ ID NO: 81 - protein, 32 aa, Artificial Sequence
  Recombinant Polypeptide GLEW-class Nanobody FW3 sequence
RFTISRDNARNTLYLQMDSLIPEDTALYYCAR

SEQ ID NO: 82 - protein, 32 aa, Artificial Sequence
  Recombinant Polypeptide GLEW-class Nanobody FW3 sequence
RFTSSRDNAKSTLYLQMNDLKPEDTALYYCAR

SEQ ID NO: 83 - protein, 32 aa, Artificial Sequence
  Recombinant Polypeptide GLEW-class Nanobody FW3 sequence
RFIISRDNAKNTLYLQMNSLGPEDTAMYYCQR

SEQ ID NO: 84 - protein, 32 aa, Artificial Sequence
  Recombinant Polypeptide GLEW-class Nanobody FW3 sequence
RFTASRDNAKNTLYLQMNSLKSEDTARYYCAR

SEQ ID NO: 85 - protein, 32 aa, Artificial Sequence
  Recombinant Polypeptide GLEW-class Nanobody FW3 sequence
RFTISRDNAKNTLYLQMDDLQSEDTAMYYCGR

SEQ ID NO: 86 - protein, 11 aa, Artificial Sequence
  Recombinant Polypeptide GLEW-class Nanobody FW4 sequence
GSQGTQVTVSS

SEQ ID NO: 87 - protein, 11 aa, Artificial Sequence
  Recombinant Polypeptide GLEW-class Nanobody FW4 sequence
LRGGTQVTVSS

SEQ ID NO: 88 - protein, 11 aa, Artificial Sequence
  Recombinant Polypeptide GLEW-class Nanobody FW4 sequence
RGQGTLVTVSS

SEQ ID NO: 89 - protein, 11 aa, Artificial Sequence
  Recombinant Polypeptide GLEW-class Nanobody FW4 sequence
RSRGIQVTVSS

SEQ ID NO: 90 - protein, 11 aa, Artificial Sequence
  Recombinant Polypeptide GLEW-class Nanobody FW4 sequence
WGKGTQVTVSS

SEQ ID NO: 91 - protein, 11 aa, Artificial Sequence
  Recombinant Polypeptide GLEW-class Nanobody FW4 sequence
WGQGTQVTVSS

SEQ ID NO: 92 - protein, 30 aa, Artificial Sequence
  Recombinant Polypeptide P,R,S 103-class Nanobody FW1 sequence
AVQLVESGGGLVQAGGSLRLSCAASGRTFS

SEQ ID NO: 93 - protein, 30 aa, Artificial Sequence
  Recombinant Polypeptide P,R,S 103-class Nanobody FW1 sequence
QVQLQESGGGMVQPGGSLRLSCAASGFDFG

SEQ ID NO: 94 - protein, 30 aa, Artificial Sequence; identical to SEQ ID NO: 65
  Recombinant Polypeptide P,R,S 103-class Nanobody FW1 sequence

SEQ ID NO: 95 - protein, 30 aa, Artificial Sequence
  Recombinant Polypeptide P,R,S 103-class Nanobody FW1 sequence
QVQLAESGGGLVQPGGSLKLSCAASRTIVS

SEQ ID NO: 96 - protein, 30 aa, Artificial Sequence
  Recombinant Polypeptide P,R,S 103-class Nanobody FW1 sequence
QEHLVESGGGLVDIGGSLRLSCAASERIFS

SEQ ID NO: 97 - protein, 30 aa, Artificial Sequence; identical to SEQ ID NO: 66
  Recombinant Polypeptide P,R,S 103-class Nanobody FW1 sequence

SEQ ID NO: 98 - protein, 30 aa, Artificial Sequence; identical to SEQ ID NO: 67
  Recombinant Polypeptide P,R,S 103-class Nanobody FW1 sequence

SEQ ID NO: 99 - protein, 30 aa, Artificial Sequence; identical to SEQ ID NO: 68
  Recombinant Polypeptide P,R,S 103-class Nanobody FW1 sequence

SEQ ID NO: 100 - protein, 22 aa, Artificial Sequence
  Recombinant Polypeptide P,R,S 103-class Nanobody FW1 sequence
VESGGGLVQAGGSLRLSCAASG

SEQ ID NO: 101 - protein, 22 aa, Artificial Sequence
  Recombinant Polypeptide P,R,S 103-class Nanobody FW1 sequence
AESGGGLVQPGGSLKLSCAASR

SEQ ID NO: 102 - protein, 14 aa, Artificial Sequence
  Recombinant Polypeptide P,R,S 103-class Nanobody FW2 sequence
WFRQAPGKEREFVA

SEQ ID NO: 103 - protein, 14 aa, Artificial Sequence
  Recombinant Polypeptide P,R,S 103-class Nanobody FW2 sequence
WVRQAPGKVLEWVS

SEQ ID NO: 104 - protein, 14 aa, Artificial Sequence
  Recombinant Polypeptide P,R,S 103-class Nanobody FW2 sequence
WVRRPPGKGLEWVS

SEQ ID NO: 105 - protein, 14 aa, Artificial Sequence
  Recombinant Polypeptide P,R,S 103-class Nanobody FW2 sequence
WIRQAPGKEREGVS

SEQ ID NO: 106 - protein, 14 aa, Artificial Sequence
  Recombinant Polypeptide P,R,S 103-class Nanobody FW2 sequence
WVRQYPGKEPEWVS

SEQ ID NO: 107 - protein, 14 aa, Artificial Sequence
  Recombinant Polypeptide P,R,S 103-class Nanobody FW2 sequence
WFRQPPGKEHEFVA

SEQ ID NO: 108 - protein, 14 aa, Artificial Sequence
  Recombinant Polypeptide P,R,S 103-class Nanobody FW2 sequence
WYRQAPGKRTELVA

SEQ ID NO: 109 - protein, 14 aa, Artificial Sequence
  Recombinant Polypeptide P,R,S 103-class Nanobody FW2 sequence
WLRQAPGQGLEWVS

SEQ ID NO: 110 - protein, 14 aa, Artificial Sequence
  Recombinant Polypeptide P,R,S 103-class Nanobody FW2 sequence
WLRQTPGKGLEWVG

SEQ ID NO: 111 - protein, 14 aa, Artificial Sequence
  Recombinant Polypeptide P,R,S 103-class Nanobody FW2 sequence
WVRQAPGKAEEFVS

SEQ ID NO: 112 - protein, 32 aa, Artificial Sequence
  Recombinant Polypeptide P,R,S 103-class Nanobody FW3 sequence
RFTISRDNAKNTVYLQMNSLKPEDTAVYYCAA

SEQ ID NO: 113 - protein, 32 aa, Artificial Sequence; identical to SEQ ID NO: 81
  Recombinant Polypeptide P,R,S 103-class Nanobody FW3 sequence

SEQ ID NO: 114 - protein, 32 aa, Artificial Sequence
  Recombinant Polypeptide P,R,S 103-class Nanobody FW3 sequence
RFTISRDNAKNEMYLQMNNLKTEDTGVYWCGA

SEQ ID NO: 115 - protein, 32 aa, Artificial Sequence
  Recombinant Polypeptide P,R,S 103-class Nanobody FW3 sequence
RFTISSDSNRNMIYLQMNNLKPEDTAVYYCAA

SEQ ID NO: 116 - protein, 32 aa, Artificial Sequence
  Recombinant Polypeptide P,R,S 103-class Nanobody FW3 sequence
RFTISRDNAKNMLYLHLNNLKSEDTAVYYCRR

SEQ ID NO: 117 - protein, 32 aa, Artificial Sequence
  Recombinant Polypeptide P,R,S 103-class Nanobody FW3 sequence
RFTISRDNAKKTVYLRLNSLNPEDTAVYSCNL

SEQ ID NO: 118 - protein, 32 aa, Artificial Sequence
  Recombinant Polypeptide P,R,S 103-class Nanobody FW3 sequence
RFKISRDNAKKTLYLQMNSLGPEDTAMYYCQR

SEQ ID NO: 119 - protein, 32 aa, Artificial Sequence
  Recombinant Polypeptide P,R,S 103-class Nanobody FW3 sequence
RFTVSRDNGKNTAYLRMNSLKPEDTADYYCAV

SEQ ID NO: 120 - protein, 11 aa, Artificial Sequence
  Recombinant Polypeptide P,R,S 103-class Nanobody FW4 sequence
RGQGTQVTVSS

SEQ ID NO: 121 - protein, 11 aa, Artificial Sequence
  Recombinant Polypeptide P,R,S 103-class Nanobody FW4 sequence
LRGGTQVTVSS

SEQ ID NO: 122 - protein, 11 aa, Artificial Sequence
  Recombinant Polypeptide P,R,S 103-class Nanobody FW4 sequence
GNKGTLVTVSS

SEQ ID NO: 123 - protein, 11 aa, Artificial Sequence
  Recombinant Polypeptide P,R,S 103-class Nanobody FW4 sequence
SSPGTQVTVSS

SEQ ID NO: 124 - protein, 11 aa, Artificial Sequence
  Recombinant Polypeptide P,R,S 103-class Nanobody FW4 sequence
SSQGTLVTVSS

SEQ ID NO: 125 - protein, 11 aa, Artificial Sequence
  Recombinant Polypeptide P,R,S 103-class Nanobody FW4 sequence
RSRGIQVTVSS

SEQ ID NO: 126 - protein, 26 aa, Artificial Sequence
  Recombinant Polypeptide Framework region 1 (FR1)
QVQLQESGGGXVQAGGSLRLSCAASG

SEQ ID NO: 127 - protein, 14 aa, Artificial Sequence
  Recombinant Polypeptide Framework region 2 (FR2)
WXRQAPGKXXEXVA

SEQ ID NO: 128 - protein, 32 aa, Artificial Sequence
  Recombinant Polypeptide Framework region 3 (FR3)
RFTISRDNAKNTVYLQMNSLXXEDTAVYYCAA

SEQ ID NO: 129 - protein, 11 aa, Artificial Sequence
  Recombinant Polypeptide Framework region 4 (FR4)
XXQGTXVTVSS

SEQ ID NO: 130 - protein, 26 aa, Artificial Sequence
  Recombinant Polypeptide Framework region 1 (FR1)
QVQLQESGGGLVQAGGSLRLSCAASG

SEQ ID NO: 131 - protein, 14 aa, Artificial Sequence
  Recombinant Polypeptide Framework region 2 (FR2)
WFRQAPGKERELVA

SEQ ID NO: 132 - protein, 14 aa, Artificial Sequence
  Recombinant Polypeptide Framework region 2 (FR2)
WFRQAPGKEREFVA

SEQ ID NO: 133 - protein, 13 aa, Artificial Sequence
  Recombinant Polypeptide Framework region 2 (FR2)
WFRQAPGKEREGA

SEQ ID NO: 134 - protein, 14 aa, Artificial Sequence
  Recombinant Polypeptide Framework region 2 (FR2)
WFRQAPGKQRELVA

SEQ ID NO: 135 - protein, 14 aa, Artificial Sequence
  Recombinant Polypeptide Framework region 2 (FR2)
WFRQAPGKQREFVA

SEQ ID NO: 136 - protein, 13 aa, Artificial Sequence
  Recombinant Polypeptide Framework region 2 (FR2)
WYRQAPGKGLEWA

SEQ ID NO: 137 - protein, 32 aa, Artificial Sequence; identical to SEQ ID NO: 112
  Recombinant Polypeptide Framework region 3 (FR3)

SEQ ID NO: 138 - protein, 11 aa, Artificial Sequence
  Recombinant Polypeptide Framework region 4 (FR4)
WGQGTQVTVSS

SEQ ID NO: 139 - protein, 11 aa, Artificial Sequence
  Recombinant Polypeptide Framework region 4 (FR4)
WGQGTLVTVSS

SEQ ID NO: 140 - protein, 5 aa, Artificial Sequence
  Recombinant Polypeptide Complementarity determining region 1 (CDR1)
SFGMS

SEQ ID NO: 141 - protein, 5 aa, Artificial Sequence
  Recombinant Polypeptide Complementarity determining region 1 (CDR1)
LNLMG

SEQ ID NO: 142 - protein, 5 aa, Artificial Sequence
  Recombinant Polypeptide Complementarity determining region 1 (CDR1)
INLLG

SEQ ID NO: 143 - protein, 5 aa, Artificial Sequence
  Recombinant Polypeptide Complementarity determining region 1 (CDR1)
NYWMY

SEQ ID NO: 144 - protein, 17 aa, Artificial Sequence
  Recombinant Polypeptide Complementarity determining region 2 (CDR2)
SISGSGSDTLYADSVKG

SEQ ID NO: 145 - protein, 16 aa, Artificial Sequence
  Recombinant Polypeptide Complementarity determining region 2 (CDR2)
TITVGDSTNYADSVKG

SEQ ID NO: 146 - protein, 16 aa, Artificial Sequence
  Recombinant Polypeptide Complementarity determining region 2 (CDR2)
TITVGDSTSYADSVKG

SEQ ID NO: 147 - protein, 17 aa, Artificial Sequence
  Recombinant Polypeptide Complementarity determining region 2 (CDR2)
SINGRGDDTRYADSVKG

SEQ ID NO: 148 - protein, 17 aa, Artificial Sequence
  Recombinant Polypeptide Complementarity determining region 2 (CDR2)
AISADSSTKNYADSVKG

SEQ ID NO: 149 - protein, 17 aa, Artificial Sequence
  Recombinant Polypeptide Complementarity determining region 2 (CDR2)
AISADSSDKRYADSVKG

SEQ ID NO: 150 - protein, 17 aa, Artificial Sequence
  Recombinant Polypeptide Complementarity determining region 2 (CDR2)
RISTGGGYSYYADSVKG

SEQ ID NO: 151 - protein, 13 aa, Artificial Sequence
  Recombinant Polypeptide Complementarity determining region 3 (CDR3)
DREAQVDTLDFDY

SEQ ID NO: 152 - protein, 6 aa, Artificial Sequence
  Recombinant Polypeptide Complementarity determining region 3 (CDR3)
GGSLSR

SEQ ID NO: 153 - protein, 8 aa, Artificial Sequence
  Recombinant Polypeptide Complementarity determining region 3 (CDR3)
RRTWHSEL

SEQ ID NO: 154 - protein, 7 aa, Artificial Sequence
  Recombinant Polypeptide Complementarity determining region 3 (CDR3)
GRSVSRS

SEQ ID NO: 155 - protein, 5 aa, Artificial Sequence
  Recombinant Polypeptide Complementarity determining region 3 (CDR3)
GRGSP

SEQ ID NO: 156 - protein, 17 aa, Artificial Sequence
  Recombinant Polypeptide myc-tag
AAAEQKLISEEDLNGAA

SEQ ID NO: 157 - protein, 115 aa, Artificial Sequence [sequence omitted]

SEQ ID NO: 158 - protein, 115 aa, Artificial Sequence [sequence omitted]

SEQ ID NO: 159 - protein, 117 aa, Artificial Sequence
  Recombinant Polypeptide Nanobody directed against human serum albumin (PMP 6A8 (ALB-2))
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 160 - protein, 125 aa, Artificial Sequence
  Recombinant Polypeptide Nanobody FC44
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSS

SEQ ID NO: 161 - protein, 122 aa, Artificial Sequence
  Recombinant Polypeptide Nanobody FC5
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 162 - protein, 30 aa, Artificial Sequence
  Recombinant Polypeptide Linker GS30
GGGGSGGGGSGGGGSGGGGSGGGGSGGGGS

SEQ ID NO: 163 - protein, 15 aa, Artificial Sequence
  Recombinant Polypeptide Linker GS15
GGGGSGGGGSGGGGS

SEQ ID NO: 164 - protein, 9 aa, Artificial Sequence
  Recombinant Polypeptide Linker GS9
GGGGSGGGS

SEQ ID NO: 165 - protein, 7 aa, Artificial Sequence
  Recombinant Polypeptide Linker GS7
SGGSGGS

SEQ ID NO: 166 - protein, 12 aa, Artificial Sequence
  Recombinant Polypeptide Llama upper long hinge region
EPKTPKPQPAAA

SEQ ID NO: 167 - protein, 5 aa, Artificial Sequence
  Recombinant Polypeptide Complementarity determining region 1 (CDR1)
PYTMG

SEQ ID NO: 168 - protein, 5 aa, Artificial Sequence
  Recombinant Polypeptide Complementarity determining region 1 (CDR1)
DYAMS

SEQ ID NO: 169 - protein, 5 aa, Artificial Sequence
  Recombinant Polypeptide Complementarity determining region 1 (CDR1)
YYAIG

SEQ ID NO: 170 - protein, 5 aa, Artificial Sequence
  Recombinant Polypeptide Complementarity determining region 1 (CDR1)
INAMG

SEQ ID NO: 171 - protein, 5 aa, Artificial Sequence
  Recombinant Polypeptide Complementarity determining region 1 (CDR1)
IYTMG

SEQ ID NO: 172 - protein, 5 aa, Artificial Sequence
  Recombinant Polypeptide Complementarity determining region 1 (CDR1)
RLAMD

SEQ ID NO: 173 - protein, 5 aa, Artificial Sequence
  Recombinant Polypeptide Complementarity determining region 1 (CDR1)
RLAMD

SEQ ID NO: 174 - protein, 5 aa, Artificial Sequence
  Recombinant Polypeptide Complementarity determining region 1 (CDR1)
FNIMG

SEQ ID NO: 175 - protein, 5 aa, Artificial Sequence
  Recombinant Polypeptide Complementarity determining region 1 (CDR1)
FNIMG

SEQ ID NO: 176 - protein, 5 aa, Artificial Sequence
  Recombinant Polypeptide Complementarity determining region 1 (CDR1)
YYGVG

SEQ ID NO: 177 - protein, 5 aa, Artificial Sequence
  Recombinant Polypeptide Complementarity determining region 1 (CDR1)
YYGVG

SEQ ID NO: 178 - protein, 5 aa, Artificial Sequence
  Recombinant Polypeptide Complementarity determining region 1 (CDR1)
YYGVG

SEQ ID NO: 179 - protein, 5 aa, Artificial Sequence
  Recombinant Polypeptide Complementarity determining region 1 (CDR1)
DSAIG

SEQ ID NO: 180 - protein, 5 aa, Artificial Sequence
  Recombinant Polypeptide Complementarity determining region 1 (CDR1)
PYTIA

SEQ ID NO: 181 - protein, 5 aa, Artificial Sequence
  Recombinant Polypeptide Complementarity determining region 1 (CDR1)
PYTIG

SEQ ID NO: 182 - protein, 5 aa, Artificial Sequence
  Recombinant Polypeptide Complementarity determining region 1 (CDR1)
INVMN

SEQ ID NO: 183 - protein, 5 aa, Artificial Sequence
  Recombinant Polypeptide Complementarity determining region 1 (CDR1)
SYAMG

SEQ ID NO: 184 - protein, 5 aa, Artificial Sequence
  Recombinant Polypeptide Complementarity determining region 1 (CDR1)
PYTMG

SEQ ID NO: 185 - protein, 5 aa, Artificial Sequence
  Recombinant Polypeptide Complementarity determining region 1 (CDR1)
PYTVG

SEQ ID NO: 186 - protein, 5 aa, Artificial Sequence
  Recombinant Polypeptide Complementarity determining region 1 (CDR1)
PYTMG

SEQ ID NO: 187 - protein, 5 aa, Artificial Sequence
  Recombinant Polypeptide Complementarity determining region 1 (CDR1)
PYTMG

SEQ ID NO: 188 - protein, 5 aa, Artificial Sequence
  Recombinant Polypeptide Complementarity determining region 1 (CDR1)
PYTMG

SEQ ID NO: 189 - protein, 5 aa, Artificial Sequence
  Recombinant Polypeptide Complementarity determining region 1 (CDR1)
INPMG

SEQ ID NO: 190 - protein, 5 aa, Artificial Sequence
  Recombinant Polypeptide Complementarity determining region 1 (CDR1)
INPMG

SEQ ID NO: 191 - protein, 5 aa, Artificial Sequence
  Recombinant Polypeptide Complementarity determining region 1 (CDR1)
INPMA

SEQ ID NO: 192 - protein, 5 aa, Artificial Sequence
  Recombinant Polypeptide Complementarity determining region 1 (CDR1)
SYPMG

SEQ ID NO: 193 - protein, 5 aa, Artificial Sequence
  Recombinant Polypeptide Complementarity determining region 1 (CDR1)
SYPMG

SEQ ID NO: 194 - protein, 5 aa, Artificial Sequence
  Recombinant Polypeptide Complementarity determining region 1 (CDR1)
SYPMG

SEQ ID NO: 195 - protein, 5 aa, Artificial Sequence
  Recombinant Polypeptide Complementarity determining region 1 (CDR1)
SYPMG

SEQ ID NO: 196 - protein, 5 aa, Artificial Sequence
  Recombinant Polypeptide Complementarity determining region 1 (CDR1)
SYPMG

SEQ ID NO: 197 - protein, 5 aa, Artificial Sequence
  Recombinant Polypeptide Complementarity determining region 1 (CDR1)
SYPMG

SEQ ID NO: 198 - protein, 5 aa, Artificial Sequence
  Recombinant Polypeptide Complementarity determining region 1 (CDR1)
SFPMG

SEQ ID NO: 199 - protein, 5 aa, Artificial Sequence
  Recombinant Polypeptide Complementarity determining region 1 (CDR1)
SFPMG

SEQ ID NO: 200 - protein, 5 aa, Artificial Sequence
  Recombinant Polypeptide Complementarity determining region 1 (CDR1)
SFPMG

SEQ ID NO: 201 - protein, 5 aa, Artificial Sequence
  Recombinant Polypeptide Complementarity determining region 1 (CDR1)
AFPMG

SEQ ID NO: 202 - protein, 5 aa, Artificial Sequence
  Recombinant Polypeptide Complementarity determining region 1 (CDR1)
AFPMG

SEQ ID NO: 203 - protein, 5 aa, Artificial Sequence
  Recombinant Polypeptide Complementarity determining region 1 (CDR1)
AFPMG

SEQ ID NO: 204 - protein, 5 aa, Artificial Sequence
  Recombinant Polypeptide Complementarity determining region 1 (CDR1)
AFPMG

SEQ ID NO: 205 - protein, 5 aa, Artificial Sequence
  Recombinant Polypeptide Complementarity determining region 1 (CDR1)
AFPMG

SEQ ID NO: 206 - protein, 5 aa, Artificial Sequence
  Recombinant Polypeptide Complementarity determining region 1 (CDR1)
TYAMG

SEQ ID NO: 207 - protein, 5 aa, Artificial Sequence
  Recombinant Polypeptide Complementarity determining region 1 (CDR1)
NYHMV

SEQ ID NO: 208 - protein, 5 aa, Artificial Sequence
  Recombinant Polypeptide Complementarity determining region 1 (CDR1)
NYAMA

SEQ ID NO: 209 - protein, 5 aa, Artificial Sequence
  Recombinant Polypeptide Complementarity determining region 1 (CDR1)
IDAMA

SEQ ID NO: 210 - protein, 6 aa, Artificial Sequence
  Recombinant Polypeptide Complementarity determining region 1 (CDR1)
KHHATG

SEQ ID NO: 211 - protein, 5 aa, Artificial Sequence
  Recombinant Polypeptide Complementarity determining region 1 (CDR1)
SYVMG

SEQ ID NO: 212 - protein, 5 aa, Artificial Sequence
  Recombinant Polypeptide Complementarity determining region 1 (CDR1)
SYVMG

SEQ ID NO: 213 - protein, 5 aa, Artificial Sequence
  Recombinant Polypeptide Complementarity determining region 1 (CDR1)
SSPMG

SEQ ID NO: 214 - protein, 5 aa, Artificial Sequence
  Recombinant Polypeptide Complementarity determining region 1 (CDR1)
SSPMG

SEQ ID NO: 215 - protein, 5 aa, Artificial Sequence
  Recombinant Polypeptide Complementarity determining region 1 (CDR1)
SSPMG

SEQ ID NO: 216 - protein, 5 aa, Artificial Sequence
  Recombinant Polypeptide Complementarity determining region 1 (CDR1)
NGPMA

SEQ ID NO: 217 - protein, 5 aa, Artificial Sequence
  Recombinant Polypeptide Complementarity determining region 1 (CDR1)
SYPIA

SEQ ID NO: 218 - protein, 16 aa, Artificial Sequence
  Recombinant Polypeptide Complementarity determining region 2 (CDR2)
RINWSGIRNYADSVKG

SEQ ID NO: 219 - protein, 17 aa, Artificial Sequence
  Recombinant Polypeptide Complementarity determining region 2 (CDR2)
AITGNGASKYYAESMKG

SEQ ID NO: 220 - protein, 17 aa, Artificial Sequence
  Recombinant Polypeptide Complementarity determining region 2 (CDR2)
CISSSVGTTYYSDSVKG

SEQ ID NO: 221 - protein, 16 aa, Artificial Sequence
  Recombinant Polypeptide Complementarity determining region 2 (CDR2)
DIMPYGSTEYADSVKG

SEQ ID NO: 222 - protein, 19 aa, Artificial Sequence
  Recombinant Polypeptide Complementarity determining region 2 (CDR2)
AAHWTVFRGNTYYVDSVKG

SEQ ID NO: 223 - protein, 16 aa, Artificial Sequence
  Recombinant Polypeptide Complementarity determining region 2 (CDR2)
SIAVSGTTMLDDSVKG

SEQ ID NO: 224 - protein, 16 aa, Artificial Sequence
  Recombinant Polypeptide Complementarity determining region 2 (CDR2)
SISRSGTTMAADSVKG

SEQ ID NO: 225 - protein, 16 aa, Artificial Sequence
  Recombinant Polypeptide Complementarity determining region 2 (CDR2)
DITNRGTTNYADSVKG

SEQ ID NO: 226 - protein, 16 aa, Artificial Sequence
  Recombinant Polypeptide Complementarity determining region 2 (CDR2)
DITNGGTTMYADSVKG

SEQ ID NO: 227 - protein, 17 aa, Artificial Sequence
  Recombinant Polypeptide Complementarity determining region 2 (CDR2)
CISSSDGDTYYADSVKG

SEQ ID NO: 228 - protein, 17 aa, Artificial Sequence
  Recombinant Polypeptide Complementarity determining region 2 (CDR2)
CISSSDGDTYYADSVKG

SEQ ID NO: 229 - protein, 17 aa, Artificial Sequence
  Recombinant Polypeptide Complementarity determining region 2 (CDR2)
CTSSSDGDTYYADSVKG

SEQ ID NO: 230 - protein, 17 aa, Artificial Sequence
  Recombinant Polypeptide Complementarity determining region 2 (CDR2)
CISSSDGDTYYDDSVKG

SEQ ID NO: 231 - protein, 23 aa, Artificial Sequence
  Recombinant Polypeptide Complementarity determining region 2 (CDR2)
TIIGSDRSTDLDGDTYYADSVRG

SEQ ID NO: 232 - protein, 23 aa, Artificial Sequence
  Recombinant Polypeptide Complementarity determining region 2 (CDR2)
TIIGSDRSTDLDGDTYYADSVRG

SEQ ID NO: 233 - protein, 16 aa, Artificial Sequence
  Recombinant Polypeptide Complementarity determining region 2 (CDR2)
AITSGGRKNYADSVKG

SEQ ID NO: 234 - protein, 17 aa, Artificial Sequence
  Recombinant Polypeptide Complementarity determining region 2 (CDR2)
AISSNGGSTRYADSVKG

SEQ ID NO: 235 - protein, 16 aa, Artificial Sequence
  Recombinant Polypeptide Complementarity determining region 2 (CDR2)
RINWSGIRNYADSVKG

SEQ ID NO: 236 - protein, 16 aa, Artificial Sequence
  Recombinant Polypeptide Complementarity determining region 2 (CDR2)
RINWSGIRNYADSVKG

SEQ ID NO: 237 - protein, 16 aa, Artificial Sequence
  Recombinant Polypeptide Complementarity determining region 2 (CDR2)
RINWSGIRNYADSVKG

SEQ ID NO: 238 - protein, 16 aa, Artificial Sequence
  Recombinant Polypeptide Complementarity determining region 2 (CDR2)
RINWSGITNYADSVKG

SEQ ID NO: 239 - protein, 16 aa, Artificial Sequence
  Recombinant Polypeptide Complementarity determining region 2 (CDR2)
RINWSGITNYADSVKG

SEQ ID NO: 240 - protein, 15 aa, Artificial Sequence
  Recombinant Polypeptide Complementarity determining region 2 (CDR2)
RIHGSITNYADSVKG

SEQ ID NO: 241 - protein, 15 aa, Artificial Sequence
  Recombinant Polypeptide Complementarity determining region 2 (CDR2)
RIHGSITNYADSVKG

SEQ ID NO: 242 - protein, 16 aa, Artificial Sequence
  Recombinant Polypeptide Complementarity determining region 2 (CDR2)
RIFGGGSTNYADSVKG

SEQ ID NO: 243 - protein, 17 aa, Artificial Sequence
  Recombinant Polypeptide Complementarity determining region 2 (CDR2)
GISQSGVGTAYSDSVKG

SEQ ID NO: 244 - protein, 17 aa, Artificial Sequence
  Recombinant Polypeptide Complementarity determining region 2 (CDR2)
GISQSGGSTAYSDSVKG

SEQ ID NO: 245 - protein, 17 aa, Artificial Sequence
  Recombinant Polypeptide Complementarity determining region 2 (CDR2)
GISQSSSSTAYSDSVKG

SEQ ID NO: 246 - protein, 17 aa, Artificial Sequence
  Recombinant Polypeptide Complementarity determining region 2 (CDR2)
GISQSGGSTAYSDSVKG

SEQ ID NO: 247 - protein, 17 aa, Artificial Sequence
  Recombinant Polypeptide Complementarity determining region 2 (CDR2)
GISQSGGSTAYSDSVKG

SEQ ID NO: 248 - protein, 17 aa, Artificial Sequence
  Recombinant Polypeptide Complementarity determining region 2 (CDR2)
GISQSGGSTAYSDSVKG

SEQ ID NO: 249 - protein, 17 aa, Artificial Sequence
  Recombinant Polypeptide Complementarity determining region 2 (CDR2)
GISQSGGSTHYSDSVKG

SEQ ID NO: 250 - protein, 17 aa, Artificial Sequence
  Recombinant Polypeptide Complementarity determining region 2 (CDR2)
GISQSGGSTHYSDSVKG

SEQ ID NO: 251 - protein, 17 aa, Artificial Sequence
  Recombinant Polypeptide Complementarity determining region 2 (CDR2)
GISQSGGSTHYSDSVKG

SEQ ID NO: 252 - protein, 17 aa, Artificial Sequence
  Recombinant Polypeptide Complementarity determining region 2 (CDR2)
GISQSGGSTHYSDSVKG

SEQ ID NO: 253 - protein, 17 aa, Artificial Sequence
  Recombinant Polypeptide Complementarity determining region 2 (CDR2)
GISQSGGSTHYSDSVKG

SEQ ID NO: 254 - protein, 17 aa, Artificial Sequence
  Recombinant Polypeptide Complementarity determining region 2 (CDR2)
GISQSGGSTHYSDSVKG

SEQ ID NO: 255 - protein, 17 aa, Artificial Sequence
  Recombinant Polypeptide Complementarity determining region 2 (CDR2)
GISQSGGSTHYSDSVKG

SEQ ID NO: 256 - protein, 17 aa, Artificial Sequence
  Recombinant Polypeptide Complementarity determining region 2 (CDR2)
GISQSGGSTHYSDSVKG

SEQ ID NO: 257 - protein, 17 aa, Artificial Sequence
  Recombinant Polypeptide Complementarity determining region 2 (CDR2)
AISWSGANTYYADSVKG

SEQ ID NO: 258 - protein, 17 aa, Artificial Sequence
  Recombinant Polypeptide Complementarity determining region 2 (CDR2)
AASGSTSSTYYADSVKG

SEQ ID NO: 259 - protein, 17 aa, Artificial Sequence
  Recombinant Polypeptide Complementarity determining region 2 (CDR2)
VISYAGGRTYYADSVKG

SEQ ID NO: 260 - protein, 17 aa, Artificial Sequence
  Recombinant Polypeptide Complementarity determining region 2 (CDR2)
TMNWSTGATYYADSVKG

SEQ ID NO: 261 - protein, 17 aa, Artificial Sequence
  Recombinant Polypeptide Complementarity determining region 2 (CDR2)
ALNWSGGNTYYTDSVKG

SEQ ID NO: 262 - protein, 17 aa, Artificial Sequence
  Recombinant Polypeptide Complementarity determining region 2 (CDR2)
TINWSGSNGYYADSVKG

SEQ ID NO: 263 - protein, 17 aa, Artificial Sequence
  Recombinant Polypeptide Complementarity determining region 2 (CDR2)
TINWSGSNKYYADSVKG

SEQ ID NO: 264 - protein, 17 aa, Artificial Sequence
  Recombinant Polypeptide Complementarity determining region 2 (CDR2)
AISGRSGNTYYADSVKG

SEQ ID NO: 265 - protein, 17 aa, Artificial Sequence
  Recombinant Polypeptide Complementarity determining region 2 (CDR2)
AISGRSGNTYYADSVKG

SEQ ID NO: 266 - protein, 17 aa, Artificial Sequence
  Recombinant Polypeptide Complementarity determining region 2 (CDR2)
AISGRSGNTYYADSVKG

SEQ ID NO: 267 - protein, 17 aa, Artificial Sequence
  Recombinant Polypeptide Complementarity determining region 2 (CDR2)
AISWRTGTTYYADSVKG

SEQ ID NO: 268 - protein, 17 aa, Artificial Sequence
  Recombinant Polypeptide Complementarity determining region 2 (CDR2)
AISWRGGNTYYADSVKG

SEQ ID NO: 269 - protein, 10 aa, Artificial Sequence
  Recombinant Polypeptide Complementarity determining region 3 (CDR3)
ASQSGSGYDS

SEQ ID NO: 270 - protein, 17 aa, Artificial Sequence
  Recombinant Polypeptide Complementarity determining region 3 (CDR3)
VAKDTGSFYYPAYEHDV

SEQ ID NO: 271 - protein, 19 aa, Artificial Sequence
  Recombinant Polypeptide Complementarity determining region 3 (CDR3)
SSWFDCGVQGRDLGNEYDY

SEQ ID NO: 272 - protein, 8 aa, Artificial Sequence
  Recombinant Polypeptide Complementarity determining region 3 (CDR3)
YDPRGDDY

SEQ ID NO: 273 - protein, 14 aa, Artificial Sequence
  Recombinant Polypeptide Complementarity determining region 3 (CDR3)
TRSTAWNSPQRYDY

SEQ ID NO: 274 - protein, 9 aa, Artificial Sequence
  Recombinant Polypeptide Complementarity determining region 3 (CDR3)
FDGYTGSDY

SEQ ID NO: 275 - protein, 9 aa, Artificial Sequence
  Recombinant Polypeptide Complementarity determining region 3 (CDR3)
FDGYSGSDY

SEQ ID NO: 276 - protein, 9 aa, Artificial Sequence
  Recombinant Polypeptide Complementarity determining region 3 (CDR3)
YYPTTGFDD

SEQ ID NO: 277 - protein, 9 aa, Artificial Sequence
  Recombinant Polypeptide Complementarity determining region 3 (CDR3)
YYPTTGFDD

SEQ ID NO: 278 - protein, 17 aa, Artificial Sequence
  Recombinant Polypeptide Complementarity determining region 3 (CDR3)
DLSDYGVCSRWPSPYDY

SEQ ID NO: 279 - protein, 17 aa, Artificial Sequence
  Recombinant Polypeptide Complementarity determining region 3 (CDR3)
DLSDYGVCSRWPSPYDY

SEQ ID NO: 280 - protein, 17 aa, Artificial Sequence
  Recombinant Polypeptide Complementarity determining region 3 (CDR3)
DLSDYGVCSRWPSPYDY

SEQ ID NO: 281 - protein, 17 aa, Artificial Sequence
  Recombinant Polypeptide Complementarity determining region 3 (CDR3)
DLSDYGVCSKWPSPYDY

SEQ ID NO: 282 - protein, 14 aa, Artificial Sequence
  Recombinant Polypeptide Complementarity determining region 3 (CDR3)
TGKGYVFTPNEYDY

SEQ ID NO: 283 - protein, 14 aa, Artificial Sequence
  Recombinant Polypeptide Complementarity determining region 3 (CDR3)
TAKGYVFTDNEYDY

SEQ ID NO: 284 - protein, 15 aa, Artificial Sequence
  Recombinant Polypeptide Complementarity determining region 3 (CDR3)
DAPLASDDDVAPADY

SEQ ID NO: 285 - protein, 14 aa, Artificial Sequence
  Recombinant Polypeptide Complementarity determining region 3 (CDR3)
DETTGWVQLADFRS

SEQ ID NO: 286 - protein, 10 aa, Artificial Sequence
  Recombinant Polypeptide Complementarity determining region 3 (CDR3)
ASQSGSGYDS

SEQ ID NO: 287 - protein, 10 aa, Artificial Sequence
  Recombinant Polypeptide Complementarity determining region 3 (CDR3)
ASQSGSGYDS

SEQ ID NO: 288 - protein, 10 aa, Artificial Sequence
  Recombinant Polypeptide Complementarity determining region 3 (CDR3)
ASRSGSGYDS

SEQ ID NO: 289 - protein, 10 aa, Artificial Sequence
  Recombinant Polypeptide Complementarity determining region 3 (CDR3)
ASRSGSGYDS

SEQ ID NO: 290 - protein, 10 aa, Artificial Sequence
  Recombinant Polypeptide Complementarity determining region 3 (CDR3)
ASQVGSGYDS

SEQ ID NO: 291 - protein, 7 aa, Artificial Sequence
  Recombinant Polypeptide Complementarity determining region 3 (CDR3)
RRWGYDY

SEQ ID NO: 292 - protein, 7 aa, Artificial Sequence
  Recombinant Polypeptide Complementarity determining region 3 (CDR3)
RRWGYDY

SEQ ID NO: 293 - protein, 7 aa, Artificial Sequence
  Recombinant Polypeptide Complementarity determining region 3 (CDR3)
RRWGYDY

SEQ ID NO: 294 - protein, 20 aa, Artificial Sequence
  Recombinant Polypeptide Complementarity determining region 3 (CDR3)
RDKTLALRDYAYTTDVGYDD

SEQ ID NO: 295 - protein, 20 aa, Artificial Sequence
  Recombinant Polypeptide Complementarity determining region 3 (CDR3)
RDKTLALRDYAYTTDVGYDD

SEQ ID NO: 296 - protein, 20 aa, Artificial Sequence
  Recombinant Polypeptide Complementarity determining region 3 (CDR3)
RGRTLALRDYAYTTEVGYDD

SEQ ID NO: 297 - protein, 20 aa, Artificial Sequence
  Recombinant Polypeptide Complementarity determining region 3 (CDR3)
RGRTLFLRDYAYTTEVGYDD

SEQ ID NO: 298 - protein, 20 aa, Artificial Sequence
  Recombinant Polypeptide Complementarity determining region 3 (CDR3)
RGRTLFLRGYAYTTEVGYDD

SEQ ID NO: 299 - protein, 20 aa, Artificial Sequence
  Recombinant Polypeptide Complementarity determining region 3 (CDR3)
RGRTIALRNYAYTTEVGYDD

SEQ ID NO: 300 - protein, 20 aa, Artificial Sequence
  Recombinant Polypeptide Complementarity determining region 3 (CDR3)
RGRTLALRNYAYTTEVGYDD

SEQ ID NO: 301 - protein, 20 aa, Artificial Sequence
  Recombinant Polypeptide Complementarity determining region 3 (CDR3)
RGRTLALRNYAYTTEVGYDD

SEQ ID NO: 302 - protein, 20 aa, Artificial Sequence
  Recombinant Polypeptide Complementarity determining region 3 (CDR3)
RGRTLALRNYAYTTEVGYDD

SEQ ID NO: 303 - protein, 20 aa, Artificial Sequence
  Recombinant Polypeptide Complementarity determining region 3 (CDR3)
RGRTLALRNYAYTTEVGYDD

SEQ ID NO: 304 - protein, 20 aa, Artificial Sequence
  Recombinant Polypeptide Complementarity determining region 3 (CDR3)
RGRTLALRNYAYTTEVGYDD

SEQ ID NO: 305 - protein, 20 aa, Artificial Sequence
  Recombinant Polypeptide Complementarity determining region 3 (CDR3)
RGRTLALRNYAYTTEVGYDD

SEQ ID NO: 306 - protein, 20 aa, Artificial Sequence
  Recombinant Polypeptide Complementarity determining region 3 (CDR3)
RGRTLALRNYAYTTEVGYDD

SEQ ID NO: 307 - protein, 20 aa, Artificial Sequence
  Recombinant Polypeptide Complementarity determining region 3 (CDR3)
RGGTLALRNYAYTTEVGYDD

SEQ ID NO: 308 - protein, 21 aa, Artificial Sequence
  Recombinant Polypeptide Complementarity determining region 3 (CDR3)
SAIIEGFQDSIVIFSEAGYDY

SEQ ID NO: 309 - protein, 15 aa, Artificial Sequence
  Recombinant Polypeptide Complementarity determining region 3 (CDR3)
VAGLLLPRVAEGMDY

SEQ ID NO: 310 - protein, 15 aa, Artificial Sequence
  Recombinant Polypeptide Complementarity determining region 3 (CDR3)
VDSPLIATHPRGYDY

SEQ ID NO: 311 - protein, 15 aa, Artificial Sequence
  Recombinant Polypeptide Complementarity determining region 3 (CDR3)
ARGLLIATDARGYDY

SEQ ID NO: 312 - protein, 15 aa, Artificial Sequence
  Recombinant Polypeptide Complementarity determining region 3 (CDR3)
GSYVFYFTVRDQYDY

SEQ ID NO: 313 - protein, 16 aa, Artificial Sequence
  Recombinant Polypeptide Complementarity determining region 3 (CDR3)
SAGGFLVPRVGQGYDY

SEQ ID NO: 314 - protein, 16 aa, Artificial Sequence
  Recombinant Polypeptide Complementarity determining region 3 (CDR3)
SAGGFLVPRVGQGYDY

SEQ ID NO: 315 - protein, 16 aa, Artificial Sequence
  Recombinant Polypeptide Complementarity determining region 3 (CDR3)
ERVGLLLTVVAEGYDY

SEQ ID NO: 316 - protein, 16 aa, Artificial Sequence
  Recombinant Polypeptide Complementarity determining region 3 (CDR3)
ERVGLLLTVVAEGYDY

SEQ ID NO: 317 - protein, 16 aa, Artificial Sequence
  Recombinant Polypeptide Complementarity determining region 3 (CDR3)
ERVGLLLTVVAEGYDY

SEQ ID NO: 318 - protein, 16 aa, Artificial Sequence
  Recombinant Polypeptide Complementarity determining region 3 (CDR3)
ERVGLLLAVVAEGYDY

SEQ ID NO: 319 - protein, 16 aa, Artificial Sequence
  Recombinant Polypeptide Complementarity determining region 3 (CDR3)
ERAGVLLTKVPEGYDY

SEQ ID NO: 320 - protein, 118 aa, Artificial Sequence
  Recombinant Polypeptide Nanobody PMP6D5
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASQSGSGYDSWGQGTQVTVSS

SEQ ID NO: 321 - protein, 126 aa, Artificial Sequence
  Recombinant Polypeptide Nanobody PMP8F2
DVQLVESGGDLVQPGGSLRLSCAASGFSFDDYAMSWLRQTPGKGLEWVGAITGNGASKYYAESMKGRFTISRDNAKNMLYLHLNNLKSEDTAVYYCRRVAKDTGSFYYPAYEHDVLGQGTQVTVSS

SEQ ID NO: 322 - protein, 128 aa, Artificial Sequence
  Recombinant Polypeptide Nanobody PMP6B12
AVQLVESGGGLVQPGGSLRLSCAASGFTLAYYAIGWFRQAPGKEREGVSCISSSVGTTYYSDSVKGRFTISRDNAKNTVYLQMNSLKPEDTAVYYCVRSSWFDCGVQGRDLGNEYDYRGQGTQVTVSS

SEQ ID NO: 323 - protein, 116 aa, Artificial Sequence
  Recombinant Polypeptide Nanobody PMP6B6
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 324 - protein, 125 aa, Artificial Sequence
  Recombinant Polypeptide Nanobody PMP11C1
EVQLVESGGGLVQTGGSLRLSCATSGLAFSIYTMGWFRQAPGKEREFVAAAHWTVFRGNTYYVDSVKGRFTISRDNAKNTVYLQMNSLKPEDSAVYYCAATRSTAWNSPQRYDYWGQGTQVTVSS

SEQ ID NO: 325 - protein, 117 aa, Artificial Sequence
  Recombinant Polypeptide Nanobody PMP23H2
AVQLVDSGGGLVQPGGSLRLSCAASGSIFSRLAMDWYRQAPGKQRELVASIAVSGTTMLDDSVKGRFTISRDNAENTVYLQMNSLKPEDTAVYYCMAFDGYTGSDYWGRGTQVTVSS

SEQ ID NO: 326 - protein, 117 aa, Artificial Sequence
  Recombinant Polypeptide Nanobody PMP7G4
AVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSS

SEQ ID NO: 327 - protein, 117 aa, Artificial Sequence
  Recombinant Polypeptide Nanobody PMP20D2
AVQLVESGGGLVQPGGSLRLSCAASGSISRFNIMGWYRQAPGKQRELVADITNRGTTNYADSVKGRFTISRDNTKNTVYLQMNSLKPDDTAVYYCHTYYPTTGFDDWGQGTQVTVSS

SEQ ID NO: 328 - protein, 117 aa, Artificial Sequence
  Recombinant Polypeptide Nanobody PMP7G5
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 329 - protein, 126 aa, Artificial Sequence
  Recombinant Polypeptide Nanobody PMP7H3
DVQLVESGGGLVQPGGSLRLSCAASGFTLDYYGVGWFRQAPGKEREGVSCISSSDGDTYYADSVKGRFTISRDNAKNTVYLQMNSLKPEDTAVYYCATDLSDYGVCSRWPSPYDYWGQGTQVTVSS

SEQ ID NO: 330 - protein, 126 aa, Artificial Sequence
  Recombinant Polypeptide Nanobody PMP7G9
QVQLVESGGGLVQPGGSLRLSCAASGFSLDYYGVGWFRQAPGKEREGVSCISSSDGDTYYADSVKGRFTISRDNAKNTVYLQMNSLKPEDTAVYYCATDLSDYGVCSRWPSPYDYWGQGTQVTVSS

SEQ ID NO: 331 - protein, 126 aa, Artificial Sequence
  Recombinant Polypeptide Nanobody PMP9A9
QVQLVESGGGLVQPGGSLRLSCAASGFSLDYYGVGWFRQAPGKEREGVSCTSSSDGDTYYADSVKGRFTISRDNAKNTVYLQMNSLKPEDTAVYYCATDLSDYGVCSRWPSPYDYWGQGTQVTVSS

SEQ ID NO: 332 - protein, 126 aa, Artificial Sequence
  Recombinant Polypeptide Nanobody PMP22E3
QVQLVESGGGLVQPGGSLRLSCAASGFTLDDSAIGWFRQAPGKEREGVSCISSSDGDTYYDDSVKGRFTISRDNVKNMVYLQMNSLKPEDTAVYFCAIDLSDYGVCSKWPSPYDYWGQGTQVTVSS

SEQ ID NO: 333 - protein, 129 aa, Artificial Sequence
  Recombinant Polypeptide Nanobody PMP6E10
QVKLEESGGGLVQAGGSLRLSCVVSGRTFSPYTIAWFRQAPGKEREFVTTIIGSDRSTDLDGDTYYADSVRGRFTISRNDAKNTVFLQMSSLKPEDTAVYYCALTGKGYVFTPNEYDYWGQGTQVTVSS

SEQ ID NO: 334 - protein, 129 aa, Artificial Sequence
  Recombinant Polypeptide Nanobody PMP6G10
QVQLVESGGGLAQAGGSLRLSCVVSGRTFSPYTIGWFSQRPGKEREWVATIIGSDRSTDLDGDTYYADSVRGRFTISRNDAKNTVSLQMNSLKPEDSAVYYCALTAKGYVFTDNEYDYWGQGTQVTVSS

SEQ ID NO: 335 - protein, 123 aa, Artificial Sequence
  Recombinant Polypeptide Nanobody NC3
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSS

SEQ ID NO: 336 - protein, 123 aa, Artificial Sequence
  Recombinant Polypeptide Nanobody NC6
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSS

SEQ ID NO: 337 - protein, 118 aa, Artificial Sequence
  Recombinant Polypeptide Nanobody PMP13A1
AVQLVDSGGGLVQAGGSLRLSCAASGRTFSPYTMGWFRQPPGKVREFVGRINWSGIRNYADSVKGRFTISRDNNNNTVYLQMNRLKPEDTAVYYCAAASQSGSGYDSWGQGTQVTVSS

SEQ ID NO: 338 - protein, 118 aa, Artificial Sequence
  Recombinant Polypeptide Nanobody PMP20G9
QVQLVESGGGLVQAGGSLRLSCAASGRTFSPYTVGWFRQPPGKVREFVGRINWSGIRNYADSVKGRFTISRDNNNNTVYLQMNRLKPEDTAVYYCAAASQSGSGYDSWGQGTQVTVSS

SEQ ID NO: 339 - protein, 118 aa, Artificial Sequence
  Recombinant Polypeptide Nanobody PMP20F4
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSS

SEQ ID NO: 340 - protein, 118 aa, Artificial Sequence
  Recombinant Polypeptide Nanobody PMP21A7
AVQLVESGGGLVQAGSSLRLSCAASGRTFSPYTMGWFRQPPGKVREFVGRINWSGITNYADSVKGRFTISRDNNKNTVYLQMNRLKPEDTAVYYCAAASRSGSGYDSWGQGTQVTVSS

SEQ ID NO: 341 - protein, 118 aa, Artificial Sequence
  Recombinant Polypeptide Nanobody PMP13D8
QVKLEESGGGLVQAGSSLRLSCAASGRTSSPYTMGWFRQPPGKVREFVGRINWSGITNYADSVKGRFTISRDNNKNTVYLQMNRLKPEDTAVYYCASASQVGSGYDSWGQGTQVTVSS

SEQ ID NO: 342 - protein, 114 aa, Artificial Sequence
  Recombinant Polypeptide Nanobody PMP21E12
AVQLVESGGGLVQPGGSLRLSCAASGSITSINPMGWYRQAPGKQRELVARIHGSITNYADSVKGRFTISRDIAKNTVYLQMNSLKPEDTAVYYCNARRWGYDYWGQGAQVTVSS

SEQ ID NO: 343 - protein, 114 aa, Artificial Sequence
  Recombinant Polypeptide Nanobody PMP21C12
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSS

SEQ ID NO: 344 - protein, 115 aa, Artificial Sequence
  Recombinant Polypeptide Nanobody PMP21C2
QVQLVESGGGLVQPGGSLRLSCAASEYITSINPMAWYRQAPGKQRDLVARIFGGGSTNYADSVKGRFTISRDIAKNTVSLQMNSLKPEDTAVYYCNARRWGYDYWGQGTQVTVSS

SEQ ID NO: 345 - protein, 129 aa, Artificial Sequence
  Recombinant Polypeptide Nanobody PMP14G4
AVQLVDSGGGLVQAGGSLRLSCAASGRTFSSYPMGWFRQGPGKERKFVAGISQSGVGTAYSDSVKGRFTISRENAKNTVYLQMNSLKPEDTAVYYCAARDKTLALRDYAYTTDVGYDDWGQGTQVTVSS

SEQ ID NO: 346 - protein, 129 aa, Artificial Sequence
  Recombinant Polypeptide Nanobody PMP14E1
QVQLVESGGGLVQAGGSLRLSCAASGRTFSSYPMGWFRQAPGKERKFVAGISQSGGSTAYSDSVKGRFTISRENAKSTVYLQMNSLKPEDTAVYYCAARDKTLALRDYAYTTDVGYDDWGQGTQVTVSS

SEQ ID NO: 347 - protein, 129 aa, Artificial Sequence
  Recombinant Polypeptide Nanobody PMP6E9
EVQLVESGGGLVQAGGSLRLSCAASGRTFSSYPMGWFRQAPGKERKFVAGISQSSSSTAYSDSVKGRFTISRENAKNTVYLQMNSLKPEDTAVYYCAARGRTLALRDYAYTTEVGYDDWGQGTQVTVSS

SEQ ID NO: 348 - protein, 129 aa, Artificial Sequence
  Recombinant Polypeptide Nanobody PMP12H3
AVQLVDSGGGLVQAGGSLRLSCAASGGTFTSYPMGWFRQAPGKERKFVAGISQSGGSTAYSDSVKGRFTISRENAKTTVYLQMNSLKPEDTAVYYCAARGRTLFLRDYAYTTEVGYDDWGQGTQVTVSS

SEQ ID NO: 349 - protein, 129 aa, Artificial Sequence
  Recombinant Polypeptide Nanobody PMP12C5
DVQLVESGGGLVQAGGSLRLSCAASGGTFTSYPMGWFRQAPGKERKFVAGISQSGGSTAYSDSVKGRFTISRENAKTTVYLQMNSLKPEDTAVYYCAARGRTLFLRGYAYTTEVGYDDWGQGTQVTVSS

SEQ ID NO: 350 - protein, 129 aa, Artificial Sequence
  Recombinant Polypeptide Nanobody PMP17G7
QVKLEESGGGLVQAGGSLRLSCAASGGTFSSYPMGWFRQAPGKEREFVTGISQSGGSTAYSDSVKGRFTISRENAKNTVYLQMNSLKPEDTAVYYCAARGRTIALRNYAYTTEVGYDDWGQGTQVTVSS

SEQ ID NO: 351 - protein, 129 aa, Artificial Sequence
  Recombinant Polypeptide Nanobody PMP14G11
QVQLVESGGGLVQAGGSLRLSCAASGGTFSSFPMGWFRQAPGKGREFVAGISQSGGSTHYSDSVKGRFTISRENAKNTVYLQMNSLKPEDTAVYYCAARGRTLALRNYAYTTEVGYDDWGQGTQVTVSS

SEQ ID NO: 352 - protein, 129 aa, Artificial Sequence
  Recombinant Polypeptide Nanobody PMP9F9
AVQLVESGGGLVQAGGSLRLSCAASGGTFSSFPMGWFRQAPGEKREFVAGISQSGGSTHYSDSVKGRFTISRENARNTVYLQMNSLKPEDTAVYYCAARGRTLALRNYAYTTEVGYDDWGQGTQVTVSS

SEQ ID NO: 353 - protein, 129 aa, Artificial Sequence
  Recombinant Polypeptide Nanobody PMP14A8
EVQLVESGGGLVQAGGSLRLSCAASGGTFSSFPMGWFRQAPGKERKFVAGISQSGGSTHYSDSVKGRFTISRENAKNTVYLQMNNLKPEDTAVYYCAARGRTLALRNYAYTTEVGYDDWGQGTQVTVSS

SEQ ID NO: 354 - protein, 129 aa, Artificial Sequence
  Recombinant Polypeptide Nanobody PMP17B5
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSS

SEQ ID NO: 355 - protein, 129 aa, Artificial Sequence
  Recombinant Polypeptide Nanobody PMP6B7
QVQLVESGGGLVQAGGSLRLSCAASGGTFSAFPMGWFRQAPGKERKFVAGISQSGGSTHYSDSVKGRFTISRENAKNTVYLQMNSLKPEDTAVYYCAARGRTLALRNYAYTTEVGYDDWGQGTQVTVSS

SEQ ID NO: 356 - protein, 129 aa, Artificial Sequence
  Recombinant Polypeptide Nanobody PMP14E9
AVQLVESGGGLVQAGGSLRLSCAASGGTFSAFPMGWFRQAPGKEREFVAGISQSGGSTHYSDSVKGRFTISKENAKSTVYLQMNSLKPEDTAVYYCAARGRTLALRNYAYTTEVGYDDWGQGTQVTVSS

SEQ ID NO: 357 - protein, 129 aa, Artificial Sequence
  Recombinant Polypeptide Nanobody PMP17D7
AVQLVDSGGGLVQAGGSLRLSCAASGGTFSAFPMGWFRQAPGKEREFVAGISQSGGSTHYSDSVKGRFTISKENAKNTVYLQMNSLKPEDTAVYYCAARGRTLALRNYAYTTEVGYDDWGQGTQVTVSS

SEQ ID NO: 358 - protein, 129 aa, Artificial Sequence
  Recombinant Polypeptide Nanobody PMP14G1
QVKLEESGGGLVQAGGSLRLSCAASGGTFSAFPMGWFRQAPGKEREFVAGISQSGGSTHYSDSVKGRFTISKENAKNTVYLQMNSLKPEDTAVYYCAARGGTLALRNYAYTTEVGYDDWGQGTQVTVSS

SEQ ID NO: 359 - protein, 130 aa, Artificial Sequence
  Recombinant Polypeptide Nanobody PMP17B11
QVQLVESGGGLVQAGGSLRLSCAASGPTFSTYAMGWFRQAPGKEREFVAAISWSGANTYYADSVKGRFTISRDNAKNTVYLRMNSLKPEDTAAYYCAASAIIEGFQDSIVIFSEAGYDYWGQGTQVTVSS

SEQ ID NO: 360 - protein, 124 aa, Artificial Sequence
  Recombinant Polypeptide Nanobody PMP10C4
AVQLVDSGGGLVQAGGSLRLSCAASGRSFSNYHMVWFRQAPGKEREFVAAASGSTSSTYYADSVKGRFTISRDNAKNTVYLQMNSLKPEDTAVYYCAAVAGLLLPRVAEGMDYWGKGTLVTVSS

SEQ ID NO: 361 - protein, 124 aa, Artificial Sequence
  Recombinant Polypeptide Nanobody PMP17C4
AVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSS

SEQ ID NO: 362 - protein, 124 aa, Artificial Sequence
  Recombinant Polypeptide Nanobody PMP21B4
QVQLVESGGGLVQAGDSLRVACAASGRTFSIDAMAWFRQAPGKEREFVSTMNWSTGATYYADSVKGRFTSSRDNAKSTSYLQMNSLKPEDTAVYYCAAARGLLIATDARGYDYWGQGTQVTVSS

SEQ ID NO: 363 - protein, 125 aa, Artificial Sequence
  Recombinant Polypeptide Nanobody PMP21H1
QVQLVESGGGLVQTGGSLRLSCAASGSTFSKHHATGWFRQAPGKEREFVAALNWSGGNTYYTDSVKGRFTISRDNAQNTVYLQMNSLKPEDTAVYYCAAGSYVFYFTVRDQYDYWGQGTQVTVSS

SEQ ID NO: 364 - protein, 125 aa, Artificial Sequence
  Recombinant Polypeptide Nanobody PMP10A6
QVQLVESGGGLVQAGGSLRLSCASSGRTFSSYVMGWFRQTPGKEREFVSTINWSGSNGYYADSVKGRFTISRDNAKNTVYLQMNNLKPEDTAVYYCAASAGGFLVPRVGQGYDYWGQGTQVTVSS

SEQ ID NO: 365 - protein, 125 aa, Artificial Sequence
  Recombinant Polypeptide Nanobody PMP13H6
QVKLEESGGGLVQAGGSLRLSCASSGRTFSSYVMGWFRQTPGKEREFVSTINWSGSNKYYADSVKGRFTISRDNAKNTVYLQMNSLKPEDTAVYYCAASAGGFLVPRVGQGYDYWGQGTQVTVSS

SEQ ID NO: 366 - protein, 125 aa, Artificial Sequence
  Recombinant Polypeptide Nanobody PMP13F12
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSS

SEQ ID NO: 367 - protein, 125 aa, Artificial Sequence
  Recombinant Polypeptide Nanobody PMP21A2
DVQLVESGGGLVQAGGSLRLSCAASGRTFSSSPMGWFRQAPGKEREFVAAISGRSGNTYYADSVKGRFTISRDNAKNTVYLQMNSLKPEDTAVYYCAGERVGLLLTVVAEGYDYWGQGTQVTVSS

SEQ ID NO: 368 - protein, 125 aa, Artificial Sequence
  Recombinant Polypeptide Nanobody PMP21F7
EVQLVESGGGLVQAGGSLRLSCAASGRTFSSSPMGWFRQAPGKEREFVAAISGRSGNTYYADSVKGRFTISRDNAKNTVYLQMNSLKPEDTAVYYCAGERVGLLLTVVAEGYDYWGRGTQVTVSS

SEQ ID NO: 369 - protein, 125 aa, Artificial Sequence
  Recombinant Polypeptide Nanobody PMP21H3
QVQLVESGGGLVQAGGSLRLSCAASGRTFSNGPMAWFRQAPGKEREFVSAISWRTGTTYYADSVKGRFTISRDNAKNTVYLQMNSLKPEDTAVYYCAAERVGLLLAVVAEGYDYWGQGTQVTVSS

SEQ ID NO: 370 - protein, 125 aa, Artificial Sequence
  Recombinant Polypeptide Nanobody PMP21E7
AVQLVESGGGLVQAGGSLRLSSVVSGGTFSSYPIAWFRQPPGKEREFVAAISWRGGNTYYADSVKGRFTISRDNAKNTVYLQMNSLKPEDTAVYYSAAERAGVLLTKVPEGYDYWGQGTQVTVSS

SEQ ID NO: 371 - protein, 264 aa, Artificial Sequence
  Recombinant Polypeptide Multivalent Nanobody Construct NC3-25GS-6B6
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGGSGGGGSGGGGSGGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 372 - protein, 264 aa, Artificial Sequence
  Recombinant Polypeptide Multivalent Nanobody Construct NC6-25GS-6B6
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGGSGGGGSGGGGSGGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 373 - protein, 259 aa, Artificial Sequence
  Recombinant Polypeptide Multivalent Nanobody Construct 20F4-25GS-6B6
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGGSGGGGSGGGGSGGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 374 - protein, 255 aa, Artificial Sequence
  Recombinant Polypeptide Multivalent Nanobody Construct 21C12-25GS-6B6
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGGSGGGGSGGGGSGGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 375 - protein, 270 aa, Artificial Sequence
  Recombinant Polypeptide Multivalent Nanobody Construct 17B5-25GS-6B6
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGGSGGGGSGGGGSGGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 376 - protein, 265 aa, Artificial Sequence
  Recombinant Polypeptide Multivalent Nanobody Construct NC3-25GS-7G5
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGGSGGGGSGGGGSGGGGSQVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 377 - protein, 265 aa, Artificial Sequence
  Recombinant Polypeptide Multivalent Nanobody Construct NC6-25GS-7G5
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGGSGGGGSGGGGSGGGGSQVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 378 - protein, 260 aa, Artificial Sequence
  Recombinant Polypeptide Multivalent Nanobody Construct 20F4-25GS-7G5
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGGSGGGGSGGGGSGGGGSQVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 379 - protein, 256 aa, Artificial Sequence
  Recombinant Polypeptide Multivalent Nanobody Construct 21C12-25GS-7G5
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGGSGGGGSGGGGSGGGGSQVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 380 - protein, 271 aa, Artificial Sequence
  Recombinant Polypeptide Multivalent Nanobody Construct 17B5-25GS-7G5
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGGSGGGGSGGGGSGGGGSQVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSS

SEQ ID NO: 381 - protein, 264 aa, Artificial Sequence
  Recombinant Polypeptide Multivalent Nanobody Construct 6B6-25GS-NC3
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGGSGGGGSGGGGSGGGGSEVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSS

SEQ ID NO: 382 - protein, 264 aa, Artificial Sequence
  Recombinant Polypeptide Multivalent Nanobody Construct 6B6-25GS-NC6
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGGSGGGGSGGGGSGGGGSEVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSS

SEQ ID NO: 383 - protein, 259 aa, Artificial Sequence
  Recombinant Polypeptide Multivalent Nanobody Construct 6B6-25GS-20F4
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGGSGGGGSGGGGSGGGGSQVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSS

SEQ ID NO: 384 - protein, 255 aa, Artificial Sequence
  Recombinant Polypeptide Multivalent Nanobody Construct 6B6-25GS-21C12
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGGSGGGGSGGGGSGGGGSAVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSS

SEQ ID NO: 385 - protein, 270 aa, Artificial Sequence
  Recombinant Polypeptide Multivalent Nanobody Construct 6B6-25GS-17B5
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGGSGGGGSGGGGSGGGGSQVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSS

SEQ ID NO: 386 - protein, 265 aa, Artificial Sequence
  Recombinant Polypeptide Multivalent Nanobody Construct 7G5-25GS-NC3
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSSGGGGSGGGGSGGGGSGGGGSGGGGSEVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSS

SEQ ID NO: 387 - protein, 265 aa, Artificial Sequence
  Recombinant Polypeptide Multivalent Nanobody Construct 7G5-25GS-NC6
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSSGGGGSGGGGSGGGGSGGGGSGGGGSEVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSS

SEQ ID NO: 388 - protein, 260 aa, Artificial Sequence
  Recombinant Polypeptide Multivalent Nanobody Construct 7G5-25GS-20F4
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSSGGGGSGGGGSGGGGSGGGGSGGGGSQVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSS

SEQ ID NO: 389 - protein, 256 aa, Artificial Sequence
  Recombinant Polypeptide Multivalent Nanobody Construct 7G5-25GS-21C12
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSSGGGGSGGGGSGGGGSGGGGSGGGGSAVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSS

SEQ ID NO: 390 - protein, 271 aa, Artificial Sequence
  Recombinant Polypeptide Multivalent Nanobody Construct 7G5-25GS-17B5
QVKLEESGGGLVQPGGSLRLSCAASGSISRFNIMGWYRQAPGKQRELVADITNGGTTMYADSVKGRFTISRDNTKNTVYLQMNSLKPEDTAVYYCHTYYPTTGFDDWGQGAQVTVSSGGGGSGGGGSGGGGSGGGGSGGGGSQVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSS

SEQ ID NO: 391 - protein, 252 aa, Artificial Sequence
  Recombinant Polypeptide Multivalent Nanobody Construct 6B12-9GS-ALB8
AVQLVESGGGLVQPGGSLRLSCAASGFTLAYYAIGWFRQAPGKEREGVSCISSSVGTTYYSDSVKGRFTISRDNAKNTVYLQMNSLKPEDTAVYYCVRSSWFDCGVQGRDLGNEYDYRGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 392 - protein, 240 aa, Artificial Sequence
  Recombinant Polypeptide Multivalent Nanobody Construct 6B6-9GS-ALB8
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 393 - protein, 241 aa, Artificial Sequence
  Recombinant Polypeptide Multivalent Nanobody Construct 7G4-9GS-ALB8
AVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 394 - protein, 247 aa, Artificial Sequence
  Recombinant Polypeptide Multivalent Nanobody Construct NC3-9GS-ALB8
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 395 - protein, 247 aa, Artificial Sequence
  Recombinant Polypeptide Multivalent Nanobody Construct NC6-9GS-ALB8
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 396 - protein, 242 aa, Artificial Sequence
  Recombinant Polypeptide Multivalent Nanobody Construct 20F4-9GS-ALB8
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 397 - protein, 238 aa, Artificial Sequence
  Recombinant Polypeptide Multivalent Nanobody Construct 21C12-9GS-ALB8
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 398 - protein, 253 aa, Artificial Sequence
  Recombinant Polypeptide 17B5-9GS-ALB8
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 399 - protein, 248 aa, Artificial Sequence
  Recombinant Polypeptide Multivalent Nanobody Construct 17C4-9GS-ALB8
AVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 400 - protein, 249 aa, Artificial Sequence
  Recombinant Polypeptide Multivalent Nanobody Construct 13F12-9GS-ALB8
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 401 - protein, 374 aa, Artificial Sequence
  Recombinant Polypeptide Multivalent Nanobody Construct 6B6-9GS-ALB8-9GS-13F12
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSS

SEQ ID NO: 402 - protein, 375 aa, Artificial Sequence
  Recombinant Polypeptide Multivalent Nanobody Construct 7G4-9GS-ALB8-9GS-13F12
AVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSS

SEQ ID NO: 403 - protein, 372 aa, Artificial Sequence
  Recombinant Polypeptide Multivalent Nanobody Construct NC3-9GS-ALB8-9GS-6B6
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 404 - protein, 372 aa, Artificial Sequence
  Recombinant Polypeptide Multivalent Nanobody Construct NC6-9GS-ALB8-9GS-6B6
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 405 - protein, 367 aa, Artificial Sequence
  Recombinant Polypeptide Multivalent Nanobody Construct 20F4-9GS-ALB8-9GS-6B6
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 406 - protein, 363 aa, Artificial Sequence
  Recombinant Polypeptide Multivalent Nanobody Construct 21C12-9GS-ALB8-9GS-6B6
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 407 - protein, 378 aa, Artificial Sequence
  Recombinant Polypeptide Multivalent Nanobody Construct 17B5-9GS-ALB8-9GS-6B6
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 408 - protein, 373 aa, Artificial Sequence
  Recombinant Polypeptide Multivalent Nanobody Construct 17C4-9GS-ALB8-9GS-6B6
AVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 409 - protein, 374 aa, Artificial Sequence
  Recombinant Polypeptide Multivalent Nanobody Construct 13F12-9GS-ALB8-9GS-6B6
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 410 - protein, 374 aa, Artificial Sequence; identical to SEQ ID NO: 409
  Recombinant Polypeptide Multivalent Nanobody Construct 13F12-9GS-ALB8-9GS-6B6

SEQ ID NO: 411 - protein, 375 aa, Artificial Sequence
  Recombinant Polypeptide Multivalent Nanobody Construct 13F12-9GS-ALB8-9GS-7G4
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSS

SEQ ID NO: 412 - protein, 372 aa, Artificial Sequence
  Recombinant Polypeptide Multivalent Nanobody Construct 6B6-9GS-ALB8-9GS-NC3
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSS

SEQ ID NO: 413 - protein, 372 aa, Artificial Sequence
  Recombinant Polypeptide Multivalent Nanobody Construct 6B6-9GS-ALB8-9GS-NC6
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSS

SEQ ID NO: 414 - protein, 367 aa, Artificial Sequence
  Recombinant Polypeptide Multivalent Nanobody Construct 6B6-9GS-ALB8-9GS-20F4
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSS

SEQ ID NO: 415 - protein, 363 aa, Artificial Sequence
  Recombinant Polypeptide Multivalent Nanobody Construct 6B6-9GS-ALB8-9GS-21C12
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSS

SEQ ID NO: 416 - protein, 378 aa, Artificial Sequence
  Recombinant Polypeptide Multivalent Nanobody Construct 6B6-9GS-ALB8-9GS-17B5
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSS

SEQ ID NO: 417 - protein, 373 aa, Artificial Sequence
  Recombinant Polypeptide Multivalent Nanobody Construct 6B6-9GS-ALB8-9GS-17C4
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSS

SEQ ID NO: 418 - protein, 374 aa, Artificial Sequence; identical to SEQ ID NO: 401
  Recombinant Polypeptide Multivalent Nanobody Construct 6B6-9GS-ALB8-9GS-13F12

SEQ ID NO: 419 - protein, 252 aa, Artificial Sequence
  Recombinant Polypeptide Multivalent Nanobody Construct 6B12-9GS-TNF30
AVQLVESGGGLVQPGGSLRLSCAASGFTLAYYAIGWFRQAPGKEREGVSCISSSVGTTYYSDSVKGRFTISRDNAKNTVYLQMNSLKPEDTAVYYCVRSSWFDCGVQGRDLGNEYDYRGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 420 - protein, 240 aa, Artificial Sequence
  Recombinant Polypeptide Multivalent Nanobody Construct 6B6-9GS-TNF30
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 421 - protein, 241 aa, Artificial Sequence
  Recombinant Polypeptide Multivalent Nanobody Construct 7G4-9GS-TNF30
AVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 422 - protein, 247 aa, Artificial Sequence
  Recombinant Polypeptide Multivalent Nanobody Construct NC3-9GS-TNF30
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 423 - protein, 247 aa, Artificial Sequence
  Recombinant Polypeptide Multivalent Nanobody Construct NC6-9GS-TNF30
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 424 - protein, 242 aa, Artificial Sequence
  Recombinant Polypeptide Multivalent Nanobody Construct 20F4-9GS-TNF30
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 425 - protein, 238 aa, Artificial Sequence
  Recombinant Polypeptide Multivalent Nanobody Construct 21C12-9GS-TNF30
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 426 - protein, 253 aa, Artificial Sequence
  Recombinant Polypeptide Multivalent Nanobody Construct 17B5-9GS-TNF30
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 427 - protein, 248 aa, Artificial Sequence
  Recombinant Polypeptide Multivalent Nanobody Construct 17C4-9GS-TNF30
AVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 428 - protein, 249 aa, Artificial Sequence
  Recombinant Polypeptide Multivalent Nanobody Construct 13F12-9GS-TNF30
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 429 - protein, 252 aa, Artificial Sequence
  Recombinant Polypeptide Multivalent Nanobody Construct TNF30-9GS-6B12
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSAVQLVESGGGLVQPGGSLRLSCAASGFTLAYYAIGWFRQAPGKEREGVSCISSSVGTTYYSDSVKGRFTISRDNAKNTVYLQMNSLKPEDTAVYYCVRSSWFDCGVQGRDLGNEYDYRGQGTQVTVSS

SEQ ID NO: 430 - protein, 240 aa, Artificial Sequence
  Recombinant Polypeptide Multivalent Nanobody Construct TNF30-9GS-6B6
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSQVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 431 - protein, 241 aa, Artificial Sequence
  Recombinant Polypeptide Multivalent Nanobody Construct TNF30-9GS-7G4
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSAVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSS

SEQ ID NO: 432 - protein, 247 aa, Artificial Sequence
  Recombinant Polypeptide Multivalent Nanobody Construct TNF30-9GS-NC3
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSEVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSS

SEQ ID NO: 433 - protein, 247 aa, Artificial Sequence
  Recombinant Polypeptide Multivalent Nanobody Construct TNF30-9GS-NC6
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSEVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSS

SEQ ID NO: 434 - protein, 242 aa, Artificial Sequence
  Recombinant Polypeptide Multivalent Nanobody Construct TNF30-9GS-20F4
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSQVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSS

SEQ ID NO: 435 - protein, 238 aa, Artificial Sequence
  Recombinant Polypeptide Multivalent Nanobody Construct TNF30-9GS-21C12
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSAVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSS

SEQ ID NO: 436 - protein, 253 aa, Artificial Sequence
  Recombinant Polypeptide Multivalent Nanobody Construct TNF30-9GS-17B5
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSQVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSS

SEQ ID NO: 437 - protein, 248 aa, Artificial Sequence
  Recombinant Polypeptide Multivalent Nanobody Construct TNF30-9GS-17C4
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSAVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSS

SEQ ID NO: 438 - protein, 249 aa, Artificial Sequence
  Recombinant Polypeptide Multivalent Nanobody Construct TNF30-9GS-13F12
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSAVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSS

SEQ ID NO: 439 - protein, 364 aa, Artificial Sequence
  Recombinant Polypeptide Multivalent Nanobody Construct 6B6-9GS-ALB8-9GS-TNF30
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 440 - protein, 365 aa, Artificial Sequence
  Recombinant Polypeptide Multivalent Nanobody Construct 7G4-9GS-ALB8-9GS-TNF30
AVQLVESGGGLVQPGGSLRLSCAASGSIFSRLAMDWYRQAPGKQRELVASISRSGTTMAADSVKGRFTISRDNAENMVYLQMNSLKPEDTAVYVCMAFDGYSGSDYWGRGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 441 - protein, 371 aa, Artificial Sequence
  Recombinant Polypeptide Multivalent Nanobody Construct NC3-9GS-ALB8-9GS-TNF30
EVQLVESGGGLVQPGGSLRLSCAASGNIAAINVMNWYRQAPGTQREFVAAITSGGRKNYADSVKGRFTISRDNAKNTVHLQMNSLKPEDTAVYYCNADAPLASDDDVAPADYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 442 - protein, 371 aa, Artificial Sequence
  Recombinant Polypeptide Multivalent Nanobody Construct NC6-9GS-ALB8-9GS-TNF30
EVQLVESGGGLVQAGGSLRLSCAASGPTFSSYAMGWFRQAPGKDREFVAAISSNGGSTRYADSVKGRFTISRDSAKNTAYLQMNSLKLEDTAVYYCAADETTGWVQLADFRSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 443 - protein, 366 aa, Artificial Sequence
  Recombinant Polypeptide Multivalent Nanobody Construct 20F4-9GS-ALB8-9GS-TNF30
QVQLVESGGGLVQAGGSLRLSCAASGRTFSPYTMGWFRQPPGKVREFVGRINWSGIRNYADSVKGRFTISRDNNNNTVYLQMNRLKPEDTAVYYCAAASRSGSGYDSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 444 - protein, 362 aa, Artificial Sequence
  Recombinant Polypeptide Multivalent Nanobody Construct 21C12-9GS-ALB8-9GS-TNF30
AVQLVESGGGLVQPGGSLRLSCAASGSITGINPMGWYRQAPGKQRELVARIHGSITNYADSVKGRFTISRDIAKNTAYLQMNSLKPEDTAVYYCNARRWGYDYWGQGAQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 445 - protein, 377 aa, Artificial Sequence
  Recombinant Polypeptide Multivalent Nanobody Construct 17B5-9GS-ALB8-9GS-TNF30
QVQLVESGGGLVQAGGSLRLSCAASGGTFSAFPMGWFRQAPGKERKFVAGISQSGGSTHYSDSVKGRFTISRENAKNTIYLQMNSLKPEDTAVYYCAARGRTLALRNYAYTTEVGYDDWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 446 - protein, 372 aa, Artificial Sequence
  Recombinant Polypeptide Multivalent Nanobody Construct 17C4-9GS-ALB8-9GS-TNF30
AVQLVDSGGGLVQAGDSLRLSCAASGRTFSNYAMAWFRQAPGKEREFVAVISYAGGRTYYADSVKGRFTISRDNAKNTVYLQMNSLKPEDTAVYYCAAVDSPLIATHPRGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 447 - protein, 373 aa, Artificial Sequence
  Recombinant Polypeptide Multivalent Nanobody Construct 13F12-9GS-ALB8-9GS-TNF30
AVQLVDSGGGLVQAGGSLRLSCAASGRTFSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 448 - protein, 30 aa, Artificial Sequence
  Recombinant Polypeptide Framework region 1 (FR1)
QVQLVESGGGLVQAGGSLRLSCAASGRTFS

SEQ ID NO: 449 - protein, 30 aa, Artificial Sequence
  Recombinant Polypeptide Framework region 1 (FR1)
DVQLVESGGDLVQPGGSLRLSCAASGFSFD

SEQ ID NO: 450 - protein, 30 aa, Artificial Sequence
  Recombinant Polypeptide Framework region 1 (FR1)
AVQLVESGGGLVQPGGSLRLSCAASGFTLA

SEQ ID NO: 451 - protein, 30 aa, Artificial Sequence
  Recombinant Polypeptide Framework region 1 (FR1)
QVQLVESAGGLVQPGGSLRLSCAASGIIFS

SEQ ID NO: 452 - protein, 30 aa, Artificial Sequence
  Recombinant Polypeptide Framework region 1 (FR1)
EVQLVESGGGLVQTGGSLRLSCATSGLAFS

SEQ ID NO: 453 - protein, 30 aa, Artificial Sequence
  Recombinant Polypeptide Framework region 1 (FR1)
AVQLVDSGGGLVQPGGSLRLSCAASGSIFS

SEQ ID NO: 454 - protein, 30 aa, Artificial Sequence
  Recombinant Polypeptide Framework region 1 (FR1)
AVQLVESGGGLVQPGGSLRLSCAASGSIFS

SEQ ID NO: 455 - protein, 30 aa, Artificial Sequence
  Recombinant Polypeptide Framework region 1 (FR1)
AVQLVESGGGLVQPGGSLRLSCAASGSISR

SEQ ID NO: 456 - protein, 30 aa, Artificial Sequence
  Recombinant Polypeptide Framework region 1 (FR1)
QVKLEESGGGLVQPGGSLRLSCAASGSISR

SEQ ID NO: 457 - protein, 30 aa, Artificial Sequence
  Recombinant Polypeptide Framework region 1 (FR1)
DVQLVESGGGLVQPGGSLRLSCAASGFTLD

SEQ ID NO: 458 - protein, 30 aa, Artificial Sequence
  Recombinant Polypeptide Framework region 1 (FR1)
QVQLVESGGGLVQPGGSLRLSCAASGFSLD

SEQ ID NO: 459 - protein, 30 aa, Artificial Sequence; identical to SEQ ID NO: 458
  Recombinant Polypeptide Framework region 1 (FR1)

SEQ ID NO: 460 - protein, 30 aa, Artificial Sequence
  Recombinant Polypeptide Framework region 1 (FR1)
QVQLVESGGGLVQPGGSLRLSCAASGFTLD

SEQ ID NO: 461 - protein, 30 aa, Artificial Sequence
  Recombinant Polypeptide Framework region 1 (FR1)
QVKLEESGGGLVQAGGSLRLSCVVSGRTFS

SEQ ID NO: 462 - protein, 30 aa, Artificial Sequence
  Recombinant Polypeptide Framework region 1 (FR1)
QVQLVESGGGLAQAGGSLRLSCVVSGRTFS

SEQ ID NO: 463 - protein, 30 aa, Artificial Sequence
  Recombinant Polypeptide Framework region 1 (FR1)
EVQLVESGGGLVQPGGSLRLSCAASGNIAA

SEQ ID NO: 464 - protein, 30 aa, Artificial Sequence
  Recombinant Polypeptide Framework region 1 (FR1)
EVQLVESGGGLVQAGGSLRLSCAASGPTFS

SEQ ID NO: 465 - protein, 30 aa, Artificial Sequence
  Recombinant Polypeptide Framework region 1 (FR1)
AVQLVDSGGGLVQAGGSLRLSCAASGRTFS

SEQ ID NO: 466 - protein, 30 aa, Artificial Sequence; identical to SEQ ID NO: 448
  Recombinant Polypeptide Framework region 1 (FR1)

SEQ ID NO: 467 - protein, 30 aa, Artificial Sequence; identical to SEQ ID NO: 448
  Recombinant Polypeptide Framework region 1 (FR1)

SEQ ID NO: 468 - protein, 30 aa, Artificial Sequence
  Recombinant Polypeptide Framework region 1 (FR1)
AVQLVESGGGLVQAGSSLRLSCAASGRTFS

SEQ ID NO: 469 - protein, 30 aa, Artificial Sequence
  Recombinant Polypeptide Framework region 1 (FR1)
QVKLEESGGGLVQAGSSLRLSCAASGRTSS

SEQ ID NO: 470 - protein, 30 aa, Artificial Sequence
  Recombinant Polypeptide Framework region 1 (FR1)
AVQLVESGGGLVQPGGSLRLSCAASGSITS

SEQ ID NO: 471 - protein, 30 aa, Artificial Sequence
  Recombinant Polypeptide Framework region 1 (FR1)
AVQLVESGGGLVQPGGSLRLSCAASGSITG

SEQ ID NO: 472 - protein, 30 aa, Artificial Sequence
  Recombinant Polypeptide Framework region 1 (FR1)
QVQLVESGGGLVQPGGSLRLSCAASEYITS

SEQ ID NO: 473 - protein, 30 aa, Artificial Sequence; identical to SEQ ID NO: 465
  Recombinant Polypeptide Framework region 1 (FR1)

SEQ ID NO: 474 - protein, 30 aa, Artificial Sequence; identical to SEQ ID NO: 448
  Recombinant Polypeptide Framework region 1 (FR1)

SEQ ID NO: 475 - protein, 30 aa, Artificial Sequence
  Recombinant Polypeptide Framework region 1 (FR1)
EVQLVESGGGLVQAGGSLRLSCAASGRTFS

SEQ ID NO: 476 - protein, 30 aa, Artificial Sequence
  Recombinant Polypeptide Framework region 1 (FR1)
AVQLVDSGGGLVQAGGSLRLSCAASGGTFT

SEQ ID NO: 477 - protein, 30 aa, Artificial Sequence
  Recombinant Polypeptide Framework region 1 (FR1)
DVQLVESGGGLVQAGGSLRLSCAASGGTFT

SEQ ID NO: 478 - protein, 30 aa, Artificial Sequence
  Recombinant Polypeptide Framework region 1 (FR1)
QVKLEESGGGLVQAGGSLRLSCAASGGTFS

SEQ ID NO: 479 - protein, 30 aa, Artificial Sequence
  Recombinant Polypeptide Framework region 1 (FR1)
QVQLVESGGGLVQAGGSLRLSCAASGGTFS

SEQ ID NO: 480 - protein, 30 aa, Artificial Sequence
  Recombinant Polypeptide Framework region 1 (FR1)
AVQLVESGGGLVQAGGSLRLSCAASGGTFS

SEQ ID NO: 481 - protein, 30 aa, Artificial Sequence
  Recombinant Polypeptide Framework region 1 (FR1)
EVQLVESGGGLVQAGGSLRLSCAASGGTFS

SEQ ID NO: 482 - protein, 30 aa, Artificial Sequence; identical to SEQ ID NO: 479
  Recombinant Polypeptide Framework region 1 (FR1)

SEQ ID NO: 483 - protein, 30 aa, Artificial Sequence; identical to SEQ ID NO: 479
  Recombinant Polypeptide Framework region 1 (FR1)

SEQ ID NO: 484 - protein, 30 aa, Artificial Sequence; identical to SEQ ID NO: 480
  Recombinant Polypeptide Framework region 1 (FR1)

SEQ ID NO: 485 - protein, 30 aa, Artificial Sequence
  Recombinant Polypeptide Framework region 1 (FR1)
AVQLVDSGGGLVQAGGSLRLSCAASGGTFS

SEQ ID NO: 486 - protein, 30 aa, Artificial Sequence; identical to SEQ ID NO: 478
  Recombinant Polypeptide Framework region 1 (FR1)

SEQ ID NO: 487 - protein, 30 aa, Artificial Sequence
  Recombinant Polypeptide Framework region 1 (FR1)
QVQLVESGGGLVQAGGSLRLSCAASGPTFS

SEQ ID NO: 488 - protein, 30 aa, Artificial Sequence
  Recombinant Polypeptide Framework region 1 (FR1)
AVQLVDSGGGLVQAGGSLRLSCAASGRSFS

SEQ ID NO: 489 - protein, 30 aa, Artificial Sequence
  Recombinant Polypeptide Framework region 1 (FR1)
AVQLVDSGGGLVQAGDSLRLSCAASGRTFS

SEQ ID NO: 490 - protein, 30 aa, Artificial Sequence
  Recombinant Polypeptide Framework region 1 (FR1)
QVQLVESGGGLVQAGDSLRVACAASGRTFS

SEQ ID NO: 491 - protein, 30 aa, Artificial Sequence
  Recombinant Polypeptide Framework region 1 (FR1)
QVQLVESGGGLVQTGGSLRLSCAASGSTFS

SEQ ID NO: 492 - protein, 30 aa, Artificial Sequence
  Recombinant Polypeptide Framework region 1 (FR1)
QVQLVESGGGLVQAGGSLRLSCASSGRTFS

SEQ ID NO: 493 - protein, 30 aa, Artificial Sequence
  Recombinant Polypeptide Framework region 1 (FR1)
QVKLEESGGGLVQAGGSLRLSCASSGRTFS

SEQ ID NO: 494 - protein, 30 aa, Artificial Sequence; identical to SEQ ID NO: 465
  Recombinant Polypeptide Framework region 1 (FR1)

SEQ ID NO: 495 - protein, 30 aa, Artificial Sequence
  Recombinant Polypeptide Framework region 1 (FR1)
DVQLVESGGGLVQAGGSLRLSCAASGRTFS

SEQ ID NO: 496 - protein, 30 aa, Artificial Sequence; identical to SEQ ID NO: 475
  Recombinant Polypeptide Framework region 1 (FR1)

SEQ ID NO: 497 - protein, 30 aa, Artificial Sequence; identical to SEQ ID NO: 448
  Recombinant Polypeptide Framework region 1 (FR1)

SEQ ID NO: 498 - protein, 30 aa, Artificial Sequence
  Recombinant Polypeptide Framework region 1 (FR1)
AVQLVESGGGLVQAGGSLRLSSVVSGGTFS

SEQ ID NO: 499 - protein, 14 aa, Artificial Sequence
  Recombinant Polypeptide Framework region 2 (FR2)
WFRQPPGKVREFVG

SEQ ID NO: 500 - protein, 14 aa, Artificial Sequence
  Recombinant Polypeptide Framework region 2 (FR2)
WLRQTPGKGLEWVG

SEQ ID NO: 501 - protein, 14 aa, Artificial Sequence
  Recombinant Polypeptide Framework region 2 (FR2)
WFRQAPGKEREGVS

SEQ ID NO: 502 - protein, 14 aa, Artificial Sequence
  Recombinant Polypeptide Framework region 2 (FR2)
WYRQAPGKRRELVA

SEQ ID NO: 503 - protein, 14 aa, Artificial Sequence
  Recombinant Polypeptide Framework region 2 (FR2)
WFRQAPGKEREFVA

SEQ ID NO: 504 - protein, 14 aa, Artificial Sequence
  Recombinant Polypeptide Framework region 2 (FR2)
WYRQAPGKQRELVA

SEQ ID NO: 505 - protein, 14 aa, Artificial Sequence
  Recombinant Polypeptide Framework region 2 (FR2)
WYRQAPGKQRELVA

SEQ ID NO: 506 - protein, 14 aa, Artificial Sequence
  Recombinant Polypeptide Framework region 2 (FR2)
WYRQAPGKQRELVA

SEQ ID NO: 507 - protein, 14 aa, Artificial Sequence
  Recombinant Polypeptide Framework region 2 (FR2)
WYRQAPGKQRELVA

SEQ ID NO: 508 - protein, 14 aa, Artificial Sequence
  Recombinant Polypeptide Framework region 2 (FR2)
WFRQAPGKEREGVS

SEQ ID NO: 509 - protein, 14 aa, Artificial Sequence
  Recombinant Polypeptide Framework region 2 (FR2)
WFRQAPGKEREGVS

SEQ ID NO: 510 - protein, 14 aa, Artificial Sequence
  Recombinant Polypeptide Framework region 2 (FR2)
WFRQAPGKEREGVS

SEQ ID NO: 511 - protein, 14 aa, Artificial Sequence
  Recombinant Polypeptide Framework region 2 (FR2)
WFRQAPGKEREGVS

SEQ ID NO: 512 - protein, 14 aa, Artificial Sequence
  Recombinant Polypeptide Framework region 2 (FR2)
WFRQAPGKEREFVT

SEQ ID NO: 513 - protein, 14 aa, Artificial Sequence
  Recombinant Polypeptide Framework region 2 (FR2)
WFSQRPGKEREWVA

SEQ ID NO: 514 - protein, 14 aa, Artificial Sequence
  Recombinant Polypeptide Framework region 2 (FR2)
WYRQAPGTQREFVA

SEQ ID NO: 515 - protein, 14 aa, Artificial Sequence
  Recombinant Polypeptide Framework region 2 (FR2)
WFRQAPGKDREFVA

SEQ ID NO: 516 - protein, 14 aa, Artificial Sequence
  Recombinant Polypeptide Framework region 2 (FR2)
WFRQPPGKVREFVG

SEQ ID NO: 517 - protein, 14 aa, Artificial Sequence
  Recombinant Polypeptide Framework region 2 (FR2)
WFRQPPGKVREFVG

SEQ ID NO: 518 - protein, 14 aa, Artificial Sequence
  Recombinant Polypeptide Framework region 2 (FR2)
WFRQPPGKVREFVG

SEQ ID NO: 519 - protein, 14 aa, Artificial Sequence
  Recombinant Polypeptide Framework region 2 (FR2)
WFRQPPGKVREFVG

SEQ ID NO: 520 - protein, 14 aa, Artificial Sequence
  Recombinant Polypeptide Framework region 2 (FR2)
WFRQPPGKVREFVG

SEQ ID NO: 521 - protein, 14 aa, Artificial Sequence
  Recombinant Polypeptide Framework region 2 (FR2)
WYRQAPGKQRELVA

SEQ ID NO: 522 - protein, 14 aa, Artificial Sequence
  Recombinant Polypeptide Framework region 2 (FR2)
WYRQAPGKQRELVA

SEQ ID NO: 523 - protein, 14 aa, Artificial Sequence
  Recombinant Polypeptide Framework region 2 (FR2)
WYRQAPGKQRDLVA

SEQ ID NO: 524 - protein, 14 aa, Artificial Sequence
  Recombinant Polypeptide Framework region 2 (FR2)
WFRQGPGKERKFVA

SEQ ID NO: 525 - protein, 14 aa, Artificial Sequence
  Recombinant Polypeptide Framework region 2 (FR2)
WFRQAPGKERKFVA

SEQ ID NO: 526 - protein, 14 aa, Artificial Sequence
  Recombinant Polypeptide Framework region 2 (FR2)
WFRQAPGKERKFVA

SEQ ID NO: 527 - protein, 14 aa, Artificial Sequence
  Recombinant Polypeptide Framework region 2 (FR2)
WFRQAPGKERKFVA

SEQ ID NO: 528 - protein, 14 aa, Artificial Sequence
  Recombinant Polypeptide Framework region 2 (FR2)
WFRQAPGKERKFVA

SEQ ID NO: 529 - protein, 14 aa, Artificial Sequence
  Recombinant Polypeptide Framework region 2 (FR2)
WFRQAPGKEREFVT

SEQ ID NO: 530 - protein, 14 aa, Artificial Sequence
  Recombinant Polypeptide Framework region 2 (FR2)
WFRQAPGKGREFVA

SEQ ID NO: 531 - protein, 14 aa, Artificial Sequence
  Recombinant Polypeptide Framework region 2 (FR2)
WFRQAPGEKREFVA

SEQ ID NO: 532 - protein, 14 aa, Artificial Sequence
  Recombinant Polypeptide Framework region 2 (FR2)
WFRQAPGKERKFVA

SEQ ID NO: 533 - protein, 14 aa, Artificial Sequence
  Recombinant Polypeptide Framework region 2 (FR2)
WFRQAPGKERKFVA

SEQ ID NO: 534 - protein, 14 aa, Artificial Sequence
  Recombinant Polypeptide Framework region 2 (FR2)
WFRQAPGKERKFVA

SEQ ID NO: 535 - protein, 14 aa, Artificial Sequence
  Recombinant Polypeptide Framework region 2 (FR2)
WFRQAPGKEREFVA

SEQ ID NO: 536 - protein, 14 aa, Artificial Sequence
  Recombinant Polypeptide Framework region 2 (FR2)
WFRQAPGKEREFVA

SEQ ID NO: 537 - protein, 14 aa, Artificial Sequence
  Recombinant Polypeptide Framework region 2 (FR2)
WFRQAPGKEREFVA

SEQ ID NO: 538 - protein, 14 aa, Artificial Sequence
  Recombinant Polypeptide Framework region 2 (FR2)
WFRQAPGKEREFVA

SEQ ID NO: 539 - protein, 14 aa, Artificial Sequence
  Recombinant Polypeptide Framework region 2 (FR2)
WFRQAPGKEREFVA

SEQ ID NO: 540 - protein, 14 aa, Artificial Sequence
  Recombinant Polypeptide Framework region 2 (FR2)
WFRQAPGKEREFVA

SEQ ID NO: 541 - protein, 14 aa, Artificial Sequence
  Recombinant Polypeptide Framework region 2 (FR2)
WFRQAPGKEREFVS

SEQ ID NO: 542 - protein, 14 aa, Artificial Sequence
  Recombinant Polypeptide Framework region 2 (FR2)
WFRQAPGKEREFVA

SEQ ID NO: 543 - protein, 14 aa, Artificial Sequence
  Recombinant Polypeptide Framework region 2 (FR2)
WFRQTPGKEREFVS

SEQ ID NO: 544 - protein, 14 aa, Artificial Sequence
  Recombinant Polypeptide Framework region 2 (FR2)
WFRQTPGKEREFVS

SEQ ID NO: 545 - protein, 14 aa, Artificial Sequence
  Recombinant Polypeptide Framework region 2 (FR2)
WFRQAPGKEREFVA

SEQ ID NO: 546 - protein, 14 aa, Artificial Sequence
  Recombinant Polypeptide Framework region 2 (FR2)
WFRQAPGKEREFVA

SEQ ID NO: 547 - protein, 14 aa, Artificial Sequence
  Recombinant Polypeptide Framework region 2 (FR2)
WFRQAPGKEREFVA

SEQ ID NO: 548 - protein, 14 aa, Artificial Sequence
  Recombinant Polypeptide Framework region 2 (FR2)
WFRQAPGKEREFVS

SEQ ID NO: 549 - protein, 14 aa, Artificial Sequence
  Recombinant Polypeptide Framework region 2 (FR2)
WFRQPPGKEREFVA

SEQ ID NO: 550 - protein, 32 aa, Artificial Sequence
  Recombinant Polypeptide Framework region 3 (FR3)
RFTISRDNNNNTVYLQMNRLKPEDTAVYYCAA

SEQ ID NO: 551 - protein, 32 aa, Artificial Sequence; identical to SEQ ID NO: 116
  Recombinant Polypeptide Framework region 3 (FR3)

SEQ ID NO: 552 - protein, 32 aa, Artificial Sequence
  Recombinant Polypeptide Framework region 3 (FR3)
RFTISRDNAKNTVYLQMNSLKPEDTAVYYCVR

SEQ ID NO: 553 - protein, 32 aa, Artificial Sequence
  Recombinant Polypeptide Framework region 3 (FR3)
RFTISRDNAKNTVYLQMNSLKPEDTAVYYCHS

SEQ ID NO: 554 - protein, 32 aa, Artificial Sequence
  Recombinant Polypeptide Framework region 3 (FR3)
RFTISRDNAKNTVYLQMNSLKPEDSAVYYCAA

SEQ ID NO: 555 - protein, 32 aa, Artificial Sequence
  Recombinant Polypeptide Framework region 3 (FR3)
RFTISRDNAENTVYLQMNSLKPEDTAVYYCMA

SEQ ID NO: 556 - protein, 32 aa, Artificial Sequence
  Recombinant Polypeptide Framework region 3 (FR3)
RFTISRDNAENMVYLQMNSLKPEDTAVYVCMA

SEQ ID NO: 557 - protein, 32 aa, Artificial Sequence
  Recombinant Polypeptide Framework region 3 (FR3)
RFTISRDNTKNTVYLQMNSLKPDDTAVYYCHT

SEQ ID NO: 558 - protein, 32 aa, Artificial Sequence
  Recombinant Polypeptide Framework region 3 (FR3)
RFTISRDNTKNTVYLQMNSLKPEDTAVYYCHT

SEQ ID NO: 559 - protein, 32 aa, Artificial Sequence
  Recombinant Polypeptide Framework region 3 (FR3)
RFTISRDNAKNTVYLQMNSLKPEDTAVYYCAT

SEQ ID NO: 560 - protein, 32 aa, Artificial Sequence; identical to SEQ ID NO: 559
  Recombinant Polypeptide Framework region 3 (FR3)

SEQ ID NO: 561 - protein, 32 aa, Artificial Sequence; identical to SEQ ID NO: 559
  Recombinant Polypeptide Framework region 3 (FR3)

SEQ ID NO: 562 - protein, 32 aa, Artificial Sequence
  Recombinant Polypeptide Framework region 3 (FR3)
RFTISRDNVKNMVYLQMNSLKPEDTAVYFCAI

SEQ ID NO: 563 - protein, 32 aa, Artificial Sequence
  Recombinant Polypeptide Framework region 3 (FR3)
RFTISRNDAKNTVFLQMSSLKPEDTAVYYCAL

SEQ ID NO: 564 - protein, 32 aa, Artificial Sequence
  Recombinant Polypeptide Framework region 3 (FR3)
RFTISRNDAKNTVSLQMNSLKPEDSAVYYCAL

SEQ ID NO: 565 - protein, 32 aa, Artificial Sequence
  Recombinant Polypeptide Framework region 3 (FR3)
RFTISRDNAKNTVHLQMNSLKPEDTAVYYCNA

SEQ ID NO: 566 - protein, 32 aa, Artificial Sequence
  Recombinant Polypeptide Framework region 3 (FR3)
RFTISRDSAKNTAYLQMNSLKLEDTAVYYCAA

SEQ ID NO: 567 - protein, 32 aa, Artificial Sequence; identical to SEQ ID NO: 550
  Recombinant Polypeptide Framework region 3 (FR3)

SEQ ID NO: 568 - protein, 32 aa, Artificial Sequence; identical to SEQ ID NO: 550
  Recombinant Polypeptide Framework region 3 (FR3)

SEQ ID NO: 569 - protein, 32 aa, Artificial Sequence; identical to SEQ ID NO: 550
  Recombinant Polypeptide Framework region 3 (FR3)

SEQ ID NO: 570 - protein, 32 aa, Artificial Sequence
  Recombinant Polypeptide Framework region 3 (FR3)
RFTISRDNNKNTVYLQMNRLKPEDTAVYYCAA

SEQ ID NO: 571 - protein, 32 aa, Artificial Sequence
  Recombinant Polypeptide Framework region 3 (FR3)
RFTISRDNNKNTVYLQMNRLKPEDTAVYYCAS

SEQ ID NO: 572 - protein, 32 aa, Artificial Sequence
  Recombinant Polypeptide Framework region 3 (FR3)
RFTISRDIAKNTVYLQMNSLKPEDTAVYYCNA

SEQ ID NO: 573 - protein, 32 aa, Artificial Sequence
  Recombinant Polypeptide Framework region 3 (FR3)
RFTISRDIAKNTAYLQMNSLKPEDTAVYYCNA

SEQ ID NO: 574 - protein, 32 aa, Artificial Sequence
  Recombinant Polypeptide Framework region 3 (FR3)
RFTISRDIAKNTVSLQMNSLKPEDTAVYYCNA

SEQ ID NO: 575 - protein, 32 aa, Artificial Sequence
  Recombinant Polypeptide Framework region 3 (FR3)
RFTISRENAKNTVYLQMNSLKPEDTAVYYCAA

SEQ ID NO: 576 - protein, 32 aa, Artificial Sequence
  Recombinant Polypeptide Framework region 3 (FR3)
RFTISRENAKSTVYLQMNSLKPEDTAVYYCAA

SEQ ID NO: 577 - protein, 32 aa, Artificial Sequence; identical to SEQ ID NO: 575
  Recombinant Polypeptide Framework region 3 (FR3)

SEQ ID NO: 578 - protein, 32 aa, Artificial Sequence
  Recombinant Polypeptide Framework region 3 (FR3)
RFTISRENAKTTVYLQMNSLKPEDTAVYYCAA

SEQ ID NO: 579 - protein, 32 aa, Artificial Sequence; identical to SEQ ID NO: 578
  Recombinant Polypeptide Framework region 3 (FR3)

SEQ ID NO: 580 - protein, 32 aa, Artificial Sequence; identical to SEQ ID NO: 575
  Recombinant Polypeptide Framework region 3 (FR3)

SEQ ID NO: 581 - protein, 32 aa, Artificial Sequence; identical to SEQ ID NO: 575
  Recombinant Polypeptide Framework region 3 (FR3)

SEQ ID NO: 582 - protein, 32 aa, Artificial Sequence
  Recombinant Polypeptide Framework region 3 (FR3)
RFTISRENARNTVYLQMNSLKPEDTAVYYCAA

SEQ ID NO: 583 - protein, 32 aa, Artificial Sequence
  Recombinant Polypeptide Framework region 3 (FR3)
RFTISRENAKNTVYLQMNNLKPEDTAVYYCAA

SEQ ID NO: 584 - protein, 32 aa, Artificial Sequence
  Recombinant Polypeptide Framework region 3 (FR3)
RFTISRENAKNTIYLQMNSLKPEDTAVYYCAA

SEQ ID NO: 585 - protein, 32 aa, Artificial Sequence; identical to SEQ ID NO: 575
  Recombinant Polypeptide Framework region 3 (FR3)

SEQ ID NO: 586 - protein, 32 aa, Artificial Sequence
  Recombinant Polypeptide Framework region 3 (FR3)
RFTISKENAKSTVYLQMNSLKPEDTAVYYCAA

SEQ ID NO: 587 - protein, 32 aa, Artificial Sequence
  Recombinant Polypeptide Framework region 3 (FR3)
RFTISKENAKNTVYLQMNSLKPEDTAVYYCAA

SEQ ID NO: 588 - protein, 32 aa, Artificial Sequence; identical to SEQ ID NO: 587
  Recombinant Polypeptide Framework region 3 (FR3)

SEQ ID NO: 589 - protein, 32 aa, Artificial Sequence
  Recombinant Polypeptide Framework region 3 (FR3)
RFTISRDNAKNTVYLRMNSLKPEDTAAYYCAA

SEQ ID NO: 590 - protein, 32 aa, Artificial Sequence; identical to SEQ ID NO: 112
  Recombinant Polypeptide Framework region 3 (FR3)

SEQ ID NO: 591 - protein, 32 aa, Artificial Sequence; identical to SEQ ID NO: 112
  Recombinant Polypeptide Framework region 3 (FR3)

SEQ ID NO: 592 - protein, 32 aa, Artificial Sequence
  Recombinant Polypeptide Framework region 3 (FR3)
RFTSSRDNAKSTSYLQMNSLKPEDTAVYYCAA

SEQ ID NO: 593 - protein, 32 aa, Artificial Sequence
  Recombinant Polypeptide Framework region 3 (FR3)
RFTISRDNAQNTVYLQMNSLKPEDTAVYYCAA

SEQ ID NO: 594 - protein, 32 aa, Artificial Sequence
  Recombinant Polypeptide Framework region 3 (FR3)
RFTISRDNAKNTVYLQMNNLKPEDTAVYYCAA

SEQ ID NO: 595 - protein, 32 aa, Artificial Sequence; identical to SEQ ID NO: 112
  Recombinant Polypeptide Framework region 3 (FR3)

SEQ ID NO: 596 - protein, 32 aa, Artificial Sequence; identical to SEQ ID NO: 112
  Recombinant Polypeptide Framework region 3 (FR3)

SEQ ID NO: 597 - protein, 32 aa, Artificial Sequence
  Recombinant Polypeptide Framework region 3 (FR3)
RFTISRDNAKNTVYLQMNSLKPEDTAVYYCAG

SEQ ID NO: 598 - protein, 32 aa, Artificial Sequence; identical to SEQ ID NO: 597
  Recombinant Polypeptide Framework region 3 (FR3)

SEQ ID NO: 599 - protein, 32 aa, Artificial Sequence; identical to SEQ ID NO: 112
  Recombinant Polypeptide Framework region 3 (FR3)

SEQ ID NO: 600 - protein, 32 aa, Artificial Sequence
  Recombinant Polypeptide Framework region 3 (FR3)
RFTISRDNAKNTVYLQMNSLKPEDTAVYYSAA

SEQ ID NO: 601 - protein, 11 aa, Artificial Sequence
  Recombinant Polypeptide Framework region 4 (FR4)
WGQGTQVTVSS

SEQ ID NO: 602 - protein, 11 aa, Artificial Sequence
  Recombinant Polypeptide Framework region 4 (FR4)
LGQGTQVTVSS

SEQ ID NO: 603 - protein, 11 aa, Artificial Sequence
  Recombinant Polypeptide Framework region 4 (FR4)
RGQGTQVTVSS

SEQ ID NO: 604 - protein, 11 aa, Artificial Sequence
  Recombinant Polypeptide Framework region 4 (FR4)
WGQGTQVTVSS

SEQ ID NO: 605 - protein, 11 aa, Artificial Sequence
  Recombinant Polypeptide Framework region 4 (FR4)
WGQGTQVTVSS

SEQ ID NO: 606 - protein, 11 aa, Artificial Sequence
  Recombinant Polypeptide Framework region 4 (FR4)
WGRGTQVTVSS

SEQ ID NO: 607 - protein, 11 aa, Artificial Sequence
  Recombinant Polypeptide Framework region 4 (FR4)
WGRGTQVTVSS

SEQ ID NO: 608 - protein, 11 aa, Artificial Sequence
  Recombinant Polypeptide Framework region 4 (FR4)
WGQGTQVTVSS

SEQ ID NO: 609 - protein, 11 aa, Artificial Sequence
  Recombinant Polypeptide Framework region 4 (FR4)
WGQGAQVTVSS

SEQ ID NO: 610 - protein, 11 aa, Artificial Sequence
  Recombinant Polypeptide Framework region 4 (FR4)
WGQGTQVTVSS

SEQ ID NO: 611 - protein, 11 aa, Artificial Sequence
  Recombinant Polypeptide Framework region 4 (FR4)
WGQGTQVTVSS

SEQ ID NO: 612 - protein, 11 aa, Artificial Sequence
  Recombinant Polypeptide Framework region 4 (FR4)
WGQGTQVTVSS

SEQ ID NO: 613 - protein, 11 aa, Artificial Sequence
  Recombinant Polypeptide Framework region 4 (FR4)
WGQGTQVTVSS

SEQ ID NO: 614 - protein, 11 aa, Artificial Sequence
  Recombinant Polypeptide Framework region 4 (FR4)
WGQGTQVTVSS

SEQ ID NO: 615 - protein, 11 aa, Artificial Sequence
  Recombinant Polypeptide Framework region 4 (FR4)
WGQGTQVTVSS

SEQ ID NO: 616 - protein, 11 aa, Artificial Sequence
  Recombinant Polypeptide Framework region 4 (FR4)
WGQGTQVTVSS

SEQ ID NO: 617 - protein, 11 aa, Artificial Sequence
  Recombinant Polypeptide Framework region 4 (FR4)
WGQGTQVTVSS

SEQ ID NO: 618 - protein, 11 aa, Artificial Sequence
  Recombinant Polypeptide Framework region 4 (FR4)
WGQGTQVTVSS

SEQ ID NO: 619 - protein, 11 aa, Artificial Sequence
  Recombinant Polypeptide Framework region 4 (FR4)
WGQGTQVTVSS

SEQ ID NO: 620 - protein, 11 aa, Artificial Sequence
  Recombinant Polypeptide Framework region 4 (FR4)
WGQGTQVTVSS

SEQ ID NO: 621 - protein, 11 aa, Artificial Sequence
  Recombinant Polypeptide Framework region 4 (FR4)
WGQGTQVTVSS

SEQ ID NO: 622 - protein, 11 aa, Artificial Sequence
  Recombinant Polypeptide Framework region 4 (FR4)
WGQGTQVTVSS

SEQ ID NO: 623 - protein, 11 aa, Artificial Sequence
  Recombinant Polypeptide Framework region 4 (FR4)
WGQGAQVTVSS

SEQ ID NO: 624 - protein, 11 aa, Artificial Sequence
  Recombinant Polypeptide Framework region 4 (FR4)
WGQGAQVTVSS

SEQ ID NO: 625 - protein, 11 aa, Artificial Sequence
  Recombinant Polypeptide Framework region 4 (FR4)
WGQGTQVTVSS

SEQ ID NO: 626 - protein, 11 aa, Artificial Sequence
  Recombinant Polypeptide Framework region 4 (FR4)
WGQGTQVTVSS

SEQ ID NO: 627 - protein, 11 aa, Artificial Sequence
  Recombinant Polypeptide Framework region 4 (FR4)
WGQGTQVTVSS

SEQ ID NO: 628 - protein, 11 aa, Artificial Sequence
  Recombinant Polypeptide Framework region 4 (FR4)
WGQGTQVTVSS

SEQ ID NO: 629 - protein, 11 aa, Artificial Sequence
  Recombinant Polypeptide Framework region 4 (FR4)
WGQGTQVTVSS

SEQ ID NO: 630 - protein, 11 aa, Artificial Sequence
  Recombinant Polypeptide Framework region 4 (FR4)
WGQGTQVTVSS

SEQ ID NO: 631 - protein, 11 aa, Artificial Sequence
  Recombinant Polypeptide Framework region 4 (FR4)
WGQGTQVTVSS

SEQ ID NO: 632 - protein, 11 aa, Artificial Sequence
  Recombinant Polypeptide Framework region 4 (FR4)
WGQGTQVTVSS

SEQ ID NO: 633 - protein, 11 aa, Artificial Sequence
  Recombinant Polypeptide Framework region 4 (FR4)
WGQGTQVTVSS

SEQ ID NO: 634 - protein, 11 aa, Artificial Sequence
  Recombinant Polypeptide Framework region 4 (FR4)
WGQGTQVTVSS

SEQ ID NO: 635 - protein, 11 aa, Artificial Sequence
  Recombinant Polypeptide Framework region 4 (FR4)
WGQGTQVTVSS

SEQ ID NO: 636 - protein, 11 aa, Artificial Sequence
  Recombinant Polypeptide Framework region 4 (FR4)
WGQGTQVTVSS

SEQ ID NO: 637 - protein, 11 aa, Artificial Sequence
  Recombinant Polypeptide Framework region 4 (FR4)
WGQGTQVTVSS

SEQ ID NO: 638 - protein, 11 aa, Artificial Sequence
  Recombinant Polypeptide Framework region 4 (FR4)
WGQGTQVTVSS

SEQ ID NO: 639 - protein, 11 aa, Artificial Sequence
  Recombinant Polypeptide Framework region 4 (FR4)
WGQGTQVTVSS

SEQ ID NO: 640 - protein, 11 aa, Artificial Sequence
  Recombinant Polypeptide Framework region 4 (FR4)
WGQGTQVTVSS

SEQ ID NO: 641 - protein, 11 aa, Artificial Sequence
  Recombinant Polypeptide Framework region 4 (FR4)
WGKGTLVTVSS

SEQ ID NO: 642 - protein, 11 aa, Artificial Sequence
  Recombinant Polypeptide Framework region 4 (FR4)
WGQGTQVTVSS

SEQ ID NO: 643 - protein, 11 aa, Artificial Sequence
  Recombinant Polypeptide Framework region 4 (FR4)
WGQGTQVTVSS

SEQ ID NO: 644 - protein, 11 aa, Artificial Sequence
  Recombinant Polypeptide Framework region 4 (FR4)
WGQGTQVTVSS

SEQ ID NO: 645 - protein, 11 aa, Artificial Sequence
  Recombinant Polypeptide Framework region 4 (FR4)
WGQGTQVTVSS

SEQ ID NO: 646 - protein, 11 aa, Artificial Sequence
  Recombinant Polypeptide Framework region 4 (FR4)
WGQGTQVTVSS

SEQ ID NO: 647 - protein, 11 aa, Artificial Sequence
  Recombinant Polypeptide Framework region 4 (FR4)
WGQGTQVTVSS

SEQ ID NO: 648 - protein, 11 aa, Artificial Sequence
  Recombinant Polypeptide Framework region 4 (FR4)
WGQGTQVTVSS

SEQ ID NO: 649 - protein, 11 aa, Artificial Sequence
  Recombinant Polypeptide Framework region 4 (FR4)
WGRGTQVTVSS

SEQ ID NO: 650 - protein, 11 aa, Artificial Sequence
  Recombinant Polypeptide Framework region 4 (FR4)
WGQGTQVTVSS

SEQ ID NO: 651 - protein, 11 aa, Artificial Sequence
  Recombinant Polypeptide Framework region 4 (FR4)
WGQGTQVTVSS

(19) United States
(12) Patent Application Publication
(10) Pub. No.: US 2011/0059101 A9
(43) Pub. Date: Mar. 10, 2011

(54) Amino Acid Sequences Directed Against Il-6 And Polypetides Comprising The Same For The Treatment Of Diseases And Disorders Associated With Il-6 Mediated Signalling

(75) Inventors: Joost Alexander Kolkman, Sint-Martens-Latem (BE); Guy Hermans, Zwijnaarde (BE); Hendricus Renerus Jacobus Matteus Hoogenboom, Maastricht (NL)

(73) Assignee: Ablynx N.V., Ghent-Zwijnaarde (BE)

(21) Appl. No.: 12/225,045

(22) PCT Filed: Mar. 13, 2007
(86) PCT No.: PCT/EP2007/002197

§ 371 Date: Mar. 16, 2009

Related U.S. Application Data
(60) Provisional application No. 60/782,243, filed on Mar. 13, 2006.
(60) Provisional application No. 60/872,541, filed on Dec. 1, 2006.

(51) Int. Cl. A61K 39/395; C07K 16/24; C07H 21/04; C12N 5/10; C12P 21/02; A61P 19/10; A61P 35/02; A61P 11/06; A61P 19/02

(52) U.S. Cl. 424/158.1; 530/389.2; 536/23.53; 435/325; 435/69.6

(57) ABSTRACT

The present invention relates to amino acid sequences that are directed against interleukin-6 (IL-6), as well as to compounds or constructs, and in particular proteins and polypeptides that comprise or essentially consist of one or more such amino acid sequences. The invention also relates to nucleic acids encoding such amino acid sequences and polypeptides to methods for preparing such amino acid sequences and polypeptides; to host cells expressing or capable of expressing such amino acid sequences or polypeptides; to compositions, and in particular to pharmaceutical compositions, that comprise such amino acid sequences, polypeptides, nucleic acids and/or host cells; and to uses of such amino acid sequences, polypeptides, nucleic acids, host cells and/or compositions, in particular for prophylactic, therapeutic or diagnostic purposes.

The present invention relates to amino acid sequences that are directed against (as defined herein) interleukin-6 (IL-6), as well as to compounds or constructs, and in particular proteins and polypeptides that comprise or essentially consist of one or more such amino acid sequences (also referred to herein as “amino acid sequences of the invention”, “compounds of the invention”, and “polypeptides of the invention”, respectively).

The invention also relates to nucleic acids encoding such amino acid sequences and polypeptides (also referred to herein as “nucleic acids of the invention” or “nucleotide sequences of the invention”); to methods for preparing such amino acid sequences and polypeptides; to host cells expressing or capable of expressing such amino acid sequences or polypeptides; to compositions, and in particular to pharmaceutical compositions, that comprise such amino acid sequences, polypeptides, nucleic acids and/or host cells; and to uses of such amino acid sequences, polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

The interaction of IL-6, a protein originally identified as a B cell differentiation factor (Hirano et al., 1985; EP0257406), with IL-6R (Yamasaki et al., 1988; EP0325474) results in the formation of the IL-6/IL-6R complex. This complex binds to gp130 (Taga et al., 1989; EP0411946), a membrane protein on a target cell, which transmits various physiological actions of IL-6. IL-6 is currently known to be involved in—amongst others—the regulation of the immune response, hematopoiesis, the acute phase response, bone metabolism, angiogenesis, and inflammation. Deregulation of IL-6 production is implicated in the pathology of several autoimmune and chronic inflammatory proliferative disease processes (Ishihara and Hirano, 2002). As a consequence, inhibitors of IL-6 induced signaling have attracted much attention in the past (Hirano et al., 1990). Polypeptides specifically binding to IL-6 (Klein et al., 1991; EP0312996), IL-6R (EP0409607) or gp130 (Saito et al., 1993; EP0572118) proved to exhibit an efficient inhibitory effect on IL-6 functioning.

IL-6 overproduction and signalling (and in particular so-called trans-signalling) are involved in various diseases and disorders, such as sepsis (Starnes et al., 1999) and various forms of cancer such as multiple myeloma disease (MM), renal cell carcinoma (RCC), plasma cell leukaemia (Klein et al., 1991), lymphoma, B-lymphoproliferative disorder (BLPD) and prostate cancer. Non-limiting examples of other diseases caused by excessive IL-6 production or signalling include bone resorption (osteoporosis) (Roodman et al., 1992; Jilka et al., 1992), cachexia (Strassman et al., 1992), psoriasis, mesangial proliferative glomerulonephritis, Kaposi's sarcoma, AIDS-related lymphoma (Emilie et al., 1994), inflammatory diseases and disorder such as rheumatoid arthritis, systemic onset juvenile idiopathic arthritis, hypergammaglobulinemia (Grau et al., 1990); Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus (Campbell et al., 1991). Other IL-6 related disorders will be clear to the skilled person.

As can for example be seen from the references above, the prior art describes antibodies and antibody fragments directed against human IL-6, against human IL-6R and against human gp130 protein for the prevention and treatment of IL-6 relates disorders. Examples are Tocilizumab (see Woo P, et al. Arthritis Res Ther. (2005) 7: 1281-8, Nishimoto N et al. Blood. (2005) 106: 2627-32, Ito H et al. Gastroenterology. (2004) 126: 989-96, Choy E H et al. Arthritis Rheum. (2002) 46: 3143-50), BE8 (see Bataille R et al. Blood (1995) 86:685-91, Emilie D et al. Blood (1994) 84:2472-9, Beck J T et al. N Engl J. Med. (1994) 330:602-5, Wendling D et al. J Rheumatol. (1993) 20:259-62) and CNTO-328 of Centocor (see Journal of Clinical Oncology, (2004) 22/14S: 2560; Journal of Clinical Oncology, (2004) 22/14S: 2608; Int J Cancer (2004) 111:592-5). Another active principle known in the art for the prevention and treatment of IL-6 related disorders is an Fc fusion of soluble gp130 (see Becker C et al. Immunity. (2004) 21: 491-501, Doganci A et al. J Clin Invest. (2005) 115:313-25, Nowell M A et al. J Immunol. (2003) 171: 3202-9, Atreya R et al. Nat. Med. (2000) 6:583-8).

The polypeptides and compositions of the present invention can generally be used to modulate, and in particular inhibit and/or prevent, binding of IL-6 to IL-6R, and thus to modulate, and in particular inhibit or prevent, the signalling that is mediated by IL-6 and/or IL-6R, to modulate the biological pathways in which IL-6 and/or IL-6R are involved, and/or to modulate the biological mechanisms, responses and effects associated with such signalling or these pathways.

As such, the polypeptides and compositions of the present invention can be used in the prevention and/or treatment (as defined herein) of diseases and disorders associated with IL-6-mediated signalling, such as diseases and disorders associated with interleukin-6 (“IL-6”) and/or with the IL-6/IL-6R complex, and/or with the signalling pathway(s) and/or the biological functions and responses in which interleukin-6 (“IL-6”) and/or the IL-6/IL-6R complex are involved. Generally, “diseases and disorders associated with IL-6-mediated signalling” can be defined as diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof) and/or of a known active principle active against IL-6 or a biological pathway or mechanism in which IL-6 is involved (and in particular, of a pharmaceutically active amount thereof). Examples of such diseases and disorders associated with IL-6-mediated signalling will be clear to the skilled person based on the disclosure herein, and for example include the following diseases and disorders: sepsis, various forms of cancer such as multiple myeloma disease (MM), renal cell carcinoma (RCC), plasma cell leukaemia, lymphoma, B-lymphoproliferative disorder (BLPD), prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus.

In particular, the polypeptides and compositions of the present invention can be used for the prevention and treatment of diseases and disorders associated with IL-6-mediated signalling which are characterized by excessive and/or unwanted signalling mediated by IL-6 or by the pathway(s) in which IL-6 is involved. Examples of such diseases and disorders associated with IL-6-mediated signalling will again be clear to the skilled person based on the disclosure herein.

In particular, the polypeptides and compositions of the present invention can be used in the prevention and/or treatment of diseases and disorders which can benefit from modulating the signaling pathway(s) and/or the biological functions and responses in which IL-6 and/or the IL-6/IL-6R complex are involved. Generally, these diseases and disorder will be characterized by abnormal, undesired, increased and/or reduced signaling associated with IL-6 and/or the IL-6/IL-6R complex.

More in particular, the polypeptides and compositions of the present invention can be used in the prevention and/or treatment of diseases and disorders which can benefit from modulating the interaction between the IL-6 and IL-6R, and/or between the IL-6/IL-6R complex and gp 130.

Examples of the diseases and disorders referred to above (herein collectively: “IL-6 related disorders” or “diseases and disorders associated with IL-6-mediated signalling” [both terms will be used interchangeably in the further description herein]) will be clear to the skilled person, for example from the prior art, such as the background art as referred to herein below.

The polypeptides and preparations of the present invention can generally be used to modulate, and in particular inhibit and/or prevent, binding of IL-6 to IL-6R and/or the binding of the IL6/IL-6R complex to gp 130, and thus to modulate, and in particular inhibit or prevent, the IL-6-mediated signalling or IL6/IL-6R complex-mediated signalling and/or to modulate the biological responses and effects associated with such signalling. As such, the polypeptides and preparations of the present invention can be used for the prevention and treatment of IL-6 relates disorders, and in particular for IL-6 related disorders which are characterized by excessive and/or unwanted IL-6-mediated signalling.

Thus, without being limited thereto, the amino acid sequences and polypeptides of the invention can for example be used to prevent or treat all diseases and disorders that are currently being prevented or treated with active principles that can modulate IL-6-mediated signalling, such as those mentioned in the prior art cited above. It is also envisaged that the polypeptides of the invention can be used to prevent or treat all diseases and disorders for which treatment with such active principles is currently being developed, has been proposed, or will be proposed or developed in future. In addition, it is envisaged that—because of their unique properties as further described herein—the polypeptides of the present invention may be used for the prevention and treatment of other diseases and disorders than those for which these known active principles are being used or will be proposed or developed; and/or that the polypeptides of the present invention may provide new methods and regimens for treating the diseases and disorders described herein.

Other applications and uses of the amino acid sequences and polypeptides of the invention will become clear to the skilled person from the further disclosure herein.

Generally, it is an object of the invention to provide pharmacologically active agents, as well as compositions comprising the same, that can be used in the diagnosis, prevention and/or treatment of IL-6 related disorders and the further diseases and disorders mentioned herein, and to provide methods for the diagnosis, prevention and/or treatment of such diseases and disorders involving the use and/or administration of such agents and compositions.

In particular, it is an object of the invention to provide such pharmacologically active agents, compositions and/or methods that provide certain advantages compared to the agents, compositions and/or methods currently used and/or known in the art. These advantages will become clear from the further description below.

More in particular, it is an object of the invention to provide therapeutic proteins that can be used as pharmacologically active agents, as well as compositions comprising the same, for the diagnosis, prevention and/or treatment of IL-6 related disorders and the further diseases and disorders mentioned herein, and to provide methods for the diagnosis, prevention and/or treatment of such diseases and disorders involving the use and/or administration of such agents and compositions. In the present invention, these therapeutic proteins are amino acid sequences, (single) domain antibodies and/or in particular Nanobodies®, and/or are polypeptides or proteins based thereon or comprising the same, as further described below.

In the invention, generally, these objects are achieved by the use of the amino acid sequences, Nanobodies and polypeptides provided herein.

Thus, it is a specific object of the present invention to provide amino acid sequences and/or Nanobodies directed against (as defined herein) IL-6, in particular against IL-6 from a warm-blooded animal, more in particular against IL-6 from a mammal, and especially against human IL-6; and to provide proteins and polypeptides comprising or essentially consisting of at least one such amino acid sequence and/or Nanobody.

In particular, it is a specific object of the present invention to provide such amino acid sequences and/or Nanobodies and such proteins and/or polypeptides that are suitable for prophylactic, therapeutic and/or diagnostic use in a warm-blooded animal, and in particular in a mammal, and more in particular in a human being.

More in particular, it is an object of the present invention to provide such amino acid sequences and/or Nanobodies and such proteins and/or polypeptides that can be used for the prevention, treatment, alleviation and/or diagnosis of one or more diseases, disorders or conditions associated with IL-6 and/or mediated by IL-6 (such as the diseases, disorders and conditions mentioned herein) in a warm-blooded animal, in particular in a mammal, and more in particular in a human being.

It is also a specific object of the invention to provide such amino acid sequences and/or Nanobodies and such proteins and/or polypeptides that can be used in the preparation of a pharmaceutical or veterinary composition for the prevention and/or treatment of one or more diseases, disorders or conditions associated with and/or mediated by IL-6 (such as the diseases, disorders and conditions mentioned herein) in a warm-blooded animal, in particular in a mammal, and more in particular in a human being.

One specific but non-limiting object of the invention is to provide amino acid sequences and/or Nanobodies, proteins and/or polypeptides against IL-6 that have improved therapeutic and/or pharmacological properties and/or other advantageous properties (such as, for example, improved ease of preparation and/or reduced costs of goods), compared to conventional antibodies against IL-6 or fragments thereof, such as Fab′ fragments, F(ab′)₂ fragments, ScFv constructs, “diabodies” and/or other classes of (single) domain antibodies, such as the “dAb's described by Ward et al (supra). These improved and advantageous properties will become clear from the further description herein, and for example include, without limitation, one or more of:
increased affinity for IL-6, either in a monovalent format, in a multivalent format (for example in a bivalent format) and/or in a multispecific format (for example one of the multispecific formats described hereinbelow);
better suitability for formatting in a multivalent format (for example in a bivalent format);
better suitability for formatting in a multispecific format (for example one of the multispecific formats described hereinbelow);
improved suitability or susceptibility for “humanizing” substitutions (as defined herein); and/or
less immunogenicity, either in a monovalent format, in a multivalent format (for example in a bivalent format) and/or in a multispecific format (for example one of the multispecific formats described hereinbelow) in a monovalent format;
increased stability, either in a monovalent format, in a multivalent format (for example in a bivalent format) and/or in a multispecific format (for example one of the multispecific formats described hereinbelow) in a monovalent format;
increased specificity towards IL-6, either in a monovalent format, in a multivalent format (for example in a bivalent format) and/or in a multispecific format (for example one of the multispecific formats described in IL-6 or hereinbelow) in a monovalent format;
decreased or where desired increased cross-reactivity with IL-6 from different species; and/or
one or more other improved properties desirable for pharmaceutical use (including prophylactic use and/or therapeutic use) and/or for diagnostic use (including but not limited to use for imaging purposes), either in a monovalent format, in a multivalent format (for example in a bivalent format) and/or in a multispecific format (for example one of the multispecific formats described hereinbelow).

In the invention, generally, these objects are achieved by the use of amino acid sequences and/or Nanobodies, proteins, polypeptides and compositions described herein. These amino acid sequences and/or Nanobodies are also referred to herein as “amino acid sequences of the invention” and/or “Nanobodies of the invention”; and these proteins and polypeptides and compositions are also collectively referred to herein “polypeptides of the invention” and “compositions of the invention”.

In general, the invention provides amino acid sequences that are directed against (as defined herein) and/or can specifically bind (as defined herein) to IL-6; as well as compounds and constructs, and in particular proteins and polypeptides, that comprise at least one such amino acid sequence.

More in particular, the invention provides amino acid sequences that can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence.

In particular, amino acid sequences and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² mole/liter or less and more preferably 10⁻⁸ to 10⁻¹² mole/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent amino acid sequence of the invention (or a polypeptide that contains only one amino acid sequence of the invention) is preferably such that it will bind to IL-6 with an affinity less than 500 nM, preferably less than 200 nM, more preferably less than 10 nM, such as less than 500 pM.

Some preferred IC₅₀ values for binding of the amino acid sequences or polypeptides of the invention to IL-6 will become clear from the further description and examples herein.

For binding to IL-6, an amino acid sequence of the invention will usually contain within its amino acid sequence one or more amino acid residues or one or more stretches of amino acid residues (i.e. with each “stretch” comprising two or amino acid residues that are adjacent to each other or in close proximity to each other, i.e. in the primary or tertiary structure of the amino acid sequence) via which the amino acid sequence of the invention can bind to IL-6, which amino acid residues or stretches of amino acid residues thus form the “site” for binding to IL-6 (also referred to herein as the “antigen binding site”).

The amino acid sequences provided by the invention are preferably in essentially isolated form (as defined herein), or form part of a protein or polypeptide of the invention (as defined herein), which may comprise or essentially consist of one or more amino acid sequences of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than IL-6), so as to provide a monovalent, multivalent or multispecific polypeptide of the invention, respectively, all as described herein. Such a protein or polypeptide may also be in essentially isolated form (as defined herein).

The amino acid sequences and polypeptides of the invention as such preferably essentially consist of a single amino acid chain that is not linked via disulphide bridges to any other amino acid sequence or chain (but that may or may not contain one or more intramolecular disulphide bridges. For example, it is known that Nanobodies—as described herein—may sometimes contain a disulphide bridge between CDR3 and CDR1 or FR2). However, it should be noted that one or more amino acid sequences of the invention may be linked to each other and/or to other amino acid sequences (e.g. via disulphide bridges) to provide peptide constructs that may also be useful in the invention (for example Fab′ fragments, F(ab′)₂ fragments, ScFv constructs, “diabodies” and other multispecific constructs. Reference is for example made to the review by Holliger and Hudson, Nat. Biotechnol. 2005 September; 23 (9): 1126-36).

Generally, when an amino acid sequence of the invention (or a compound, construct or polypeptide comprising the same) is intended for administration to a subject (for example for therapeutic and/or diagnostic purposes as described herein), it is preferably either an amino acid sequence that does not occur naturally in said subject; or, when it does occur naturally in said subject, in essentially isolated form (as defined herein).

Thus, in a first aspect, the invention relates to an amino acid sequence and/or Nanobody against IL-6, and in particular to an amino acid sequence and/or Nanobody against IL-6 from a warm-blooded animal, and more in particular to a Nanobody against IL-6 from a mammal, and especially to a Nanobody against human IL-6.

In another aspect, the invention relates to a protein or polypeptide that comprises or essentially consists of at least one such amino acid sequence and/or Nanobody against IL-6.

It will be clear to the skilled person that for pharmaceutical use, the amino acid sequences and/or Nanobodies of the invention (as well as compounds, constructs and polypeptides of the invention comprising the same) are preferably directed against human IL-6; whereas for veterinary purposes, the amino acid sequences and/or Nanobodies and polypeptides of the invention are preferably directed against IL-6 from the species to be treated, or at least cross-reactive with IL-6 from the species to be treated.

Furthermore, an amino acid sequence of the invention may optionally, and in addition to the at least one binding site for binding against IL-6, contain one or more further binding sites for binding against other antigens, proteins or targets.

The efficacy of the amino acid sequences and/or Nanobodies and polypeptides of the invention, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person, and for example include proliferation assays using IL6-dependent cell lines including B9, XG1 and 7TD1, collagen induced arthritis model, transplant model of synovial tissue in SCID mice, xenograft models of various human cancers, including lymphoma, myeloma, prostate cancer and renal cell carcinoma, IBD models including TNBS, DSS and IL10 knockout models, as well as the assays and animal models used in the experimental part below and in the prior art cited herein.

The amino acid sequences and/or Nanobodies provided by the invention are preferably in essentially isolated form (as defined herein), or form part of a protein or polypeptide of the invention (as defined herein), which may comprise or essentially consist of one or more amino acid sequences and/or Nanobodies of the invention and which may optionally further comprise one or more further amino acid sequences and/or Nanobodies (all optionally linked via one or more suitable linkers). For example, and without limitation, one or more further Nanobodies that can serve as a binding unit (i.e. against one or more other targets than IL-6), so as to provide a monovalent, multivalent or multispecific polypeptide of the invention, respectively, all as described herein. Such a protein or polypeptide may also be in essentially isolated form (as defined herein).

Also, according to the invention, Nanobodies and polypeptides that are directed against IL-6 from a first species of warm-blooded animal may or may not show cross-reactivity with IL-6 from one or more other species of warm-blooded animal. For example, Nanobodies and polypeptides directed against human IL-6 may or may not show cross reactivity with IL-6 from one or more other species of primates (such as, without limitation, monkeys from the genus Macaca (such as, and in particular, cynomologus monkeys (Macaca fascicularis) and/or rhesus monkeys (Macaca mulatta)) and baboon (Papio ursinus)) and/or with IL-6 from one or more species of animals that are often used in animal models for diseases (for example mouse, rat, rabbit, pig or dog), and in particular in animal models for diseases and disorders associated with IL-6 (such as the species and animal models mentioned herein). In this respect, it will be clear to the skilled person that such cross-reactivity, when present, may have advantages from a drug development point of view, since it allows the Nanobodies and polypeptides against human IL-6 to be tested in such disease models.

More generally, amino acid sequences and/or Nanobodies and polypeptides of the invention that are cross-reactive with IL-6 from multiple species of mammal will usually be advantageous for use in veterinary applications, since with will allow the same Nanobody or polypeptide to be used across multiple species. Thus, it is also encompassed within the scope of the invention that Nanobodies and polypeptides directed against IL-6 from one species of animal (such as Nanobodies and polypeptides against human IL-6 can be used in the treatment of another species of animal, as long as the use of the Nanobodies and/or polypeptides provide the desired effects in the species to be treated.

The present invention is in its broadest sense also not particularly limited to or defined by a specific antigenic determinant, epitope, part, domain, subunit or confirmation (where applicable) of IL-6 against which the amino acid sequences and/or Nanobodies and polypeptides of the invention are directed. With advantage, the invention provides a range of amino acid sequences and/or Nanobodies directed against different epitopes or binding sites of IL-6.

Thus, the invention provides:
Non-inhibiting Nanobodies PMP6D5 (SEQ ID NO: 320) and PMP8F2 (SEQ ID NO: 321).
Inhibiting Nanobodies interacting with the IL-6/IL-6R interaction site: PMP6B12 (SEQ ID NO: 322), PMP6B6 (SEQ ID NO: 323), PMP11C1 (SEQ ID NO: 324), PMP23H2 (SEQ ID NO: 325), PMP7G4 (SEQ ID NO: 326), PMP20D2 (SEQ ID NO: 327), PMP7G5 (SEQ ID NO: 328), PMP7H3 (SEQ ID NO: 329), PMP7G9 (SEQ ID NO: 330), PMP9A9 (SEQ ID NO: 331), PMP22E3 (SEQ ID NO: 332), PMP6E10 (SEQ ID NO: 333) and PMP6G10 (SEQ ID NO: 334);
Inhibiting Nanobodies interacting with the gp130 binding site II NC3 (SEQ ID NO: 335), NC6 (SEQ ID NO: 336), PMP13A1 (SEQ ID NO: 337), PMP20G9 (SEQ ID NO: 338), PMP20F4 (SEQ ID NO: 339), PMP21A7 (SEQ ID NO: 340), PMP13D8 (SEQ ID NO: 341), PMP21E12 (SEQ ID NO: 342), PMP21C12 (SEQ ID NO: 343), PMP21C2 (SEQ ID NO: 344), PMP14G4 (SEQ ID NO: 345), PMP14E1 (SEQ ID NO: 346), PMP6E9 (SEQ ID NO: 347), PMP12H3 (SEQ ID NO: 348), PMP12C5 (SEQ ID NO: 349), PMP17G7 (SEQ ID NO: 350), PMP14G11 (SEQ ID NO: 351), PMP9F9 (SEQ ID NO: 352), PMP14A8 (SEQ ID NO: 353), PMP17B5 (SEQ ID NO: 354), PMP6B7 (SEQ ID NO: 355), PMP14E9 (SEQ ID NO: 356), PMP17D7 (SEQ ID NO: 357) and PMP14G1 (SEQ ID NO: 358).
Inhibiting Nanobodies interacting with the gp130 binding site III: PMP10C4 (SEQ ID NO: 360), PMP17C4 (SEQ ID NO: 361), PMP21B4 (SEQ ID NO: 362), PMP21H1 (SEQ ID NO: 363), PMP10A6 (SEQ ID NO: 364), PMP13H6 (SEQ ID NO: 365), PMP13F12 (SEQ ID NO: 366), PMP21A2 (SEQ ID NO: 367), PMP21F7 (SEQ ID NO: 368), PMP21H3 (SEQ ID NO: 369) and PMP21E7 (SEQ ID NO: 370).

For therapeutic application, usually (polypeptides containing one or more) inhibiting Nanobodies will be preferred, whereas non-inhibiting Nanobodies may for example be preferred for diagnostic and/or imaging applications.

The invention also provides a range of multivalent and multispecific polypeptides based on the above Nanobodies. Some preferred, but non-limiting examples are the multivalent and multispecific polypeptides of SEQ ID NO's 371-447.

Particular embodiments of the present invention relate to:
Polypeptides comprising at least one binding site (e.g. a binding unit such as a Nanobody) interacting with the IL-6/IL-6R interaction site and at least one binding site (e.g. a binding unit such as a Nanobody) interacting with the gp 130 binding site II;
Polypeptides comprising at least one binding site (e.g. a binding unit such as a Nanobody) interacting with the IL-6/IL-6R interaction site and at least one binding site (e.g. a binding unit such as a Nanobody) interacting with the gp 130 binding site III;
Polypeptides comprising at least one binding site (e.g. a binding unit such as a Nanobody) interacting with the gp 130 binding site II and at least one binding site (e.g. a binding unit such as a Nanobody) interacting with the gp 130 binding site III;
   in which said polypeptides may optionally contain one or more further binding units and/or amino acid sequences and in which the binding units and amino acid sequences present in said polypeptides may optionally be suitably linked via one or more linker sequences.

It is also within the scope of the invention that, where applicable, an amino acid sequence and/or Nanobody of the invention can bind to two or more antigenic determinants, epitopes, parts, domains, subunits or confirmations of IL-6. In such a case, the antigenic determinants, epitopes, parts, domains or subunits of IL-6 to which the amino acid sequences and/or Nanobodies and/or polypeptides of the invention bind may be the essentially same (for example, if IL-6 contains repeated structural motifs or is present as a multimer) or may be different (and in the latter case, the amino acid sequences and/or Nanobodies and polypeptides of the invention may bind to such different antigenic determinants, epitopes, parts, domains, subunits of IL-6 with an affinity and/or specificity which may be the same or different). Also, for example, when IL-6 exists in an activated conformation and in an inactive conformation, the amino acid sequences and/or Nanobodies and polypeptides of the invention may bind to either one of these conformations, or may bind to both these conformations (i.e. with an affinity and/or specificity which may be the same or different). Also, for example, the amino acid sequences and/or Nanobodies and polypeptides of the invention may bind to a conformation of IL-6 in which it is bound to a pertinent ligand, may bind to a conformation of IL-6 in which it not bound to a pertinent ligand, or may bind to both such conformations (again with an affinity and/or specificity which may be the same or different).

It is also expected that the amino acid sequences and/or Nanobodies and polypeptides of the invention will generally bind to all naturally occurring or synthetic analogs, variants, mutants, alleles, parts and fragments of IL-6, or at least to those analogs, variants, mutants, alleles, parts and fragments of IL-6 that contain one or more antigenic determinants or epitopes that are essentially the same as the antigenic determinant(s) or epitope(s) to which the Nanobodies and polypeptides of the invention bind in IL-6 (e.g. in wild-type IL-6). Again, in such a case, the amino acid sequences and/or Nanobodies and polypeptides of the invention may bind to such analogs, variants, mutants, alleles, parts and fragments with an affinity and/or specificity that are the same as, or that different from (i.e. higher than or lower than), the affinity and specificity with which the amino acid sequences and/or Nanobodies of the invention bind to (wild-type) IL-6. It is also included within the scope of the invention that the Nanobodies and polypeptides of the invention bind to some analogs, variants, mutants, alleles, parts and fragments of IL-6, but not to others.

When IL-6 exists in a monomeric form and in one or more multimeric forms, it is within the scope of the invention that the amino acid sequences and/or Nanobodies and polypeptides of the invention only bind to IL-6 in monomeric form, only bind to IL-6 in multimeric form, or bind to both the monomeric and the multimeric form. Again, in such a case, the amino acid sequences and polypeptides of the invention may bind to the monomeric form with an affinity and/or specificity that are the same as, or that are different from (i.e. higher than or lower than), the affinity and specificity with which the amino acid sequences of the invention bind to the multimeric form.

Also, when IL-6 can associate with other proteins or polypeptides to form protein complexes (e.g. with multiple subunits), it is within the scope of the invention that the amino acid sequences and/or Nanobodies and polypeptides of the invention bind to IL-6 in its non-associated state, bind to IL-6 in its associated state, or bind to both. In all these cases, the amino acid sequences and/or Nanobodies and polypeptides of the invention may bind to such multimers or associated protein complexes with an affinity and/or specificity that may be the same as or different from (i.e. higher than or lower than) the affinity and/or specificity with which the amino acid sequences and/or Nanobodies and polypeptides of the invention bind to IL-6 in its monomeric and non-associated state.

Also, as will be clear to the skilled person, proteins or polypeptides that contain two or more amino acid sequences directed against IL-6 may bind with higher avidity to IL-6 than the corresponding monomeric amino acid sequence(s). For example, and without limitation, proteins or polypeptides that contain two or more amino acid sequences directed against different epitopes of IL-6 may (and usually will) bind with higher avidity than each of the different monomers, and proteins or polypeptides that contain two or more amino acid sequences directed against IL-6 may (and usually will) bind also with higher avidity to a multimer of IL-6.

Generally, the amino acid sequences and/or Nanobodies and polypeptides of the invention will at least bind to those forms (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the amino acid sequences and/or Nanobodies and polypeptides of the invention, and/or to use proteins or polypeptides comprising or essentially consisting of one or more of such parts, fragments, analogs, mutants, variants, alleles and/or derivatives, as long as these are suitable for the uses envisaged herein. Such parts, fragments, analogs, mutants, variants, alleles and/or derivatives will usually contain (at least part of) a functional antigen-binding site for binding against IL-6; and more preferably capable of specific binding to IL-6, and even more preferably capable of binding to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Some non-limiting examples of such parts, fragments, analogs, mutants, variants, alleles, derivatives, proteins and/or polypeptides will become clear from the further description herein. Additional fragments or polypeptides of the invention may also be provided by suitably combining (i.e. by linking or genetic fusion) one or more (smaller) parts or fragments as described herein.

In one specific, but non-limiting aspect of the invention, which will be further described herein, such analogs, mutants, variants, alleles, derivatives have an increased half-life in serum (as further described herein) compared to the amino acid sequence and/or Nanobody from which they have been derived. For example, an amino acid sequence and/or Nanobody of the invention may be linked (chemically or otherwise) to one or more groups or moieties that extend the half-life (such as PEG), so as to provide a derivative of an amino acid sequence and/or Nanobody of the invention with increased half-life.

In one specific, but non-limiting aspect, the amino acid sequence of the invention may be an amino acid sequence that comprises an immunoglobulin fold or may be an amino acid sequence that, under suitable conditions (such as physiological conditions) is capable of forming an immunoglobulin fold (i.e. by folding). Reference is inter alia made to the review by Halaby et al., J. (1999) Protein Eng. 12, 563-71. Preferably, when properly folded so as to form an immunoglobulin fold, such an amino acid sequence is capable of specific binding (as defined herein) to IL-6; and more preferably capable of binding to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Also, parts, fragments, analogs, mutants, variants, alleles and/or derivatives of such amino acid sequences are preferably such that they comprise an immunoglobulin fold or are capable for forming, under suitable conditions, an immunoglobulin fold.

In particular, but without limitation, the amino acid sequences of the invention may be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR's, as further described herein).

The amino acid sequences of the invention may in particular be an immunoglobulin sequence or a suitable fragment thereof, and more in particular be an immunoglobulin variable domain sequence or a suitable fragment thereof, such as light chain variable domain sequence (e.g. a V_{L}-sequence) or a suitable fragment thereof; or a heavy chain variable domain sequence (e.g. a V_{H}-sequence) or a suitable fragment thereof. When the amino acid sequence of the invention is a heavy chain variable domain sequence, it may be a heavy chain variable domain sequence that is derived from a conventional four-chain antibody (such as, without limitation, a V_{H} sequence that is derived from a human antibody) or be a so-called V_{HH}-sequence (as defined herein) that is derived from a so-called “heavy chain antibody” (as defined herein).

However, it should be noted that the invention is not limited as to the origin of the amino acid sequence and/or Nanobody of the invention (or of the nucleotide sequence of the invention used to express it), nor as to the way that the amino acid sequence and/or Nanobody or nucleotide sequence of the invention is (or has been) generated or obtained. Thus, the amino acid sequences and/or Nanobodies of the invention may be naturally occurring amino acid sequences and/or Nanobodies (from any suitable species) or synthetic or semi-synthetic amino acid sequences and/or Nanobodies, including but not limited to “humanized” (as defined herein) immunoglobulin sequences (such as partially or fully humanized mouse or rabbit immunoglobulin sequences, and in particular partially or fully humanized V_{HH} sequences or Nanobodies), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Reference is for example made to the standard handbooks, and well as to the further description and prior art mentioned herein.

Similarly, the nucleotide sequences of the invention may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

The amino acid sequence of the invention may in particular be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody™ (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21 (11):484-490; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).

In particular, the amino acid sequence of the invention may be a Nanobody™ (as defined herein) or a suitable fragment thereof. [Note: Nanobody™, Nanobodies™ and Nanoclone™ are trademarks of Ablynx N.V.] Such Nanobodies directed against IL-6 will also be referred to herein as “Nanobodies of the invention”.

For a general description of Nanobodies, reference is made to the further description below, as well as to the prior art cited herein. In this respect, it should however be noted that this description and the prior art mainly described Nanobodies of the so-called “V_{H}3 class” (i.e. Nanobodies with a high degree of sequence homology to human germline sequences of the V_{H}3 class such as DP-47, DP-51 or DP-29), which Nanobodies form a preferred aspect of this invention. It should however be noted that the invention in its broadest sense generally covers any type of Nanobody directed against IL-6, and for example also covers the Nanobodies belonging to the so-called “V_{H}4 class” (i.e. Nanobodies with a high degree of sequence homology to human germline sequences of the V_{H}4 class such as DP-78), as for example described in the U.S. provisional application 60/792,279 by Ablynx N.V. entitled “DP-78-like Nanobodies” filed on Apr. 14, 2006.

Generally, Nanobodies (in particular V_{HH} sequences and partially humanized Nanobodies) can in particular be characterized by the presence of one or more “Hallmark residues” (as described herein) in one or more of the framework sequences (again as further described herein).

Thus, generally, a Nanobody can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below;
   and in which:
ii) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded.

In these Nanobodies, the CDR sequences are generally as further defined herein.

Thus, the invention also relates to such Nanobodies that can bind to (as defined herein) and/or are directed against IL-6, to suitable fragments thereof, as well as to polypeptides that comprise or essentially consist of one or more of such Nanobodies and/or suitable fragments.

SEQ ID NO's 320 to 370 give the amino acid sequences of a number of V_{HH} sequences that have been raised against IL-6.

Accordingly, some particularly preferred Nanobodies of the invention are Nanobodies which can bind (as further defined herein) to and/or are directed against to IL-6 and which:
i) have 80% amino acid identity with at least one of the amino acid sequences of SEQ ID NO's: 320 to 370, in which for the purposes of determining the degree of amino acid identity, the amino acid residues that form the CDR sequences are disregarded. In this respect, reference is also made to Table A-1, which lists the framework 1 sequences (SEQ ID NO's: 448 to 498), framework 2 sequences (SEQ ID NO's: 499 to 549), framework 3 sequences (SEQ ID NO's: 550 to 600) and framework 4 sequences (SEQ ID NO's: 601 to 651) of the Nanobodies of SEQ ID NO's: 320 to 370 (with respect to the amino acid residues at positions 1 to 4 and 27 to 30 of the framework 1 sequences, reference is also made to the comments made below. Thus, for determining the degree of amino acid identity, these residues are preferably disregarded);
   and in which:
ii) preferably one or more of the amino acid residues at positions 11, 37, 44, 45, 47, 83, 84, 103, 104 and 108 according to the Kabat numbering are chosen from the Hallmark residues mentioned in Table A-3 below.

In these Nanobodies, the CDR sequences are generally as further defined herein.

Again, such Nanobodies may be derived in any suitable manner and from any suitable source, and may for example be naturally occurring V_{HH} sequences (i.e. from a suitable species of Camelid) or synthetic or semi-synthetic amino acid sequences, including but not limited to “humanized” (as defined herein) Nanobodies, “camelized” (as defined herein) immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

In particular, humanized Nanobodies may be amino acid sequences that are as generally defined for Nanobodies in the previous paragraphs, but in which at least one amino acid residue is present (and in particular, in at least one of the framework residues) that is and/or that corresponds to a humanizing substitution (as defined herein). Some preferred, but non-limiting humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Some particularly preferred humanized Nanobodies of the invention are humanized variants of the Nanobodies of SEQ ID NO's: 320 to 370.

Thus, some other preferred Nanobodies of the invention are Nanobodies which can bind (as further defined herein) to IL-6 and which:
i) are a humanized variant of one of the amino acid sequences of SEQ ID NO's: 320 to 370; and/or
ii) have 80% amino acid identity with at least one of the amino acid sequences of SEQ ID NO's: 320 to 370, in which for the purposes of determining the degree of amino acid identity, the amino acid residues that form the CDR sequences are disregarded;
   and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below.

As discussed above and in more detail herein, the Nanobodies of the invention generally comprise a single amino acid chain, that can be considered to comprise “framework sequences” or “FR” (which are generally as described herein) and “complementarity determining regions” or CDR's. Some preferred CDR's present in the Nanobodies of the invention are as described herein. More generally, and with reference to the further definitions given herein, the CDR sequences present in the Nanobodies of the invention are obtainable/can be obtained by a method comprising the steps of:
a) providing at least one V_{HH} domain directed against IL-6, by a method generally comprising the steps of (i) immunizing a mammal belonging to the Camelidae with IL-6 or a part or fragment thereof, so as to raise an immune response and/or antibodies (and in particular heavy chain antibodies) against IL-6; (ii) obtaining a biological sample from the mammal thus immunized, wherein said sample comprises heavy chain antibody sequences and/or V_{HH} sequences that are directed against IL-6; and (iii) obtaining (e.g isolating) heavy chain antibody sequences and/or V_{HH} sequences that are directed against IL-6 from said biological sample; and/or by a method generally comprising the steps of (i) screening a library comprising heavy chain antibody sequences and/or V_{HH} sequences for heavy chain antibody sequences and/or V_{HH} sequences that are directed against IL-6 or against at least one part or fragment thereof; and (ii) obtaining (e.g. isolating) heavy chain antibody sequences and/or V_{HH} sequences that are directed against IL-6 from said library;
b) optionally subjecting the heavy chain antibody sequences and/or V_{HH} sequences against IL-6 thus obtained to affinity maturation, to mutagenesis (e.g. random mutagenesis or site-directed mutagenesis) and/or any other technique(s) for increasing the affinity and/or specificity of the heavy chain antibody sequences and/or V_{HH} sequences for IL-6;
c) determining the sequences of the CDR's of the heavy chain antibody sequences and/or V_{HH} sequences against IL-6 thus obtained; and optionally
d) providing a Nanobody in which at least one, preferably at least two, and more preferably all three of the CDR's (i.e. CDR1, CDR2 and CDR3, and in particular at least CDR3) has a sequence that has been determined in step c).

Usually, in step d), all CDR sequences present in a Nanobody of the invention will be derived from the same heavy chain antibody or V_{HH} sequence. However, the invention in its broadest sense is not limited thereto. It is for example also possible (although often less preferred) to suitably combine, in a Nanobody of the invention, CDR's from two or three different heavy chain antibodies or V_{HH} sequences against IL-6 and/or to suitably combine, in a Nanobody of the invention, one or more CDR's derived from heavy chain antibodies or V_{HH} sequences (an in particular at least CDR3) with one or more CDR's derived from a different source (for example synthetic CDR's or CDR's derived from a human antibody or VH domain).

More in particular, the invention provides Nanobodies that can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such Nanobody.

In particular, Nanobodies and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻ⁱ and 10⁷ M⁻ⁱs⁻ⁱ; and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent Nanobody of the invention (or a polypeptide that contains only one Nanobody of the invention) is preferably such that it will bind to IL-6 with an affinity less than 500 nM, preferably less than 200 nM, more preferably less than 10 nM, such as less than 500 pM.

Some preferred IC50 values for binding of the Nanobodies or polypeptides of the invention to IL-6 will become clear from the further description and examples herein.

The affinity of the Nanobody of the invention against IL-6 can be determined in a manner known per se, for example using the assay described herein.

In a preferred but non-limiting aspect, the invention relates to a Nanobody (as defined herein) against IL-6, which consist of 4 framework regions (FR1 to FR4 respectively) and 3 complementarity determining regions (CDR1 to CDR3 respectively), in which:

(a) CDR1 is an amino acid sequence chosen from the group consisting of:

| | |
|---|---|
| SEQ ID NO: 167 | PYTMG |
| SEQ ID NO: 168 | DYAMS |
| SEQ ID NO: 169 | YYAIG |
| SEQ ID NO: 170 | INAMG |
| SEQ ID NO: 171 | IYTMG |
| SEQ ID NO: 172 | RLAMD |
| SEQ ID NO: 173 | RLAMD |
| SEQ ID NO: 174 | FNIMG |
| SEQ ID NO: 175 | FNIMG |
| SEQ ID NO: 176 | YYGVG |
| SEQ ID NO: 177 | YYGVG |
| SEQ ID NO: 178 | YYGVG |
| SEQ ID NO: 179 | DSAIG |
| SEQ ID NO: 180 | PYTIA |
| SEQ ID NO: 181 | PYTIG |
| SEQ ID NO: 182 | INVMN |
| SEQ ID NO: 183 | SYAMG |
| SEQ ID NO: 184 | PYTMG |
| SEQ ID NO: 185 | PYTVG |
| SEQ ID NO: 186 | PYTMG |
| SEQ ID NO: 187 | PYTMG |
| SEQ ID NO: 188 | PYTMG |
| SEQ ID NO: 189 | INPMG |
| SEQ ID NO: 190 | INPMG |
| SEQ ID NO: 191 | INPMA |
| SEQ ID NO: 192 | SYPMG |
| SEQ ID NO: 193 | SYPMG |
| SEQ ID NO: 194 | SYPMG |
| SEQ ID NO: 195 | SYPMG |
| SEQ ID NO: 196 | SYPMG |
| SEQ ID NO: 197 | SYPMG |
| SEQ ID NO: 198 | SFPMG |
| SEQ ID NO: 199 | SFPMG |
| SEQ ID NO: 200 | SFPMG |
| SEQ ID NO: 201 | AFPMG |
| SEQ ID NO: 202 | AFPMG |
| SEQ ID NO: 203 | AFPMG |
| SEQ ID NO: 204 | AFPMG |
| SEQ ID NO: 205 | AFPMG |
| SEQ ID NO: 206 | TYAMG |
| SEQ ID NO: 207 | NYHMV |
| SEQ ID NO: 208 | NYAMA |
| SEQ ID NO: 209 | IDAMA |
| SEQ ID NO: 210 | KHHATG |
| SEQ ID NO: 211 | SYVMG |
| SEQ ID NO: 212 | SYVMG |
| SEQ ID NO: 213 | SSPMG |
| SEQ ID NO: 214 | SSPMG |
| SEQ ID NO: 215 | SSPMG |
| SEQ ID NO: 216 | NGPMA |
| SEQ ID NO: 217 | SYPIA |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
   (b) CDR2 is an amino acid sequence chosen from the group consisting of:

| | |
|---|---|
| SEQ ID NO: 218 | RINWSGIRNYADSVKG |
| SEQ ID NO: 219 | AITGNGASKYYAESMKG |
| SEQ ID NO: 220 | CISSSVGTTYYSDSVKG |
| SEQ ID NO: 221 | DIMPYGSTEYADSVKG |
| SEQ ID NO: 222 | AAHWTVFRGNTYYVDSVKG |
| SEQ ID NO: 223 | SIAVSGTTMLDDSVKG |
| SEQ ID NO: 224 | SISRSGTTMAADSVKG |
| SEQ ID NO: 225 | DITNRGTTNYADSVKG |
| SEQ ID NO: 226 | DITNGGTTMYADSVKG |
| SEQ ID NO: 227 | CISSSDGDTYYADSVKG |
| SEQ ID NO: 228 | CISSSDGDTYYADSVKG |
| SEQ ID NO: 229 | CTSSSDGDTYYADSVKG |
| SEQ ID NO: 230 | CISSSDGDTYYDDSVKG |
| SEQ ID NO: 231 | TIIGSDRSTDLDGDTYYADSVRG |
| SEQ ID NO: 232 | TIIGSDRSTDLDGDTYYADSVRG |
| SEQ ID NO: 233 | AITSGGRKNYADSVKG |
| SEQ ID NO: 234 | AISSNGGSTRYADSVKG |
| SEQ ID NO: 235 | RINWSGIRNYADSVKG |
| SEQ ID NO: 236 | RINWSGIRNYADSVKG |
| SEQ ID NO: 237 | RINWSGIRNYADSVKG |
| SEQ ID NO: 238 | RINWSGITNYADSVKG |
| SEQ ID NO: 239 | RINWSGITNYADSVKG |
| SEQ ID NO: 240 | RIHGSITNYADSVKG |
| SEQ ID NO: 241 | RIHGSITNYADSVKG |
| SEQ ID NO: 242 | RIFGGGSTNYADSVKG |
| SEQ ID NO: 243 | GISQSGVGTAYSDSVKG |
| SEQ ID NO: 244 | GISQSGGSTAYSDSVKG |
| SEQ ID NO: 245 | GISQSSSSTAYSDSVKG |
| SEQ ID NO: 246 | GISQSGGSTAYSDSVKG |
| SEQ ID NO: 247 | GISQSGGSTAYSDSVKG |
| SEQ ID NO: 248 | GISQSGGSTAYSDSVKG |
| SEQ ID NO: 249 | GISQSGGSTHYSDSVKG |
| SEQ ID NO: 250 | GISQSGGSTHYSDSVKG |
| SEQ ID NO: 251 | GISQSGGSTHYSDSVKG |
| SEQ ID NO: 252 | GISQSGGSTHYSDSVKG |
| SEQ ID NO: 253 | GISQSGGSTHYSDSVKG |
| SEQ ID NO: 254 | GISQSGGSTHYSDSVKG |
| SEQ ID NO: 255 | GISQSGGSTHYSDSVKG |
| SEQ ID NO: 256 | GISQSGGSTHYSDSVKG |
| SEQ ID NO: 257 | AISWSGANTYYADSVKG |
| SEQ ID NO: 258 | AASGSTSSTYYADSVKG |
| SEQ ID NO: 259 | VISYAGGRTYYADSVKG |
| SEQ ID NO: 260 | TMNWSTGATYYADSVKG |
| SEQ ID NO: 261 | ALNWSGGNTYYTDSVKG |
| SEQ ID NO: 262 | TINWSGSNGYYADSVKG |
| SEQ ID NO: 263 | TINWSGSNKYYADSVKG |
| SEQ ID NO: 264 | AISGRSGNTYYADSVKG |
| SEQ ID NO: 265 | AISGRSGNTYYADSVKG |
| SEQ ID NO: 266 | AISGRSGNTYYADSVKG |
| SEQ ID NO: 267 | AISWRTGITYYADSVKG |
| SEQ ID NO: 268 | AISWRGGNTYYADSVKG |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
   (c) CDR3 is an amino acid sequence chosen from the group consisting of:

| | |
|---|---|
| SEQ ID NO: 269 | ASQSGSGYDS |
| SEQ ID NO: 270 | VAKDTGSFYYPAYEHDV |
| SEQ ID NO: 271 | SSWFDCGVQGRDLGNEYDY |
| SEQ ID NO: 272 | YDPRGDDY |
| SEQ ID NO: 273 | TRSTAWNSPQRYDY |
| SEQ ID NO: 274 | FDGYTGSDY |
| SEQ ID NO: 275 | FDGYSGSDY |
| SEQ ID NO: 276 | YYPTTGFDD |
| SEQ ID NO: 277 | YYPTTGFDD |
| SEQ ID NO: 278 | DLSDYGVCSRWPSPYDY |
| SEQ ID NO: 279 | DLSDYGVCSRWPSPYDY |
| SEQ ID NO: 280 | DLSDYGVCSRWPSPYDY |
| SEQ ID NO: 281 | DLSDYGVCSKWPSPYDY |
| SEQ ID NO: 282 | TGKGYVFTPNEYDY |
| SEQ ID NO: 283 | TAKGYVFTDNEYDY |
| SEQ ID NO: 284 | DAPLASDDDVAPADY |
| SEQ ID NO: 285 | DETTGWVQLADFRS |
| SEQ ID NO: 286 | ASQSGSGYDS |
| SEQ ID NO: 287 | ASQSGSGYDS |
| SEQ ID NO: 288 | ASRSGSGYDS |
| SEQ ID NO: 289 | ASRSGSGYDS |
| SEQ ID NO: 290 | ASQVGSGYDS |
| SEQ ID NO: 291 | RRWGYDY |
| SEQ ID NO: 292 | RRWGYDY |
| SEQ ID NO: 293 | RRWGYDY |
| SEQ ID NO: 294 | RDKTLALRDYAYTTDVGYDD |
| SEQ ID NO: 295 | RDKTLALRDYAYTTDVGYDD |
| SEQ ID NO: 296 | RGRTLALRDYAYTTEVGYDD |
| SEQ ID NO: 297 | RGRTLFLRDYAYTTEVGYDD |
| SEQ ID NO: 298 | RGRTLFLRGYAYTTEVGYDD |
| SEQ ID NO: 299 | RGRTIALRNYAYTTEVGYDD |
| SEQ ID NO: 300 | RGRTLALRNYAYTTEVGYDD |
| SEQ ID NO: 301 | RGRTLALRNYAYTTEVGYDD |
| SEQ ID NO: 302 | RGRTLALRNYAYTTEVGYDD |
| SEQ ID NO: 303 | RGRTLALRNYAYTTEVGYDD |
| SEQ ID NO: 304 | RGRTLALRNYAYTTEVGYDD |
| SEQ ID NO: 305 | RGRTLALRNYAYTTEVGYDD |
| SEQ ID NO: 306 | RGRTLALRNYAYTTEVGYDD |
| SEQ ID NO: 307 | RGGTLALRNYAYTTEVGYDD |
| SEQ ID NO: 308 | SAIIEGFQDSIVIFSEAGYDY |
| SEQ ID NO: 309 | VAGLLLPRVAEGMDY |
| SEQ ID NO: 310 | VDSPLIATHPRGYDY |
| SEQ ID NO: 311 | ARGLLIATDARGYDY |
| SEQ ID NO: 312 | GSYVFYFTVRDQYDY |
| SEQ ID NO: 313 | SAGGFLVPRVGQGYDY |
| SEQ ID NO: 314 | SAGGFLVPRVGQGYDY |
| SEQ ID NO: 315 | ERVGLLLTVVAEGYDY |
| SEQ ID NO: 316 | ERVGLLLTVVAEGYDY |
| SEQ ID NO: 317 | ERVGLLLTVVAEGYDY |
| SEQ ID NO: 318 | ERVGLLLAVVAEGYDY |
| SEQ ID NO: 319 | ERAGVLLTKVPEGYDY |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s).

Thus, some particularly preferred, but non-limiting CDR sequences and combinations of CDR sequences that are present in the Nanobodies of the invention are as listed in Table A-1 below (see detailed description).

Thus, in the Nanobodies of the invention, at least one of the CDR1, CDR2 and CDR3 sequences present is chosen from the group consisting of the CDR1, CDR2 and CDR3 sequences, respectively, listed in Table A-1; or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-1.

In particular, in the Nanobodies of the invention, at least the CDR3 sequence present is chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1; and/or from the group consisting of the CDR3 sequences that have 3, 2 or only 1 amino acid difference(s) with at least one of the CDR3 sequences listed in Table A-1.

Preferably, in the Nanobodies of the invention, at least two of the CDR1, CDR2 and CDR3 sequences present are chosen from the group consisting of the CDR1, CDR2 and CDR3 sequences, respectively, listed in Table A-1 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-1.

In particular, in the Nanobodies of the invention, at least the CDR3 sequence present is chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1, respectively; and at least one of the CDR1 and CDR2 sequences present is chosen from the group consisting of the CDR1 and CDR2 sequences, respectively, listed in Table A-1 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Most preferably, in the Nanobodies of the invention, all three CDR1, CDR2 and CDR3 sequences present are chosen from the group consisting of the CDR1, CDR2 and CDR3 sequences, respectively, listed in Table A-1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-1.

Even more preferably, in the Nanobodies of the invention, at least one of the CDR1, CDR2 and CDR3 sequences present is chosen from the group consisting of the CDR1, CDR2 and CDR3 sequences, respectively, listed in Table A-1. Preferably, in this embodiment, at least one or preferably both of the other two CDR sequences present are chosen from CDR sequences that that have at least 80%, preferably at least 90%, more preferably at least 95%, even more preferably at least 99% sequence identity with at least one of the corresponding CDR sequences, respectively, listed in Table A-1; and/or from the group consisting of the CDR sequences that have 3, 2 or only 1 amino acid difference(s) with at least one of the corresponding sequences, respectively, listed in Table A-1.

In particular, in the Nanobodies of the invention, at least the CDR3 sequence present is chosen from the group consisting of the CDR3 listed in Table A-1. Preferably, in this embodiment, at least one and preferably both of the CDR1 and CDR2 sequences present are chosen from the groups of CDR1 and CDR2 sequences, respectively, that that have at least 80%, preferably at least 90%, more preferably at least 95%, even more preferably at least 99% sequence identity with the CDR1 and CDR2 sequences, respectively, listed in listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Even more preferably, in the Nanobodies of the invention, at least two of the CDR1, CDR2 and CDR3 sequences present are chosen from the group consisting of the CDR1, CDR2 and CDR3 sequences, respectively, listed in Table A-1. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with at least one of the corresponding sequences listed in Table A-1.

In particular, in the Nanobodies of the invention, at least the CDR3 sequence is chosen from the group consisting of the CDR3 sequences listed in Table A-1, and either the CDR1 sequence or the CDR2 sequence is chosen from the group consisting of the CDR1 and CDR2 sequences, respectively, listed in Table A-1. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with the corresponding CDR sequences listed in Table A-1.

Even more preferably, in the Nanobodies of the invention, all three CDR1, CDR2 and CDR3 sequences present are chosen from the group consisting of the CDR1, CDR2 and CDR3 sequences, respectively, listed in Table A-1.

Also, generally, the combinations of CDR's listed in Table A-1 (i.e. those mentioned on the same line in Table A-1) are preferred. Thus, it is generally preferred that, when a CDR in a Nanobody of the invention is a CDR sequence mentioned in Table A-1 or is chosen from the group of CDR sequences that have at least 80%, preferably at least 90%, more preferably at least 95%, even more preferably at least 99% sequence identity with a CDR sequence listed in Table A-1; and/or from the group consisting of CDR sequences that have 3, 2 or only 1 amino acid difference(s) with a CDR sequence listed in Table A-1, that at least one and preferably both of the other CDR's are chosen from the CDR sequences that belong to the same combination in Table A-1 (i.e. mentioned on the same line in Table A-1) or are chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1; or (3) a CDR1 sequence; a CDR2 sequence that has more than 80% sequence identity with one of the CDR2 sequence listed in Table A-1; and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 and a CDR3 sequence listed in Table A-1 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and more than 80% sequence identity with the CDR3 sequence listed in Table A-1 that belongs to same different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same.

In the most preferred in the Nanobodies of the invention, the CDR1, CDR2 and CDR3 sequences present are chosen from the one of the combinations of CDR1, CDR2 and CDR3 sequences, respectively, listed in Table A-1.

Preferably, when a CDR sequence is chosen from the group of CDR sequences that have at least 80%, preferably at least 90%, more preferably at least 95%, even more preferably at least 99% sequence identity (as defined herein) with one of the CDR sequences listed in Table A-1; and/or when a CDR sequence is chosen from the group consisting of CDR sequences that have 3, 2 or only 1 amino acid difference(s) with one of the CDR sequences listed in Table A-1:
i) any amino acid substitution is preferably a conservative amino acid substitution (as defined herein); and/or
ii) said amino acid sequence preferably only contains amino acid substitutions, and no amino acid deletions or insertions, compared to the CDR sequence listed in Table A-1.

More in particular, the invention provides Nanobodies that can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such Nanobody.

In particular, Nanobodies and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s and 10⁻⁶ s⁻¹.

Preferably, a monovalent Nanobody of the invention (or a polypeptide that contains only one Nanobody of the invention) is preferably such that it will bind to IL-6 with an affinity less than 500 nM, preferably less than 200 nM, more preferably less than 10 nM, such as less than 500 pM.

Some preferred IC50 values for binding of the Nanobodies or polypeptides of the invention to IL-6 will become clear from the further description and examples herein.

The affinity of the Nanobody of the invention against IL-6 can be determined in a manner known per se, for example using the assay described herein.

According to another preferred, but non-limiting embodiment of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

Nanobodies with the above CDR sequences preferably have framework sequences that are as further defined herein.

In another aspect, the invention relates to a Nanobody with an amino acid sequence that is chosen from the group consisting of SEQ ID NO's: 320 to 370 or from the group consisting of from amino acid sequences that have more than 80%, preferably more than 90%, more preferably more than 95%, such as 99% or more sequence identity (as defined herein) with one or more of the amino acid sequences of SEQ ID NO's: 320 to 370.

According to a specific, but non-limiting embodiment, the latter amino acid sequences have been “humanized”, as further described herein. Preferred humanizing substitutions are as defined below.

Again, such Nanobodies may be derived in any suitable manner and from any suitable source, and may for example be naturally occurring V_{HH} sequences (i.e. from a suitable species of Camelid) or synthetic or semi-synthetic Nanobodies, including but not limited to “humanized” (as defined herein) Nanobodies, “camelized” (as defined herein) immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

In particular, humanized Nanobodies may be Nanobodies that are as generally defined for Nanobodies in the previous paragraphs, but in which at least one amino acid residue is present (and in particular, in at least one of the framework residues) that is and/or that corresponds to a humanizing substitution (as defined herein). Some preferred, but non-limiting humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Again, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these “Expedite fragments”, reference is again made to WO 03/050531)

The polypeptides of the invention comprise or essentially consist of at least one amino acid sequence comprising or essentially consisting of an immunoglobulin variable domain or an antigen binding fragment thereof and/or a Nanobody or suitable fragments thereof that are directed to IL-6. Some preferred, but non-limiting examples of polypeptides of the invention are given in SEQ ID NO's: 371 to 447.

In a first aspect, the invention provides amino acid sequences comprising or essentially consisting of an immunoglobulin variable domain or an antigen binding fragment thereof and/or Nanobodies (as defined herein) that can bind to IL-6 in such a way that they modulate the interaction between IL-6 and IL-6R. Preferably, these amino acid sequences and/or Nanobodies are such that they can compete with IL-6R for binding to IL-6. More preferably, these amino acid sequences and/or Nanobodies are such that they can bind to an epitope of IL-6 which lies in, comprises, or fully or partially overlaps with the IL-6R interaction site of IL-6 (for which reference is made to the prior art cited herein).

In a second aspect, the invention provides amino acid sequences comprising or essentially consisting of an immunoglobulin variable domain or an antigen binding fragment thereof and/or Nanobodies (as defined herein) that can bind to IL-6 in such a way that they can modulate the interaction between IL-6/IL-6R complex and gp130. In the context of the present invention “modulating the interaction between IL-6/IL-6R complex and gp130” can for example mean:
binding to IL-6 (i.e. as such or as present in the IL-6/IL-6R complex) in such a way that the formation of the IL-6/IL-6R complex is inhibited or affected (e.g. fully or partially disrupted) in such a way that the binding of the complex to—e.g. its affinity for—gp130 is reduced (or reversely, that the binding of gp 130 to—e.g. its affinity for—the complex is reduced), so that the signaling induced/mediated by the binding of the complex to gp130 is modulated (e.g. reduced);
   or
binding to IL-6 (i.e. as such or as present in the IL-6/IL-6R complex) in such a way that the formation of the IL-6/IL-6R complex essentially is not affected but that the binding of said complex to gp130 is modulated (e.g. inhibited), so that the signalling induced/mediated by the binding of the complex to gp130 is modulated (e.g. reduced);
   both compared to the formation of the complex and its binding to gp 130 without the presence of the amino acid sequence or Nanobody of the invention.

In this aspect, amino acid sequences or Nanobodies according to the invention preferably compete with gp130 for binding to either the gp130 interaction site II of IL-6 (or of the IL-6/IL-6R complex) or the gp130 interaction site III of IL-6 (or of the IL-6/IL-6R complex).
In a third aspect, the invention relates to amino acid sequences comprising or essentially consisting of an immunoglobulin variable domain or an antigen binding fragment thereof wherein said immunoglobulin variable domain or an antigen binding fragment thereof binds to IL-6 with a dissociation constant (Kd) of 10⁻⁵ to 10⁻¹² mole/liter or less, and preferably 10⁻⁷ to 10⁻¹² moles/liter or less and more preferably 10⁻⁸ to 10⁻¹² moles/liter. Preferably, the amino acid sequences comprise or essentially consist of an immunoglobulin variable domain, which is a light chain variable domain, a heavy chain variable domain, a (single) domain antibody, a Nanobody®, or a humanized Nanobody. Amino acid sequences according to the invention comprising or essentially consisting of a Nanobody can comprise or consist of 4 framework regions (FR1 to FR4 respectively) and 3 complementarity determining regions (CDR1 to CDR3 respectively), in which:
   CDR1 is an amino acid sequence chosen from the group consisting of:

| | |
|---|---|
| SEQ ID NO: 167 | PYTMG |
| SEQ ID NO: 168 | DYAMS |
| SEQ ID NO: 169 | YYAIG |
| SEQ ID NO: 170 | INAMG |
| SEQ ID NO: 171 | IYTMG |
| SEQ ID NO: 172 | RLAMD |
| SEQ ID NO: 173 | RLAMD |
| SEQ ID NO: 174 | FNIMG |
| SEQ ID NO: 175 | FNIMG |
| SEQ ID NO: 176 | YYGVG |
| SEQ ID NO: 177 | YYGVG |
| SEQ ID NO: 178 | YYGVG |
| SEQ ID NO: 179 | DSAIG |
| SEQ ID NO: 180 | PYTIA |
| SEQ ID NO: 181 | PYTIG |
| SEQ ID NO: 182 | INVMN |
| SEQ ID NO: 183 | SYAMG |
| SEQ ID NO: 184 | PYTMG |
| SEQ ID NO: 185 | PYTVG |
| SEQ ID NO: 186 | PYTMG |
| SEQ ID NO: 187 | PYTMG |
| SEQ ID NO: 188 | PYTMG |
| SEQ ID NO: 189 | INPMG |
| SEQ ID NO: 190 | INPMG |
| SEQ ID NO: 191 | INPMA |
| SEQ ID NO: 192 | SYPMG |
| SEQ ID NO: 193 | SYPMG |
| SEQ ID NO: 194 | SYPMG |
| SEQ ID NO: 195 | SYPMG |
| SEQ ID NO: 196 | SYPMG |
| SEQ ID NO: 197 | SYPMG |
| SEQ ID NO: 198 | SFPMG |
| SEQ ID NO: 199 | SFPMG |
| SEQ ID NO: 200 | SFPMG |
| SEQ ID NO: 201 | AFPMG |
| SEQ ID NO: 202 | AFPMG |
| SEQ ID NO: 203 | AFPMG |
| SEQ ID NO: 204 | AFPMG |
| SEQ ID NO: 205 | AFPMG |
| SEQ ID NO: 206 | TYAMG |
| SEQ ID NO: 207 | NYHMV |
| SEQ ID NO: 208 | NYAMA |
| SEQ ID NO: 209 | IDAMA |
| SEQ ID NO: 210 | KHHATG |
| SEQ ID NO: 211 | SYVMG |
| SEQ ID NO: 212 | SYVMG |
| SEQ ID NO: 213 | SSPMG |
| SEQ ID NO: 214 | SSPMG |
| SEQ ID NO: 215 | SSPMG |
| SEQ ID NO: 216 | NGPMA |
| SEQ ID NO: 217 | SYPIA |

or from the group consisting of amino acid sequences that have at least 80%, preferably at least 90%, more preferably at least 95%, even more preferably at least 99% sequence identity (as defined herein) with one of the above amino acid sequences; in which
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s);
      and/or in which:
      CDR2 is an amino acid sequence chosen from the group consisting of:

| | |
|---|---|
| SEQ ID NO: 218 | RINWSGIRNYADSVKG |
| SEQ ID NO: 219 | AITGNGASKYYAESMKG |
| SEQ ID NO: 220 | CISSSVGTTYYSDSVKG |
| SEQ ID NO: 221 | DIMPYGSTEYADSVKG |
| SEQ ID NO: 222 | AAHWTVFRGNTYYVDSVKG |
| SEQ ID NO: 223 | SIAVSGTTMLDDSVKG |
| SEQ ID NO: 224 | SISRSGTTMAADSVKG |
| SEQ ID NO: 225 | DITNRGTTNYADSVKG |
| SEQ ID NO: 226 | DITNGGTTMYADSVKG |
| SEQ ID NO: 227 | CISSSDGDTYYADSVKG |
| SEQ ID NO: 228 | CISSSDGDTYYADSVKG |
| SEQ ID NO: 229 | CTSSSDGDTYYADSVKG |
| SEQ ID NO: 230 | CISSSDGDTYYDDSVKG |
| SEQ ID NO: 231 | TIIGSDRSTDLDGDTYYADSVRG |
| SEQ ID NO: 232 | TIIGSDRSTDLDGDTYYADSVRG |
| SEQ ID NO: 233 | AITSGGRKNYADSVKG |
| SEQ ID NO: 234 | AISSNGGSTRYADSVKG |
| SEQ ID NO: 235 | RINWSGIRNYADSVKG |
| SEQ ID NO: 236 | RINWSGIRNYADSVKG |
| SEQ ID NO: 237 | RINWSGIRNYADSVKG |
| SEQ ID NO: 238 | RINWSGITNYADSVKG |
| SEQ ID NO: 239 | RINWSGITNYADSVKG |
| SEQ ID NO: 240 | RIHGSITNYADSVKG |
| SEQ ID NO: 241 | RIHGSITNYADSVKG |
| SEQ ID NO: 242 | RIFGGGSTNYADSVKG |
| SEQ ID NO: 243 | GISQSGVGTAYSDSVKG |
| SEQ ID NO: 244 | GISQSGGSTAYSDSVKG |
| SEQ ID NO: 245 | GISQSSSSTAYSDSVKG |
| SEQ ID NO: 246 | GISQSGGSTAYSDSVKG |
| SEQ ID NO: 247 | GISQSGGSTAYSDSVKG |
| SEQ ID NO: 248 | GISQSGGSTAYSDSVKG |
| SEQ ID NO: 249 | GISQSGGSTHYSDSVKG |
| SEQ ID NO: 250 | GISQSGGSTHYSDSVKG |
| SEQ ID NO: 251 | GISQSGGSTHYSDSVKG |
| SEQ ID NO: 252 | GISQSGGSTHYSDSVKG |
| SEQ ID NO: 253 | GISQSGGSTHYSDSVKG |
| SEQ ID NO: 254 | GISQSGGSTHYSDSVKG |
| SEQ ID NO: 255 | GISQSGGSTHYSDSVKG |
| SEQ ID NO: 256 | GISQSGGSTHYSDSVKG |
| SEQ ID NO: 257 | AISWSGANTYYADSVKG |
| SEQ ID NO: 258 | AASGSTSSTYYADSVKG |
| SEQ ID NO: 259 | VISYAGGRTYYADSVKG |
| SEQ ID NO: 260 | TMNWSTGATYYADSVKG |
| SEQ ID NO: 261 | ALNWSGGNTYYTDSVKG |
| SEQ ID NO: 262 | TINWSGSNGYYADSVKG |
| SEQ ID NO: 263 | TINWSGSNKYYADSVKG |
| SEQ ID NO: 264 | AISGRSGNTYYADSVKG |
| SEQ ID NO: 265 | AISGRSGNTYYADSVKG |
| SEQ ID NO: 266 | AISGRSGNTYYADSVKG |
| SEQ ID NO: 267 | AISWRTGTTYYADSVKG |
| SEQ ID NO: 268 | AISWRGGNTYYADSVKG |

or from the group consisting of amino acid sequences that have at least 80%, preferably at least 90%, more preferably at least 95%, even more preferably at least 99% sequence identity (as defined herein) with one of the above amino acid sequences; in which
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s);
      and/or in which:
      CDR3 is an amino acid sequence chosen from the group consisting of:

| | |
|---|---|
| SEQ ID NO: 269 | ASQSGSGYDS |
| SEQ ID NO: 270 | VAKDTGSFYYPAYEHDV |
| SEQ ID NO: 271 | SSWFDCGVQGRDLGNEYDY |
| SEQ ID NO: 272 | YDPRGDDY |
| SEQ ID NO: 273 | TRSTAWNSPQRYDY |
| SEQ ID NO: 274 | FDGYTGSDY |
| SEQ ID NO: 275 | FDGYSGSDY |
| SEQ ID NO: 276 | YYPTTGFDD |
| SEQ ID NO: 277 | YYPTTGFDD |
| SEQ ID NO: 278 | DLSDYGVCSRWPSPYDY |
| SEQ ID NO: 279 | DLSDYGVCSRWPSPYDY |
| SEQ ID NO: 280 | DLSDYGVCSRWPSPYDY |
| SEQ ID NO: 281 | DLSDYGVCSKWPSPYDY |
| SEQ ID NO: 282 | TGKGYVFTPNEYDY |
| SEQ ID NO: 283 | TAKGYVFTDNEYDY |
| SEQ ID NO: 284 | DAPLASDDDVAPADY |
| SEQ ID NO: 285 | DETTGWVQLADFRS |
| SEQ ID NO: 286 | ASQSGSGYDS |
| SEQ ID NO: 287 | ASQSGSGYDS |
| SEQ ID NO: 288 | ASRSGSGYDS |
| SEQ ID NO: 289 | ASRSGSGYDS |
| SEQ ID NO: 290 | ASQVGSGYDS |
| SEQ ID NO: 291 | RRWGYDY |
| SEQ ID NO: 292 | RRWGYDY |
| SEQ ID NO: 293 | RRWGYDY |
| SEQ ID NO: 294 | RDKTLALRDYAYTTDVGYDD |
| SEQ ID NO: 295 | RDKTLALRDYAYTTDVGYDD |
| SEQ ID NO: 296 | RGRTLALRDYAYTTEVGYDD |
| SEQ ID NO: 297 | RGRTLFLRDYAYTTEVGYDD |
| SEQ ID NO: 298 | RGRTLFLRGYAYTTEVGYDD |
| SEQ ID NO: 299 | RGRTIALRNYAYTTEVGYDD |
| SEQ ID NO: 300 | RGRTLALRNYAYTTEVGYDD |
| SEQ ID NO: 301 | RGRTLALRNYAYTTEVGYDD |
| SEQ ID NO: 302 | RGRTLALRNYAYTTEVGYDD |
| SEQ ID NO: 303 | RGRTLALRNYAYTTEVGYDD |
| SEQ ID NO: 304 | RGRTLALRNYAYTTEVGYDD |
| SEQ ID NO: 305 | RGRTLALRNYAYTTEVGYDD |
| SEQ ID NO: 306 | RGRTLALRNYAYTTEVGYDD |
| SEQ ID NO: 307 | RGGTLALRNYAYTTEVGYDD |
| SEQ ID NO: 308 | SAIIEGFQDSIVIFSEAGYDY |
| SEQ ID NO: 309 | VAGLLLPRVAEGMDY |
| SEQ ID NO: 310 | VDSPLIATHPRGYDY |
| SEQ ID NO: 311 | ARGLLIATDARGYDY |
| SEQ ID NO: 312 | GSYVFYFTVRDQYDY |
| SEQ ID NO: 313 | SAGGFLVPRVGQGYDY |
| SEQ ID NO: 314 | SAGGFLVPRVGQGYDY |
| SEQ ID NO: 315 | ERVGLLLTVVAEGYDY |
| SEQ ID NO: 316 | ERVGLLLTVVAEGYDY |
| SEQ ID NO: 317 | ERVGLLLTVVAEGYDY |
| SEQ ID NO: 318 | ERVGLLLAVVAEGYDY |
| SEQ ID NO: 319 | ERAGVLLTKVPEGYDY |

or from the group consisting of amino acid sequences that have at least 80%, preferably at least 90%, more preferably at least 95%, even more preferably at least 99% sequence identity (as defined herein) with one of the above amino acid sequences; in which
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
   a) any amino acid substitution is preferably a conservative amino acid substitution (as defined herein); and/or
   b) said amino acid sequence preferably only contains amino acid substitutions, and no amino acid deletions or insertions, compared to the above amino acid sequence(s).

According to another specific aspect of the invention, the invention provides a number of stretches of amino acid residues (i.e. small peptides) that are particularly suited for binding to IL-6. These stretches of amino acid residues may be present in, and/or may be corporated into, an amino acid sequence of the invention, in particular in such a way that they form (part of) the antigen binding site of an amino acid sequence of the invention. As these stretches of amino acid residues were first generated as CDR sequences of heavy chain antibodies or V_{HH} sequences that were raised against IL-6 (or may be based on and/or derived from such CDR sequences, as further described herein), they will also generally be referred to herein as “CDR sequences” (i.e. as CDR1 sequences, CDR2 sequences and CDR3 sequences, respectively). It should however be noted that the invention in its broadest sense is not limited to a specific structural role or function that these stretches of amino acid residues may have in an amino acid sequence of the invention, as long as these stretches of amino acid residues allow the amino acid sequence of the invention to bind to IL-6. Thus, generally, the invention in its broadest sense comprises any amino acid sequence that is capable of binding to IL-6 and that comprises one or more CDR sequences as described herein, and in particular a suitable combination of two or more such CDR sequences, that are suitably linked to each other via one or more further amino acid sequences, such that the entire amino acid sequence forms a binding domain and/or binding unit that is capable of binding to IL-6. It should however also be noted that the presence of only one such CDR sequence in an amino acid sequence of the invention may by itself already be sufficient to provide an amino acid sequence of the invention that is capable of binding to IL-6; reference is for example again made to the so-called “Expedite fragments” described in WO 03/050531.

Thus, in another specific, but non-limiting aspect, the amino acid sequence of the invention may be an amino acid sequence that comprises at least one amino acid sequence that is chosen from the group consisting of the CDR1 sequences, CDR2 sequences and CDR3 sequences that are described herein (or any suitable combination thereof). In particular, an amino acid sequence of the invention may be an amino acid sequence that comprises at least one antigen binding site, wherein said antigen binding site comprises at least one amino acid sequence that is chosen from the group consisting of the CDR1 sequences, CDR2 sequences and CDR3 sequences that are described herein (or any suitable combination thereof).

Generally, in this aspect of the invention, the amino acid sequence of the invention may be any amino acid sequence that comprises at least one stretch of amino acid residues, in which said stretch of amino acid residues has an amino acid sequence that corresponds to the sequence of at least one of the CDR sequences described herein. Such an amino acid sequence may or may not comprise an immunoglobulin fold. For example, and without limitation, such an amino acid sequence may be a suitable fragment of an immunoglobulin sequence that comprises at least one such CDR sequence, but that is not large enough to form a (complete) immunoglobulin fold (reference is for example again made to the “Expedite fragments” described in WO 03/050531). Alternatively, such an amino acid sequence may be a suitable “protein scaffold” that comprises least one stretch of amino acid residues that corresponds to such a CDR sequence (i.e. as part of its antigen binding site). Suitable scaffolds for presenting amino acid sequences will be clear to the skilled person, and for example comprise, without limitation, to binding scaffolds based on or derived from immunoglobulins (i.e. other than the immunoglobulin sequences already described herein), protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9 (8):619-32).

Again, any amino acid sequence of the invention that comprises one or more of these CDR sequences is preferably such that it can specifically bind (as defined herein) to IL-6, and more in particular such that it can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein), that is as defined herein.

More in particular, the amino acid sequences according to this aspect of the invention may be any amino acid sequence that comprises at least one antigen binding site, wherein said antigen binding site comprises at least two amino acid sequences that are chosen from the group consisting of the CDR1 sequences described herein, the CDR2 sequences described herein and the CDR3 sequences described herein, such that (i) when the first amino acid sequence is chosen from the CDR1 sequences described herein, the second amino acid sequence is chosen from the CDR2 sequences described herein or the CDR3 sequences described herein; (ii) when the first amino acid sequence is chosen from the CDR2 sequences described herein, the second amino acid sequence is chosen from the CDR1 sequences described herein or the CDR3 sequences described herein; or (iii) when the first amino acid sequence is chosen from the CDR3 sequences described herein, the second amino acid sequence is chosen from the CDR1 sequences described herein or the CDR3 sequences described herein.

Even more in particular, the amino acid sequences of the invention may be amino acid sequences that comprise at least one antigen binding site, wherein said antigen binding site comprises at least three amino acid sequences that are chosen from the group consisting of the CDR1 sequences described herein, the CDR2 sequences described herein and the CDR3 sequences described herein, such that the first amino acid sequence is chosen from the CDR1 sequences described herein, the second amino acid sequence is chosen from the CDR2 sequences described herein, and the third amino acid sequence is chosen from the CDR3 sequences described herein. Preferred combinations of CDR1, CDR2 and CDR3 sequences will become clear from the further description herein. As will be clear to the skilled person, such an amino acid sequence is preferably an immunoglobulin sequence (as further described herein), but it may for example also be any other amino acid sequence that comprises a suitable scaffold for presenting said CDR sequences.

Thus, in one specific, but non-limiting aspect, the invention relates to an amino acid sequence directed against IL-6, that comprises one or more stretches of amino acid residues chosen from the group consisting of:
a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319;
   or any suitable combination thereof.

When an amino acid sequence of the invention contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
   and/or
ii) the amino acid sequence according to b) and/or c) preferably only contains amino acid substitutions, and no amino acid deletions or insertions, compared to the corresponding amino acid sequence according to a);
   and/or
iii) the amino acid sequence according to b) and/or c) may be an amino acid sequence that is derived from an amino acid sequence according to a) by means of affinity maturation using one or more techniques of affinity maturation known per se.

Similarly, when an amino acid sequence of the invention contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
   and/or
ii) the amino acid sequence according to e) and/or f) preferably only contains amino acid substitutions, and no amino acid deletions or insertions, compared to the corresponding amino acid sequence according to d);
   and/or
iii) the amino acid sequence according to e) and/or f) may be an amino acid sequence that is derived from an amino acid sequence according to d) by means of affinity maturation using one or more techniques of affinity maturation known per se.

Also, similarly, when an amino acid sequence of the invention contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
   and/or
ii) the amino acid sequence according to h) and/or i) preferably only contains amino acid substitutions, and no amino acid deletions or insertions, compared to the corresponding amino acid sequence according to g);
   and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any amino acid sequences of the invention that comprise one or more amino acid sequences according to b), c), e), f), h) or i), respectively.

In this specific aspect, the amino acid sequence preferably comprises one or more stretches of amino acid residues chosen from the group consisting of:
i) the amino acid sequences of SEQ ID NO's: 167 to 217;
ii) the amino acid sequences of SEQ ID NO's: 218 to 268; and
iii) the amino acid sequences of SEQ ID NO's: 269 to 319;
   or any suitable combination thereof.

Also, preferably, in such an amino acid sequence, at least one of said stretches of amino acid residues forms part of the antigen binding site for binding against IL-6.

In a more specific, but again non-limiting aspect, the invention relates to an amino acid sequence directed against IL-6, that comprises two or more stretches of amino acid residues chosen from the group consisting of:
a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319;
   such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), e) or f).

In this specific aspect, the amino acid sequence preferably comprises two or more stretches of amino acid residues chosen from the group consisting of:
i) the amino acid sequences of SEQ ID NO's: 167 to 217;
ii) the amino acid sequences of SEQ ID NO's: 218 to 268; and
iii) the amino acid sequences of SEQ ID NO's: 269 to 319;
   such that, (i) when the first stretch of amino acid residues corresponds to one of the amino acid sequences of SEQ ID NO's: 167 to 217, the second stretch of amino acid residues corresponds to one of the amino acid sequences of SEQ ID NO's: 218 to 268 or of SEQ ID NO's: 269 to 319; (ii) when the first stretch of amino acid residues corresponds to one of the amino acid sequences of SEQ ID NO's: 218 to 268, the second stretch of amino acid residues corresponds to one of the amino acid sequences of SEQ ID NO's: 167 to 217 or of SEQ ID NO's: 269 to 319; or (iii) when the first stretch of amino acid residues corresponds to one of the amino acid sequences of SEQ ID NO's: 269 to 319, the second stretch of amino acid residues corresponds to one of the amino acid sequences of SEQ ID NO's: 167 to 217 or of SEQ ID NO's: 218 to 268.

Also, in such an amino acid sequence, the at least two stretches of amino acid residues again preferably form part of the antigen binding site for binding against IL-6.

In an even more specific, but non-limiting aspect, the invention relates to an amino acid sequence directed against IL-6, that comprises three or more stretches of amino acid residues, in which the first stretch of amino acid residues is chosen from the group consisting of:
a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
   the second stretch of amino acid residues is chosen from the group consisting of:
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
   and the third stretch of amino acid residues is chosen from the group consisting of:
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319.

Preferably, in this specific aspect, the first stretch of amino acid residues is chosen from the group consisting of the amino acid sequences of SEQ ID NO's: 167 to 217; the second stretch of amino acid residues is chosen from the group consisting of the amino acid sequences of SEQ ID NO's: 218 to 268; and the third stretch of amino acid residues is chosen from the group consisting of the amino acid sequences of SEQ ID NO's: 269 to 319.

Again, preferably, in such an amino acid sequence, the at least three stretches of amino acid residues forms part of the antigen binding site for binding against IL-6.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 320 to 370. This degree of amino acid identity can for example be determined by determining the degree of amino acid identity (in a manner described herein) between said amino acid sequence and one or more of the sequences of SEQ ID NO's: 320 to 370, in which the amino acid residues that form the framework regions are disregarded. Also, such amino acid sequences of the invention can be as further described herein.

Also, such amino acid sequences are preferably such that they can specifically bind (as defined herein) to IL-6; and more in particular bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
   and/or

CDR2 is chosen from the group consisting of:
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
   and/or

CDR3 is chosen from the group consisting of:
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319.

In particular, such an amino acid sequence of the invention may be such that CDR1 is chosen from the group consisting of the amino acid sequences of SEQ ID NO's: 167 to 217; and/or CDR2 is chosen from the group consisting of the amino acid sequences of SEQ ID NO's: 218 to 268; and/or CDR3 is chosen from the group consisting of the amino acid sequences of SEQ ID NO's: 269 to 319.

In particular, when the amino acid sequence of the invention essentially consists of 4 framework regions (FR1 to FR4, respectively) and 3 complementarity determining regions (CDR1 to CDR3, respectively), the amino acid sequence of the invention is preferably such that:

CDR1 is chosen from the group consisting of:
a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
   and

CDR2 is chosen from the group consisting of:
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
   and

CDR3 is chosen from the group consisting of:
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319; or any suitable fragment of such an amino acid sequence

In particular, such an amino acid sequence of the invention may be such that CDR1 is chosen from the group consisting of the amino acid sequences of SEQ ID NO's: 167 to 217; and CDR2 is chosen from the group consisting of the amino acid sequences of SEQ ID NO's: 218 to 268; and CDR3 is chosen from the group consisting of the amino acid sequences of SEQ ID NO's: 269 to 319.

Again, preferred combinations of CDR sequences will become clear from the further description herein.

Also, such amino acid sequences are preferably such that they can specifically bind (as defined herein) to IL-6; and more in particular bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 320 to 370. This degree of amino acid identity can for example be determined by determining the degree of amino acid identity (in a manner described herein) between said amino acid sequence and one or more of the sequences of SEQ ID NO's: 320 to 370, in which the amino acid residues that form the framework regions are disregarded. Such amino acid sequences of the invention can be as further described herein.

In such an amino acid sequence of the invention, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

The framework sequences are preferably such that the amino acid sequence of the invention is a domain antibody (or an amino acid sequence that is suitable for use as a domain antibody); is a single domain antibody (or an amino acid sequence that is suitable for use as a single domain antibody); is a “dAb” (or an amino acid sequence that is suitable for use as a dAb); or is a Nanobody™ (including but not limited to V_{HH} sequence). Again, suitable framework sequences will be clear to the skilled person, for example on the basis the standard handbooks and the further disclosure and prior art mentioned herein.

In particular, the framework sequences present in the amino acid sequences of the invention may contain one or more of Hallmark residues (as defined herein), such that the amino acid sequence of the invention is a Nanobody™. Some preferred, but non-limiting examples of (suitable combinations of) such framework sequences will become clear from the further disclosure herein.

Again, as generally described herein for the amino acid sequences of the invention, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these “Expedite fragments”, reference is again made to WO 03/050531, as well as to the US provisional application of Ablynx N.V. entitled “Peptides capable of binding to serum proteins” of Ablynx N.V. (inventors: Revets, Hilde Adi Pierrette; Kolkman, Joost Alexander; and Hoogenboom, Hendricus Renerus Jacobus Mattheus) filed on Dec. 5, 2006.

In another aspect, the invention relates to a compound or construct, and in particular a protein or polypeptide (also referred to herein as a “compound of the invention” or “polypeptide of the invention”, respectively) that comprises or essentially consists of one or more amino acid sequences and/or Nanobodies of the invention (or suitable fragments thereof), and optionally further comprises one or more other groups, residues, moieties or binding units. As will become clear to the skilled person from the further disclosure herein, such further groups, residues, moieties, binding units or Nanobodies may or may not provide further functionality to the amino acid sequence and/or Nanobody of the invention (and/or to the compound or construct in which it is present) and may or may not modify the properties of the amino acid sequence and/or Nanobody of the invention.

For example, such further groups, residues, moieties or binding units may be one or more additional amino acid sequences and/or Nanobodies, such that the compound or construct is a (fusion) protein or (fusion) polypeptide. In a preferred but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more amino acid sequences of the invention so as to provide a “derivative” of an amino acid sequence or polypeptide of the invention, as further described herein.

Also within the scope of the present invention are compounds or constructs, that comprises or essentially consists of one or more derivatives as described herein, and optionally further comprises one or more other groups, residues, moieties or binding units, optionally linked via one or more linkers. Preferably, said one or more other groups, residues, moieties or binding units are amino acid sequences.

In such a compound or construct, the one or more amino acid sequences and/or Nanobodies of the invention and the one or more groups, residues, moieties or binding units may be linked to directly to each other and/or via one or more suitable linkers or spacers. For example, when the one or more groups, residues, moieties or binding units are Nanobodies, the linkers may also be amino acid sequences and/or Nanobodies, so that the resulting compound or construct is a fusion (protein) or fusion (polypeptide).

The compounds or polypeptides of the invention can generally be prepared by a method which comprises at least one step of suitably linking the one or more amino acid sequences and/or Nanobodies of the invention to the one or more further groups, residues, moieties or binding units, optionally via the one or more suitable linkers, so as to provide the compound or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein.

The process of designing/selecting and/or preparing a compound or polypeptide of the invention, starting from an amino acid sequence and/or Nanobody of the invention, is also referred to herein as “formatting” said amino acid sequence and/or Nanobody of the invention; and an amino acid of the invention that is made part of a compound or polypeptide of the invention is said to be “formatted” or to be “in the format of” said compound or polypeptide of the invention. Examples of ways in which an amino acid sequence and/or Nanobody of the invention can be formatted and examples of such formats will be clear to the skilled person based on the disclosure herein; and such formatted amino acid sequences and/or Nanobodies form a further aspect of the invention.

In one specific aspect of the invention, a compound of the invention, a Nanobody of the invention or a polypeptide of the invention may have an increased half-life, compared to the corresponding amino acid sequence and/or Nanobody of the invention. Some preferred, but non-limiting examples of such compounds and polypeptides will become clear to the skilled person based on the further disclosure herein, and for example comprise amino acid sequences and/or Nanobodies or polypeptides of the invention that have been chemically modified to increase the half-life thereof (for example, by means of pegylation); amino acid sequences and/or Nanobodies of the invention that comprise at least one additional binding site for binding to a serum protein (such as serum albumin); or polypeptides of the invention that comprise at least one amino acid sequence and/or Nanobody of the invention that is linked to at least one moiety (and in particular at least one amino acid sequence and/or Nanobody) that increases the half-life of the amino acid sequence and/or Nanobody of the invention. Examples of polypeptides of the invention that comprise such half-life extending moieties or amino acid sequences and/or Nanobodies will become clear to the skilled person based on the further disclosure herein; and for example include, without limitation, polypeptides in which the one or more amino acid sequences and/or Nanobodies of the invention are suitable linked to one or more serum proteins or fragments thereof (such as serum albumin or suitable fragments thereof) or to one or more binding units that can bind to serum proteins (such as, for example, domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies that can bind to serum proteins such as serum albumin (such as human serum albumin), serum immunoglobulins such as IgG, or transferrine; reference is made to the further description and references mentioned herein); polypeptides in which an amino acid sequence and/or Nanobody of the invention is linked to an Fc portion (such as a human Fc) or a suitable part or fragment thereof; or polypeptides in which the one or more amino acid sequences and/or Nanobodies of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006).

Generally, the compounds or polypeptides of the invention with increased half-life preferably have a half-life that is at least 1.5 times, preferably at least 2 times, such as at least 5 times, for example at least 10 times or more than 20 times, greater than the half-life of the corresponding amino acid sequence and/or Nanobody of the invention per se. For example, the compounds or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence and/or Nanobody of the invention per se.

In a preferred, but non-limiting aspect of the invention, such compounds or polypeptides of the invention have a serum half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In another preferred, but non-limiting aspect of the invention, such compounds or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), at preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

Generally, proteins or polypeptides that comprise or essentially consist of a single amino acid sequence and/or Nanobody (such as a single amino acid sequence and/or Nanobody of the invention) will be referred to herein as “monovalent” proteins or polypeptides or as “monovalent constructs”. Proteins and polypeptides that comprise or essentially consist of two or more amino acid sequences and/or Nanobodies (such as at least two amino acid sequences and/or Nanobodies of the invention or at least one amino acid sequence and/or Nanobody of the Invention and at least one other amino acid sequence and/or Nanobody) will be referred to herein as “multivalent” proteins or polypeptides or as “multivalent constructs”, and these may provide certain advantages compared to the corresponding monovalent amino acid sequences and/or Nanobodies of the invention. Some non-limiting examples of such multivalent constructs will become clear from the further description herein.

According to one specific, but non-limiting embodiment, a polypeptide of the invention comprises or essentially consists of at least two amino acid sequences and/or Nanobodies of the invention, such as two or three amino acid sequences and/or Nanobodies of the invention. As further described herein, such multivalent constructs can provide certain advantages compared to a protein or polypeptide comprising or essentially consisting of a single amino acid sequence and/or Nanobody of the invention, such as a much improved affinity and/or specificity for IL-6. As mentioned above, in such multivalent polypeptides of the invention, the amino acid sequences and/or Nanobodies may be directed against the same epitopes/binding sites or against different epitopes/binding sites.

According to another specific, but non-limiting embodiment, a polypeptide of the invention comprises or essentially consists of at least one amino acid sequence and/or Nanobody of the invention and at least one other amino acid sequence and/or Nanobody (i.e. directed against another epitope, antigen, target, protein or polypeptide). Such proteins or polypeptides are also referred to herein as “multispecific” proteins or polypeptides or as “multispecific constructs”, and these may provide certain advantages compared to the corresponding monovalent amino acid sequences and/or Nanobodies of the invention. Again, some non-limiting examples of such multispecific constructs will become clear from the further description herein.

According to yet another specific, but non-limiting embodiment, a polypeptide of the invention comprises or essentially consists of at least one amino acid sequence and/or Nanobody of the invention, optionally one or more further amino acid sequences and/or Nanobodies, and at least one other amino acid sequence (such as a protein or polypeptide) that confers at least one desired property to the amino acid sequence and/or Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

According to another embodiment of the invention, the polypeptides of the invention comprise at least one binding site (e.g. a binding unit such as an amino acid sequence and/or Nanobody) directed against IL-6, at least one binding site (e.g. a binding unit such as an amino acid sequence and/or Nanobody) directed against TNF-alpha, and optionally at least one binding site (e.g. a binding unit such as an amino acid sequence and/or Nanobody) that provides for increased half-life (such as an amino acid sequence and/or Nanobody directed against a serum protein such as IgG or serum albumin), optionally linked via one or more suitable linkers. For this purpose, for example, the Nanobodies described in the international application WO 04/041862 of applicant or in the non-prepublished U.S. provisional application 60/682,332 by applicant (filing date May 18, 2005) may be used in the polypeptides of the invention. SEQ ID NO's 419 to 447 provide some non-limiting examples of such bispecific and trispecific constructs.

Thus, another embodiment of the invention relates to a polypeptide comprising at least one domain antibody or single domain antibody against IL-6, least one domain antibody or single domain antibody against TNF-alpha, and optionally one or more further binding domains or amino acid sequences, optionally linked via one or more suitable linkers.

It is also possible to combine two or more of the above embodiments, for example to provide a trivalent bispecific construct comprising two amino acid sequences and/or Nanobodies of the invention and one other amino acid sequence and/or Nanobody, and optionally one or more other amino acid sequences. Further non-limiting examples of such constructs, as well as some constructs that are particularly preferred within the context of the present invention, will become clear from the further description herein.

In the above constructs, the one or more amino acid sequences and/or Nanobodies and/or other amino acid sequences may be directly linked or linked via one or more linker sequences. Some suitable but non-limiting examples of such linkers will become clear from the further description herein.

Preferably, a polypeptide of the invention either comprises two or three amino acid sequences and/or Nanobodies of the invention, optionally linked via one or two linkers, or is a multispecific polypeptide, comprising one or two, and preferably two, amino acid sequences and/or Nanobodies of the invention and at least one amino acid sequence and/or Nanobody that provides an increased half-life (such as a amino acid sequence and/or Nanobody directed against a serum protein, and in particular against a human serum protein, such as against human serum albumin), in which said amino acid sequences and/or Nanobodies again optionally linked via one or more linkers.

In one preferred embodiment of the invention, a polypeptide of the invention comprises one or more (such as two or preferably one) amino acid sequences and/or Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) amino acid sequences and/or Nanobodies, such as the amino acid sequences and/or Nanobodies described in WO 02/057445, of which FC44 (SEQ ID NO: 160) and FC5 (SEQ ID NO: 161) are some preferred non-limiting examples.

In another preferred embodiment of the invention, a polypeptide of the invention comprises one or more (such as two or preferably one) amino acid sequences and/or Nanobodies of the invention linked (optionally via one or more suitable linker sequences) to one or more (such as two and preferably one) amino acid sequences that confer an increased half-life in vivo to the resulting polypeptide of the invention. In particular, said amino acid sequences that confer an increased half-life in vivo to the resulting polypeptide of the invention may be one or more (such as two and preferably one) amino acid sequences and/or Nanobodies, and in particular amino acid sequences and/or Nanobodies directed against a human serum protein such as human serum albumin, of which PMP6A6 (“ALB-1”, SEQ ID NO: 157), ALB-8 (a humanized version of AlB-1, SEQ ID NO: 158) and PMP6A8 (“ALB-2”, SEQ ID NO: 159) are some preferred non-limiting examples. Other examples of suitable amino acid sequences and/or Nanobodies against mouse or human serum albumin are described in the applications by applicant referred to below.

In yet another preferred embodiment of the invention, a polypeptide of the invention comprises one or more (such as two or preferably one) amino acid sequences and/or Nanobodies of the invention, one or more (such as two and preferably one) amino acid sequences that allow the resulting polypeptide of the invention to cross the blood brain barrier, and one or more (such as two and preferably one) amino acid sequences that confer an increased half-life in vivo to the resulting polypeptide of the invention (optionally linked via one or more suitable linker sequences). Again, said one or more amino acid sequences that allow the resulting polypeptides of the invention to cross the blood brain barrier may be one or more (such as two and preferably one) amino acid sequences and/or Nanobodies (as mentioned herein), and said amino acid sequences that confer an increased half-life in vivo to the resulting polypeptide of the invention may be one or more (such as two and preferably one) amino acid sequences and/or Nanobodies (also as mentioned herein).

More in particular, the invention provides amino acid sequences and/or Nanobodies can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence and/or Nanobody.

In particular, Nanobodies, amino acid sequences and/or and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent Nanobody of the invention (or a polypeptide that contains only one amino acid sequence and/or Nanobody of the invention) is preferably such that it will bind to IL-6 with an affinity less than 500 nM, preferably less than 200 nM, more preferably less than 10 nM, such as less than 500 pM.

Some preferred IC₅₀ values for binding of the amino acid sequences and/or Nanobodies or polypeptides of the invention to IL-6 will become clear from the further description and examples herein.

The affinity of the polypeptide of the invention against IL-6 can be determined in a manner known per se, for example using the assay described herein.

Some preferred, but non-limiting examples of polypeptides of the invention are the polypeptides of SEQ ID NO's: 371 to 447, in which:
SEQ ID NO's: 371 to 390 are some non-limiting examples of multivalent (and in particular bivalent) polypeptides of the invention;
SEQ ID NO's: 391 to 418 are some non-limiting examples of bispecific polypeptides of the invention, comprising one or two amino acid sequences and/or Nanobodies of the invention and an amino acid sequence and/or Nanobody directed against human serum albumin;
SEQ ID NO's: 419 to 438 are some examples of bispecific polypeptides of the invention, comprising one or two amino acid sequences and/or Nanobodies of the invention and an amino acid sequence and/or Nanobody against TNF; and
SEQ ID NO's: 439 to 447 are some examples of trispecific polypeptides of the invention, comprising one or two amino acid sequences and/or Nanobodies of the invention, an amino acid sequence and/or Nanobody directed against human serum albumin, and an amino acid sequences and/or Nanobody against TNF.

Other polypeptides of the invention may for example be chosen from the group consisting of amino acid sequences that have more than 80%, preferably more than 90%, more preferably more than 95%, such as 99% or more “sequence identity” (as defined herein) with one or more of the amino acid sequences of SEQ ID NO's: 371 to 447, in which the amino acid sequences and/or Nanobodies comprised within said amino acid sequences are preferably as defined herein.

In another aspect, the invention relates to a nucleic acid that encodes an amino acid sequence and/or Nanobody of the invention and/or a polypeptide of the invention. Such a nucleic acid will also be referred to herein as a “nucleic acid of the invention” and may for example be in the form of a genetic construct, as defined herein.

In another aspect, the invention relates to host or host cell that expresses or that is capable of expressing an amino acid sequence and/or Nanobody of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one amino acid sequence and/or Nanobody of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention also relates to the use of an amino acid sequence, Nanobody or polypeptide of the invention, or of a composition comprising the same, in (methods or compositions for) modulating IL-6, either in vitro (e.g. in an in vitro or cellular assay) or in vivo (e.g. in an a single cell or in a multicellular organism, and in particular in a mammal, and more in particular in a human being, such as in a human being that is at risk of or suffers from a disease and/or disorder associated with IL-6-mediated signalling).

The invention also relates to methods for modulating IL-6, either in vitro (e.g. in an in vitro or cellular assay) or in vivo (e.g. in an a single cell or multicellular organism, and in particular in a mammal, and more in particular in a human being, such as in a human being that is at risk of or suffers from a disease and/or disorder associated with IL-6-mediated signalling), which method comprises at least the step of contacting IL-6 with at least one amino acid sequence, Nanobody or polypeptide of the invention, or with a composition comprising the same, in a manner and in an amount suitable to modulate IL-6, with at least one amino acid sequence, Nanobody or polypeptide of the invention.

The invention also relates to the use of an one amino acid sequence, Nanobody or polypeptide of the invention in the preparation of a composition (such as, without limitation, a pharmaceutical composition or preparation as further described herein) for modulating IL-6, either in vitro (e.g. in an in vitro or cellular assay) or in vivo (e.g. in an a single cell or multicellular organism, and in particular in a mammal, and more in particular in a human being, such as in a human being that is at risk of or suffers from a disease and/or disorder associated with IL-6-mediated signalling).

In the context of the present invention, “modulating” or “to modulate” generally means either reducing or inhibiting the activity of, or alternatively increasing the activity of, IL-6, as measured using a suitable in vitro, cellular or in vivo assay (such as those mentioned herein). In particular, “modulating” or “to modulate” may mean either reducing or inhibiting the activity of, or alternatively increasing the activity of, IL-6, as measured using a suitable in vitro, cellular or in vivo assay (such as those mentioned herein), by at least 1%, preferably at least 5%, such as at least 10% or at least 25%, for example by at least 50%, at least 60%, at least 70%, at least 80%, or 90% or more, compared to activity of IL-6 in the same assay under the same conditions but without the presence of the amino acid sequence, Nanobody or polypeptide of the invention.

As will be clear to the skilled person, “modulating” may also involve effecting a change (which may either be an increase or a decrease) in affinity, avidity, specificity and/or selectivity of IL-6 for one or more of its targets, ligands or substrates; and/or effecting a change (which may either be an increase or a decrease) in the sensitivity of IL-6 for one or more conditions in the medium or surroundings in which IL-6 is present (such as pH, ion strength, the presence of co-factors, etc.), compared to the same conditions but without the presence of the amino acid sequence, Nanobody or polypeptide of the invention. As will be clear to the skilled person, this may again be determined in any suitable manner and/or using any suitable assay known per se, such as the assays described herein or in the prior art cited herein.

“Modulating” may also mean effecting a change (i.e. an activity as an agonist or as an antagonist, respectively) with respect to one or more biological or physiological mechanisms, effects, responses, functions, pathways or activities in which IL-6 (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, such as the assays described herein or in the prior art cited herein. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the amino acid sequence, Nanobody or polypeptide of the invention.

Modulating may for example involve reducing or inhibiting the binding of IL-6 to one of its substrates or ligands and/or competing with a natural ligand, substrate for binding to IL-6. Modulating may also involve activating IL-6 or the mechanism or pathway in which it is involved. Modulating may be reversible or irreversible, but for pharmaceutical and pharmacological purposes will usually be in a reversible manner.

The invention further relates to methods for preparing or generating the amino acid sequences, polypeptides, nucleic acids, host cells, products and compositions described herein. Some preferred but non-limiting examples of such methods will become clear from the further description herein.

Generally, these methods may comprise the steps of:
a) providing a set, collection or library of amino acid sequences; and
b) screening said set, collection or library of amino acid sequences for amino acid sequences that can bind to and/or have affinity for IL-6;
   and
c) isolating the amino acid sequence(s) that can bind to and/or have affinity for IL-6.

In such a method, the set, collection or library of amino acid sequences may be any suitable set, collection or library of amino acid sequences. For example, the set, collection or library of amino acid sequences may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of amino acid sequences may be a set, collection or library of heavy chain variable domains (such as V_{H} domains or V_{HH} domains) or of light chain variable domains. For example, the set, collection or library of amino acid sequences may be a set, collection or library of domain antibodies or single domain antibodies, or may be a set, collection or library of amino acid sequences that are capable of functioning as a domain antibody or single domain antibody.

In a preferred aspect of this method, the set, collection or library of amino acid sequences may be an immune set, collection or library of immunoglobulin sequences, for example derived from a mammal that has been suitably immunized with IL-6 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In another aspect, the method for generating amino acid sequences comprises at least the steps of:
a) providing a collection or sample of cells expressing amino acid sequences;
b) screening said collection or sample of cells for cells that express an amino acid sequence that can bind to and/or have affinity for IL-6;
   and
c) either (i) isolating said amino acid sequence; or (ii) isolating from said cell a nucleic acid sequence that encodes said amino acid sequence, followed by expressing said amino acid sequence.

For example, when the desired amino acid sequence is an immunoglobulin sequence, the collection or sample of cells may for example be a collection or sample of B-cells. Also, in this method, the sample of cells may be derived from a mammal that has been suitably immunized with IL-6 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820 (2001).

In another aspect, the method for generating an amino acid sequence directed against IL-6 may comprise at least the steps of:
a) providing a set, collection or library of nucleic acid sequences encoding amino acid sequences;
b) screening said set, collection or library of nucleic acid sequences for nucleic acid sequences that encode an amino acid sequence that can bind to and/or has affinity for IL-6;
   and
c) isolating said nucleic acid sequence, followed by expressing said amino acid sequence.

In such a method, the set, collection or library of nucleic acid sequences encoding amino acid sequences may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

Also, in such a method, the set, collection or library of nucleic acid sequences may encode a set, collection or library of heavy chain variable domains (such as V_{H} domains or V_{HH} domains) or of light chain variable domains. For example, the set, collection or library of nucleic acid sequences may encode a set, collection or library of domain antibodies or single domain antibodies, or a set, collection or library of amino acid sequences that are capable of functioning as a domain antibody or single domain antibody.

In a preferred aspect of this method, the set, collection or library of amino acid sequences may be an immune set, collection or library of nucleic acid sequences, for example derived from a mammal that has been suitably immunized with IL-6 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The set, collection or library of nucleic acid sequences may for example encode an immune set, collection or library of heavy chain variable domains or of light chain variable domains. In one specific aspect, the set, collection or library of nucleotide sequences may encode a set, collection or library of V_{HH} sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to amino acid sequences that are obtained by the above methods, or alternatively by a method that comprises the one of the above methods and in addition at least the steps of determining the nucleotide sequence or amino acid sequence of said immunoglobulin sequence; and of expressing or synthesizing said amino acid sequence in a manner known per se, such as by expression in a suitable host cell or host organism or by chemical synthesis.

Also, following the steps above, one or more amino acid sequences of the invention may be suitably humanized (or alternatively camelized); and/or the amino acid sequence(s) thus obtained may be linked to each other or to one or more other suitable amino acid sequences (optionally via one or more suitable linkers) so as to provide a polypeptide of the invention. Also, a nucleic acid sequence encoding an amino acid sequence of the invention may be suitably humanized (or alternatively camelized) and suitably expressed; and/or one or more nucleic acid sequences encoding an amino acid sequence of the invention may be linked to each other or to one or more nucleic acid sequences that encode other suitable amino acid sequences (optionally via nucleotide sequences that encode one or more suitable linkers), after which the nucleotide sequence thus obtained may be suitably expressed so as to provide a polypeptide of the invention.

The invention further relates to applications and uses of the amino acid sequences and/or Nanobodies, polypeptides, nucleic acids, host cells, products and compositions described herein, as well as to methods for the prevention and/or treatment for diseases and disorders associated with IL-6. Some preferred but non-limiting applications and uses will become clear from the further description herein.

Other aspects, embodiments, advantages and applications of the invention will also become clear from the further description herein, in which the invention will be described and discussed in more detail with reference to the Nanobodies of the invention and polypeptides of the invention comprising the same, which form some of the preferred aspects of the invention.

As will become clear from the further description herein, Nanobodies generally offer certain advantages (outlined herein) compared to “dAb's” or similar (single) domain antibodies or immunoglobulin sequences, which advantages are also provided by the Nanobodies of the invention. However, it will be clear to the skilled person that the more general aspects of the teaching below can also be applied (either directly or analogously) to other amino acid sequences of the invention.

### DETAILED DESCRIPTION OF THE INVENTION

In the present description, examples and claims:
a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
b) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2 (1): 49-57; Irving et al., J. Immunuol. Methods, 200, 248 (1-2), 3′-45; Schmitz et al., Placenta, 2000, 21 Suppl. A, S106-12, Gonzales et al., Tumour Biol., 2005, 26 (1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code, as mentioned in Table A-2;
   1

TABLE A-2
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residu can generally be considered essentially uncharged at a pH of about 6.5.

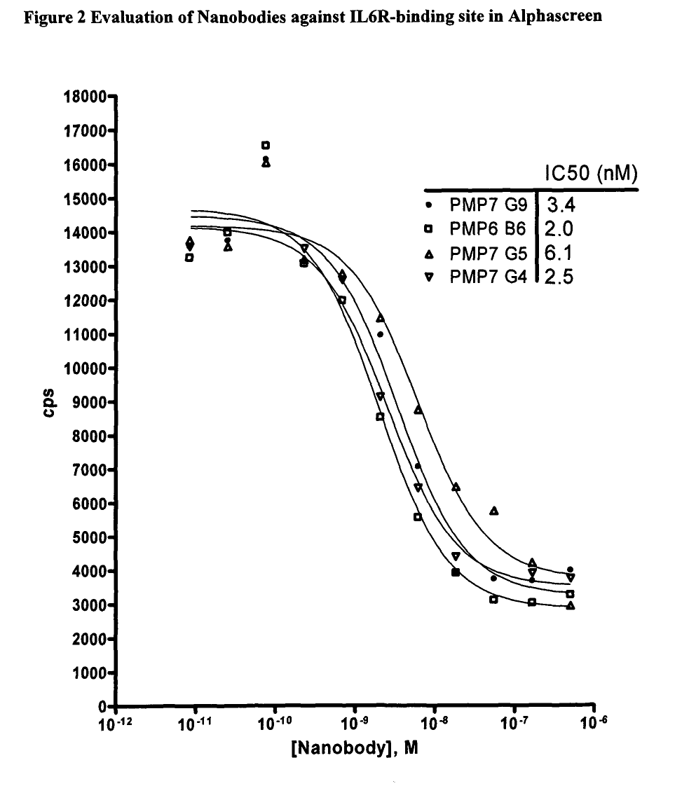

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).
   Alternatively, the degree of sequence identity between two or more nucleotide sequences may be calculated using a known computer algorithm for sequence alignment such as NCBI Blast v2.0, using standard settings.
   Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.
   Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence (also referred to herein as “amino acid sequence identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.
   Alternatively, the degree of sequence identity between two amino acid sequences may be calculated using a known computer algorithm, such as those mentioned above for determining the degree of sequence identity for nucleotide sequences, again using standard settings.
   Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.
   Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.
   Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when a Nanobody of the invention is said to comprise a CDR sequence, this may mean that said CDR sequence has been incorporated into the Nanobody of the invention, but more usually this generally means that the Nanobody of the invention contains within its sequence a stretch of amino acid residues with the same amino acid sequence as said CDR sequence, irrespective of how said Nanobody of the invention has been generated or obtained. It should also be noted that when the latter amino acid sequence has a specific biological or structural function, it preferably has essentially the same, a similar or an equivalent biological or structural function in the first mentioned amino acid sequence (in other words, the first mentioned amino acid sequence is preferably such that the latter sequence is capable of performing essentially the same, a similar or an equivalent biological or structural function). For example, when a Nanobody of the invention is said to comprise a CDR sequence or framework sequence, respectively, the CDR sequence and framework are preferably capable, in said Nanobody, of functioning as a CDR sequence or framework sequence, respectively. Also, when a nucleotide sequence is said to comprise another nucleotide sequence, the first mentioned nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first mentioned, larger nucleotide sequence).
j) A nucleic acid sequence or amino acid sequence is considered to be “(in) essentially isolated form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
k) The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds. The term “binding domain” refers to such a domain that is directed against an antigenic determinant (as defined herein);
l) The term ‘antigenic determinant’ refers to the epitope on the antigen recognized by the antigen-binding molecule (such as a Nanobody or a polypeptide of the invention) and more in particular by the antigen-binding site of said molecule. The terms “antigenic determinant” and “epitope’ may also be used interchangeably herein.
m) An amino acid sequence (such as a Nanobody, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
n) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the amino acid sequences, Nanobodies and/or polypeptides of the invention) will bind to their antigen with a dissociation constant (K_{D}) of 10⁻⁵ to 10⁻¹² mole/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired serum protein with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.
   The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e,g, of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].
   The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as an amino acid sequence, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RT·ln(K_{D}) (equivalently DG=−RT·ln(K_{A})), where R equals the gas constant, T equals the absolute temperature and In denotes the natural logarithm.
   The K_{D} for biological interactions which are considered meaningful (e.g. specific) are typically in the range of 10⁻¹⁰ M (0.1 nM) to 10⁻⁵M (10000 nM). The stronger an interaction is, the lower is its K_{D}.
   The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1 s⁻¹ (t_{1/2}=0.69 s).
   The affinity of a molecular interaction between two molecules can be measured via different techniques known per se, such as the well the known surface plasmon resonance (SPR) biosensor technique (se for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments.
   It will also be clear to the skilled person that the measured K_{D} may correspond to the apparent K_{D} if the measuring process somehow influences the intrinsic binding affinity of the implied molecules for example by artifacts related to the coating on the biosensor of one molecule. Also, an apparent K_{D} may be measured if one molecule contains more than one recognition sites for the other molecule. In such situation the measured affinity may be affected by the avidity of the interaction by the two molecules.
   Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artifacts relating to adsorption of one of the molecules on a support such as plastic.
   However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance. Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labeled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of B is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if cref<<K_{D} ref, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.
o) The half-life of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomologus monkeys (Macaca fascicularis) and/or rhesus monkeys (Macaca mulatta)) and baboon (Papio ursinus)) a suitable dose of the amino acid sequence, compound or polypeptide of the invention; collecting blood samples or other samples from said animal; determining the level or concentration of the amino acid sequence, compound or polypeptide of this aspect in said blood sample; and calculating, from (a plot of) the data thus obtained, the time until the level or concentration of the amino acid sequence, compound or polypeptide of the invention has been reduced by 50% compared to the initial level upon dosing. Reference is for example made to the Experimental Part below, as well as to the standard handbooks, such as Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2nd Rev. edition (1982).
   As will also be clear to the skilled person (see for example pages 6 and 7 of WO 04/003019 and in the further references cited therein), the half-life can be expressed using parameters such as the t1/2-alpha, t1/2-beta and the area under the curve (AUC). In the present specification, an “increase in half-life” refers to an increase in any one of these parameters, such as any two of these parameters, or essentially all three these parameters. As used herein “increase in half-life” or “increased half-life” in particular refers to an increase in the t1/2-beta, either with or without an increase in the t1/2-alpha and/or the AUC or both.
p) As also further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
q) The amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to herein (see for example FIG. 2 of said reference). According to this numbering, FR1 of a Nanobody comprises the amino acid residues at positions 1-30, CDR1 of a Nanobody comprises the amino acid residues at positions 31-35, FR2 of a Nanobody comprises the amino acids at positions 3649, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].
   Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
r) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the prior art cited herein, to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference.

In accordance with the terminology used in the above references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spatial conformation and configuration (i.e. through the use of especially designed linkers, as with scFv's);
V_{HH} domains and Nanobodies can be expressed from a single gene and require no post-translational folding or modifications;
V_{HH} domains and Nanobodies can easily be engineered into multivalent and multispecific formats (as further discussed herein);
V_{HH} domains and Nanobodies are highly soluble and do not have a tendency to aggregate (as with the mouse-derived antigen-binding domains described by Ward et al., Nature, Vol. 341, 1989, p. 544);
V_{HH} domains and Nanobodies are highly stable to heat, pH, proteases and other denaturing agents or conditions (see for example Ewert et al, supra);
V_{HH} domains and Nanobodies are easy and relatively cheap to prepare, even on a scale required for production. For example, V_{HH} domains, Nanobodies and proteins/polypeptides containing the same can be produced using microbial fermentation (e.g. as further described below) and do not require the use of mammalian expression systems, as with for example conventional antibody fragments;
V_{HH} domains and Nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies can show so-called cavity-binding properties (inter alia due to their extended CDR3 loop, compared to conventional V_{H} domains) and can therefore also access targets and epitopes not accessible to conventional 4-chain antibodies and antigen-binding fragments thereof. For example, it has been shown that V_{HH} domains and Nanobodies can inhibit enzymes (see for example WO 97/49805; Transue et al., (1998), supra; Lauwereys et al., (1998), supra).

In a specific and preferred aspect, the invention provides Nanobodies against II-6, and in particular Nanobodies against IL-6 from a warm-blooded animal, and more in particular Nanobodies against IL-6 from a mammal, and especially Nanobodies against human IL-6; as well as proteins and/or polypeptides comprising at least one such Nanobody.

In particular, the invention provides Nanobodies against IL-6, and proteins and/or polypeptides comprising the same, that have improved therapeutic and/or pharmacological properties and/or other advantageous properties (such as, for example, improved ease of preparation and/or reduced costs of goods), compared to conventional antibodies against IL-6 or fragments thereof, compared to constructs that could be based on such conventional antibodies or antibody fragments (such as Fab′ fragments, F(ab′)₂ fragments, ScFv constructs, “diabodies” and other multispecific constructs (see for example the review by Holliger and Hudson, Nat. Biotechnol. 2005 September; 23 (9): 1126-36)), and also compared to the so-called “dAb's” or similar (single) domain antibodies that may be derived from variable domains of conventional antibodies. These improved and advantageous properties will become clear from the further description herein, and for example include, without limitation, one or more of:
increased affinity and/or avidity for IL-6, either in a monovalent format, in a multivalent format (for example in a bivalent format) and/or in a multispecific format (for example one of the multispecific formats described hereinbelow);
better suitability for formatting in a multivalent format (for example in a bivalent format);
better suitability for formatting in a multispecific format (for example one of the multispecific formats described hereinbelow);
improved suitability or susceptibility for “humanizing” substitutions (as defined herein);
less immunogenicity, either in a monovalent format, in a multivalent format (for example in a bivalent format) and/or in a multispecific format (for example one of the multispecific formats described hereinbelow);
increased stability, either in a monovalent format, in a multivalent format (for example in a bivalent format) and/or in a multispecific format (for example one of the multispecific formats described hereinbelow);
increased specificity towards IL-6, either in a monovalent format, in a multivalent format (for example in a bivalent format) and/or in a multispecific format (for example one of the multispecific formats described hereinbelow);
decreased or where desired increased cross-reactivity with IL-6 from different species;
   and/or
one or more other improved properties desirable for pharmaceutical use (including prophylactic use and/or therapeutic use) and/or for diagnostic use (including but not limited to use for imaging purposes), either in a monovalent format, in a multivalent format (for example in a bivalent format) and/or in a multispecific format (for example one of the multispecific formats described hereinbelow).

As generally described herein for the amino acid sequences of the invention, the Nanobodies of the invention are preferably in essentially isolated form (as defined herein), or form part of a protein or polypeptide of the invention (as defined herein), which may comprise or essentially consist of one or more Nanobodies of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than IL-6), so as to provide a monovalent, multivalent or multispecific polypeptide of the invention, respectively, all as described herein. In particular, such a protein or polypeptide may comprise or essentially consist of one or more Nanobodies of the invention and optionally one or more (other) Nanobodies (i.e. directed against other targets than IL-6), all optionally linked via one or more suitable linkers, so as to provide a monovalent, multivalent or multispecific Nanobody construct, respectively, as further described herein. Such proteins or polypeptides may also be in essentially isolated form (as defined herein).

In a Nanobody of the invention, the binding site for binding against IL-6 is preferably formed by the CDR sequences. Optionally, a Nanobody of the invention may also, and in addition to the at least one binding site for binding against IL-6, contain one or more further binding sites for binding against other antigens, proteins or targets. For methods and positions for introducing such second binding sites, reference is for example made to Keck and Huston, Biophysical Journal, 71, October 1996, 2002-2011; EP 0 640 130; WO 06/07260 and the US provisional application by Ablynx N.V. entitled “Immunoglobulin domains with multiple bindings sites” filed on Nov. 27, 2006.

As generally described herein for the amino acid sequences of the invention, when a Nanobody of the invention (or a polypeptide of the invention comprising the same) is intended for administration to a subject (for example for therapeutic and/or diagnostic purposes as described herein), it is preferably directed against human IL-6; whereas for veterinary purposes, it is preferably directed against IL-6 from the species to be treated. Also, as with the amino acid sequences of the invention, a Nanobody of the invention may or may not be cross-reactive (i.e. directed against IL-6 from two or more species of mammal, such as against human IL-6 and IL-6 from at least one of the species of mammal mentioned herein).

Also, again as generally described herein for the amino acid sequences of the invention, the Nanobodies of the invention may generally be directed against any antigenic determinant, epitope, part, domain, subunit or confirmation (where applicable) of IL-6.

As already described herein, the amino acid sequence and structure of a Nanobody can be considered—without however being limited thereto—to be comprised of four framework regions or “FR's” (or sometimes also referred to as “FW's”), which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. Some preferred framework sequences and CDR's (and combinations thereof) that are present in the Nanobodies of the invention are as described herein. Other suitable CDR sequences can be obtained by the methods described herein.

According to a non-limiting but preferred aspect of the invention, (the CDR sequences present in) the Nanobodies of the invention are such that:

the Nanobodies can bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² mole/liter (i.e. with an association constant (K_{A}) of 10⁵ to 10¹² liter/moles or more, and preferably 10⁷ to 10¹² liter/moles or more and more preferably 10⁸ to 10¹² liter/moles);

and/or such that:

the Nanobodies can bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;

and/or such that they:

the Nanobodies can bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, (the CDR sequences present in) the Nanobodies of the invention are such that: a monovalent Nanobody of the invention (or a polypeptide that contains only one Nanobody of the invention) is preferably such that it will bind to IL-6 with an affinity less than 500 nM, preferably less than 200 nM, more preferably less than 10 nM, such as less than 500 pM.

The affinity of the Nanobody of the invention against IL-6 can be determined in a manner known per se, for example using the general techniques for measuring K_{D}. K_{A}, k_{off} or kₒₙ mentioned herein, as well as some of the specific assays described herein.

Some preferred IC50 values for binding of the Nanobodies of the invention (and of polypeptides comprising the same) to IL-6 will become clear from the further description and examples herein.

In a preferred but non-limiting aspect, the invention relates to a Nanobody (as defined herein) against IL-6, which consists of 4 framework regions (FR1 to FR4 respectively) and 3 complementarity determining regions (CDR1 to CDR3 respectively), in which:

CDR1 is chosen from the group consisting of:

a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
and/or

CDR2 is chosen from the group consisting of:

d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
and/or

CDR3 is chosen from the group consisting of:

g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319;
or any suitable fragment of such an amino acid sequence.

In particular, according to this preferred but non-limiting aspect, the invention relates to a Nanobody (as defined herein) against IL-6, which consists of 4 framework regions (FR1 to FR4 respectively) and 3 complementarity determining regions (CDR1 to CDR3 respectively), in which:

CDR1 is chosen from the group consisting of:

a) the amino acid sequences of SEQ ID NO's: 167 to 217;
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
and

CDR2 is chosen from the group consisting of:

d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
and

CDR3 is chosen from the group consisting of:

g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319;
or any suitable fragment of such an amino acid sequences.

As generally mentioned herein for the amino acid sequences of the invention, when a Nanobody of the invention contains one or more CDR1 sequences according to b) and/or c):

i) any amino acid substitution in such a CDR according to b) and/or c) is preferably, and compared to the corresponding CDR according to a), a conservative amino acid substitution (as defined herein);
and/or
ii) the CDR according to b) and/or c) preferably only contains amino acid substitutions, and no amino acid deletions or insertions, compared to the corresponding CDR according to a);
and/or
iii) the CDR according to b) and/or c) may be a CDR that is derived from a CDR according to a) by means of affinity maturation using one or more techniques of affinity maturation known per se.

Similarly, when a Nanobody of the invention contains one or more CDR2 sequences according to e) and/or f):

i) any amino acid substitution in such a CDR according to e) and/or f) is preferably, and compared to the corresponding CDR according to d), a conservative amino acid substitution (as defined herein);
and/or
ii) the CDR according to e) and/or f) preferably only contains amino acid substitutions, and no amino acid deletions or insertions, compared to the corresponding CDR according to d);
and/or
iii) the CDR according to e) and/or f) may be a CDR that is derived from a CDR according to d) by means of affinity maturation using one or more techniques of affinity maturation known per se.

Also, similarly, when a Nanobody of the invention contains one or more CDR3 sequences according to h) and/or i):

i) any amino acid substitution in such a CDR according to h) and/or i) is preferably, and compared to the corresponding CDR according to g), a conservative amino acid substitution (as defined herein);
and/or
ii) the CDR according to h) and/or i) preferably only contains amino acid substitutions, and no amino acid deletions or insertions, compared to the corresponding CDR according to g);
and/or
iii) the CDR according to h) and/or i) may be a CDR that is derived from a CDR according to g) by means of affinity maturation using one or more techniques of affinity maturation known per se.

It should be understood that the last three paragraphs generally apply to any Nanobody of the invention that comprises one or more CDR1 sequences, CDR2 sequences and/or CDR3 sequences according to b), c), e), f), h) or i), respectively.

Of the Nanobodies of the invention, Nanobodies comprising one or more of the CDR's explicitly listed above are particularly preferred; Nanobodies comprising two or more of the CDR's explicitly listed above are more particularly preferred; and Nanobodies comprising three of the CDR's explicitly listed above are most particularly preferred.

Some particularly preferred, but non-limiting combinations of CDR sequences, as well as preferred combinations of CDR sequences and framework sequences, are mentioned in Table A-1 below, which lists the CDR sequences and framework sequences that are present in a number of preferred (but non-limiting) Nanobodies of the invention. As will be clear to the skilled person, a combination of CDR1, CDR2 and CDR3 sequences that occur in the same clone (i.e. CDR1, CDR2 and CDR3 sequences that are mentioned on the same line in Table A-1) will usually be preferred (although the invention in its broadest sense is not limited thereto, and also comprises other suitable combinations of the CDR sequences mentioned in Table A-1). Also, a combination of CDR sequences and framework sequences that occur in the same clone (i.e. CDR sequences and framework sequences that are mentioned on the same line in Table A-1) will usually be preferred (although the invention in its broadest sense is not limited thereto, and also comprises other suitable combinations of the CDR sequences and framework sequences mentioned in Table A-1, as well as combinations of such CDR sequences and other suitable framework sequences, e.g. as further described herein).

Also, in the Nanobodies of the invention that comprise the combinations of CDR's mentioned in Table A-1, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which:

i) any amino acid substitution in such a CDR is preferably, and compared to the corresponding CDR sequence mentioned in Table A-1, a conservative amino acid substitution (as defined herein);
and/or
ii) any such CDR sequence preferably only contains amino acid substitutions, and no amino acid deletions or insertions, compared to the corresponding CDR sequence mentioned in Table A-1;
and/or
iii) any such CDR sequence is a CDR that is derived by means of a technique for affinity maturation known per se, and in particular starting from the corresponding CDR sequence mentioned in Table A-1.

However, as will be clear to the skilled person, the (combinations of) CDR sequences, as well as (the combinations of) CDR sequences and framework sequences mentioned in Table A-1 will generally be preferred.

TABLE A-1

Preferred combinations of CDR sequences, preferred combinations of framework sequences, and
preferred combinations of framework and CDR sequences.
(“ID” refers to the SEQ ID NO in the attached sequence listing)

| | | | | | | | | | | | | | | | |
|---|---|---|---|---|---|---|---|---|---|---|---|---|---|---|---|
| Clone | ID | FRI | ID | CDR1 | ID | FR2 | ID | CDR2 | ID | FR3 | ID | CDR3 | ID | FR4 | ID |

| | | | | | | | | | | | | | | | |
|---|---|---|---|---|---|---|---|---|---|---|---|---|---|---|---|
| PMP6D5 | 320 | QVQLVESGGG | 448 | PYTMG | 167 | WFRQPP | 499 | RINWSGIRN | 218 | RFTISRDNNNNT | 550 | ASQSGSG | 269 | WGQGT | 601 |
| | | LVQAGGSLRL | | | | GKVREF | | YADSVKG | | VYLQMNRLKPE | | YDS | | QVTVSS | |
| | | SCAASGRTFS | | | | VG | | | | DTAVYYCAA | | | | | |
| PMP8F2 | 321 | DVQLVESGGD | 449 | DYAMS | 168 | WLRQTP | 500 | AITGNGASK | 219 | RFTISRDNAKN | 551 | VAKDTGS | 270 | LGQGTQ | 602 |
| | | LVQPGGSLRLS | | | | GKGLEW | | YYAESMKG | | MLYLHLNNLKS | | FYYPAYE | | VTVSS | |
| | | CAASGFSFD | | | | VG | | | | EDTAVYYCRR | | HDV | | | |
| PMP6B12 | 322 | AVQLVESGGG | 450 | YYAIG | 169 | WFRQAP | 501 | CISSSVGTTY | 220 | RFTISRDNAKNT | 552 | SSWFDCG | 271 | RGQGTQ | 603 |
| | | LVQPGGSLRLS | | | | GKEREG | | YSDSVKG | | VYLQMNSLKPE | | VQGRDLG | | VTVSS | |
| | | CAASGFTLA | | | | VS | | | | DTAVYYCVR | | NEYDY | | | |
| PMP6B6 | 323 | QVQLVESAGG | 451 | INAMG | 170 | WYRQAP | 502 | DIMPYGSTE | 221 | RFTISRDNAKNT | 553 | YDPRGDD | 272 | WGQGT | 604 |
| | | LVQPGGSLRLS | | | | GKRREL | | YADSVKG | | VYLQMNSLKPE | | Y | | QVTVSS | |
| | | CAASGIIFS | | | | VA | | | | DTAVYYCHS | | | | | |
| PMP11C1 | 324 | EVQLVESGGG | 452 | IYTMG | 171 | WFRQAP | 503 | AAHWTVFR | 222 | RFTISRDNAKNT | 554 | TRSTAWN | 273 | WGQGT | 605 |
| | | LVQTGGSLRL | | | | GKEREFV | | GNTYYVDS | | VYLQMNSLKPE | | SPQRYDY | | QVTVSS | |
| | | SCATSGLAFS | | | | A | | VKG | | DSAVYYCAA | | | | | |
| PMP23H2 | 325 | AVQLVDSGGG | 453 | RLAMD | 172 | WYRQAP | 504 | SIAVSGTTM | 223 | RFTISRDNAENT | 555 | FDGYTGS | 274 | WGRGT | 606 |
| | | LVQPGGSLRLS | | | | GKQREL | | LDDSVKG | | VYLQMNSLKPE | | DY | | QVTVSS | |
| | | CAASGSIFS | | | | VA | DTAVYYCMA | | | | | | | | |
| PMP7G4 | 326 | AVQLVESGGG | 454 | RLAMD | 173 | WYRQAP | 505 | SISRSGTTM | 224 | RFTISRDNAENM | 556 | FDGYSGS | 275 | WGRGT | 607 |
| | | LVQPGGSLRLS | | | | GKQREL | | AADSVKG | | VYLQMNSLKPE | | DY | | QVTVSS | |
| | | CAASGSIFS | | | | VA | | | | DTAVYVCMA | | | | | |
| PMP20D2 | 327 | AVQLVESGGG | 455 | FNIMG | 174 | WYRQAP | 506 | DITNRGTTN | 225 | RFTISRDNTKNT | 557 | YYPTTGF | 276 | WGQGT | 608 |
| | | LVQPGGSLRLS | | | | GKQREL | | YADSVKG | | VYLQMNSLKPD | | DD | | QVTVSS | |
| | | CAASGSISR | | | | VA | | | | DTAVYYCHT | | | | | |
| PMP7G5 | 328 | QVKLEESGGG | 456 | FNIMG | 175 | WYRQAP | 507 | DITNGGTTM | 226 | RFTISRDNTKNT | 558 | YYPTTGF | 277 | WGQGA | 609 |
| | | LVQPGGSLRLS | | | | GKQREL | | YADSVKG | | VYLQMNSLKPE | | DD | | QVTVSS | |
| | | CAASGSISR | | | | VA | | | | DTAVYYCHT | | | | | |
| PMP7H3 | 329 | DVQLVESGGG | 457 | YYGVG | 176 | WFRQAP | 508 | CISSSDGDT | 227 | RFTISRDNAKNT | 559 | DLSDYGV | 278 | WGQGT | 610 |
| | | LVQPGGSLRLS | | | | GKEREG | | YYADSVKG | | VYLQMNSLKPE | | CSRWPSP | | QVTVSS | |
| | | CAASGFTLD | | | | VS | | | | DTAVYYCAT | | YDY | | | |
| PMP7G9 | 330 | QVQLVESGGG | 458 | YYGVG | 177 | WFRQAP | 509 | CISSSDGDT | 228 | RFTISRDNAKNT | 560 | DLSDYGV | 279 | WGQGT | 611 |
| | | LVQPGGSLRLS | | | | GKEREG | | YYADSVKG | | VYLQMNSLKPE | | CSRWPSP | | QVTVSS | |
| | | CAASGFSLD | | | | VS | | | | DTAVYYCAT | | YDY | | | |
| PMP9A9 | 331 | QVQLVESGGG | 459 | YYGVG | 178 | WFRQAP | 510 | CISSSDGDT | 229 | RFTISRDNAKNT | 561 | DLSDYGV | 280 | WGQGT | 612 |
| | | LVQPGGSLRLS | | | | GKEREG | | YYADSVKG | | VYLQMNSLKPE | | CSRWPSP | | QVTVSS | |
| | | CAASGFSLD | | | | VS | | | | DTAVYYCAT | | YDY | | | |
| PMP22E3 | 332 | QVQLVESGGG | 460 | DSAIG | 179 | WFRQAP | 511 | CISSSDGDT | 230 | RFTISRDNVKN | 562 | DLSDYGV | 281 | WGQGT | 613 |
| | | LVQPGGSLRLS | | | | GKEREG | | YYDDSVKG | | MVYLQMNSLKP | | CSKWPSP | | QVTYSS | |
| | | CAASGFTLD | | | | VS | | | | EDTAVYFCAI | | YDY | | | |
| PMP6E10 | 333 | QVKLEESGGG | 461 | PYTIA | 180 | WFRQAP | 512 | TIIGSDRSTD | 231 | RFTISRNDAKNT | 563 | TGKGYVF | 282 | WGQGT | 614 |
| | | LVQAGGSLRL | | | | GKEREFV | | LDGDTYYA | | VFLQMSSLKPE | | TPNEYDY | | QVTVSS | |
| | | SCVVSGRTFS | | | | T | | DSVRG | | DTAVYYCAL | | | | | |
| PMP6G10 | 334 | QVQLVESGGG | 462 | PYTIG | 181 | WFSQRP | 513 | TIIGSDRSTD | 232 | RFTISRNDAKNT | 564 | TAKGYVF | 283 | WGQGT | 615 |
| | | LAQAGGSLRL | | | | GKEREW | | LDGDTYYA | | VSLQMNSLKPE | | TDNEYDY | | QVTVSS | |
| | | SCVVSGRTFS | | | | VA | | DSVRG | | DSAVYYCAL | | | | | |
| NC3 | 335 | EVQLVESGGG | 463 | INVMN | 182 | WYRQAP | 514 | AITSGGRKN | 233 | RFTISRDNAKNT | 565 | DAPLASD | 284 | WGQGT | 616 |
| | | LVQPGGSLRLS | | | | GTQREFV | | YADSVKG | | VHLQMNSLKPE | | DDVAPAD | | QVTVSS | |
| | | CAASGNIAA | | | | A | | | | DTAVYYCNA | | Y | | | |
| NC6 | 336 | EVQLVESGGG | 464 | SYAMG | 183 | WFRQAP | 515 | AISSNGGST | 234 | RFTISRDSAKNT | 566 | DETTGWV | 285 | WGQGT | 617 |
| | | LVQAGGSLRL | | | | GKDREF | | RYADSVKG | | AYLQMNSLKLE | | QLADFRS | | QVTVSS | |
| | | SCAASGPTFS | | | | VA | | | | DTAVYYCAA | | | | | |
| PMP13A1 | 337 | AVQLVDSGGG | 465 | PYTMG | 184 | WFRQPP | 516 | RINWSGIRN | 235 | RFTISRDNNNNT | 567 | ASQSGSG | 286 | WGQGT | 618 |
| | | LVQAGGSLRL | | | | GKVREF | | YADSVKG | | VYLQMNRLKPE | | YDS | | QVTVSS | |
| | | SCAASGRTFS | | | | VG | | | | DTAVYYCAA | | | | | |
| PMP20G9 | 338 | QVQLVESGGG | 466 | PYTVG | 185 | WFRQPP | 517 | RINWSGIRN | 236 | RFTISRDNNNNT | 568 | ASQSGSG | 287 | WGQGT | 619 |
| | | LVQAGGSLRL | | | | GKVREF | | YADSVKG | | VYLQMNRLKPE | | YDS | | QVTVSS | |
| | | SCAASGRTFS | | | | VG | | | | DTAVYYCAA | | | | | |
| PMP20F4 | 339 | QVQLVESGGG | 467 | PYTMG | 186 | WFRQPP | 518 | RINWSGIRN | 237 | RFTISRDNNNNT | 569 | ASRSGSG | 288 | WGQGT | 620 |
| | | LVQAGGSLRL | | | | GKVREF | | YADSVKG | | VYLQMNRLKPE | | YDS | | QVTVSS | |
| | | SCAASGRTFS | | | | VG | | | | DTAVYYCAA | | | | | |
| PMP21A7 | 340 | AVQLVESGGG | 468 | PYTMG | 187 | WFRQPP | 519 | RINWSGITN | 238 | RFTISRDNNKNT | 570 | ASRSGSG | 289 | WGQGT | 621 |
| | | LVQAGSSLRLS | | | | GKVREF | | YADSVKG | | VYLQMNRLKPE | | YDS | | QVTVSS | |
| | | CAASGRTFS | | | | VG | | | | DTAVYYCAA | | | | | |
| PMP13D8 | 341 | QVKLEESGGG | 469 | PYTMG | 188 | WFRQPP | 520 | RINWSGITN | 239 | RFTISRDNNKNT | 571 | ASQVGSG | 290 | WGQGT | 622 |
| | | LVQAGSSLRLS | | | | GKVREF | | ADSVKG | | VYLQMNRLKPE | | YDS | | QVTVSS | |
| | | CAASGRTSS | | | | VG | | | | DTAVYYCAS | | | | | |
| PMP21E12 | 342 | AVQLVESGGG | 470 | INPMG | 189 | WYRQAP | 521 | RIHGSITNY | 240 | RFTISRDIAKNT | 572 | RRWGYD | 291 | WGQGA | 623 |
| | | LVQPGGSLRLS | | | | GKQREL | | ADSVKG | | VYLQMNSLKPE | | Y | | QVTVSS | |
| | | CAASGSITS | | | | VA | | | | DTAVYYCNA | | | | | |
| PMP21C12 | 343 | AVQLVESGGG | 471 | INPMG | 190 | WYRQAP | 522 | RIHGSITNY | 241 | RFTISRDIAKNT | 573 | RRWGYD | 292 | WGQGA | 624 |
| | | LVQPGGSLRLS | | | | GKQREL | | ADSVKG | | AYLQMNSLKPE | | Y | | QVTVSS | |
| | | CAASGSITG | | | | VA | | | | DTAVYYCNA | | | | | |
| PMP21C2 | 344 | QVQLVESGGG | 472 | INPMA | 191 | WYRQAP | 523 | RIFGGGSTN | 242 | RFTISRDIAKNT | 574 | RRWGYD | 293 | WGQGT | 625 |
| | | LVQPGGSLRLS | | | | GKQRDL | | YADSVKG | | VSLQMNSLKPE | | Y | | QVTVSS | |
| | | CAASEYITS | | | | VA | | | | DTAVYYCNA | | | | | |
| PMP14G4 | 345 | AVQLVDSGGG | 473 | SYPMG | 192 | WFRQGP | 524 | GISQSGVGT | 243 | RFTISRENAKNT | 575 | RDKTLAL | 294 | WGQGT | 626 |
| | | LVQACGSLRL | | | | GKERKF | | AYSDSVKG | | VYLQMNSLKPE | | RDYAYTT | | QVTVSS | |
| | | SCAASGRTFS | | | | VA | | | | DTAVYYCAA | | DVGYDD | | | |
| PMP14E1 | 346 | QVQLVESGGG | 474 | SYPMG | 193 | WFRQAP | 525 | GISQSGGST | 244 | RFTISRENAKST | 576 | RDKTLAL | 295 | WGQGT | 627 |
| | | LVQAGGSLRL | | | | GKERKF | | AYSDSVKG | | VYLQMNSLKPE | | RDYAYTT | | QVTVSS | |
| | | SCAASGRTFS | | | | VA | | | | DTAVYYCAA | | DVGYDD | | | |
| PMP6E9 | 347 | EVQLVESGGG | 475 | SYPMG | 194 | WFRQAP | 526 | GISQSSSSTA | 245 | RFTISRENAKNT | 577 | RGRTLAL | 296 | WGQGT | 628 |
| | | LVQAGGSLRL | | | | GKERKF | | YSDSVKG | | VYLQMNSLKPE | | RDYAYTT | | QVTVSS | |
| | | SCAASGRTFS | | | | VA | | | | DTAVYYCAA | | EVGYDD | | | |
| PMP12H3 | 348 | AVQLVDSGGG | 476 | SYPMG | 195 | WFRQAP | 527 | GISQSGGST | 246 | RFTISRENAKTT | 578 | RGRTLFL | 297 | WGQGT | 629 |
| | | LVQAGGSLRL | | | | GKERKF | | AYSDSVKG | | VYLQMNSLKPE | | RDYAYTT | | QVTVSS | |
| | | SCAASGGTFT | | | | VA | | | | DTAVYYCAA | | EVGYDD | | | |
| PMP12C5 | 349 | DVQLVESGGG | 477 | SYPMG | 196 | WFRQAP | 528 | GISQSGGST | 247 | RFTISRENAKTT | 579 | RGRTLFL | 298 | WGQGT | 630 |
| | | LVQAGGSLRL | | | | GKERKF | | AYSDSVKG | | VYLQMNSLKPE | | RGYAYTT | | QVTVSS | |
| | | SCAASGGTFT | | | | VA | | | | DTAVYYCAA | | EVGYDD | | | |
| PMP17G7 | 350 | QVKLEESOGC | 478 | SYPMG | 197 | WFRQAP | 529 | GISQSGGST | 248 | RFTISRENAKNT | 580 | RGRTIAL | 299 | WGQGT | 631 |
| | | LVQAGGSLRL | | | | GKEREFV | | AYSDSVKG | | VYLQMNSLKPE | | RNYAYTT | | QVTVSS | |
| | | SCAASGGTFS | | | | T | | | | DTAVYYCAA | | EVGYDD | | | |
| PMP14G11 | 351 | QVQLVESGGG | 479 | SFPMG | 198 | WFRQAP | 530 | GISQSGGST | 249 | RFTISRENAKNT | 581 | RGRTLAL | 300 | WGQGT | 632 |
| | | LVQAGGSLRL | | | | GKGREF | | HYSDSVKG | | VYLQMNSLKPE | | RNYAYYT | | QVTVSS | |
| | | SCAASCGTFS | | | | VA | | | | DTAVYYCAA | | EVGYDD | | | |
| PMP9F9 | 352 | AVQLVESGGG | 480 | SFPMG | 199 | WFRQAP | 531 | GISQSGGST | 250 | RFTISRENARNT | 582 | RGRTLAL | 301 | WGQGT | 633 |
| | | LVQAGGSLRL | | | | GEKREFV | | HYSDSVKG | | VYLQMNSLKPE | | RNYAYTT | | QVTVSS | |
| | | SCAASGGTFS | | | | A | | | | DTAVYYCAA | | EVGYDD | | | |
| PMP14A8 | 353 | EVQLVESGGG | 481 | SFPMG | 200 | WFRQAP | 532 | GISQSGGST | 251 | RFTISRENAKNT | 583 | RGRTLAL | 302 | WGQGT | 634 |
| | | LVQAGGSLRL | | | | GKERKF | | HYSDSVKG | | VYLQMNNLKPE | | RNYAYTT | | QVTVSS | |
| | | SCAASGGTFS | | | | VA | | | | DTAVYYCAA | | EVGYDD | | | |
| PMP17B5 | 354 | QVQLVESGGG | 482 | AFPMG | 201 | WFRQAP | 533 | GISQSGGST | 252 | RFTISRENAKNT | 584 | RGRTLAL | 303 | WGQGT | 635 |
| | | LVQAGGSLRL | | | | GKERKF | | HYSDSVKG | | IYLQMNSLKPED | | RNYAYTT | | QVTVSS | |
| | | SCAASGGTFS | | | | VA | | | | TAVYYCAA | | EVGYDD | | | |
| PMP6B7 | 355 | QVQLVESGGG | 483 | AFPMG | 202 | WFRQAP | 534 | GISQSGGST | 253 | RFTISRENAKNT | 585 | RGRTLAL | 304 | WGQGT | 636 |
| | | LVQAGGSLRL | | | | GKERKF | | HYSDSVKG | | VYLQMNSLKPE | | RNYAYTT | | QVTVSS | |
| | | SCAASGGTFS | | | | VA | | | | DTAVYYCAA | | EVGYDD | | | |
| PMP14E9 | 356 | AVQLVESGGG | 484 | AFPMG | 203 | WFRQAP | 535 | CISQSGGST | 254 | RFTISKENAKST | 586 | RGRTLAL | 305 | WGQGT | 637 |
| | | LVQAGGSLRL | | | | GKEREFV | | HYSDSVKG | | VYLQMNSLKPE | | RNYAYTT | | QVTVSS | |
| | | SCAASGGTFS | | | | A | | | | DTAVYYCAA | | EVGYDD | | | |
| PMP17D7 | 357 | AVQLVDSGGG | 485 | AFPMG | 204 | WFRQAP | 536 | GISQSGGST | 255 | RFTISKENAKNT | 587 | RGRTLAL | 306 | WGQGT | 638 |
| | | LVQAGGSLRL | | | | GKEREFV | | HYSDSVKG | | VYLQMNSLKPE | | RNYAYTT | | QVTVSS | |
| | | SCAASGGTFS | | | | A | | | | DTAVYYCAA | | EVGYDD | | | |
| PMP14G1 | 358 | QVKLEESGGG | 486 | AFPMG | 205 | WFRQAP | 537 | GISQSGGST | 256 | RFTISKENAKNT | 588 | RGGTLAL | 307 | WGQGT | 639 |
| | | LVQAGGSLRL | | | | GKEREFV | | HYSDSVKG | | VYLQMNSLKPE | | RNYAYTT | | QVTVSS | |
| | | SCAASGGTFS | | | | A | | | | DTAVYYCAA | | EVGYDD | | | |
| PMP17B11 | 359 | QVQLVESGGG | 487 | TYAMG | 206 | WFRQAP | 538 | AISWSGANT | 257 | RFTISRDNAKNT | 589 | SAIIEGFQ | 308 | WGQGT | 640 |
| | | LVQAGGSLRL | | | | GKEREFV | | YYADSVKG | | VYLRMNSLKPE | | DSIVIFSE | | QVTVSS | |
| | | SCAASGPTFS | | | | A | | | | DTAAYYCAA | | AGYDY | | | |
| PMP10C4 | 360 | AVQLVDSGGG | 488 | NYHMV | 207 | WFRQAP | 539 | AASGSTSST | 258 | RFTISRDNAKNT | 590 | VAGLLLP | 309 | WGKGT | 641 |
| | | LVQAGGSLRL | | | | GKEREFV | | YYADSVKG | | VYLQMNSLKPE | | RVAEGM | | LVTVSS | |
| | | SCAASGRSFS | | | | A | | | | DTAVYYCAA | | DY | | | |
| PMP17C4 | 361 | AVQLVDSGGG | 489 | NYAMA | 208 | WFRQAP | 540 | VISYAGGRT | 259 | RFFISRDNAKNT | 591 | VDSPLIAT | 310 | WGQGT | 642 |
| | | LVQAGDSLRL | | | | GKEREFV | | YYADSVKG | | VYLQMNSLKPE | | HPRGYDY | | QVTVSS | |
| | | SCAASGRTFS | | | | A | | | | DTAVYYCAA | | | | | |
| PMP21B4 | 362 | QVQLVESGGG | 490 | IDAMA | 209 | WFRQAP | 541 | TMNWSTGA | 260 | RFTSSRDNAKST | 592 | ARGLLIA | 311 | WGQGT | 643 |
| | | LVQAGDSLRV | | | | GKEREFV | | TYYADSVK | | SYLQMNSLKPE | | TDARGYD | | QVTVSS | |
| | | ACAASGRTFS | | | | S | | G | | DTAVYYCAA | | Y | | | |
| PMP21H1 | 363 | QVQLVESCGG | 491 | KHHATG | 210 | WFRQAP | 542 | ALNWSGGN | 261 | RFTISRDNAQNT | 593 | GSYVFYF | 312 | WGQGT | 644 |
| | | LVQTGGSLRL | | | | GKEREFV | | TYYTDSVK | | VYLQMNSLKPE | | TVRDQYD | | QVTVSS | |
| | | SCAASGSTFS | | | | A | | G | | DTAVYYCAA | | Y | | | |
| PMP10A6 | 364 | QVQLVESGGG | 492 | SYVMG | 211 | WFRQTP | 543 | TINWSGSNG | 262 | RFTISRDNAKNT | 594 | SAGGFLY | 313 | WGQGT | 645 |
| | | LVQAGGSLRL | | | | GKEREFV | | YYADSVKC | | VYLQMNNLKPE | | PRVGQGY | | QVTVSS | |
| | | SCASSGRTFS | | | | S | | | | DTAVYYCAA | | DY | | | |
| PMP13H6 | 365 | QVKLEESGGG | 493 | SYVMG | 212 | WFRQTP | 544 | TINWSGSNK | 263 | RFTISRDNAKNT | 595 | SAGGFLV | 314 | WGQGT | 646 |
| | | LVQAGGSLRL | | | | GKEREFV | | YYADSVKG | | VYLQMNSLKPE | | PRVGQGY | | QVTVSS | |
| | | SCASSGRTFS | | | | S | | | | DTAVYYCAA | | DY | | | |
| PMP13F12 | 366 | AVQLVDSGGG | 494 | SSPMG | 213 | WFRQAP | 545 | AISGRSGNT | 264 | RFTISRDNAKNT | 596 | ERVGLLL | 315 | WGQGT | 647 |
| | | LVQAGGSLRL | | | | GKEREFV | | YYADSVKG | | VYLQMNSLKPE | | TVVAEGY | | QVTVSS | |
| | | SCAASGRTFS | | | | A | | | | DTAVYYCAA | | DY | | | |
| PMP21A2 | 367 | DVQLVESGGG | 495 | SSPMG | 214 | WFRQAP | 546 | AISGRSGNT | 265 | RFTISRDNAKNT | 597 | ERVGLLL | 316 | WGQGT | 648 |
| | | LVQAGGSLRL | | | | GKEREFY | | YYADSVKG | | VYLQMNSLKPE | | TVVAEGY | | QVTVSS | |
| | | SCAASGRTFS | | | | A | | | | DTAVYYCAG | | DY | | | |
| PMP21F7 | 368 | EVQLVESGGG | 496 | SSPMG | 215 | WFRQAP | 547 | AISGRSGNT | 266 | RFTISRDNAKNT | 598 | ERVGLLL | 317 | WGRGT | 649 |
| | | LVQAGGSLRL | | | | GKEREFV | | YYADSVKG | | VYLQMNSLKPE | | TVVAEGY | | QVTVSS | |
| | | SCAASGRTFS | | | | A | | | | DTAVYYCAG | | DY | | | |
| PMP21H3 | 369 | QVQLVESGGG | 497 | NGPMA | 216 | WFRQAP | 548 | AISWRTGTT | 267 | RFTISRDNAKNT | 599 | ERVGLLL | 318 | WGQGT | 650 |
| | | LVQAGGSLRL | | | | GKFREFV | | YYADSVKG | | VYLQMNSLKPE | | AVVAEGY | | QVTVSS | |
| | | SCAASGRTFS | | | | S | | | | DTAVYYCAA | | DY | | | |
| PMP21E7 | 370 | AVQLVESGGG | 498 | SYPIA | 217 | WFRQPP | 549 | AISWRGGNT | 268 | RFTISRDNAKNT | 600 | ERAGVLL | 319 | WGQGT | 651 |
| | | LVQAGGSLRL | | | | GKEREFV | | YYADSVKG | | VYLQMNSLKPE | | TKVPEGY | | QVTVSS | |
| | | SSVVSGGTFS | | | | A | | | | DTAVYYSAA | | DY | | | |

Thus, in the Nanobodies of the invention, at least one of the CDR1, CDR2 and CDR3 sequences present is suitably chosen from the group consisting of the CDR1, CDR2 and CDR3 sequences, respectively, listed in Table A-1; or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-1.

In this context, by “suitably chosen” is meant that, as applicable, a CDR1 sequence is chosen from suitable CDR1 sequences (i.e. as defined herein), a CDR2 sequence is chosen from suitable CDR2 sequences (i.e. as defined herein), and a CDR3 sequence is chosen from suitable CDR3 sequence (i.e. as defined herein), respectively. More in particular, the CDR sequences are preferably chosen such that the Nanobodies of the invention bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1; and/or from the group consisting of the CDR3 sequences that have 3, 2 or only 1 amino acid difference(s) with at least one of the CDR3 sequences listed in Table A-1.

Preferably, in the Nanobodies of the invention, at least two of the CDR1, CDR2 and CDR3 sequences present are suitably chosen from the group consisting of the CDR1, CDR2 and CDR3 sequences, respectively, listed in Table A-1 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-1.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1, respectively; and at least one of the CDR1 and CDR2 sequences present is suitably chosen from the group consisting of the CDR1 and CDR2 sequences, respectively, listed in Table A-1 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Most preferably, in the Nanobodies of the invention, all three CDR1, CDR2 and CDR3 sequences present are suitably chosen from the group consisting of the CDR1, CDR2 and CDR3 sequences, respectively, listed in Table A-1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-1.

Even more preferably, in the Nanobodies of the invention, at least one of the CDR1, CDR2 and CDR3 sequences present is suitably chosen from the group consisting of the CDR1, CDR2 and CDR3 sequences, respectively, listed in Table A-1. Preferably, in this aspect, at least one or preferably both of the other two CDR sequences present are suitably chosen from CDR sequences that have at least 80%, preferably at least 90%, more preferably at least 95%, even more preferably at least 99% sequence identity with at least one of the corresponding CDR sequences, respectively, listed in Table A-1; and/or from the group consisting of the CDR sequences that have 3, 2 or only 1 amino acid difference(s) with at least one of the corresponding sequences, respectively, listed in Table A-1.

In particular, in the Nanobodies of the invention, at least the CDR3 sequence present is suitably chosen from the group consisting of the CDR3 listed in Table A-1. Preferably, in this aspect, at least one and preferably both of the CDR1 and CDR2 sequences present are suitably chosen from the groups of CDR1 and CDR2 sequences, respectively, that have at least 80%, preferably at least 90%, more preferably at least 95%, even more preferably at least 99% sequence identity with the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Even more preferably, in the Nanobodies of the invention, at least two of the CDR1, CDR2 and CDR3 sequences present are suitably chosen from the group consisting of the CDR1, CDR2 and CDR3 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with at least one of the corresponding sequences listed in Table A-1.

In particular, in the Nanobodies of the invention, at least the CDR3 sequence is suitably chosen from the group consisting of the CDR3 sequences listed in Table A-1, and either the CDR1 sequence or the CDR2 sequence is suitably chosen from the group consisting of the CDR1 and CDR2 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with the corresponding CDR sequences listed in Table A-1.

Even more preferably, in the Nanobodies of the invention, all three CDR1, CDR2 and CDR3 sequences present are suitably chosen from the group consisting of the CDR1, CDR2 and CDR3 sequences, respectively, listed in Table A-1.

Also, generally, the combinations of CDR's listed in Table A-1 (i.e. those mentioned on the same line in Table A-1) are preferred. Thus, it is generally preferred that, when a CDR in a Nanobody of the invention is a CDR sequence mentioned in Table A-1 or is suitably chosen from the group of CDR sequences that have at least 80%, preferably at least 90%, more preferably at least 95%, even more preferably at least 99% sequence identity with a CDR sequence listed in Table A-1; and/or from the group consisting of CDR sequences that have 3, 2 or only 1 amino acid difference(s) with a CDR sequence listed in Table A-1, that at least one and preferably both of the other CDR's are suitably chosen from the CDR sequences that belong to the same combination in Table A-1 (i.e. mentioned on the same line in Table A-1) or are suitably chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1; or (3) a CDR1 sequence; a CDR2 sequence that has more than 80% sequence identity with one of the CDR2 sequence listed in Table A-1; and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 and a CDR3 sequence listed in Table A-1 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and a CDR3 sequence that has more than 80% sequence identity with the CDR3 sequence listed in Table A-1 that belongs to the same or a different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same combination.

In the most preferred Nanobodies of the invention, the CDR1, CDR2 and CDR3 sequences present are suitably chosen from one of the combinations of CDR1, CDR2 and CDR3 sequences, respectively, listed in Table A-1.

According to another preferred, but non-limiting aspect of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

In another preferred, but non-limiting aspect, the invention relates to a Nanobody in which the CDR sequences (as defined herein) have more than 80%, preferably more than 90%, more preferably more than 95%, such as 99% or more sequence identity (as defined herein) with the CDR sequences of at least one of the amino acid sequences of SEQ ID NO's: 320 to 370.

Generally, Nanobodies with the above CDR sequences may be as further described herein, and preferably have framework sequences that are also as further described herein. Thus, for example and as mentioned herein, such Nanobodies may be naturally occurring Nanobodies (from any suitable species), naturally occurring V_{HH} sequences (i.e. from a suitable species of Camelid) or synthetic or semi-synthetic amino acid sequences or Nanobodies, including but not limited to partially humanized Nanobodies or V_{HH} sequences, fully humanized Nanobodies or V_{HH} sequences, camelized heavy chain variable domain sequences, as well as Nanobodies that have been obtained by the techniques mentioned herein.

Thus, in one specific, but non-limiting aspect, the invention relates to a humanized Nanobody, which consists of 4 framework regions (FR1 to FR4 respectively) and 3 complementarity determining regions (CDR1 to CDR3 respectively), in which CDR1 to CDR3 are as defined herein and in which said humanized Nanobody comprises at least one humanizing substitution (as defined herein), and in particular at least one humanizing substitution in at least one of its framework sequences (as defined herein).

In another preferred, but non-limiting aspect, the invention relates to a Nanobody in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 320 to 370. This degree of amino acid identity can for example be determined by determining the degree of amino acid identity (in a manner described herein) between said Nanobody and one or more of the sequences of SEQ ID NO's: 320 to 370, in which the amino acid residues that form the framework regions are disregarded. Such Nanobodies can be as further described herein.

In another preferred, but non-limiting aspect, the invention relates to a Nanobody with an amino acid sequence that is chosen from the group consisting of SEQ ID NO's: 320 to 370 or from the group consisting of from amino acid sequences that have more than 80%, preferably more than 90%, more preferably more than 95%, such as 99% or more sequence identity (as defined herein) with at least one of the amino acid sequences of SEQ ID NO's: 320 to 370.

Another preferred, but non-limiting aspect of the invention relates to humanized variants of the Nanobodies of SEQ ID NO's: 320 to 370, that comprise, compared to the corresponding native V_{HH} sequence, at least one humanizing substitution (as defined herein), and in particular at least one humanizing substitution in at least one of its framework sequences (as defined herein).

The polypeptides of the invention comprise or essentially consist of at least one Nanobody of the invention. Some preferred, but non-limiting examples of polypeptides of the invention are given in SEQ ID NO's: 371 to 447.

It will be clear to the skilled person that the Nanobodies that are mentioned herein as “preferred” (or “more preferred”, “even more preferred”, etc.) are also preferred (or more preferred, or even more preferred, etc.) for use in the polypeptides described herein. Thus, polypeptides that comprise or essentially consist of one or more “preferred” Nanobodies of the invention will generally be preferred, and polypeptides that comprise or essentially consist of one or more “more preferred” Nanobodies of the invention will generally be more preferred, etc.

Generally, proteins or polypeptides that comprise or essentially consist of a single Nanobody (such as a single Nanobody of the invention) will be referred to herein as “monovalent” proteins or polypeptides or as “monovalent constructs”. Proteins and polypeptides that comprise or essentially consist of two or more Nanobodies (such as at least two Nanobodies of the invention or at least one Nanobody of the invention and at least one other Nanobody) will be referred to herein as “multivalent” proteins or polypeptides or as “multivalent constructs”, and these may provide certain advantages compared to the corresponding monovalent Nanobodies of the invention. Some non-limiting examples of such multivalent constructs will become clear from the further description herein.

According to one specific, but non-limiting aspect, a polypeptide of the invention comprises or essentially consists of at least two Nanobodies of the invention, such as two or three Nanobodies of the invention. As further described herein, such multivalent constructs can provide certain advantages compared to a protein or polypeptide comprising or essentially consisting of a single Nanobody of the invention, such as a much improved avidity for IL-6. Such multivalent constructs will be clear to the skilled person based on the disclosure herein; some preferred, but non-limiting examples of such multivalent Nanobody constructs are the constructs of SEQ ID NO's: 371 to 447.

According to another specific, but non-limiting aspect, a polypeptide of the invention comprises or essentially consists of at least one Nanobody of the invention and at least one other binding unit (i.e. directed against another epitope, antigen, target, protein or polypeptide), which is preferably also a Nanobody. Such proteins or polypeptides are also referred to herein as “multispecific” proteins or polypeptides or as “multispecific constructs”, and these may provide certain advantages compared to the corresponding monovalent Nanobodies of the invention (as will become clear from the further discussion herein of some preferred, but-nonlimiting multispecific constructs). Such multispecific constructs will be clear to the skilled person based on the disclosure herein; some preferred, but non-limiting examples of such multispecific Nanobody constructs are the constructs of SEQ ID NO's: 371 to 447.

According to yet another specific, but non-limiting aspect, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above aspects, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein.

In the above constructs, the one or more Nanobodies and/or other amino acid sequences may be directly linked to each other and/or suitably linked to each other via one or more linker sequences. Some suitable but non-limiting examples of such linkers will become clear from the further description herein.

In one specific aspect of the invention, a Nanobody of the invention or a compound, construct or polypeptide of the invention comprising at least one Nanobody of the invention may have an increased half-life, compared to the corresponding amino acid sequence of the invention. Some preferred, but non-limiting examples of such Nanobodies, compounds and polypeptides will become clear to the skilled person based on the further disclosure herein, and for example comprise Nanobodies sequences or polypeptides of the invention that have been chemically modified to increase the half-life thereof (for example, by means of pegylation); amino acid sequences of the invention that comprise at least one additional binding site for binding to a serum protein (such as serum albumin. Reference is for example made to the US provisional application by Ablynx N.V. entitled “Immunoglobulin domains with multiple binding sites” filed on Nov. 27, 2006); or polypeptides of the invention that comprise at least one Nanobody of the invention that is linked to at least one moiety (and in particular at least one amino acid sequence) that increases the half-life of the Nanobody of the invention. Examples of polypeptides of the invention that comprise such half-life extending moieties or amino acid sequences will become clear to the skilled person based on the further disclosure herein; and for example include, without limitation, polypeptides in which the one or more Nanobodies of the invention are suitable linked to one or more serum proteins or fragments thereof (such as serum albumin or suitable fragments thereof) or to one or more binding units that can bind to serum proteins (such as, for example, Nanobodies or (single) domain antibodies that can bind to serum proteins such as serum albumin, serum immunoglobulins such as IgG, or transferrine); polypeptides in which a Nanobody of the invention is linked to an Fc portion (such as a human Fc) or a suitable part or fragment thereof; or polypeptides in which the one or more Nanobodies of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006.

Again, as will be clear to the skilled person, such Nanobodies, compounds, constructs or polypeptides may contain one or more additional groups, residues, moieties or binding units, such as one or more further amino acid sequences and in particular one or more additional Nanobodies (i.e. not directed against IL-6), so as to provide a tri- of multispecific Nanobody construct.

Generally, the Nanobodies of the invention (or compounds, constructs or polypeptides comprising the same) with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the Nanobodies, compounds, constructs or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another one aspect of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

In particular, polypeptides comprising one or more Nanobodies of the invention are preferably such that they:

bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);

and/or such that they:

bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;

and/or such that they:

bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a polypeptide that contains only one amino acid sequence of the invention is preferably such that it will bind to IL-6 with an affinity less than 500 nM, preferably less than 200 nM, more preferably less than 10 nM, such as less than 500 pM. In this respect, it will be clear to the skilled person that a polypeptide that contains two or more Nanobodies of the invention may bind to IL-6 with an increased avidity, compared to a polypeptide that contains only one amino acid sequence of the invention.

Some preferred IC₅₀ values for binding of the amino acid sequences or polypeptides of the invention to IL-6 will become clear from the further description and examples herein.

Other polypeptides according to this preferred aspect of the invention may for example be chosen from the group consisting of amino acid sequences that have more than 80%, preferably more than 90%, more preferably more than 95%, such as 99% or more “sequence identity” (as defined herein) with one or more of the amino acid sequences of SEQ ID NO's: 371 to 447, in which the Nanobodies comprised within said amino acid sequences are preferably as further defined herein.

Another aspect of this invention relates to a nucleic acid that encodes a Nanobody of the invention or a polypeptide of the invention comprising the same. Again, as generally described herein for the nucleic acids of the invention, such a nucleic acid may be in the form of a genetic construct, as defined herein.

In another aspect, the invention relates to host or host cell that expresses or that is capable of expressing a Nanobody of the invention and/or a polypeptide of the invention comprising the same; and/or that contains a nucleic acid of the invention. Some preferred but non-limiting examples of such hosts or host cells will become clear from the further description herein.

Another aspect of the invention relates to a product or composition containing or comprising at least one Nanobody of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein. The invention further relates to methods for preparing or generating the Nanobodies, polypeptides, nucleic acids, host cells, products and compositions described herein. Some preferred but non-limiting examples of such methods will become clear from the further description herein.

The invention further relates to applications and uses of the Nanobodies, polypeptides, nucleic acids, host cells, products and compositions described herein, as well as to methods for the prevention and/or treatment for diseases and disorders associated with IL-6. Some preferred but non-limiting applications and uses will become clear from the further description herein.

Other aspects, embodiments, advantages and applications of the invention will also become clear from the further description hereinbelow.

Generally, it should be noted that the term Nanobody as used herein in its broadest sense is not limited to a specific biological source or to a specific method of preparation. For example, as will be discussed in more detail below, the Nanobodies of the invention can generally be obtained: (1) by isolating the V_{HH} domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

One preferred class of Nanobodies corresponds to the V_{HH} domains of naturally occurring heavy chain antibodies directed against IL-6. As further described herein, such V_{HH} sequences can generally be generated or obtained by suitably immunizing a species of Camelid with IL-6 (i.e. so as to raise an immune response and/or heavy chain antibodies directed against IL-6), by obtaining a suitable biological sample from said Camelid (such as a blood sample, serum sample or sample of B-cells), and by generating V_{HH} sequences directed against IL-6, starting from said sample, using any suitable technique known per se. Such techniques will be clear to the skilled person and/or are further described herein.

Alternatively, such naturally occurring V_{HH} domains against IL-6, can be obtained from naïve libraries of Camelid V_{HH} sequences, for example by screening such a library using IL-6, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Thus, in another aspect, the invention relates to a method for generating Nanobodies, that are directed against IL-6. In one aspect, said method at least comprises the steps of:
a) providing a set, collection or library of Nanobody sequences; and
b) screening said set, collection or library of Nanobody sequences for Nanobody sequences that can bind to and/or have affinity for IL-6;
   and
c) isolating the amino acid sequence(s) that can bind to and/or have affinity for IL-6.

In such a method, the set, collection or library of Nanobody sequences may be a naïve set, collection or library of Nanobody sequences; a synthetic or semi-synthetic set, collection or library of Nanobody sequences; and/or a set, collection or library of Nanobody sequences that have been subjected to affinity maturation.

In a preferred aspect of this method, the set, collection or library of Nanobody sequences may be an immune set, collection or library of Nanobody sequences, and in particular an immune set, collection or library of V_{HH} sequences, that have been derived from a species of Camelid that has been suitably immunized with IL-6 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of Nanobody or V_{HH} sequences may be displayed on a phage, phagemid, ribosome or suitable micro-organism (such as yeast), such as to facilitate screening. Suitable methods, techniques and host organisms for displaying and screening (a set, collection or library of) Nanobody sequences will be clear to the person skilled in the art, for example on the basis of the further disclosure herein. Reference is also made to WO 03/054016 and to the review by Hoogenboom in Nature Biotechnology, 23, 9, 1105-1116 (2005).

In another aspect, the method for generating Nanobody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for IL-6; and (ii) cells that express heavy chain antibodies, in which substeps (i) and (ii) can be performed essentially as a single screening step or in any suitable order as two separate screening steps, so as to provide at least one cell that expresses a heavy chain antibody that can bind to and/or has affinity for IL-6;
   and
c) either (i) isolating from said cell the V_{HH} sequence present in said heavy chain antibody; or (ii) isolating from said cell a nucleic acid sequence that encodes the V_{HH} sequence present in said heavy chain antibody, followed by expressing said V_{HH} domain.

In the method according to this aspect, the collection or sample of cells may for example be a collection or sample of B-cells. Also, in this method, the sample of cells may be derived from a Camelid that has been suitably immunized with IL-6 or a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820. Particular reference is made to the so-called “Nanoclone™” technique described in International application WO 06/079372 by Ablynx N.V.

In another aspect, the method for generating an amino acid sequence directed against IL-6 may comprise at least the steps of:
a) providing a set, collection or library of nucleic acid sequences encoding heavy chain antibodies or Nanobody sequences;
b) screening said set, collection or library of nucleic acid sequences for nucleic acid sequences that encode a heavy chain antibody or a Nanobody sequence that can bind to and/or has affinity for IL-6;
   and
c) isolating said nucleic acid sequence, followed by expressing the V_{HH} sequence present in said heavy chain antibody or by expressing said Nanobody sequence, respectively.

In such a method, the set, collection or library of nucleic acid sequences encoding heavy chain antibodies or Nanobody sequences may for example be a set, collection or library of nucleic acid sequences encoding a naïve set, collection or library of heavy chain antibodies or V_{HH} sequences; a set, collection or library of nucleic acid sequences encoding a synthetic or semi-synthetic set, collection or library of Nanobody sequences; and/or a set, collection or library of nucleic acid sequences encoding a set, collection or library of Nanobody sequences that have been subjected to affinity maturation.

In a preferred aspect of this method, the set, collection or library of amino acid sequences may be an immune set, collection or library of nucleic acid sequences encoding heavy chain antibodies or V_{HH} sequences derived from a Camelid that has been suitably immunized with IL-6 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational, or semi-empirical approaches such as computer modelling techniques or biostatics or datamining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library)), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Yet another technique for obtaining V_{HH} sequences directed against IL-6, involves suitably immunizing a transgenic mammal that is capable of expressing heavy chain antibodies (i.e. so as to raise an immune response and/or heavy chain antibodies directed against IL-6), obtaining a suitable biological sample from said transgenic mammal that is capable of expressing heavy chain antibodies (i.e. so as to raise an immune response and/or heavy chain antibodies against IL-6), obtaining a suitable biological sample from said transgenic mammal that contains (nucleic acid sequences encoding) said V_{HH} sequences or Nanobody sequences (such as a blood sample, serum sample or sample of B-cells), and then generating V_{HH} sequences directed against IL-6, starting from said sample, using any suitable technique known per se. For example, for this purpose, the heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103 (41):15130-5 can be used. For example, such heavy chain antibody expressing mice can express heavy chain antibodies with any suitable (single) variable domain, such as (single) variable domains from natural sources (e.g. human (single) variable domains, Camelid (single) variable domains or shark (single) variable domains), as well as for example synthetic or semi-synthetic (single) variable domains.

The invention also relates to the V_{HH} sequences or Nanobody sequences that are obtained by the above methods, or alternatively by a method that comprises the one of the above methods and in addition at least the steps of determining the nucleotide sequence or amino acid sequence of said V_{HH} sequence or Nanobody sequence; and of expressing or synthesizing said V_{HH} sequence or Nanobody sequence in a manner known per se, such as by expression in a suitable host cell or host organism or by chemical synthesis.

As mentioned herein, a particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized Nanobodies of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized Nanobody is preferably a V_{H} sequence from a mammal, more preferably the V_{H} sequence of a human being, such as a V_{H}3 sequence. However, it should be noted that such camelized Nanobodies of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” Nanobody of the invention, respectively. This nucleic acid can then be expressed in a manner known per se, so as to provide the desired Nanobody of the invention. Alternatively, based on the amino acid sequence of a naturally occurring V_{HH} domain or V_{H} domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired Nanobody of the invention.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable mariner, so as to provide a Nanobody of the invention or a nucleotide sequence or nucleic acid encoding the same (which may then be suitably expressed). Nucleotide sequences encoding framework sequences of V_{HH} sequences or Nanobodies will be clear to the skilled person based on the disclosure herein and/or the further prior art cited herein (and/or may alternatively be obtained by PCR starting from the nucleotide sequences obtained using the methods described herein) and may be suitably combined with nucleotide sequences that encode the desired CDR's (for example, by PCR assembly using overlapping primers), so as to provide a nucleic acid encoding a Nanobody of the invention.

Optionally, a Nanobody of the invention may also, and in addition to the at least one binding site for binding against IL-6, contain one or more further binding sites for binding against other antigens, proteins or targets. For methods and positions for introducing such second binding sites, reference is for example made to Keck and Huston, Biophysical Journal, 71, October 1996, 2002-2011; EP 0 640 130; WO 06/07260 and the US provisional application by Ablynx N.V. entitled “Immunoglobulin domains with multiple binding sites” filed on Nov. 27, 2006.

As mentioned herein, Nanobodies may in particular be characterized by the presence of one or more “Hallmark residues” (as described herein) in one or more of the framework sequences.

According to one preferred, but non-limiting aspect of the invention, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q;
   and/or:
(b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E;
   and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q;
   and/or in which:
(b) the amino acid residue at position 45 according to the Kabat numbering is a charged amino acid or a cysteine and the amino acid residue at position 44 according to the Kabat numbering is preferably E;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q;
   and/or:
(b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R;
   and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(e) the amino acid residue at position 108 according to the Kabat numbering is Q;
   and/or in which:
(f) the amino acid residue at position 44 according to the Kabat numbering is E and in which the amino acid residue at position 45 according to the Kabat numbering is an R;
   and/or in which:
(g) the amino acid residue at position 103 according to the Kabat numbering is chosen from the group consisting of P, R and S, and is in particular chosen from the group consisting of R and S;
   and in which:
(h) CDR1, CDR2 and CDR3 are as defined herein, and are preferably as defined according to one of the preferred embodiments herein, and are more preferably as defined according to one of the more preferred aspects herein.

In particular, a Nanobody against IL-6, according to the invention may have the structure:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q;
   and/or in which:
(b) the amino acid residue at position 44 according to the Kabat numbering is E and in which the amino acid residue at position 45 according to the Kabat numbering is an R;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, according to one preferred, but non-limiting aspect of the aspect of the invention, a Nanobody can generally be defined as a polypeptide comprising an amino acid sequence that is comprised of four framework regions/sequences interrupted by three complementarity determining regions/sequences, in which;
(a-1) the amino acid residue at position 44 according to the Kabat numbering is chosen from the group consisting of A, G, E, D, G, Q, R, S, L; and is preferably chosen from the group consisting of G, E or Q; and
(a-2) the amino acid residue at position 45 according to the Kabat numbering is chosen from the group consisting of L, R or C; and is preferably chosen from the group consisting of L or R; and
(a-3) the amino acid residue at position 103 according to the Kabat numbering is chosen from the group consisting of W, R or S; and is preferably W or R, and is most preferably W;
(a-4) the amino acid residue at position 108 according to the Kabat numbering is Q; or in which:
(b-1) the amino acid residue at position 44 according to the Kabat numbering is chosen from the group consisting of E and Q; and
(b-2) the amino acid residue at position 45 according to the Kabat numbering is R; and
(b-3) the amino acid residue at position 103 according to the Kabat numbering is chosen from the group consisting of W, R and S; and is preferably W;
(b-4) the amino acid residue at position 108 according to the Kabat numbering is chosen from the group consisting of Q and L; and is preferably Q;
   or in which:
(c-1) the amino acid residue at position 44 according to the Kabat numbering is chosen from the group consisting of A, G, E, D, Q, R, S and L; and is preferably chosen from the group consisting of G, E and Q; and
(c-2) the amino acid residue at position 45 according to the Kabat numbering is chosen from the group consisting of L, R and C; and is preferably chosen from the group consisting of L and R; and
(c-3) the amino acid residue at position 103 according to the Kabat numbering is chosen from the group consisting of P, R and S; and is in particular chosen from the group consisting of R and S; and
(c-4) the amino acid residue at position 108 according to the Kabat numbering is chosen from the group consisting of Q and L; is preferably Q;
   and in which
(d) CDR1, CDR2 and CDR3 are as defined herein, and are preferably as defined according to one of the preferred embodiments herein, and are more preferably as defined according to one of the more preferred embodiments herein.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, G, Q, R, S, L; and is preferably chosen from the group consisting of G, E or Q;
   and in which:
(b) the amino acid residue at position 45 according to the Kabat numbering is chosen from the group consisting of L, R or C; and is preferably chosen from the group consisting of L or R;
   and in which:
(c) the amino acid residue at position 103 according to the Kabat numbering is chosen from the group consisting of W, R or S; and is preferably W or R, and is most preferably W;
   and in which
(d) the amino acid residue at position 108 according to the Kabat numbering is Q;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of E and Q;
   and in which:
(b) the amino acid residue at position 45 according to the Kabat numbering is R;
   and in which:
(c) the amino acid residue at position 103 according to the Kabat numbering is chosen from the group consisting of W, R and S; and is preferably W;
   and in which:
(d) the amino acid residue at position 108 according to the Kabat numbering is chosen from the group consisting of Q and L; and is preferably Q;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, Q, R, S and L; and is preferably chosen from the group consisting of G, E and Q;
   and in which:
(b) the amino acid residue at position 45 according to the Kabat numbering is chosen from the group consisting of L, R and C; and is preferably chosen from the group consisting of L and R;
   and in which:
(c) the amino acid residue at position 103 according to the Kabat numbering is chosen from the group consisting of P, R and S; and is in particular chosen from the group consisting of R and S;
   and in which:
(d) the amino acid residue at position 108 according to the Kabat numbering is chosen from the group consisting of Q and L; is preferably Q;
   and in which:
(e) CDR1, CDR2 and CDR3 are as defined herein, and are preferably as defined according to one of the preferred embodiments herein, and are more preferably as defined according to one of the more preferred embodiments herein.

Two particularly preferred, but non-limiting groups of the Nanobodies of the invention are those according to a) above; according to (a-1) to (a-4) above; according to b) above; according to (b-1) to (b-4) above; according to (c) above; and/or according to (c-1) to (c-4) above, in which;
a) the amino acid residues at positions 4447 according to the Kabat numbering form the sequence GLEW (or a GLEW-like sequence as defined herein) and the amino acid residue at position 108 is Q;
   or in which:
b) the amino acid residues at positions 4346 according to the Kabat numbering form the sequence KERE or KQRE (or a KERE-like sequence) and the amino acid residue at position 108 is Q or L, and is preferably Q.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 44-47 according to the Kabat numbering form the sequence GLEW (or a GLEW-like sequence as defined herein) and the amino acid residue at position 108 is Q;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 43-46 according to the Kabat numbering form the sequence KERE or KQRE (or a KERE-like sequence) and the amino acid residue at position 108 is Q or L, and is preferably Q;
   and in which:
(b) CDR1, CDR2 and CDR3 are as defined herein, and are preferably as defined according to one of the preferred embodiments herein, and are more preferably as defined according to one of the more preferred embodiments herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably V.

Thus, without being limited hereto in any way, on the basis of the amino acid residues present on the positions mentioned above, the Nanobodies of the invention can generally be classified is on the basis of the following three groups:
a) The “GLEW-group”: Nanobodies with the amino acid sequence GLEW at positions 44-47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below;
b) The “KERE-group”: Nanobodies with the amino acid sequence KERE or KQRE (or another KERE-like sequence) at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103;
c) The “103 P, R, S-group”: Nanobodies with a P, R or S at position 103. These Nanobodies can have either the amino acid sequence GLEW at positions 44-47 of the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Also, where appropriate, Nanobodies may belong to (i.e. have characteristics of) two or more of these classes. For example, one specifically preferred group of Nanobodies has GLEW or a GLEW-like sequence at positions 44-47; P, R or S (and in particular R) at position 103; and Q at position 108 (which may be humanized to L).

More generally, it should be noted that the definitions referred to above describe and apply to Nanobodies in the form of a native (i.e. non-humanized) V_{HH} sequence, and that humanized variants of these Nanobodies may contain other amino acid residues than those indicated above (i.e. one or more humanizing substitutions as defined herein). For example, and without limitation, in some humanized Nanobodies of the GLEW-group or the 103 P, R, S-group, Q at position 108 may be humanized to 108L. As already mentioned herein, other humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred embodiments herein, and are more preferably as defined according to one of the more preferred aspects herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred embodiments herein, and are more preferably as defined according to one of the more preferred aspects herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred embodiments herein, and are more preferably as defined according to one of the more preferred aspects herein.

Also, more generally and in addition to the 108Q, 43E/44R and 103P, R, S residues mentioned above, the Nanobodies of the invention can contain, at one or more positions that in a conventional V_{H} domain would form (part of) the V_{H}/V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2). Such substitutions include, but are not limited to, the GLEW-like sequences mentioned in Table A-3 below; as well as the substitutions that are described in the International Application WO 00/29004 for so-called “microbodies”, e.g. so as to obtain a Nanobody with Q at position 108 in combination with KLEW at positions 44-47. Other possible substitutions at these positions will be clear to the skilled person based upon the disclosure herein.

In one embodiment of the Nanobodies of the invention, the amino acid residue at position 83 is chosen from the group consisting of L, M, S, V and W; and is preferably L.

Also, in one embodiment of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for “humanized” Nanobodies, as described herein). The amino acid residue at position 84 is chosen from the group consisting of P, A, R, S, D T, and V in one embodiment, and is most preferably P (for Nanobodies corresponding to naturally occurring V_{HH} domains) or R (for “humanized” Nanobodies, as described herein).

Furthermore, in one embodiment of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring V_{HH} domains are mentioned in Table A-4. For comparison, the corresponding amino acid residues of the human V_{H}3 called DP-47 have been indicated in italics.

TABLE A-3
Hallmark Residues in Nanobodies

| Position | Human V_{H}3 | Hallmark Residues |
|---|---|---|
| 11 | L, V; predominantly L | L, M, S, V, W; preferably L |
| 37 | V, I, F; usually V | F⁽¹⁾, Y, H, I, L or V, preferably F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾ |
| 45⁽⁸⁾ | L | L⁽²⁾, R⁽³⁾, C, I, L, P, Q, V; preferably L⁽²⁾ |
| | | or R⁽³⁾ |
| 47⁽⁸⁾ | W, Y | W⁽²⁾, L⁽¹⁾ or F⁽¹⁾, A, G, I, M, R, S, V or |
| | | Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; predominantly A | P⁽⁵⁾, A, L, R, S, T, D, V; preferably P |
| 103 | W | W⁽⁴⁾, P⁽⁶⁾, R⁽⁶⁾, S; preferably W |
| 104 | G | G or D; preferably G |
| 108 | L, M or T; predominantly L | Q, L⁽⁷⁾ or R; preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q (in non-humanized) V_{HH} sequences that also contain a W at position 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-4
Some preferred but non-limiting combinations of Hallmark
Residues in naturally occurring Nanobodies.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

For humanization of these combinations, reference is made to the specification.

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables A-5-A-8 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering Chothia (supra) that the residues at these positions already form part of CDR1).

In Tables A-5-A-8, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Table A-5 also contains data on the V_{HH} entropy (“V_{HH} Ent.”) and V_{HH} variability (“V_{HH} Var.”) at each amino acid position for a representative sample of 1118 V_{HH} sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have vary little variability (and in case of position 9 is G in all 1118 sequences analysed), whereas for residues that form part of the CDR's generally values of 1.5 or more are found (data not shown). Note that (1) the amino acid residues listed in the second column of Table A-5 are based on a bigger sample than the 1118 V_{HH} sequences that were analysed for determining the V_{HH} entropy and V_{HH} variability referred to in the last two columns; and (2) the data represented below supports the hypothesis that the amino acid residues at positions 27-30 and maybe even also at positions 93 and 94 already form part of the CDR's (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure, Function and Genetics, 52: 544-552 (2003).

TABLE A-5
Non-limiting examples of amino acid residues in FR1
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

TABLE A-6
Non-limiting examples of amino acid residues in FR2
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, preferably F⁽¹⁾ or Y | | 1.1 | 6 |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, R, S, L: preferably G⁽²⁾, E⁽³⁾ or | | 1.3 | 5 |
| | Q; most preferably G⁽²⁾ or E⁽³⁾. | | | |
| 45 | Hallmark residue: L⁽²⁾, R⁽³⁾, C, I, L, P, Q, V: preferably L⁽²⁾ or R⁽³⁾ | | 0.6 | 4 |
| 46 | E, V | E, D, K, Q, V | 0.4 | 2 |
| 47 | Hallmark residue: W⁽²⁾, L⁽¹⁾ or F⁽¹⁾, A, G, I, M, R, S, V or Y: | | 1.9 | 9 |
| | preferably W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

TABLE A-7
Non-limiting examples of amino acid residues in FR3
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, Q or T; preferably K or | | 0.9 | 7 |
| | R; most preferably K | | | |
| 84 | Hallmark residue: P⁽⁵⁾, A, D, L, R, S, T, V; preferably P | | 0.7 | 6 |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-8
Non-limiting examples of amino acid residues in FR4
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 103 | Hallmark residue: W⁽⁴⁾, P⁽⁶⁾, R⁽⁶⁾, S; preferably W | | 0.4 | 2 |
| 104 | Hallmark residue: G or D; preferably G | | 0.1 | 1 |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R: preferably Q or L⁽⁷⁾ | | 0.4 | 3 |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the Hallmark residues are as defined herein;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

[SEQ ID NO: 126]

[1] QVQLQESGGGXVQAGGSLRLSCAASG [26]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
   and in which:
(b) FR2 is chosen from the group consisting of the amino acid sequence:

| | |
|---|---|
| [36] WXRQAPGKXXEXVA [49] | [SEQ ID NO: 127] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s);
   and in which:
(c) FR3 is chosen from the group consisting of the amino acid sequence:

[SEQ ID NO: 128]

[66] RFTISRDNAKNTVYLQMNSLXXEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
   and in which:
(d) FR4 is chosen from the group consisting of the amino acid sequence:

| | |
|---|---|
| [103] XXQGTXVTVSS [113] | [SEQ ID NO: 129] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
   and in which:
(e) CDR1, CDR2 and CDR3 are as defined herein, and are preferably as defined according to one of the preferred embodiments herein, and are more preferably as defined according to one of the more preferred embodiments herein;
   in which the Hallmark Residues are indicated by “X” and are as defined hereinabove and in which the numbers between brackets refer to the amino acid positions according to the Kabat numbering.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

[SEQ ID NO: 130]

[1] QVQLQESGGOLVQAGGSLRLSCAASG [26]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above; and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequences:

| | | | |
|---|---|---|---|
| [36] | WFRQAPGKERELVA | [49] | [SEQ ID NO: 131] |
| [36] | WFRQAPGKEREFVA | [49] | [SEQ ID NO: 132] |
| [36] | WFRQAPGKEREGA | [49] | [SEQ ID NO: 133] |
| [36] | WFRQAPGKQRELVA | [49] | [SEQ ID NO: 134] |
| [36] | WFRQAPGKQREFVA | [49] | [SEQ ID NO: 135] |
| [36] | WYRQAPGKGLEWA | [49] | [SEQ ID NO: 136] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 137]

[66] RFTISRDNAKNTVYLQMNSLKPEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | | | |
|---|---|---|---|
| [103] | WGQGTQVTVSS | [113] | [SEQ ID NO: 138] |
| [103] | WGQGTLVTVSS | [113] | [SEQ ID NO: 139] |

or from the group consisting of amino acid sequences that have at least 80%, preferably at least 90%, more preferably at least 95%, even more preferably at least 99% sequence identity (as defined herein) with one of the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

[SEQ ID NO: 130]

[1] QVQLQESGGGLVQAGGSLRLSCAASG [26]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequences:

| | | | |
|---|---|---|---|
| [36] | WFRQAPGKERELVA | [49] | [SEQ ID NO: 131] |
| [36] | WFRQAPGKEREFVA | [49] | [SEQ ID NO: 132] |
| [36] | WFRQAPGKEREGA | [49] | [SEQ ID NO: 133] |
| [36] | WFRQAPGKQRELVA | [49] | [SEQ ID NO: 134] |
| [36] | WFRQAPGKQREFVA | [49] | [SEQ ID NO: 135] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO:. 137]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | | | |
|---|---|---|---|
| [103] | WGQGTQVTVSS | [113] | [SEQ ID NO: 138] |
| [103] | WGQGTLVTVSS | [113] | [SEQ ID NO: 139] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

[SEQ ID NO: 130]

[1] QVQLQESGGGLVQAGGSLRLSCAASG [26]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequence:

| | |
|---|---|
| [36] WYRQAPGKGLEWA [49] | [SEQ ID NO: 136] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 137]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequence:

| | |
|---|---|
| [103] WGQGTQVTVSS [113] | [SEQ ID NO: 138] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

Some other framework sequences that can be present in the Nanobodies of the invention can be found in the European patent EP 656 946 mentioned above (see for example also the granted U.S. Pat. No. 5,759,808).

Thus, in another preferred, but not limiting aspect, a Nanobody of the invention can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) one or more of the amino acid residues at positions 11, 37, 44, 45, 47, 83, 84, 103, 104 and 108 according to the Kabat numbering are chosen from the Hallmark residues mentioned in Table A-3;
   and in which:
ii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In particular, a Nanobody of the invention can be an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
i) (preferably) one or more of the amino acid residues at positions 11, 37, 44, 45, 47, 83, 84, 103, 104 and 108 according to the Kabat numbering are chosen from the Hallmark residues mentioned in Table A-3 (it being understood that V_{HH} sequences will contain one or more Hallmark residues; and that partially humanized Nanobodies will usually, and preferably, [still] contain one or more Hallmark residues [although it is also within the scope of the invention to provide—where suitable in accordance with the invention—partially humanized Nanobodies in which all Hallmark residues, but not one or more of the other amino acid residues, have been humanized]; and that in fully humanized Nanobodies, where suitable in accordance with the invention, all amino acid residues at the positions of the Hallmark residues will be amino acid residues that occur in a human V_{H}3 sequence. As will be clear to the skilled person based on the disclosure herein that such V_{HH} sequences, such partially humanized Nanobodies with at least one Hallmark residue, such partially humanized Nanobodies without Hallmark residues and such fully humanized Nanobodies all form aspects of this invention);
   and in which:
ii) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded;
   and in which:
iii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

TABLE A-9

Representative amino acid sequences for Nanobodies of the KERE, GLEW and P, R, S 103 group.
The CDR's are indicated with XXXX

| | | |
|---|---|---|
| KERE sequence no. 1 | SEQ ID NO: 1 | EVQLVESGGGLVQPGGSLRLSCAASGIPFSXXXXXWFRQAPGKQRDSVAXXXXXRFTI |
| | | SRDNAKNTVYLQMNSLKPEDTAVYRCYFXXXXXWGQGTQVTVSS |
| KERE sequence no. 2 | SEQ ID NO: 2 | QVKLEESGGGLVQAGGSLRLSCVGSGRTFSXXXXXWFRLAPGKEREFVAXXXXXRFTI |
| | | SRDTASNRGYLHMNNLTPEDTAVYYCAAXXXXXWGQGTQVTVSS |
| KERE sequence no. 3 | SEQ ID NO: 3 | AVQLVDSGGGLVQAGDSLKLSCALTGGAFTXXXXXWFRQTPGREREFVAXXXXXRFTI |
| | | SRDNAKNMVYLRMNSLIPEDAAVYSCAAXXXXXWGQGTLVTVSS |
| KERE sequence no. 4 | SEQ ID NO: 4 | QVQLVESGGGLVEAGGSLRLSCTASESPFRXXXXXWFRQTSGQEREFVAXXXXXRFTI |
| | | SRDDAKNTVWLHGSTLKPEDTAVYYCAAXXXXXWGQGTQVTVSS |
| KERE sequence no. 5 | SEQ ID NO: 5 | AVQLVESGGGLVQGGGSLRLACAASERIFDXXXXXWYRQGPGNERELVAXXXXXRFTI |
| | | SMDYTKQTVYLHMNSLRPEDTGLYYCKIXXXXXWGQGTQVTVSS |
| KERE sequence no. 6 | SEQ ID NO: 6 | DVKFVESGGGLVQAGGSLRLSCVASGFNFDXXXXXWFRQAPGKEREEVAXXXXXRFT |
| | | ISSEKDKNSVYLQMNSLKPEDTALYICAGXXXXXWGRGTQVTVSS |
| KERE sequence no. 7 | SEQ ID NO: 7 | QVRLAESGGGLVQSGGSLRLSCVASGSTYTXXXXXWYRQYPGKGRALVAXXXXXRFT |
| | | IARDSTKDTFCLQMNNLKPEDTAVYYCYAXXXXXWGQGTQVTVSS |
| KERE sequence no. 8 | SEQ ID NO: 8 | EVQLVESGGGLVQAGGSLRLSCAASGFTSDXXXXXWFRQAPGKPREGVSXXXXXRFT |
| | | ISTDNAKNTVHLLMNRVNAEDTALYYCAVXXXXXWGRGTRVTVSS |
| KERE sequence no. 9 | SEQ ID NO: 9 | QVQLVESGGGLVQPGGSLRLSCQASGDISTXXXXXWYRQVPGKLREFVAXXXXXRFTI |
| | | SGDNAKRAIYLQMNNLKPDDTAVYYCNRXXXXXWGQGTQVTVSP |
| KERE sequence no. 10 | SEQ ID NO: 10 | QVPVVESGGGLVQAGDSLRLFCAVPSFTSTXXXXXWFRQAPGKEREFVAXXXXXRFTI |
| | | SRNATKNTLTLRMDSLKPEDTAVYYCAAXXXXXWGQGTQVTVSS |
| KERE sequence no. 11 | SEQ ID NO: 11 | EVQLVESGGGLVQAGDSLRLFCTVSGGTASXXXXXWFRQAPGEKREFVAXXXXXRFTI |
| | | ARENAGNMVYLQMNNLKPDDTALYTCAAXXXXXWGRGTQVTVSS |
| KERE sequence no. 12 | SEQ ID NO: 12 | AVQLVESGGDSVQPGDSQTLSCAASGRTNSXXXXXWFRQAPGKERVFLAXXXXXRFT |
| | | ISRDSAKNMMYLQMNNLKPQDTAVYYCAAXXXXXWGQGTQVTVSS |
| KERE sequence no. 13 | SEQ ID NO: 13 | AVQLVESGGGLVQAGGSLRLSCVVSGLTSSXXXXXWFRQTPWQERDFVAXXXXXRFT |
| | | ISRDNYKDTVLLEMNFLKPEDTAIYYCAAXXXXXWGQGTQVTVSS |
| KERE sequence no. 14 | SEQ ID NO: 14 | AVQLVESGGGLVQAGASLRLSCATSTRTLDXXXXXWFRQAPGRDREFVAXXXXXRFT |
| | | VSRDSAENTVALQMNSLKPEDTAVYYCAAXXXXXWGQGTRVTVSS |
| KERE sequence no. 15 | SEQ ID NO: 15 | QVQLVESGGGLVQPGGSIRLSCTVSRLTAHXXXXXWFRQAPGKEREAVSXXXXXRFTI |
| | | SRDYAGNTAFLQMDSLKPEDTGVYYCATXXXXXWGQGTQVTVSS |
| KERE sequence no. 16 | SEQ ID NO: 16 | EVQLVESGGELVQAGGSLKLSCTASGRNFVXXXXXWFRRAPGKEREFVAXXXXXRFT |
| | | VSRDNGKNTAYLRMNSLKPEDTADYYCAVXXXXXLGSGTQVTVSS |
| GLEW sequence no. 1 | SEQ ID NO: 17 | AVQLVESGGGLVQPGGSLRLSCAASGFTFSXXXXXWVRQAPGKVLEWVSXXXXXRFT |
| | | ISRDNAKNTLYLQMNSLKPEDTAVYYCVKXXXXXGSQGTQVTVSS |
| GLEW sequence no. 2 | SEQ ID NO: 18 | EVQLVESGGGLVQPGGSLRLSCVCVSSGCTXXXXXWVRQAPGKAEEWVSXXXXXRF |
| | | KISRDNAKKTLYLQMNSLGPEDTAMYYCQRXXXXXRGQGTQVTVSS |
| GLEW sequence no. 3 | SEQ ID NO: 19 | EVQLVESGGGLALPGGSLTLSCVFSGSTFSXXXXXWVRHTPGKAEEWVSXXXXXRFTI |
| | | SRDNAKNTLYLEMNSLSPEDTAMYYCGRXXXXXRSKGIQVTVSS |
| P, R, S 103 sequence no. 1 | SEQ ID NO: 20 | AVQLVESGGGLVQAGGSLRLSCAASGRTFSXXXXXWFRQAPGKEREFVAXXXXXRFTI |
| | | SRDNAKNTVYLQMNSLKPEDTAVYYCAAXXXXXRGQGTQVTVSS |
| P, R, S 103 sequence no. 2 | SEQ ID NO: 21 | DVQLVESGGDLVQPGGSLRLSCAASGFSFDXXXXXWLRQTPGKGLEWVGXXXXXRFT |
| | | ISRDNAKNMLYLHLNNLKSEDTAVYYCRRXXXXXLGQGTQVTVSS |
| P, R, S 103 sequence no. 3 | SEQ ID NO: 22 | EVQLVESGGGLVQPGGSLRLSCVCVSSGCTXXXXXWVRQAPGKAEEWVSXXXXXRF |
| | | KISRDNAKKTLYLQMNSLGPEDTAMYYCQRXXXXXRGQGTQVTVSS |

In particular, a Nanobody of the invention of the KERE group can be an amino acid sequence with the (general) structure

FR1-CDR1-FR2-CDR2-FR3-CDR3-FR4

in which:
i) the amino acid residue at position 45 according to the Kabat numbering is a charged amino acid (as defined herein) or a cysteine residue, and position 44 is preferably an E;
   and in which:
ii) FR1 is an amino acid sequence that has at least 80% amino acid identity with at least one of the following amino acid sequences:

TABLE A-10
Representative FW1 sequences for Nanobodies of the KERE-group.

| | | |
|---|---|---|
| KERE FW1 sequence no. 1 | SEQ ID NO: 23 | QVQRVESGGGLVQAGGSLRLSCAASGRTSS |
| KERE FW1 sequence no. 2 | SEQ ID NO: 24 | QVQLVESGGGLVQTGDSLSLSCSASGRTFS |
| KERE FW1 sequence no. 3 | SEQ ID NO: 25 | QVKLEESGGGLVQAGDSLRLSCAATGRAFG |
| KERE FW1 sequence no. 4 | SEQ ID NO: 26 | AVQLVESGGGLVQPGESLGLSCVASGRDFV |
| KERE FW1 sequence no. 5 | SEQ ID NO: 27 | EVQLVESGGGLVQAGGSLRLSCEVLGRTAG |
| KERE FW1 sequence no. 6 | SEQ ID NO: 28 | QVQLVESGGGWVQPGGSLRLSCAASETILS |
| KERE FW1 sequence no. 7 | SEQ ID NO: 29 | QVQLVESGGGTVQPGGSLNLSCVASGNTFN |
| KERE FW1 sequence no. 8 | SEQ ID NO: 30 | EVQLVESGGGLAQPGGSLQLSCSAPGFTLD |
| KERE FW1 sequence no. 9 | SEQ ID NO: 31 | AQELEESGGGLVQAGGSLRLSCAASGRTFN |

and in which:
iii) FR2 is an amino acid sequence that has at least 80% amino acid identity with at least one of the following amino acid sequences:

TABLE A-11

Representative FW2 sequences for Nanobodies of
the KERE-group.

| | | |
|---|---|---|
| KERE FW2 | SEQ ID NO: 41 | WFRQAPGKEREFVA |
| sequence no. 1 | | |
| KERE FW2 | SEQ ID NO: 42 | WFRQTPGREREFVA |
| sequence no. 2 | | |
| KERE FW2 | SEQ ID NO: 43 | WYRQAPGKQREMVA |
| sequence no. 3 | | |
| KERE FW2 | SEQ ID NO: 44 | WYRQGPGKQRELVA |
| sequence no. 4 | | |
| KERE FW2 | SEQ ID NO: 45 | WIRQAPGKEREGVS |
| sequence no. 5 | | |
| KERE FW2 | SEQ ID NO: 46 | WFREAPGKEREGIS |
| sequence no. 6 | | |
| KERE FW2 | SEQ ID NO: 47 | WYRQAPGKERDLVA |
| sequence no. 7 | | |
| KERE FW2 | SEQ ID NO: 48 | WFRQAPGKQREEVS |
| sequence no. 8 | | |
| KERE FW2 | SEQ ID NO: 49 | WFRQPPGKVREFVG |
| sequence no. 9 | | |

and in which:
iv) FR3 is an amino acid sequence that has at least 80% amino acid identity with at least one of the following amino acid sequences:

TABLE A-12
Representative FW3 sequences for Nanobodies of the KERE-group.

| | | |
|---|---|---|
| KERE FW3 sequence no. 1 | SEQ ID NO: 50 | RFTISRDNAKNTVYLQMNSLKPEDTAVYRCYF |
| KERE FW3 sequence no. 2 | SEQ ID NO: 51 | RFAISRDNNKNTGYLQMNSLEPEDTAVYYCAA |
| KERE FW3 sequence no. 3 | SEQ ID NO: 52 | RFTVARNNAKNTVNLEMNSLKPEDTAVYYCAA |
| KERE FW3 sequence no. 4 | SEQ ID NO: 53 | RFTISRDIAKNTVDLLMNNLEPEDTAVYYCAA |
| KERE FW3 sequence no. 5 | SEQ ID NO: 54 | RLTISRDNAVDTMYLQMNSLKPEDTAVYYCAA |
| KERE FW3 sequence no. 6 | SEQ ID NO: 55 | RFTISRDNAKNTVYLQMDNVKPEDTAIYYCAA |
| KERE FW3 sequence no. 7 | SEQ ID NO: 56 | RFTISKDSGKNTVYLQMTSLKPEDTAVYYCAT |
| KERE FW3 sequence no. 8 | SEQ ID NO: 57 | RFTISRDSAKNMMYLQMNNLKPQDTAVYYCAA |
| KERE FW3 sequence no. 9 | SEQ ID NO: 58 | RFTISRENDKSTVYLQLNSLKPEDTAVYYCAA |
| KERE FW3 sequence no. 10 | SEQ ID NO: 59 | RFTISRDYAGNTAYLQMNSLKPEDTGVYYCAT |

and in which:
v) FR4 is an amino acid sequence that has at least 80% amino acid identity with at least one of the following amino acid sequences:

TABLE A-13

Representative FW4 sequences for Nanobodies of
the KERE-group.

| | | |
|---|---|---|
| KERE FW4 | SEQ ID NO: 60 | WGQGTQVTVSS |
| sequence no. 1 | | |
| KERE FW4 | SEQ ID NO: 61 | WGKGTLVTVSS |
| sequence no. 2 | | |
| KERE FW4 | SEQ ID NO: 62 | RGQGTRVTVSS |
| sequence no. 3 | | |
| KERE FW4 | SEQ ID NO: 63 | WGLGTQVTISS |
| sequence no. 4 | | |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

Also, the above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

With regard to framework 1, it will be clear to the skilled person that, when an amino acid sequence as outlined above is generated by expression of a nucleotide sequence, the first four amino acid sequences (i.e. amino acid residues 1-4 according to the Kabat numbering) may often be determined by the primer(s) that have been used to generate said nucleic acid. Thus, for determining the degree of amino acid identity, the first four amino acid residues are preferably disregarded.

Also, with regard to framework 1, and although amino acid positions 27 to 30 are according to the Kabat numbering considered to be part of the framework regions (and not the CDR's), it has been found by analysis of a database of more than 1000 V_{HH} sequences that the positions 27 to 30 have a variability (expressed in terms of V_{HH} entropy and V_{HH} variability—see Tables A-5 to A-8) that is much greater than the variability on positions 1 to 26. Because of this, for determining the degree of amino acid identity, the amino acid residues at positions 27 to 30 are preferably also disregarded.

In view of this, a Nanobody of the KERE class may be an amino acid sequence that is comprised of four framework regions/sequences interrupted by three complementarity determining regions/sequences, in which:
i) the amino acid residue at position 45 according to the Kabat numbering is a charged amino acid (as defined herein) or a cysteine residue, and position 44 is preferably an E;
   and in which:
ii) FR1 is an amino acid sequence that, on positions 5 to 26 of the Kabat numbering, has at least 80% amino acid identity with at least one of the following amino acid sequences:

TABLE A-14

Representative FW1 sequences (amino acid residues
5 to 26) for Nanobodies of the KERR-group.

| | | |
|---|---|---|
| KERE FW1 se- | SEQ ID NO: 32 | VESGGGLVQPGGSLRLSCAASG |
| quence no. 10 | | |
| KERE FW1 se- | SEQ ID NO: 33 | VDSGGGLVQAGDSLKLSCALTG |
| quence no. 11 | | |
| KERE FW1 se- | SEQ ID NQ: 34 | VDSGGGLVQAGDSLRLSCAASG |
| quence no. 12 | | |
| KERE FW1 se- | SEQ ID NO: 35 | VDSGGGLVEAGGSLRLSCQVSE |
| quence no. 13 | | |
| KERE FW1 se- | SEQ ID NO: 36 | QDSGGGSVQAGGSLKLSCAASG |
| quence no. 14 | | |
| KERE FW1 se- | SEQ ID NO: 37 | VQSGGRLVQAGDSLRLSCAASE |
| quence no. 15 | | |
| KERE FW1 se- | SEQ ID NQ: 38 | VESGGTLVQSGDSLKLSCASST |
| quence no. 16 | | |
| KERE FW1 se- | SEQ ID NO: 39 | MESGGDSVQSGGSLTLSCVASG |
| quence no. 17 | | |
| KERE FW1 se- | SEQ ID NO: 40 | QASGGGLVQAGGSLRLSCSASV |
| quence no. 18 | | |

and in which:
iii) FR2, FR3 and FR4 are as mentioned herein for FR2, FR3 and FR4 of Nanobodies of the KERE-class;
   and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

A Nanobody of the GLEW class may be an amino acid sequence that is comprised of four framework regions/sequences interrupted by three complementarity determining regions/sequences, in which
i) preferably, when the Nanobody of the GLEW-class is a non-humanized Nanobody, the amino acid residue in position 108 is Q;
ii) FR1 is an amino acid sequence that has at least 80% amino acid identity with at least one of the following amino acid sequences:

TABLE A-15
Representative FW1 sequences for Nanobodies of the GLEW-group.

| | | |
|---|---|---|
| GLEW FW1 sequence no. 1 | SEQ ID NO: 64 | QVQLVESGGGLVQPGGSLRLSCAASGFTFS |
| GLEW FW1 sequence no. 2 | SEQ ID NO: 65 | EVHLVESGGGLVRPGGSLRLSCAAFGFIFK |
| GLEW FW1 sequence no. 3 | SEQ ID NO: 66 | QVKLEESGGGLAQPGGSLRLSCVASGFTFS |
| GLEW FW1 sequence no. 4 | SEQ ID NO: 67 | EVQLVESGGGLVQPGGSLRLSCVCVSSGCT |
| GLEW FW1 sequence no. 5 | SEQ ID NO: 68 | EVQLVESGGGLALPGGSLTLSCVFSGSTFS |

and in which:
iii) FR2 is an amino acid sequence that has at least 80% amino acid identity with at least one of the following amino acid sequences:

TABLE A-16

Representative FW2 sequences for Nanobodies of
the GLEW-group.

| | | |
|---|---|---|
| GLEW FW2 | SEQ ID NO: 72 | WVRQAPGKVLEWVS |
| sequence no. 1 | | |
| GLEW FW2 | SEQ ID NO: 73 | WVRRPPGKGLEWVS |
| sequence no. 2 | | |
| GLEW FW2 | SEQ ID NO: 74 | WVRQAPGMGLEWVS |
| sequence no. 3 | | |
| GLEW FW2 | SEQ ID NO: 75 | WVRQAPGKEPEWVS |
| sequence no. 4 | | |
| GLEW FW2 | SEQ ID NO: 76 | WVRQAPGKDQEWVS |
| sequence no. 5 | | |
| GLEW FW2 | SEQ ID NO: 77 | WVRQAPGKAEEWVS |
| sequence no. 6 | | |
| GLEW FW2 | SEQ ID NO: 78 | WVRQAPGKGLEWVA |
| sequence no. 7 | | |
| GLEW FW2 | SEQ ID NO: 79 | WVRQAPGRATEWVS |
| sequence no. 8 | | |

and in which:
iv) FR3 is an amino acid sequence that has at least 80% amino acid identity with at least one of the following amino acid sequences:

TABLE A-17
Representative FW3 sequences for Nanobodies of the GLEW-group.

| | | |
|---|---|---|
| GLEW FW3 sequence no. 1 | SEQ ID NO: 80 | RFTISRDNAKNTLYLQMNSLKPEDTAVYYCVK |
| GLEW FW3 sequence no. 2 | SEQ ID NO: 81 | RFTISRDNARNTLYLQMDSLIPEDTALYYCAR |
| GLEW FW3 sequence no. 3 | SEQ ID NO: 82 | RFTSSRDNAKSTLYLQMNDLKPEDTALYYCAR |
| GLEW FW3 sequence no. 4 | SEQ ID NO: 83 | RFIISRDNAKNTLYLQMNSLGPEDTAMYYCQR |
| GLEW FW3 sequence no. 5 | SEQ ID NO: 84 | RFTASRDNAKNTLYLQMNSLKSEDTARYYCAR |
| GLEW FW3 sequence no. 6 | SEQ ID NO: 85 | RFTISRDNAKNTLYLQMDDLQSEDTAMYYCGR |

and in which:
v) FR4 is an amino acid sequence that has at least 80% amino acid identity with at least one of the following amino acid sequences:

TABLE A-18

Representative FW4 sequences for Nanobodies of
the GLEW-group.

| | | |
|---|---|---|
| GLEW FW4 sequence no. 1 | SEQ ID NO: 86 | GSQGTQVTVSS |
| GLEW FW4 sequence no. 2 | SEQ ID NO: 87 | LRGGTQVTVSS |
| GLEW FW4 sequence no. 3 | SEQ ID NO: 88 | RGQGTLVTVSS |
| GLEW FW4 sequence no. 4 | SEQ ID NO: 89 | RSRGIQVTVSS |
| GLEW FW4 sequence no. 5 | SEQ ID NO: 90 | WGKGTQVTVSS |
| GLEW FW4 sequence no. 6 | SEQ ID NO: 91 | WGOGTQVTVSS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the GLEW class may be an amino acid sequence that is comprised of four framework regions/sequences interrupted by three complementarity determining regions/sequences, in which:
i) preferably, when the Nanobody of the GLEW-class is a non-humanized Nanobody, the amino acid residue in position 108 is Q;
   and in which:
ii) FR1 is an amino acid sequence that, on positions 5 to 26 of the Kabat numbering, has at least 80% amino acid identity with at least one of the following amino acid sequences:

TABLE A-19

Representative FW1 sequences (amino acid residues
5 to 26) for Nanobodies of the KERE-group.

| | | |
|---|---|---|
| GLEW FW1 | SEQ ID NO: 69 | VESGGGLVQPGGSLRLSCAASG |
| sequence no. 6 | | |
| GLEW FW1 | SEQ ID NO: 70 | EESGGGLAQPGGSLRLSCVASG |
| sequence no. 7 | | |
| GLEW FW1 | SEQ ID NO: 71 | VESGGGLALPGGSLTLSCVFSG |
| sequence no. 8 | | |

and in which:
iii) FR2, FR3 and FR4 are as mentioned herein for FR2, FR3 and FR4 of Nanobodies of the GLEW-class;
   and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein. In the above Nanobodies, one or more of the further Hallmark residues are preferably as described herein (for example, when they are V_{HH} sequences or partially humanized Nanobodies).

A Nanobody of the P, R, S 103 class may be an amino acid sequence that is comprised of four framework regions/sequences interrupted by three complementarity determining regions/sequences, in which
i) the amino acid residue at position 103 according to the Kabat numbering is different from W;
   and in which:
ii) preferably the amino acid residue at position 103 according to the Kabat numbering is P, R or S, and more preferably R;
   and in which:
iii) FR1 is an amino acid sequence that has at least 80% amino acid identity with at least one of the following amino acid sequences:

TABLE A-20
Representative FW1 sequences for Nanobodies of the P, R, S 103-group.

| | | |
|---|---|---|
| P, R, S 103 FW1 sequence no. 1 | SEQ ID NO: 92 | AVQLVESGGGLVQAGGSLRLSCAASGRTFS |
| P, R, S 103 FW1 sequence no. 2 | SEQ ID NO: 93 | QVQLQESGGGMVQPGGSLRLSCAASGFDFG |
| P, R, S 103 FW1 sequence no. 3 | SEQ ID NO: 94 | EVHLVESGGGLVRPGGSLRLSCAAFGFIFK |
| P, R, S 103 FW1 sequence no. 4 | SEQ ID NO: 95 | QVQLAESGGGLVQPGGSLKLSCAASRTIVS |
| P, R, S 103 FW1 sequence no. 5 | SEQ ID NO: 96 | QEHLVESGGGLVDIGGSLRLSCAASERIFS |
| P, R, S 103 FW1 sequence no. 6 | SEQ ID NO: 97 | QVKLEESGGGLAQPGGSLRLSCVASGFTFS |
| P, R, S 103 EW1 sequence no. 7 | SEQ ID NO: 98 | EVQLVESGGGLVQPGGSLRLSCVCVSSGCT |
| P, R, S 103 FW1 sequence no. 8 | SEQ ID NO: 99 | EVQLVESGGGLALPGGSLTLSCVFSGSTFS |

and in which
iv) FR2 is an amino acid sequence that has at least 80% amino acid identity with at least one of the following amino acid sequences:

TABLE A-21

Representative FW2 sequences for Nanobodies of the
P, R, S 103-group.

| | | |
|---|---|---|
| P, R, S 103 FW2 | SEQ ID NO: 102 | WFRQAPGKEREFVA |
| sequence no. 1 | | |
| P, R, S 103 FW2 | SEQ ID NO: 103 | WVRQAPGKVLEWVS |
| sequence no. 2 | | |
| P, R, S 103 FW2 | SEQ ID NO: 104 | WVRRPPGKGLEWVS |
| sequence no. 3 | | |
| P, R, S 103 FW2 | SEQ ID NO: 105 | WIRQAPGKEREGVS |
| sequence no. 4 | | |
| P, R, S 103 FW2 | SEQ ID NO: 106 | WVRQYPGKEPEWVS |
| sequence no. 5 | | |
| P, R, S 103 FW2 | SEQ ID NO: 107 | WFRQPPGKEHEFVA |
| sequence no. 6 | | |
| P, R, S 103 FW2 | SEQ ID NO: 108 | WYRQAPGKRTELVA |
| sequence no. 7 | | |
| P, R, S 103 FW2 | SEQ ID NO: 109 | WLRQAPGQGLEWVS |
| sequence no. 8 | | |
| P, R, S 103 FW2 | SEQ ID NO: 110 | WLRQTPGKGLEWVG |
| sequence no. 9 | | |
| P, R, S 103 FW2 | SEQ ID NO: 111 | WVRQAPGKAEEFVS |
| sequence no. 10 | | |

and in which:
v) FR3 is an amino acid sequence that has at least 80% amino acid identity with at least one of the following amino acid sequences:

TABLE A-22
Representative FW3 sequences for Nanobodies of the P, R, S 103-group.

| | | |
|---|---|---|
| P, R, S 103 FW3 sequence no. 1 | SEQ ID NO: 112 | RFTISRDNAKNTVYLQMNSLKPEDTAVYYCAA |
| P, R, S 103 FW3 sequence no. 2 | SEQ ID NO: 113 | RFTISRDNARNTLYLQMDSLIPEDTALYYCAR |
| P, R, S 103 FW3 sequence no. 3 | SEQ ID NO: 114 | RFTISRDNAKNEMYLQMNNLKTEDTGVYWCGA |
| P, R, S 103 FW3 sequence no. 4 | SEQ ID NO: 115 | RFTISSDSNRNMIYLQMNNLKPEDTAVYYCAA |
| P, R, S 103 FW3 sequence no. 5 | SEQ ID NO: 116 | RFTISRDNAKNMLYLHLNNLKSEDTAVYYCRR |
| P, R, S 103 FW3 sequence no. 6 | SEQ ID NO: 117 | RFTISRDNAKKTVYLRLNSLNPEDTAVYSCNL |
| P, R, S 103 FW3 sequence no. 7 | SEQ ID NO: 118 | RFKISRDNAKKTLYLQMNSLGPEDTAMYYCQR |
| P, R. S 103 FW3 sequence no. 8 | SEQ ID NO: 119 | RFTVSRDNGKNTAYLRMNSLKPEDTADYYCAV |

and in which:
vi) FR4 is an amino acid sequence that has at least 80% amino acid identity with at least one of the following amino acid sequences:

TABLE A-23

Representative FW4 sequences for Nanobodies of the
P, R, S 103-group.

| | | |
|---|---|---|
| P, R. S 103 FW4 | SEQ ID NO: 120 | IRGQGTQVTVSS |
| sequence no. 1 | | |
| P, R, S 103 FW4 | SEQ ID NO: 121 | LRGGTQVTVSS |
| sequence no. 2 | | |
| P, R, S 103 FW4 | SEQ ID NO: 122 | GNKGTLVTVSS |
| sequence no. 3 | | |
| P, R, S 103 FW4 | SEQ ID NO: 123 | SSPGTQVTVSS |
| sequence no. 4 | | |
| P, R, S 103 FW4 | SEQ ID NO: 124 | SSQGTLVTVSS |
| sequence no. 5 | | |
| P, R, S 103 FW4 | SEQ ID NO: 125 | RSRGIQVTVSS |
| sequence no. 6 | | |

and in which:
vii) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the P, R, S 103 class may be an amino acid sequence that is comprised of four framework regions/sequences interrupted by three complementarity determining regions/sequences, in which:
i) the amino acid residue at position 103 according to the Kabat numbering is different from W;
   and in which:
ii) preferably the amino acid residue at position 103 according to the Kabat numbering is P, R or S, and more preferably R;
   and in which:
iii) FR1 is an amino acid sequence that, on positions 5 to 26 of the Kabat numbering, has at least 80% amino acid identity with at least one of the following amino acid sequences:

TABLE A-24

Representative FW1 sequences (amino acid residues 5 to 26) for
Nanobodies of the P, R, S 103-group.

| | | |
|---|---|---|
| P, R, S 103 FW1 sequence no. 9 | SEQ ID NO: 100 | VESGGGLVQAGGSLRLSCAASG |
| P, R, S 103 FW1 sequence no. 10 | SEQ ID NO: 101 | AESGGGLVQPGGSLKLSCAASR |

and in which:
iv) FR2, FR3 and FR4 are as mentioned herein for FR2, FR3 and FR4 of Nanobodies of the P, R, S 103 class;
   and in which:
v) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In the above Nanobodies, one or more of the further Hallmark residues are preferably as described herein (for example, when they are V_{HH} sequences or partially humanized Nanobodies).

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the FR1 sequences present in the Nanobodies of SEQ ID NO's: 320 to 370,
   or from the group consisting of amino acid sequences that have at least 80%, preferably at least 90%, more preferably at least 95%, even more preferably at least 99% sequence identity (as defined herein) with one of said FR1 sequences; in which
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position is as indicated in said FR1 sequence;
   and/or from the group consisting of amino acid sequences that have 3, 2 or only 1 “amino acid difference(s)” (as defined herein) with one of said FR1 sequences, in which:
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position is as indicated in said FR1 sequence;
      and in which:
(b) FR2 is chosen from the group consisting of the FR2 sequences present in the Nanobodies of SEQ ID NO's: 320 to 370,
   or from the group consisting of amino acid sequences that have at least 80%, preferably at least 90%, more preferably at least 95%, even more preferably at least 99% sequence identity (as defined herein) with one of said FR2 sequences; in which
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
   and/or from the group consisting of amino acid sequences that have 3, 2 or only 1 “amino acid difference(s)” (as defined herein) with one of said FR2 sequences, in which:
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
      and in which:
(c) FR3 is chosen from the group consisting of the FR3 sequences present in the Nanobodies of SEQ ID NO's: 320 to 370,
   or from the group consisting of amino acid sequences that have at least 80%, preferably at least 90%, more preferably at least 95%, even more preferably at least 99% sequence identity (as defined herein) with one of said FR3 sequences; in which
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
   and/or from the group consisting of amino acid sequences that have 3, 2 or only 1 “amino acid difference(s)” (as defined herein) with one of said FR3 sequences, in which:
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
      and in which:
(d) FR4 is chosen from the group consisting of the FR4 sequences present in the Nanobodies of SEQ ID NO's: 320 to 370,
   or from the group consisting of amino acid sequences that have at least 80%, preferably at least 90%, more preferably at least 95%, even more preferably at least 99% sequence identity (as defined herein) with one of said FR4 sequences; in which
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR3 sequence;
   and/or from the group consisting of amino acid sequences that have 3, 2 or only 1 “amino acid difference(s)” (as defined herein) with one of said FR4 sequences, in which:
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR4 sequence;
      and in which:
(e) CDR1, CDR2 and CDR3 are as defined herein, and are preferably as defined according to one of the preferred embodiments herein, and are more preferably as defined according to one of the more preferred embodiments herein.

Some particularly preferred Nanobodies of the invention can be chosen from the group consisting of the amino acid sequences of SEQ ID NO's: 320 to 370, or from the group consisting of amino acid sequences that have at least 80%, preferably at least 90%, more preferably at least 95%, even more preferably at least 99% sequence identity (as defined herein) with one of said amino acid sequences; in which
i) the Hallmark residues can be as indicated in Table A-3 above;
ii) any amino acid substitution at any position other than a Hallmark position is preferably either a conservative amino acid substitution (as defined herein) and/or an amino acid substitution as defined in Tables A-5-A-8; and/or
iii) said amino acid sequence preferably only contains amino acid substitutions, and no amino acid deletions or insertions, compared to the above amino acid sequence(s).

Some even more particularly preferred Nanobodies of the invention can be chosen from the group consisting of the amino acid sequences of SEQ ID NO's 320 to 370, or from the group consisting of amino acid sequences that have at least 80%, preferably at least 90%, more preferably at least 95%, even more preferably at least 99% sequence identity (as defined herein) with one of said amino acid sequences; in which
(1) the Hallmark residues are as indicated in the pertinent sequence from SEQ ID NO's 320 to 370;
(2) any amino acid substitution at any position other than a Hallmark position is preferably either a conservative amino acid substitution (as defined herein) and/or an amino acid substitution as defined in Tables A-5-A-8; and/or
(3) said amino acid sequence preferably only contains amino acid substitutions, and no amino acid deletions or insertions, compared to the pertinent sequence chosen from SEQ ID NO's 320 to 370.

Some of the most preferred Nanobodies of the invention against IL-6 can be chosen from the group consisting of the amino acid sequences of SEQ ID NO's 320 to 370.

Preferably, the CDR sequences and FR sequences in the Nanobodies of the invention are such that the Nanobody of the invention binds to IL-6, with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence.

In another preferred, but non-limiting aspect, the invention relates to a Nanobody as described above, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 320 to 370. This degree of amino acid identity can for example be determined by determining the degree of amino acid identity (in a manner described herein) between said Nanobody and one or more of the sequences of SEQ ID NO's: 320 to 370, in which the amino acid residues that form the framework regions are disregarded. Such Nanobodies can be as further described herein.

As already mentioned herein, another preferred but non-limiting aspect of the invention relates to a Nanobody with an amino acid sequence that is chosen from the group consisting of SEQ ID NO's: 320 to 370 or from the group consisting of from amino acid sequences that have more than 80%, preferably more than 90%, more preferably more than 95%, such as 99% or more sequence identity (as defined herein) with at least one of the amino acid sequences of SEQ ID NO's: 320 to 370.

Also, in the above Nanobodies:
i) any amino acid substitution (when it is not a humanizing substitution as defined herein) is preferably, and compared to the corresponding amino acid sequence of SEQ ID NO's: 320 to 370, a conservative amino acid substitution, (as defined herein);
   and/or:
ii) its amino acid sequence preferably contains either only amino acid substitutions, or otherwise preferably no more than 5, preferably no more than 3, and more preferably only 1 or 2 amino acid deletions or insertions, compared to the corresponding amino acid sequence of SEQ ID NO's: 320 to 370;
   and/or
iii) the CDR's may be CDR's that are derived by means of affinity maturation, for example starting from the CDR's of to the corresponding amino acid sequence of SEQ ID NO's: 320 to 370.

Preferably, the CDR sequences and FR sequences in the Nanobodies of the invention are such that the Nanobodies of the invention (and polypeptides of the invention comprising the same):
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, CDR sequences and FR sequences present in the Nanobodies of the invention are such that the Nanobodies of the invention will bind to IL-6 with an affinity less than 500 nM, preferably less than 200 nM, more preferably less than 10 nM, such as less than 500 pM.

The affinity of the Nanobody of the invention against IL-6, can be determined in a manner known per se, for example using the assay described herein.

According to one non-limiting aspect of the invention, a Nanobody may be as defined herein, but with the proviso that it has at least “one amino acid difference” (as defined herein) in at least one of the framework regions compared to the corresponding framework region of a naturally occurring human VH domain, and in particular compared to the corresponding framework region of DP-47. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring human VH domain, and in particular compared to the corresponding framework region of DP-47. Usually, a Nanobody will have at least one such amino acid difference with a naturally occurring VH domain in at least one of FR2 and/or FR4, and in particular at least one of the Hallmark residues in FR2 and/or FR4 (again, (including those at positions 108, 103 and/or 45).

Also, a humanized Nanobody of the invention may be as defined herein, but with the proviso that it has at least “one amino acid difference” (as defined herein) in at least one of the framework regions compared to the corresponding framework region of a naturally occurring VHH domain. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring VHH domain. Usually, a Nanobody will have at least one such amino acid difference with a naturally occurring VHH domain in at least one of FR2 and/or FR4, and in particular at least one of the Hallmark residues in FR2 and/or FR4 (again, (including those at positions 108, 103 and/or 45).

As will be clear from the disclosure herein, it is also within the scope of the invention to use natural or synthetic analogs, mutants, variants, alleles, homologs and orthologs (herein collectively referred to as “analogs”) of the Nanobodies of the invention as defined herein, and in particular analogs of the Nanobodies of SEQ ID NO's 320 to 370. Thus, according to one embodiment of the invention, the term “Nanobody of the invention” in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables A-5-A-8 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art.

Alternatively, substitutions or insertions may be designed so as to introduce one or more sites for attachment of functional groups (as described herein), for example to allow site-specific pegylation (again as described herein).

As can be seen from the data on the V_{HH} entropy and V_{HH} variability given in Tables A-5-A-8 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to IL-6, with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

In particular, amino acid sequences and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a monovalent amino acid sequence of the invention (or a polypeptide that contains only one amino acid sequence of the invention) is preferably such that it will bind to IL-6 with an affinity less than 500 nM, preferably less than 200 nM, more preferably less than 10 nM, such as less than 500 pM.

The affinity of the analog against IL-6, can be determined in a manner known per se, for example using the assay described herein.

The analogs are preferably also such that they retain the favourable properties the Nanobodies, as described herein.

Also, according to one preferred embodiment, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID NOs 320 to 370.

Also, the framework sequences and CDR's of the analogs are preferably such that they are in accordance with the preferred embodiments defined herein. More generally, as described herein, the analogs will have (a) a Q at position 108; and/or (b) a charged amino acid or a cysteine residue at position 45 and preferably an E at position, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparison between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The Nanobodies of the invention may be suitably humanized at any framework residue(s), such as at one or more Hallmark residues (as defined herein) or at one or more other framework residues (i.e. non-Hallmark residues) or any suitable combination thereof. One preferred humanizing substitution for Nanobodies of the “P, R, S-103 group” or the “KERE group” is Q108 into L108. Nanobodies of the “GLEW class” may also be humanized by a Q108 into L108 substitution, provided at least one of the other Hallmark residues contains a camelid (camelizing) substitution (as defined herein). For example, as mentioned above, one particularly preferred class of humanized Nanobodies has GLEW or GLEW-like sequence at positions 4447; P, R or S (and in particular R) at position 103 and an L at position 108; another particularly preferred class of humanized Nanobodies has KERE, KQRE or another KERE-like sequence at positions 4346 and a Q at position 108 (and optionally one or more of the other Hallmark residues for the KERE-group as defined herein).

Another class of humanized Nanobodies has P, R or S (and in particular R) at position 103 and a Q at position 108 (and optionally one or more of the other Hallmark residues for the P, R, S 103-group as defined herein).

The humanized and other analogs, and nucleic acid sequences encoding the same, can be provided in any manner known per se. For example, the analogs can be obtained by providing a nucleic acid that encodes a naturally occurring V_{HH} domain, changing the codons for the one or more amino acid residues that are to be substituted into the codons for the corresponding desired amino acid residues (e.g. by site-directed mutagenesis or by PCR using suitable mismatch primers), expressing the nucleic acid/nucleotide sequence thus obtained in a suitable host or expression system; and optionally isolating and/or purifying the analog thus obtained to provide said analog in essentially isolated form (e.g. as further described herein). This can generally be performed using methods and techniques known per se, which will be clear to the skilled person, for example from the handbooks and references cited herein, the background art cited herein and/or from the further description herein. Alternatively, a nucleic acid encoding the desired analog can be synthesized in a manner known per se (for example using an automated apparatus for synthesizing nucleic acid sequences with a predefined amino acid sequence) and can then be expressed as described herein. Yet another technique may involve combining one or more naturally occurring and/or synthetic nucleic acid sequences each encoding a part of the desired analog, and then expressing the combined nucleic acid sequence as described herein. Also, the analogs can be provided using chemical synthesis of the pertinent amino acid sequence using techniques for peptide synthesis known per se, such as those mentioned herein.

In this respect, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

Some preferred, but non-limiting camelizing substitutions can be derived from Tables A-5-A-8. It will also be clear that camelizing substitutions are one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original V_{H} domain).

Generally, however, such camelizing substitutions are preferably such that the resulting an amino acid sequence at least contains (a) a Q at position 108; and/or (b) a charged amino acid or a cysteine residue at position 45 and preferably also an E at position 44, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO's 320 to 370. Thus, according to one embodiment of the invention, the term “Nanobody of the invention” in its broadest sense also covers such pats or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

More in particular, the invention provides parts or fragments of the Nanobodies of the invention (including analogs thereof) that can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such Nanobody.

In particular, parts or fragments (including analogs thereof) of the Nanobodies and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹ s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, parts or fragments (including analogs thereof) of a monovalent Nanobody of the invention (or a polypeptide that contains only one Nanobody of the invention) are preferably such that they will bind to IL-6 with an affinity less than 500 nM, preferably less than 200 nM, more preferably less than 10 nM, such as less than 500 pM.

Some preferred IC50 values for binding of parts or fragments (including analogs thereof) of the Nanobodies or polypeptides of the invention to IL-6 will become clear from the further description and examples herein.

Any part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting embodiment, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred embodiment, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 320 to 370.

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g enzymatical) modification, of the Nanobodies of the invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody of the invention, or optionally via a suitable linker or spacer, as will again be clear to the skilled person.

One of the most widely used techniques for increasing the half-life and/or the reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody of the invention, a Nanobody of the invention may be modified so as to suitably introduce one or more cysteine residues for attachment of PEG, or an amino acid sequence comprising one or more cysteine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga ad ⁶⁸Ga or other metals or metallic cations that are particularly suited for use in in vivo, in vitro or in situ diagnosis and imaging, such as (¹⁵⁷Gd, ⁵⁵Mn, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the Nanobody of the invention.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a toxin or to a toxic residue or moiety. Examples of toxic moieties, compounds or residues which can be linked to a Nanobody of the invention to provide—for example—a cytotoxic compound will be clear to the skilled person and can for example be found in the prior art cited above and/or in the further description herein. One example is the so-called ADEPT™ technology WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

More in particular, the invention provides derivatives of Nanobodies and polypeptides that can bind to IL-6 with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

In particular, derivatives of Nanobodies and polypeptides of the invention are preferably such that they:
bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
   and/or such that they:
bind to IL-6 with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or such that they:
bind to IL-6 with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, derivatives of a monovalent Nanobody of the invention (or a polypeptide that contains only one Nanobody of the invention) are preferably such that they will bind to IL-6 with an affinity less than 500 nM, preferably less than 200 nM, more preferably less than 10 nM, such as less than 500 pM.

Some preferred IC50 values for binding of derivatives of the Nanobodies or polypeptides of the invention to IL-6 will become clear from the further description and examples herein.

As mentioned above, the invention also relates to proteins or polypeptides that essentially consist of or comprise at least one Nanobody of the invention. By “essentially consist of” is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
a) can comprise an N-terminal Met residue, for example as result of expression in a heterologous host cell or host organism.
b) may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
c) may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising a Nanobody of the invention, as mentioned below;
d) may form a “tag”, for example an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the Nanobody sequence (for this purpose, the tag may optionally be linked to the Nanobody sequence via a cleavable linker sequence or contain a cleavable motif). Some preferred, but non-limiting examples of such residues are multiple histidine residues, glutatione residues and a myc-tag such as AAAEQKLISEEDLNGAA [SEQ ID NO: 156];
e) may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention.

According to another aspect, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a “Nanobody fusion”.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

For example, the further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope).

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005),

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

The further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope). For example, the further amino acid sequence may provide a second binding site that is directed against a serum protein (such as, for example, human serum albumin or another serum protein such as IgG), so as to provide increased half-life in serum. Reference is for example made to EP 0 368 684, WO 91/01743, WO 01/45746 and WO 04/003019 (in which various serum proteins are mentioned), the International application by applicant entitled “Nanobodies® against amyloid-beta and polypeptides comprising the same for the treatment of degenerative neural diseases such as Alzheimer's disease” (in which various other proteins are mentioned), as well as to Harmsen et al., Vaccine, 23 (41); 4926-42.

In one specific aspect of the invention, a Nanobody of the invention or a compound, construct or polypeptide of the invention comprising at least one Nanobody of the invention may have an increased half-life, compared to the corresponding Nanobody of the invention. Some preferred, but non-limiting examples of such Nanobodies, compounds and polypeptides will become clear to the skilled person based on the further disclosure herein, and for example comprise Nanobodies sequences or polypeptides of the invention that have been chemically modified to increase the half-life thereof (for example, by means of pegylation); Nanobodies of the invention that comprise at least one additional binding site for binding to a serum protein (such as serum albumin. Reference is for example made to the US provisional application by Ablynx N.V. entitled “Immunoglobulin domains with multiple binding sites” filed on Nov. 27, 2006); or polypeptides of the invention that comprise at least one Nanobody of the invention that is linked to at least one moiety (and in particular at least one Nanobody) that increases the half-life of the Nanobody of the invention. Examples of polypeptides of the invention that comprise such half-life extending moieties or Nanobodies will become clear to the skilled person based on the further disclosure herein; and for example include, without limitation, polypeptides in which the one or more Nanobodies of the invention are suitable linked to one or more serum proteins or fragments thereof (such as serum albumin or suitable fragments thereof) or to one or more binding units that can bind to serum proteins (such as, for example, Nanobodies or (single) domain antibodies that can bind to serum proteins such as serum albumin, serum immunoglobulins such as IgG, or transferrine); polypeptides in which a Nanobody of the invention is linked to an Fc portion (such as a human Fc) or a suitable part or fragment thereof; or polypeptides in which the one or more Nanobodies of the invention are suitable linked to one or more small proteins or peptides that can bind to serum proteins (such as, without limitation, the proteins and peptides described in WO 91/01743, WO 01/45746, WO 02/076489).

Generally, the Nanobodies of the invention (or compounds, constructs or polypeptides comprising the same) with increased half-life preferably have a half-life that is at least 1.5 times, preferably at least 2 times, such as at least 5 times, for example at least 10 times or more than 20 times, greater than the half-life of the corresponding Nanobody of the invention per se. For example, the Nanobodies, compounds, constructs or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding Nanobody of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), at preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another one aspect of the invention, a polypeptide of the invention comprises one or more (such as two or preferably one) Nanobodies of the invention linked (optionally via one or more suitable linker sequences) to one or more (such as two and preferably one) Nanobodies that allow the resulting polypeptide of the invention to cross the blood brain barrier. In particular, said one or more Nanobodies that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

In particular, it has been described in the art that linking fragments of immunoglobulins (such as V_{H} domains) to serum albumin or to fragments thereof can be used to increase the half-life. Reference is for made to WO 00/27435 and WO 01/077137). According to the invention, the Nanobody of the invention is preferably either directly linked to serum albumin (or to a suitable fragment thereof) or via a suitable linker, and in particular via a suitable peptide linked so that the polypeptide of the invention can be expressed as a genetic fusion (protein). According to one specific aspect, the Nanobody of the invention may be linked to a fragment of serum albumin that at least comprises the domain III of serum albumin or part thereof. Reference is for example made to the U.S. provisional application 60/788,256 of Ablynx N.V. entitled “Albumin derived Nanobody, use thereof for increasing the half-life of therapeutic proteins and of other therapeutic proteins and entities, and constructs comprising the same” filed on Mar. 31, 2006.

Alternatively, the further Nanobody may provide a second binding site or binding unit that is directed against a serum protein (such as, for example, human serum albumin or another serum protein such as IgG), so as to provide increased half-life in serum. Such Nanobodies for example include the Nanobodies described below, as well as the small peptides and binding proteins described in WO 91/01743, WO 01/45746 and WO 02/076489 and the dAb's described in WO 03/002609 and WO 04/003019. Reference is also made to Harmsen et al., Vaccine, 23 (41); 492642, 2005 as well as to EP 0 368 684, as well as to the following the U.S. provisional applications 60/843,349, 60/850,774, 60/850,775 by Ablynx N.V. mentioned herein and US provisional application of Ablynx N.V. entitled “Peptides capable of binding to serum proteins” filed on Dec. 5, 2006 (also mentioned herein).

Such amino acid sequences and/or Nanobodies may in particular be directed against serum albumin (and more in particular human serum albumin) and/or against IgG (and more in particular human IgG). For example, such amino acid sequences and/or Nanobodies may be amino acid sequences and/or Nanobodies that are directed against (human) serum albumin and amino acid sequences and/or Nanobodies that can bind to amino acid residues on (human) serum albumin that are not involved in binding of serum albumin to FcRn (see for example WO 06/0122787) and/or amino acid sequences and/or Nanobodies that are capable of binding to amino acid residues on serum albumin that do not form part of domain III of serum albumin (see again see for example WO 06/0122787); amino acid sequences and/or Nanobodies that have or can provide an increased half-life (see for example the U.S. provisional application 60/843,349 by Ablynx N.V. entitled “Serum albumin binding proteins with long half-lives” filed on Sep. 8, 2006); amino acid sequences and/or Nanobodies against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus), reference is again made to the U.S. provisional application 60/843,349); amino acid sequences and/or Nanobodies that can bind to serum albumin in a pH independent manner (see for example the U.S. provisional application 60/850,774 by Ablynx N.V. entitled “Nanobodies that bind to serum proteins in a manner that is essentially independent of the pH, compounds comprising the same, and uses thereof”, filed on Oct. 11, 2006) and/or amino acid sequences and/or Nanobodies that are conditional binders (see for example the US provisional application 60/850,775 by Ablynx N.V. entitled “Nanobodies that bind to a desired molecule in a conditional manner”, filed on Oct. 11, 2006).

According to another aspect, the one or more further amino acid sequences may comprise one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L} domain or to a natural or synthetic analog of a V_{H} or V_{L} domain, again optionally via a linker sequence (including but not limited to other (single) domain antibodies, such as the dAb's described by Ward et al.).

The at least one Nanobody may also be linked to one or more (preferably human) CH₁, CH₂ and/or CH₃ domains, optionally via a linker sequence. For instance, a Nanobody linked to a suitable CH₁ domain could for example be used—together with suitable light chains—to generate antibody fragments/structures analogous to conventional Fab fragments or F(ab′)2 fragments, but in which one or (in case of an F(ab′)2 fragment) one or both of the conventional V_{H} domains have been replaced by a Nanobody of the invention. Also, two Nanobodies could be linked to a CH3 domain (optionally via a linker) to provide a construct with increased half-life in vivo.

According to one specific aspect of a polypeptide of the invention, one or more Nanobodies of the invention may linked to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more CH₂ and/or CH₃ domains of an antibody, such as from a heavy chain antibody (as described herein) and more preferably from a conventional human 4-chain antibody; and/or may form (part of) and Fc region, for example from IgG, from IgE or from another human Ig. For example, WO 94/04678 describes heavy chain antibodies comprising a Camelid V_{HH} domain or a humanized derivative thereof (i.e. a Nanobody), in which the Camelidae CH₂ and/or CH₃ domain have been replaced by human CH₂ and CH₃ domains, so as to provide an immunoglobulin that consists of 2 heavy chains each comprising a Nanobody and human CH2 and CH3 domains (but no CH1 domain), which immunoglobulin has the effector function provided by the CH2 and CH3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077 and WO 05/017148, as well as the review by Holliger and Hudson, supra. Coupling of a Nanobody of the invention to an Fc portion may also lead to an increased half-life, compared to the corresponding Nanobody of the invention. For some applications, the use of an Fc portion and/or of constant domains (i.e. CH₂ and/or CH₃ domains) that confer increased half-life without any biologically significant effector function may also be suitable or even preferred. Other suitable constructs comprising one or more Nanobodies and one or more constant domains with increased half-life in vivo will be clear to the skilled person, and may for example comprise, two Nanobodies linked to a CH3 domain, optionally via a linker sequence. Generally, any fusion protein or derivatives with increased half-life will preferably have a molecular weight of more than 50 kD, the cut-off value for renal absorption.

The further amino acid sequences may also form a signal sequence or leader sequence that directs secretion of the Nanobody or the polypeptide of the invention from a host cell upon synthesis (for example to provide a pre-, pro- or prepro- form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the “Peptrans” vectors mentioned above, the sequences described by Cardinale et al. and the amino acid sequences and antibody fragments known per se that can be used to express or produce the Nanobodies and polypeptides of the invention as so-called “intrabodies”, for example as described in WO 94/02610, WO 95/22618, U.S. Pat. No. 6,004,940, WO 03/014960, WO 99/07414; WO 05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a (cyto)toxic protein or polypeptide. Examples of such toxic proteins and polypeptides which can be linked to a Nanobody of the invention to provide—for example—a cytotoxic polypeptide of the invention will be clear to the skilled person and can for example be found in the prior art cited above and/or in the further description herein. One example is the so-called ADEPT™ technology WO 03/055527.

According to one preferred, but non-limiting embodiment, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). Polypeptides of the invention that comprise two or more Nanobodies, of which at least one is a Nanobody of the invention, will also be referred to herein as “multivalent” polypeptides of the invention, and the Nanobodies present in such polypeptides will also be referred to herein as being in a “multivalent format”. For example a “bivalent” polypeptide of the invention comprises two Nanobodies, optionally linked via a linker sequence, whereas a “trivalent” polypeptide of the invention comprises three Nanobodies, optionally linked via two linker sequences; etc.; in which at least one of the Nanobodies present in the polypeptide, and up to all of the Nanobodies present in the polypeptide, is/are a Nanobody of the invention.

In a multivalent polypeptide of the invention, the two or more Nanobodies may be the same or different, and may be directed against the same antigen or antigenic determinant (for example against the same part(s) or epitope(s) or against different parts or epitopes) or may alternatively be directed against different antigens or antigenic determinants; or any suitable combination thereof. For example, a bivalent polypeptide of the invention may comprise (a) two identical Nanobodies; (b) a first Nanobody directed against a first antigenic determinant of a protein or antigen and a second Nanobody directed against the same antigenic determinant of said protein or antigen which is different from the first Nanobody; (c) a first Nanobody directed against a first antigenic determinant of a protein or antigen and a second Nanobody directed against another antigenic determinant of said protein or antigen; or (d) a first Nanobody directed against a first protein or antigen and a second Nanobody directed against a second protein or antigen (i.e. different from said first antigen). Similarly, a trivalent polypeptide of the invention may, for example and without being limited thereto. comprise (a) three identical Nanobodies; (b) two identical Nanobody against a first antigenic determinant of an antigen and a third Nanobody directed against a different antigenic determinant of the same antigen; (c) two identical Nanobody against a first antigenic determinant of an antigen and a third Nanobody directed against a second antigen different from said first antigen; (d) a first Nanobody directed against a first antigenic determinant of a first antigen, a second Nanobody directed against a second antigenic determinant of said first antigen and a third Nanobody directed against a second antigen different from said first antigen; or (e) a first Nanobody directed against a first antigen, a second Nanobody directed against a second antigen different from said first antigen, and a third Nanobody directed against a third antigen different from said first and second antigen.

Polypeptides of the invention that contain at least two Nanobodies, in which at least one Nanobody is directed against a first antigen (i.e. against IL-6), and at least one Nanobody is directed against a second antigen (i.e. different from IL-6), will also be referred to as “multispecific” polypeptides of the invention, and the Nanobodies present in such polypeptides will also be referred to herein as being in a “multivalent format”. Thus, for example, a “bispecific” polypeptide of the invention is a polypeptide that comprises at least one Nanobody directed against a first antigen (i.e. IL-6), and at least one further Nanobody directed against a second antigen (i.e. different from IL-6), whereas a “trispecific” polypeptide of the invention is a polypeptide that comprises at least one Nanobody directed against a first antigen (i.e. IL-6), at least one further Nanobody directed against a second antigen (i.e. different from IL-6), and at least one further Nanobody directed against a third antigen (i.e. different from both IL-6, and the second antigen); etc.

Accordingly, in its simplest form, a bispecific polypeptide of the invention is a bivalent polypeptide of the invention (as defined herein), comprising a first Nanobody directed against IL-6, and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against IL-6, a second Nanobody directed against a second antigen and a third Nanobody directed against a third antigen, in which said first, second and third Nanobody may optionally be linked via one or more, and in particular one and more in particular two, linker sequences.

However, as will be clear from the description hereinabove, the invention is not limited thereto, in the sense that a multispecific polypeptide of the invention may comprise at least one Nanobody against IL-6, and any number of Nanobodies directed against one or more antigens different from IL-6.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for IL-6, or against the one or more other antigens), said order or arrangement is usually not critical and may be suitably chosen by the skilled person, optionally after on some limited routine experiments based on the disclosure herein. Thus, when reference is made to a specific multivalent or multispecific polypeptide of the invention, it should be noted that this encompasses any order or arrangements of the relevant Nanobodies, unless explicitly indicated otherwise.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221. Some other examples of some specific multispecific and/or multivalent polypeptide of the invention can be found in the applications by ABLYNX N.V. referred to herein.

One preferred, but non-limiting example of a multispecific polypeptide of the invention comprises at least one Nanobody of the invention and at least one Nanobody that provides for an increased half-life. Some preferred, but non-limiting examples of such Nanobodies include Nanobodies directed against serum proteins, such as human serum albumin, thyroxine-binding protein, (human) transferrin, fibrinogen, an immunoglobulin such as IgG, IgE or IgM, or one of the other serum proteins listed in WO 04/003019. Of these, Nanobodies that can bind to serum albumin (and in particular human serum albumin) or to IgG (and in particular human IgG, see for example Nanobody VH-1 described in the review by Muyldermans, supra) are particularly preferred (although for example, for experiments in mice or primates, Nanobodies against or cross-reactive with mouse serum albumin (MSA) or serum albumin from said primate, respectively, can be used. However, for pharmaceutical use, Nanobodies against human serum albumin or human IgG will usually be preferred). Nanobodies that provide for increased half-life and that can be used in the polypeptides of the invention include the Nanobodies directed against serum albumin that are described in WO 04/041865, in WO 06/122787 and in the further patent applications by Ablynx N.V., such as those mentioned above.

For example, for experiments in mice, Nanobodies against mouse serum albumin (MSA) can be used, whereas for pharmaceutical use, Nanobodies against human serum albumin can be used.

For example, the some preferred Nanobodies that provide for increased half-life for use in the present invention include Nanobodies that can bind to amino acid residues on (human) serum albumin that are not involved in binding of serum albumin to FcRn (see for example WO 06/0122787); Nanobodies that are capable of binding to amino acid residues on serum albumin that do not form part of domain III of serum albumin (see for example WO 06/0122787); Nanobodies that have or can provide an increased half-life (see for example the U.S. provisional application 60/843,349 by Ablynx N.V mentioned herein); Nanobodies against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus)) (see for example the U.S. provisional application 60/843,349 by Ablynx N.V); Nanobodies that can bind to serum albumin in a pH independent manner (see for example the U.S. provisional application 60/850,774 by Ablynx N.V. mentioned herein) and/or Nanobodies that are conditional binders (see for example the U.S. provisional application 60/850,775 by Ablynx N.V.).

Some particularly preferred Nanobodies that provide for increased half-life and that can be used in the polypeptides of the invention include the Nanobodies ALB-1 to ALB-10 disclosed in WO 06/122787 (see Tables II and III) of which ALB-8 (SEQ ID NO: 62 in WO 06/122787) is particularly preferred.

Another embodiment of the present invention is a polypeptide construct as described above wherein said at least one (human) serum protein is any of (human) serum albumin, (human) serum immunoglobulins, (human) thyroxine-binding protein, (human) transferrin, (human) fibrinogen, etc.

According to a specific, but non-limiting aspect of the invention, the polypeptides of the invention contain, besides the one or more Nanobodies of the invention, at least one Nanobody against human serum albumin. Although these Nanobodies against human serum albumin may be as generally described in the applications by applicant cited above (see for example W04/062551), according to a particularly preferred, but non-limiting embodiment, said Nanobody against human serum albumin consists of 4 framework regions (FR1 to FR4 respectively) and 3 complementarity determining regions (CDR1 to CDR3 respectively), in which:

i) CDR1 is an amino acid sequence chosen from the group consisting of:

| | |
|---|---|
| SFGMS | [SEQ ID NO: 140] |
| LNLMG | [SEQ ID NO: 141] |
| INLLG | [SEQ ID NO: 142] |
| NYWMY; | [SEQ ID NO: 143] |

and/or from the group consisting of amino acid sequences that have 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and in which:
ii) CDR2 is an amino acid sequence chosen from the group consisting of:

| | |
|---|---|
| SISGSGSDTLYADSVKG | [SEQ ID NO: 144] |
| TITVGDSTNYADSVKG | [SEQ ID NO: 145] |
| TITVGDSTSYADSVKG | [SEQ ID NO: 146] |
| SINGRGDDTRYADSVKG | [SEQ ID NO: 147] |
| AISADSSTKNYADSVKG | [SEQ ID NO: 148] |
| AISADSSDKRYADSVKG | [SEQ ID NO: 149] |
| RISTGGGYSYYADSVKG | [SEQ ID NO: 150] |

or from the group consisting of amino acid sequences that have at least 80%, preferably at least 90%, more preferably at least 95%, even more preferably at least 99% sequence identity (as defined herein) with one of the above amino acid sequences; in which
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences;
and in which:
iii) CDR3 is an amino acid sequence chosen from the group consisting of:

| | |
|---|---|
| DREAQVDTLDFDY | [SEQ ID NO: 151] |

or from the group consisting of amino acid sequences that have at least 80%, preferably at least 90%, more preferably at least 95%, even more preferably at least 99% sequence identity (as defined herein) with one of the above amino acid sequences; in which
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences;

or from the group consisting of:

| | |
|---|---|
| GGSLSR | [SEQ ID NO: 152] |
| RRTWHSEL | [SEQ ID NO: 153] |
| GRSVSRS | [SEQ ID NO: 154] |
| GRGSP | [SEQ ID NO: 155] |

and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences.

In another aspect, the invention relates to a Nanobody against human serum albumin, which consist of 4 framework regions (FR1 to FR4 respectively) and 3 complementarity determining regions (CDR1 to CDR3 respectively), which is chosen from the group consisting of Nanobodies with the one of the following combinations of CDR1, CDR2 and CDR3, respectively:

| | | | | | |
|---|---|---|---|---|---|
| CDR1: | SFGMS; | CDR2: | SISGSGSDTLYADSVKG; | CDR3: | GGSLSR; |
| CDR1: | LNLMG; | CDR2: | TITVGDSTNYADSVKG; | CDR3: | RRTWHSEL; |
| CDR1: | INLLG; | CDR2: | TITVGDSTSYADSVKG; | CDR3: | RRTWHSEL; |
| CDR1: | SFGMS; | CDR2: | SINGRGDDTRYADSVKG; | CDR3: | GRSVSRS; |
| CDR1: | SFGMS; | CDR2: | AISADSSDKRYADSVKG; | CDR3: | GRGSP; |
| CDR1: | SFGMS; | CDR2: | AISADSSDKRYADSVKG; | CDR3: | GRGSP; |
| CDR1: | NYWMY; | CDR2: | RISTGGGYSYYADSVKG; | CDR3: | DREAQVDTLDFDY. |

In the Nanobodies of the invention that comprise the combinations of CDR's mentioned above, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which

(1) any amino acid substitution is preferably a conservative amino acid substitution (as defined herein); and/or
(2) said amino acid sequence preferably only contains amino acid substitutions, and no amino acid deletions or insertions, compared to the above amino acid sequences;
and/or chosen from the group consisting of amino acid sequences that have 3, 2 or only 1 (as indicated in the preceding paragraph) “amino acid difference(s)” (as defined herein) with the mentioned CDR(s) one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences.

However, of the Nanobodies of the invention that comprise the combinations of CDR's mentioned above, Nanobodies comprising one or more of the CDR's listed above are particularly preferred; Nanobodies comprising two or more of the CDR's listed above are more particularly preferred; and Nanobodies comprising three of the CDR's listed above are most particularly preferred.

In these Nanobodies against human serum albumin, the Framework regions FR1 to FR4 are preferably as defined hereinabove for the Nanobodies of the invention.

Some preferred, but non-limiting examples of Nanobodies directed against human serum albumin that can be used in the polypeptides of the invention are listed in Table A-9 below. ALB-8 is a humanized version of ALB-1.

TABLE A-9

Preferred, but non-limiting examples of albumin-binding
Nanobodies

<Name, SEQ ID #; PRT (protein); ->
Sequence
<PMP 6A6(ALB-1), SEQ ID NO: 157; PRT; ->
AVQLVESGGGLVQPGNSLRLSCAASGFTFRSFGMSWVRQAPGKEPEWVSSISGSGSDTLYADSVKGRFTISR
DNAKTTLYLQMNSLKPEDTAVYYCTIGGSLSRSSQGTQVTVSS
<ALB-8(humanized ALB-1), SEQ ID NO: 158; PRT; ->
EVQLVESGGGLVQPGNSLRLSCAASGFTFSSFGMSWVRQAPGKGLEWVSSISGSGSDTLYADSVKGRFTISR
DNAKTTLYLQMNSLRPEDTAVYYCTIGGSLSRSSQGTLVTVSS
<PMP 6A8(ALB-2), SEQ ID NO: 159; PRT; ->
AVQLVESGGGLVQGGGSLRLACAASERIFDLNLMGWYRQGPGNERELVATCITVGDSTNYADSVKGRFTISM
DYTKQTVYLHMNSLRPEDTGLYYCKIRRTWHSELWGQGTQVTVSS

Generally, any derivatives and/or polypeptides of the invention with increased half-life (for example pegylated Nanobodies or polypeptides of the invention, multispecific Nanobodies directed against IL-6, and (human) serum albumin, or Nanobodies fused to an Fc portion, all as described herein) have a half-life that is at least 1.5 times, preferably at least 2 times, such as at least 5 times, for example at least 10 times or more than 20 times, the half-life of the corresponding Nanobody of the invention per se. For example, such a derivative or polypeptide with increased half-life may have a half-life that is increased with more than 1 hour, preferably more than 2 hours, more preferably of more than 6 hours, such as of more than 12 hours, or even more than 24, 48 or 72 hours, compared to the corresponding Nanobody of the invention per se.

In a preferred, but non-limiting aspect of the invention, such derivatives or polypeptides may exhibit a serum half-life in human of at least about 12 hours, preferably at least 24 hours, more preferably at least 48 hours, even more preferably at least 72 hours or more. For example, such derivatives or polypeptides may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Half-life can generally be defined as the time taken for the serum concentration of the polypeptide to be reduce by 50%, in vivo, for example due to degradation of the ligand and/or clearance or sequestration of the ligand by natural mechanisms. Methods for pharmacokinetic analysis and determination of half-life are familiar to those skilled in the art. Details may be found in Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2 nd Rev. ex edition (1982).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445, of which FC44 (SEQ ID NO 160) and FC5 (SEQ ID NO: 161) are preferred examples.

TABLE A-10

Sequence listing of FC44 and FC5

<Name, SEQ ID #; PRT (protein); ->
Sequence
< FC44, SEQ ID NO: 160; PRT; ->
EVQLQASGGGLVQAGGSLRLSCSASVRTFSIYAMGWFRQAPGKEREFVAGINRSGDVTKYADFVKGRFSISR
DNAKNMVYLQMNSLKPEDTALYYCAATWAYDTVGALTSGYNFWGQGTQVTVSS
< FC5, SEQ ID NO: 161; PRT; ->
EVQLQASGGGLVQAGGSLRLSCAASGFKITHYTMGWFRQAPGKEREFVSRITWGGDNTFYSNSVKGRFTISR
DNAKNTVYLQMNSLKPEDTADYYCAAGSTSTATPLRVDYWGKGTQVTVSS

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077, hinge-like regions such as the hinge regions of naturally occurring heavy chain antibodies or similar sequences (such as described in WO 94/04678).

Some other particularly preferred linkers are poly-alanine (such as AAA), as well as the linkers mentioned in Table A-11, of which AAA, GS-7 and GS-9 are particularly preferred.

TABLE A-11

Sequence listing of linkers

<Name, SEQ ID #; PRT (protein); ->
Sequence
< GS30, SEQ ID NO: 162; PRT; ->
GGGGSGGGGSGGGGSGGGGSGGGGSGGGGS
< GS15, SEQ ID NO: 163; PRT; ->
GGGGSGGGGSGGGGS
< GS9, SEQ ID NO: 164; PRT; ->
GGGGSGGGS
< GS7, SEQ ID NO: 165; PRT; ->
SGGSGGS
< Llama upper long hinge region, SEQ ID NO: 166;
PRT; ->
EPKTPKPQPAAA

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for IL-6, or against the one or more of the other antigens. Based on the disclosure herein, the skilled person will be able to determine the optimal linker(s) for use in a specific polypeptide of the invention, optionally after on some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after on some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after on some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after on some limited routine experiments.

Usually, for easy of expression and production, a polypeptide of the invention will be a linear polypeptide. However, the invention in its broadest sense is not limited thererto. For example, when a polypeptide of the invention comprises three of more Nanobodies, it is possible to link them use a linker with three or more “arms”, which each “arm” being linked to a Nanobody, so as to provide a “star-shaped” construct. It is also possible, although usually less preferred, to use circular constructs.

The invention also comprises derivatives of the polypeptides of the invention, which may be essentially analogous to the derivatives of the Nanobodies of the invention, i.e. as described herein.

The invention also comprises proteins or polypeptides that “essentially consist” of a polypeptide of the invention (in which the wording “essentially consist of” has essentially the same meaning as indicated hereinabove).

According to one embodiment of the invention, the polypeptide of the invention is in essentially isolated from, as defined herein.

The amino acid sequences and/or Nanobodies, polypeptides and nucleic acids of the invention can be prepared in a manner known per se, as will be clear to the skilled person from the further description herein. For example, the amino acid sequences and/or Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the amino acid sequences and/or Nanobodies, polypeptides and nucleic acids include the methods and techniques described herein.

As will be clear to the skilled person, one particularly useful method for preparing an amino acid sequence and/or Nanobody and/or a polypeptide of the invention generally comprises the steps of:
the expression, in a suitable host cell or host organism (also referred to herein as a “host of the invention”) or in another suitable expression system of a nucleic acid that encodes said amino acid sequence and/or Nanobody or polypeptide of the invention (also referred to herein as a “nucleic acid of the invention”), optionally followed by:
isolating and/or purifying the amino acid sequence and/or Nanobody or polypeptide of the invention thus obtained.

In particular, such a method may comprise the steps of:
cultivating and/or maintaining a host of the invention under conditions that are such that said host of the invention expresses and/or produces at least one amino acid sequence and/or Nanobody and/or polypeptide of the invention; optionally followed by:
isolating and/or purifying the amino acid sequence and/or Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises
a) at least one nucleic acid of the invention; operably connected to
b) one or more regulatory elements, such as a promoter and optionally a suitable terminator;
   and optionally also
c) one or more further elements of genetic constructs known per se;
   in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promoter). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example derived a cell or cell line derived from a human, from the mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The amino acid sequences and/or Nanobodies and polypeptides of the invention can also be introduced and expressed in one or more cells, tissues or organs of a multicellular organism, for example for prophylactic and/or therapeutic purposes (e.g. as a gene therapy). For this purpose, the nucleotide sequences of the invention may be introduced into the cells or tissues in any suitable way, for example as such (e.g. using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 5,5895,466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

For expression of the Nanobodies in a cell, they may also be expressed as so-called or as so-called “intrabodies”, as for example described in WO 94/02610, WO 95/22618 and U.S. Pat. No. 6,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

For production, the amino acid sequences and/or Nanobodies and polypeptides of the invention can for example also be produced in the milk of transgenic mammals, for example in the milk of rabbits, cows, goats or sheep (see for example U.S. Pat. No. 5,741,957, U.S. Pat. No. 5,304,489 and U.S. Pat. No. 5,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the amino acid sequences and/or Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, Polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast are usually leads to a glycosylation pattern that differs from human glycosylation. Nevertheless, it should be understood that all the foregoing host cells and expression systems can be used in the invention, depending on the desired Nanobody or protein to be obtained.

Thus, according to one non-limiting embodiment of the invention, the Nanobody or polypeptide of the invention is glycosylated. According to another non-limiting embodiment of the invention, the Nanobody or polypeptide of the invention is non-glycosylated.

According to one preferred, but non-limiting embodiment of the invention, the Nanobody or polypeptide of the invention is produced in a bacterial cell, in particular a bacterial cell suitable for large scale pharmaceutical production, such as cells of the strains mentioned above.

According to another preferred, but non-limiting embodiment of the invention, the Nanobody or polypeptide of the invention is produced in a yeast cell, in particular a yeast cell suitable for large scale pharmaceutical production, such as cells of the species mentioned above.

According to yet another preferred, but non-limiting embodiment of the invention, the Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the Nanobodies and the proteins of the invention, the Nanobodies and proteins of the invention can be produced either intracellularly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic hosts cells are used, extracellular production is usually preferred since this considerably facilitates the further isolation and downstream processing of the Nanobodies and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm. Another advantage is that correct disulfide bonds may form because the periplasm provides a more oxidative environment than the cytoplasm. Proteins overexpressed in E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular a Nanobody or a polypeptide of the invention, can be used.

Thus, according to one non-limiting embodiment of the invention, the Nanobody or polypeptide of the invention is a Nanobody or polypeptide that has been produced intracellularly and that has been isolated from the host cell, and in particular from a bacterial cell or from an inclusion body in a bacterial cell. According to another non-limiting embodiment of the invention, the Nanobody or polypeptide of the invention is a Nanobody or polypeptide that has been produced extracellularly, and that has been isolated from the medium in which the host cell is cultivated.

Some preferred, but non-limiting promoters for use with these host cells include,
for expression in E. coli: lac promoter (and derivatives thereof such as the lacUV5 promoter); arabinose promoter; left- (PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC1 (cytochrome c iso-1), GAPDH (glyceraldehydes-3-phosphate dehydrogenase); PGK1 (phosphoglycerate kinase), PYK1 (pyruvate kinase); regulated: GAL1, 10, 7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in Pichia pastoris: the AOX1 promoter (alcohol oxidase I)
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1α: (hEF-1α) promoter from human, chimpanzee, mouse or rat; the SV40 early promoter; HIV-1 long terminal repeat promoter; β-actin promoter;

Some preferred, but non-limiting vectors for use with these host cells include:
vectors for expression in mammalian cells: pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565), as well as viral-based expression systems, such as those based on adenovirus;
vectors for expression in bacterials cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen) and other baculovirus vectors
vectors for expression in plants or plant cells: for example vectors based on cauliflower mosaic virus or tobacco mosaic virus, suitable strains of Agrobacterium, or Ti-plasmid based vectors.

Some preferred, but non-limiting secretory sequences for use with these host cells include:
for use in bacterial cells such as E. coli: PelB, Bla, OmpA, OmpC, OmpF, OmpT, StII, PhoA, PhoE, MalE, Lpp, LamB, and the like; TAT signal peptide, hemolysin C-terminal secretion signal
for use in yeast: α-mating factor prepro-sequence, phosphatase (pho1), invertase (Suc), etc.;
for use in mammalian cells: indigenous signal in case the target protein is of eukaryotic origin; murine Ig κ-chain V-J2-C signal peptide; etc.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

Generally, for pharmaceutical use, the polypeptides of the invention may be formulated as a pharmaceutical preparation comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one Nanobody of the invention or at least one polypeptide of the invention and at least one suitable carrier, diluent or excipient (i.e. suitable for pharmaceutical use), and optionally one or more further active substances.

Generally, the Nanobodies and polypeptides of the invention can be formulated and administered in any suitable manner known per se, for which reference is for example made to the general background art cited above (and in particular to WO 04/041862, WO 04/041863, WO 04/041865 and WO 04/041867) as well as to the standard handbooks, such as Remington's Pharmaceutical Sciences, 18^{th} Ed., Mack Publishing Company, USA (1990) or Remington, the Science and Practice of Pharmacy, 21th Edition, Lippincott Williams and Wilkins (2005).

For example, the Nanobodies and polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

The Nanobodies and polypeptides of the invention can also be administered using gene therapy methods of delivery. See, e.g., U.S. Pat. No. 5,399,346, which is incorporated by reference in its entirety. Using a gene therapy method of delivery, primary cells transfected with the gene encoding a Nanobody or polypeptide of the invention can additionally be transfected with tissue specific promoters to target specific organs, tissue, grafts, tumors, or cells and can additionally be transfected with signal and stabilization sequences for subcellularly localized expression.

Thus, the Nanobodies and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the Nanobodies and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the Nanobody or polypeptide of the invention. The percentage of the compositions and preparations may, of course, be varied and may conveniently be between about 2 to about 60% of the weight of a given unit dosage form. The amount of the Nanobody or polypeptide of the invention in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the Nanobodies and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the Nanobodies and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The amino acid sequences, Nanobodies and polypeptides of the invention may also be administered intravenously or intraperitoneally by infusion or injection. Solutions of the Nanobodies and polypeptides of the invention or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the Nanobodies and polypeptides of the invention in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the Nanobodies and polypeptides of the invention may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the Nanobodies and polypeptides of the invention can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the Nanobodies and polypeptides of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the Nanobodies and polypeptides of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the Nanobodies and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the Nanobodies and polypeptides of the invention required for use in treatment will vary not only with the particular salt selected but also with the route of administration, the nature of the condition being treated and the age and condition of the patient and will be ultimately at the discretion of the attendant physician or clinician. Also the dosage of the Nanobodies and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

In another aspect, the invention relates to a method for the prevention and/or treatment of at least one IL-6 related disorders, said method comprising administering, to a subject in need thereof, a pharmaceutically active amount of a Nanobody of the invention, of a polypeptide of the invention, and/or of a pharmaceutical composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention relates to a method for the prevention and/or treatment of at least one disease or disorder that is associated with IL-6, with its biological or pharmacological activity, and/or with the biological pathways or signalling in which IL-6 is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, the invention relates to a method for the prevention and/or treatment of at least one disease or disorder that can be treated by modulating IL-6, its biological or pharmacological activity, and/or the biological pathways or signalling in which IL-6 is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, said pharmaceutically effective amount may be an amount that is sufficient to modulate IL-6, its biological or pharmacological activity, and/or the biological pathways or signalling in which IL-6 is involved; and/or an amount that provides a level of the amino acid sequence of the invention, of a Nanobody of the invention, of a polypeptide of the invention in the circulation that is sufficient to modulate IL-6, its biological or pharmacological activity, and/or the biological pathways or signalling in which IL-6 is involved.

The invention also relates to a method for the prevention and/or treatment of at least one disease or disorder that can be prevented and/or treated by administering an amino acid sequence, Nanobody or polypeptide of the invention to a patient, said method comprising administering, to a subject in need thereof, a pharmaceutically active amount of an amino acid sequence, a Nanobody of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of an amino acid sequence, a Nanobody of the invention, of a polypeptide of the invention, and/or of a pharmaceutical composition comprising the same.

In another aspect, the invention relates to a method for immunotherapy, and in particular for passive immunotherapy, which method comprises administering, to a subject suffering from or at risk of the diseases and disorders mentioned herein, a pharmaceutically active amount of an amino acid sequence, a Nanobody of the invention, of a polypeptide of the invention, and/or of a pharmaceutical composition comprising the same.

In the above methods, the amino acid sequences and/or Nanobodies and/or polypeptides of the invention and/or the compositions comprising the same can be administered in any suitable manner, depending on the specific pharmaceutical formulation or composition to be used. Thus, the amino acid sequences and/or Nanobodies and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The amino acid sequences and/or Nanobodies and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific amino acid sequence and/or Nanobody or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences and/or Nanobodies and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific amino acid sequence and/or Nanobody and polypeptide of the invention to be used, the specific route of administration and the specific pharmaceutical formulation or composition used, the amino acid sequences and/or Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single amino acid sequence and/or Nanobody or polypeptide of the invention will be used. It is however within the scope of the invention to use two or more amino acid sequences and/or Nanobodies and/or polypeptides of the invention in combination.

The amino acid sequences and/or Nanobodies and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement. For example, the amino acid sequences and/or Nanobodies and polypeptides of the invention may be used in a combined treatment or administration regimen with one or more active principles directed against TNF-alpha, such as known antibodies or antibody fragments against TNF including but not limited to HUMIRA™ and REMICADE™ or the anti-TNF polypeptides described in WO 04/041862 of applicant or in the non-prepublished U.S. provisional application 60/682,332 by applicant (filing date May 18, 2005). Other active principles against TNF-alpha (such as ENBREL™) will be clear to the skilled person.

In particular, the amino acid sequences and/or Nanobodies and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are administered to be simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of an amino acid sequence and/or Nanobody or polypeptide of the invention in the preparation of a pharmaceutical composition for prevention and/or treatment of at least one IL-6 related disorders.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention also relates to the use of an amino acid sequence and/or Nanobody or polypeptide of the invention in the preparation of a pharmaceutical composition for the prevention and/or treatment of at least one disease or disorder that can be prevented and/or treated by administering an amino acid sequence and/or Nanobody or polypeptide of the invention to a patient.

More in particular, the invention relates to the use of an amino acid sequence and/or Nanobody or polypeptide of the invention in the preparation of a pharmaceutical composition for the prevention and/or treatment of at least one neurodegenerative disease or disorder, and in particular for the prevention and treatment of one or more of the diseases and disorders listed herein.

Again, in such a pharmaceutical composition, the one or more amino acid sequences and/or Nanobodies or polypeptides of the invention may also be suitably combined with one or more other active principles, such as those mentioned herein.

Finally, although the use of the amino acid sequences and/or Nanobodies of the invention (as defined herein) and of the polypeptides of the invention is much preferred, it will be clear that on the basis of the description herein, the skilled person will also be able to design and/or generate, in an analogous manner, other (single) domain antibodies against IL-6, as well as polypeptides comprising such (single) domain antibodies (in which the terms “domain antibody” and “single domain antibody” have their usual meaning in the art).

Thus, one further aspect of the invention relates to domain antibodies or single domain antibodies against IL-6, and to polypeptides that comprise at least one such (single) domain antibody and/or that essentially consist of such a (single) domain antibody.

In particular, such a (single) domain antibody against IL-6 may comprise 3 CDR's, in which said CDR's are as defined above for the Nanobodies of the invention. For example, such (single) domain antibodies may be the single domain antibodies known as “dAb's”, which are for example as described by Ward et al, supra, but which have CDR's that are as defined above for the Nanobodies of the invention. However, as mentioned above, the use of such “dAb's” will usually have several disadvantages compared to the use of the corresponding Nanobodies of the invention. Thus, any (single) domain antibodies against IL-6 according to this aspect of the invention will preferably have framework regions that provide these (single) domain antibodies against IL-6 with properties that make them substantially equivalent to the Nanobodies of the invention.

This aspect of the invention also encompasses nucleic acids that encode such (single) domain antibodies and/or polypeptides, compositions that comprise such (single) domain antibodies, polypeptides or nucleic acids, host cells that (can) express such (single) domain antibodies or polypeptides, and methods for preparing and using such (single) domain antibodies, polypeptides or nucleic acids, which may be essentially analogous to the polypeptides, nucleic acids, compositions, host cells, methods and uses described above for the Nanobodies of the invention.

Furthermore, it will also be clear to the skilled person that it may be possible to “graft” one or more of the CDR's mentioned above for the Nanobodies of the invention onto other “scaffolds”, including but not limited to human scaffolds or non-immunoglobulin scaffolds.

Suitable scaffolds and techniques for such CDR grafting will be clear to the skilled person and are well known in the art, see for example U.S. Pat. No. 6,180,370, WO 01/27160, EP 0 605 522, EP 0 460 167, U.S. Pat. No. 6,054,297, Nicaise et al., Protein Science (2004), 13:1882-1891; Ewert et al., Methods, 2004 October; 34 (2):184-199; Kettleborough et al., Protein Eng. 1991 October; 4 (7): 773-783; O'Brien and Jones, Methods Mol. Biol. 2003:207:81-100; and Skerra, J. Mol. Recognit. 2000:13:167-187, and Saerens et al., J. Mol. Biol. 2005 Sep. 23; 352 (3):597-607, and the further references cited therein. For example, techniques known per se for grafting mouse or rat CDR's onto human frameworks and scaffolds can be used in an analogous manner to provide chimeric proteins comprising one or more of the CDR's of the Nanobodies of the invention and one or human framework regions or sequences.

Thus, in another embodiment, the invention comprises a chimeric polypeptide comprising at least one CDR sequence chosen from the group consisting of CDR1 sequences, CDR2 sequences and CDR3 sequences mentioned herein for the Nanobodies of the invention. Preferably, such a chimeric polypeptide comprises at least one CDR sequence chosen from the group consisting of the CDR3 sequences mentioned herein for the Nanobodies of the invention, and optionally also at least one CDR sequence chosen from the group consisting of the CDR1 sequences and CDR2 sequences mentioned herein for the Nanobodies of the invention. For example, such a chimeric polypeptide may comprise one CDR sequence chosen from the group consisting of the CDR3 sequences mentioned herein for the Nanobodies of the invention, one CDR sequence chosen from the group consisting of the CDR1 sequences mentioned herein for the Nanobodies of the invention and one CDR sequence chosen from the group consisting of the CDR1 sequences and CDR2 sequences mentioned herein for the Nanobodies of the invention. The combinations of CDR's that are mentioned herein as being preferred for the Nanobodies of the invention will usually also be preferred for these chimeric polypeptides.

In said chimeric polypeptides, the CDR's may be linked to further amino acid sequences and/or may be linked to each other via amino acid sequences, in which said amino acid sequences are preferably framework sequences or are amino acid sequences that act as framework sequences, or together form a scaffold for presenting the CDR's. Reference is again made to the prior art mentioned in the last paragraph. According to one preferred embodiment, the amino acid sequences are human framework sequences, for example V_{H}3 framework sequences. However, non-human, synthetic, semi-synthetic or non-immunoglobulin framework sequences may also be used. Preferably, the framework sequences used are such that (1) the chimeric polypeptide is capable of binding IL-6, i.e. with an affinity that is at least 1%, preferably at least 5%, more preferably at least 10%, such as at least 25% and up to 50% or 90% or more of the affinity of the corresponding Nanobody of the invention; (2) the chimeric polypeptide is suitable for pharmaceutical use; and (3) the chimeric polypeptide is preferably essentially non-immunogenic under the intended conditions for pharmaceutical use (i.e. indication, mode of administration, doses and treatment regimen) thereof (which may be essentially analogous to the conditions described herein for the use of the Nanobodies of the invention).

According to one non-limiting embodiment, the chimeric polypeptide comprises at least two CDR sequences (as mentioned above) linked via at least one framework sequence, in which preferably at least one of the two CDR sequences is a CDR3 sequence, with the other CDR sequence being a CDR1 or CDR2 sequence. According to a preferred, but non-limiting embodiment, the chimeric polypeptide comprises at least two CDR sequences (as mentioned above) linked at least two framework sequences, in which preferably at least one of the three CDR sequences is a CDR3 sequence, with the other two CDR sequences being CDR1 or CDR2 sequences, and preferably being one CDR1 sequence and one CDR2 sequence. According to one specifically preferred, but non-limiting embodiment, the chimeric polypeptides have the structure FR1′-CDR1-FR2′-CDR2-FR3′-CDR3-FR4′, in which CDR1, CDR2 and CDR3 are as defined herein for the CDR's of the Nanobodies of the invention, and FR1′, FR2′, FR3′ and FR4′ are framework sequences. FR1′, FR2′, FR3′ and FR4′ may in particular be Framework 1, Framework 2, Framework 3 and Framework 4 sequences, respectively, of a human antibody (such as V_{H}3 sequences) and/or parts or fragments of such Framework sequences. It is also possible to use parts or fragments of a chimeric polypeptide with the structure FR1′-CDR1-FR2′-CDR2-FR3′-CDR3-FR4. Preferably, such parts or fragments are such that they meet the criteria set out in the preceding paragraph.

The invention also relates to proteins and polypeptides comprising and/or essentially consisting of such chimeric polypeptides, to nucleic acids encoding such proteins or polypeptides; to methods for preparing such proteins and polypeptides; to host cells expressing or capable of expressing such proteins or polypeptides; to compositions, and in particular to pharmaceutical compositions, that comprise such proteins or polypeptides, nucleic acids or host cells; and to uses of such proteins or polypeptides, such nucleic acids, such host cells and/or such compositions, in particular for prophylactic, therapeutic or diagnostic purposes, such as the prophylactic, therapeutic or diagnostic purposes mentioned herein. For example, such proteins, polypeptides, nucleic acids, methods, host cells, compositions and uses may be analogous to the proteins, polypeptides, nucleic acids, methods, host cells, compositions and use described herein for the Nanobodies of the invention.

It should also be noted that, when the amino acid sequences and/or Nanobodies of the invention contain one or more other CDR sequences than the preferred CDR sequences mentioned above, these CDR sequences can be obtained in any manner known per se, for example from Nanobodies (preferred), V_{H} domains from conventional antibodies (and in particular from human antibodies), heavy chain antibodies, conventional 4-chain antibodies (such as conventional human 4-chain antibodies) or other immunoglobulin sequences directed against IL-6. Such immunoglobulin sequences directed against IL-6 can be generated in any manner known per se, as will be clear to the skilled person, i.e. by immunization with IL-6 or by screening a suitable library of immunoglobulin sequences with IL-6, or any suitable combination thereof. Optionally, this may be followed by techniques such as random or site-directed mutagenesis and/or other techniques for affinity maturation known per se. Suitable techniques for generating such immunoglobulin sequences will be clear to the skilled person, and for example include the screening techniques reviewed by Hoogenboom, Nature Biotechnology, 23, 9, 1105-1116 (2005). Other techniques for generating immunoglobulins against a specified target include for example the Nanoclone technology (as for example described in the published US patent application 2006-0211088), so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). All these techniques can be used to generate immunoglobulins against IL-6, and the CDR's of such immunoglobulins can be used in the Nanobodies of the invention, i.e. as outlined above. For example, the sequence of such a CDR can be determined, synthesized and/or isolated, and inserted into the sequence of a Nanobody of the invention (e.g. so as to replace the corresponding native CDR), all using techniques known per se such as those described herein, or Nanobodies of the invention containing such CDR's (or nucleic acids encoding the same) can be synthesized de novo, again using the techniques mentioned herein.

Further uses of the amino acid sequences, Nanobodies, polypeptides, nucleic acids, genetic constructs and hosts and host cells of the invention will be clear to the skilled person based on the disclosure herein. For example, and without limitation, the amino acid sequences of the invention can be linked to a suitable carrier or solid support so as to provide a medium than can be used in a manner known per se to purify IL-6 from compositions and preparations comprising the same. Derivatives of the amino acid sequences of the invention that comprise a suitable detectable label can also be used as markers to determine (qualitatively or quantitatively) the presence of IL-6 in a composition or preparation or as a marker to selectively detect the presence of IL-6 on the surface of a cell or tissue (for example, in combination with suitable cell sorting techniques).

The invention will now be further described by means of the following non-limiting examples and figures, in which the Figures show:

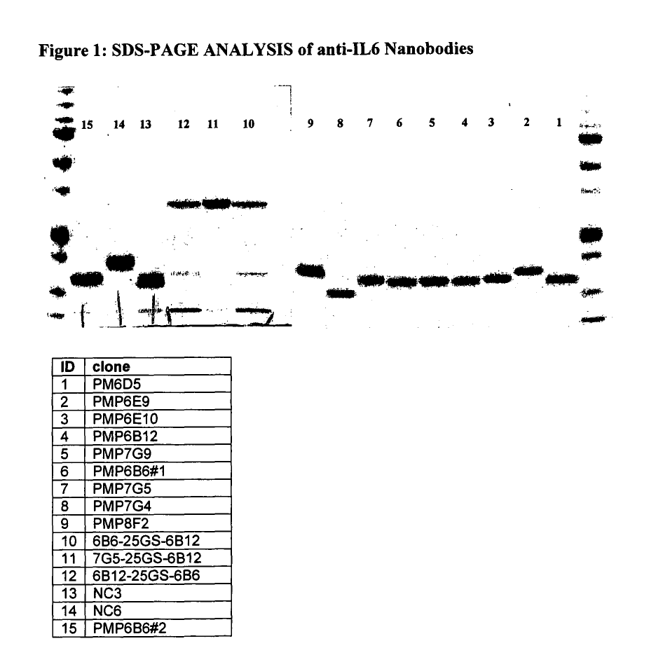

FIG. 1: SDS-PAGE ANALYSIS of anti-IL6 Nanobodies;

FIG. 2: Evaluation of Nanobodies against IL6R-binding site in Alphascreen

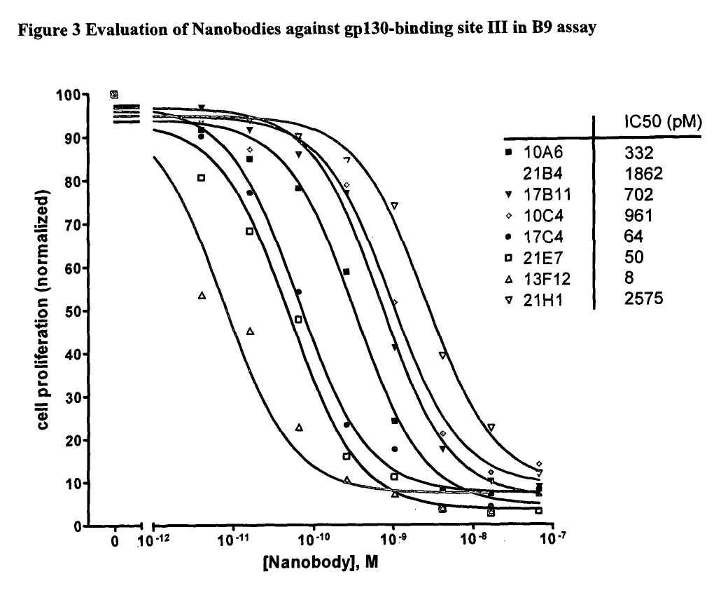

FIG. 3: Evaluation of Nanobodies against gp130-binding site III in B9 assay

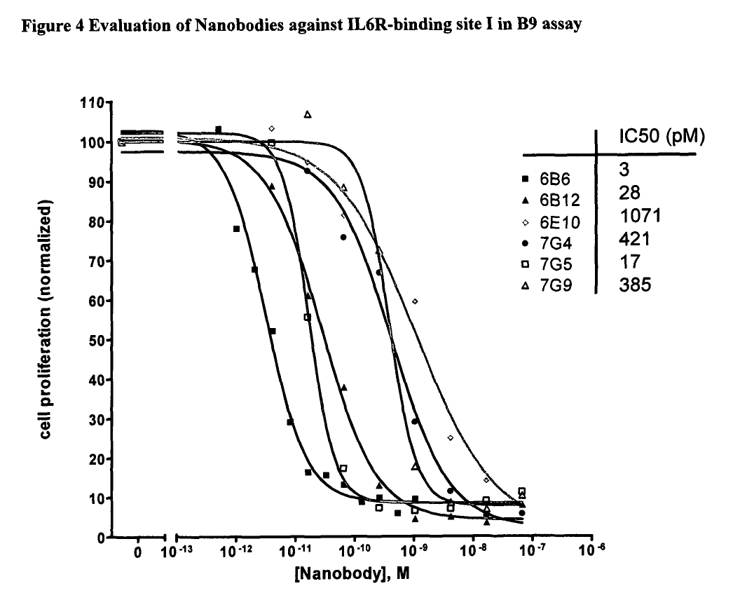

FIG. 4: Evaluation of Nanobodies against IL6R-binding site I in B9 assay

### EXAMPLE 1

### Immunization

With approval of the Ethical Committee of the Faculty of Veterinary Medicine (University Ghent, Belgium), 3 llamas were immunized with recombinant human IL6 according to all current animal welfare regulations. For immunization, the antigen was formulated as an emulsion with an appropriate, animal-friendly adjuvant (Specoll, CEDI Diagnostics B.V.). The antigen was administered by double-spot injections intramuscularly in the neck. Each animal received 2 injections of the emulsion, containing 100 μg of IL-6 and 4 subsequent injections containing 50 ug of antigen at weekly intervals. At different time points during immunization, 10-ml blood samples were collected from the animal and sera were prepared. The induction of an antigen specific humoral immune response was verified using the serum samples in an ELISA-based experiment using immobilized IL6. Five days after the last immunization, a blood sample of 150 ml was collected. Peripheral blood lymphocytes (PBLs), as the genetic source of the llama heavy chain immunoglobulins (HcAbs), were isolated from the 150-ml blood sample using a Ficoll-Paque gradient (Amersham Biosciences) yielding 5×10⁸ PBLs. The maximal diversity of antibodies is expected to be equal to the number of sampled B-lymphocytes, which is about 10% of the number of PBLs (5×10⁷). The fraction of heavy-chain antibodies in llama is up to 20% of the number of B-lymphocytes. Therefore, the maximal diversity of HcAbs in the 150 ml blood sample is calculated as 10⁷ different molecules.

### EXAMPLE 2

### Cloning of Nanobodies™ Derived from Llamas Immunized with Human IL6

Cloning of Nanobodies™ from llamas immunized with human IL6 were carried out using one of the two hereinbelow described methods:

a) Repertoire Cloning Combined with Phage Display

“Repertoire cloning” and “phage display” techniques can be used for the cloning of immunoglobulin sequences, as for example described in EP 0 589 877, U.S. Pat. No. 5,969,108, U.S. Pat. No. 6,248,516 and Reiter et al., 1999. Generally, the selection and cloning of immunoglobulin sequences (also referred to below as “binders”) by means of these techniques involves the steps of:
a) providing “total” mRNA from a cell using a method described by Chomczynski and Sacchi (1987), wherein said cell can express the entire immune “repertoire” from a animal (such as B-cell) and wherein said mRNA contains the entire immune repertoire of said animal;
b) synthesizing cDNA out of said mRNA with MMLV Reverse Transcriptase (Superscript III, Invitrogen) using oligo d(T) oligonucleotides (de Haard et al., 1999).
c) selectively amplifying the nucleotide sequences that encode the immune repertoire using specific primers (EP 0 368 684; WO03/054016); in a first PCR, the repertoire of both conventional (1.6 kb) and heavy chain (1.3 kb) antibody gene segments is amplified using a leader specific primer and an oligo d(T) primer. The resulting DNA fragments are separated by agarose gel electrophoresis. The amplified 1.3 kb fragment, encoding heavy-chain antibody segments is purified from the agarose gel and used as template in a nested PCR using a FR1 specific primer containing a SfiI restriction site and an oligo d(T) primer. The PCR products are subsequently digested with SfiI and BstEII (naturally occurring in FR4);
d) preparing phage particles that express the binders encoded by said amplified sequences on their surface; using a suitable micro-organism, such as E. coli: following gel electrophoresis, a DNA fragment of approximately 400 basepairs is purified from gel and 330 ng of amplified VHH repertoire is ligated into the corresponding restriction sites of one microgram of phagemid vector to obtain a library after electroporation of Escherichia coli TG1. The phagemid vector allows for production of phage particles, expressing the individual VHHs as a fusion protein with the geneIII product;
e) selecting phage particles that express binder sequences that can bind to IL6: Different concentrations between 0 and 1 nM of biotinylated IL-6 were incubated with 10 ul phage in PBS containing 0.1% casein and 0.1% Tween-20. After 1 hour incubation at RT, the samples are transferred to microtiter plate wells which are coated with 5 ug/ml streptavidin and subsequently blocked with PBS containing 1% casein for 3 hours at room temperature. After 5 min incubation, the wells were washed 10 times with PBS-Tween and 10 times with PBS. Phage are eluted by addition of 1 mg/ml trypsin followed by a 30 min incubation at 37° C. or by addition of a 100 ug/ml mixture of anti-IL6 antibodies CLB8 (Sanquin, Amsterdam) and BE-8 (Diaclone) followed by overnight incubation at 4° C. Eluted phage are allowed to infect exponentially growing TG1 cells, and are then plated on LB agar plates containing 100 μg/ml ampicillin and 2% glucose.

### EXAMPLE 3

### Cloning, Expression and Preparation of Periplasmic Extracts

DNA fragments encoding anti-IL6 Nanobodies were digested with SfiI and BsteII and ligated into the corresponding restriction sites of pAX051. The ligation mixtures were subsequently transformed into TG1 electrocompetent cells. Carbenicillin resistant clones were analyzed for the presence of insert and positive clones were stored as glycerol stocks at −80° C.

For protein expression, LB medium containing Carbenicillin (100 μg/ml) and 2% glucose is inoculated with the Nanobody expressing clone and incubated overnight at 37° C. This starter culture is then used to inoculate the production culture at a 1/100 dilution (TB medium+Carbenicillin (100 μg/ml)+0.1% Glucose). After growing for 3 hours at 37° C., Nanobody expression is induced by adding IPTG (1 mM final concentration). Protein expression is allowed to continue for 4 hours, at which point cells are collected by centrifugation and stored as wet cell paste at −20° C.

Periplasmic extracts of the −20° C. stored wet cell paste are prepared by resuspending the pellet in PBS followed by centrifugation to pellet the cells. The supernatant, which represents the periplasmic fraction, is removed and was used for further experiments.

### EXAMPLE 4

### Identification of Inhibitory Anti-IL6 Nanobodies

Nanobodies capable of inhibiting the interaction between IL6 and IL6R were identified by Alphascreen. In this assay, periplasmic extracts prepared from anti-IL6 Nanobody expressing E. coli cells (25-fold diluted) were incubated with 3 nM biotinylated human IL6 in a 384-wells plate for 15 min. Subsequently a mixture of IL6R (1 nM) and acceptor beads (20 ug/ml) coated with anti-IL6R MAb BN-12 (Diaclone) were added and incubated for 30 min. Finally, streptavidin coated donor beads (20 ug/ml) were added. After 1 hour of incubation the plates were read on the Envision Alphascreen reader (PerkinElmer).

Nanobodies against the gp130 binding sites on IL6 were identified by an indirect Alphascreen assay in which MAbs BE4 (Diaclone) and CLB16 (Sanquin, Amsterdam) were employed. These two anti-IL6 antibodies recognize gp130 binding site II and III, respectively. In this assay, periplasmic extracts were incubated for 15 min with 1 nM biotinylated IL6. Acceptor beads (20 ug/ml) coated with either BE-4 or CLB16 were added and after 30 min streptavidin coated donor beads (20 ug/ml) were added. Reaction mixtures were incubated for 1 hour and then read on the Envison Alphascreen reader (PerkinElmer).

### EXAMPLE 5

### Off-Rate Analysis of Anti-IL6 Nanobodies on Biacore

Off-rate analysis of Nanobodies binding to IL6 was done by surface plasmon resonance on a Biacore 3000 instrument. Recombinant human IL6 was covalently bound to a CM5 sensor chip via amine coupling at a density of ˜500 RU. Remaining reactive groups were inactivated. Periplasmic extracts prepared from E. coli cells expressing anti-IL6 Nanobodies were diluted 10 or 15-fold and injected for 4 min to allow for binding to IL6 immobilized on the chip. Buffer without Nanobody was sent over the chip for 30 min to allow for spontaneous dissociation of bound Nanobody. The dissociation phase was used to calculate the koff values for each individual Nanobody (table B-1).

TABLE B-1
Off-rates of monovalent anti-IL6 Nanobodies

| Clone | k_{off} (s⁻¹) |
|---|---|
| PMP6D5 | 5.11E−04 |
| PMP8F2 | ND |
| PMP6B12 | 2.70E−04 |
| PMP6B6 | 4.33E−04 |
| PMP11C1 | ND |
| PMP23H2 | 1.60E−03 |
| PMP7G4 | 2.08E−03 |
| PMP20D2 | 2.43E−04 |
| PMP7G5 | 4.12E−04 |
| PMP7H3 | 3.09E−03 |
| PMP7G9 | 2.87E−03 |
| PMP9A9 | 6.05E−03 |
| PMP22E3 | 5.19E−03 |
| PMP6E10 | 5.45E−04 |
| PMP6G10 | 4.33E−04 |
| NC3 | 5.60E−04 |
| NC6 | 8.20E−04 |
| PMP13A1 | 3.96E−04 |
| PMP20G9 | 3.18E−04 |
| PMP20F4 | 1.95E−04 |
| PMP21A7 | 5.26E−04 |
| PMP13D8 | 2.51E−04 |
| PMP21E12 | 2.22E−03 |
| PMP21C12 | 8.97E−04 |
| PMP21C2 | 1.16E−03 |
| PMP14G4 | 3.12E−04 |
| PMP14E1 | 5.70E−04 |
| PMP6E9 | 6.31E−04 |
| PMP12H3 | 2.43E−04 |
| PMP12C5 | 2.11E−04 |
| PMP17G7 | 6.69E−04 |
| PMP14G11 | 2.29E−04 |
| PMP9F9 | 1.90E−04 |
| PMP14A8 | 1.31E−04 |
| PMP17B5 | 1.50E−04 |
| PMP6B7 | 1.99E−04 |
| PMP14E9 | 5.31E−04 |
| PMP17D7 | 1.16E−03 |
| PMP14G1 | 7.90E−04 |
| PMP17B11 | 1.33E−03 |
| PMP10C4 | 8.20E−04 |
| PMP17C4 | 1.37E−03 |
| PMP21B4 | 6.58E−04 |
| PMP21H1 | 1.24E−03 |
| PMP10A6 | 1.04E−03 |
| PMP13H6 | 1.89E−03 |
| PMP13F12 | 3.66E−05 |
| PMP21A2 | ND |
| PMP21F7 | ND |
| PMP21H3 | ND |
| PMP21E7 | 7.72E−04 |

### EXAMPLE 6

### Purification of Nanobodies

The His6-tagged Nanobodies are purified from periplasmic extracts by Immobilized Metal Affinity Chromatography (IMAC). The TALON resin (Clontech) is processed according to the manufacturer's instructions. Periplasmic extracts prepared as described in example 3 are incubated with the resin for 30 min at RT on a rotator. The resin is washed with PBS and transferred to a column. The packed resin is washed with 15 mM Imidazole. The Nanobodies are eluted from the column using 150 mM Imidazole. The eluted fractions are analyzed by spotting on Hybond Membrane and visualization with Ponceau. Fractions containing protein are pooled and dialyzed against PBS. Dialyzed proteins are collected, filter sterilized, concentration determined and stored at −20° C.

### EXAMPLE 7

### SDS-PAGE Analysis

To determine the purity, protein samples were analyzed on a 15% SDS-PAGE gel. 10 μl Laemmli sample buffer was added to 10 μl (1 ug) purified protein, the sample was heated for 10 minutes at 95° C., cooled and loaded on a 15% SDS-PAGE gel. The gel was processed according to general procedures and stained with Coomassie Brilliant Blue (CBB). SDS-PAGE of monovalent and bivalent anti-IL6 Nanobodies is shown in FIG. 1.

### EXAMPLE 8

### Expression Levels

Expression levels were calculated for various mono- and multivalent Nanobodies and are listed in Table B-2.

TABLE B-2
Expression levels of various Nanobodies in
mg of protein per liter of culture medium

| Clone | yield |
|---|---|
| PM6D5 | 10.2 |
| MP6E9 | 5.6 |
| PMP6E10 | 6.2 |
| PMP6B12 | 2.4 |
| PMP7G9 | 3.1 |
| PMP6B6#1 | 3.0 |
| PMP7G5 | 10.8 |
| PMP7G4 | 11.1 |
| PMP8F2 | 2.1 |
| PMP6B6#2 | 10.3 |
| NC3 | 8.6 |
| NC6 | 5.4 |
| 6B6-25GS-6B12 | 1.2 |
| 7G5-25GS-6B12 | 6.7 |
| 6B12-25GS-6B6 | 0.9 |

### EXAMPLE 9

### Evaluation of Monovalent Nanobodies Targeting the IL6R-Binding Site in Alphascreen

Purified samples of Nanobodies PMP6B6, PMP7G5, PMP7G9 and PMP7G4 were analyzed in Alphascreen for their ability to inhibit the interaction between IL6 and IL6R. In this assay, various concentrations of anti-IL6 Nanobodies ranging from 1 uM to 10 pM were incubated with 3 nM biotinylated human IL6 for 15 min in a 384-wells plate. Subsequently a mixture of IL6R (1 nM) and acceptor beads (20 ug/ml) coated with anti-IL6R MAb BN-12 (Diaclone) were added and incubated for 30 min. Finally, streptavidin coated donor beads (20 ug/ml) were added. After 1 hour of incubation plates were read on the Envison Alphascreen reader (PerkinElmer). All experiments were performed in duplicate. Inhibition curves and IC₅₀ values are shown in FIG. 2

### EXAMPLE 10

### Analysis of Monovalent Anti-IL6 Nanobodies in B9 Assay

Purified samples of Nanobodies PMP10A6, PMP21B4, PMP17B11, PMP10C4, PMP17C4, PMP21E7, PMP13F12, PMP21H1, PMP6E10, PMP6B12, PMP6B6, PMP7G5, PMP7G9 and PMP7G4 were tested in the B9 assay. This proliferation assay employs the murine hybridoma cell line B9 and was performed essentially as described by Aarden et al. (Eur J Immunol. 17 (1987):1411-1416). Inhibition curves and IC₅₀ values are shown in FIGS. 3 and 4.

### EXAMPLE 11

### Construction of Multivalent Nanobodies

A subset of inhibitory anti-IL6 Nanobodies was used for the construction of multivalent Nanobodies. As spacer between the building blocks either a 9 amino acid Gly/Ser-linker (SEQ ID No 164) or a corresponding 25 amino acid Gly/Ser-linker was used. Generated constructs are shown in Table B-3 below (SEQ ID No 371-447).

### EXAMPLE 12

### Humanization

DNA fragments encoding humanized versions of Nanobodies® are assembled from oligonucleotides using a PCR overlap extension method (Stemmer et al., 1995).

i) Antagonistic Activity in Alpha Screen

Humanized clones are tested in Alphascreen for inhibition of the IL6/IL6R interaction and/or the IL6/IL6R complex/gp130 interaction. Serial dilutions of purified proteins (concentration range: 500 nM-10 pM) are added to IL-6 (0.3 nM) and incubated for 15 min. Subsequently 3 nM bio-IL6R or bio-gp130 and BN12-coated acceptor beads are added and this mixture is incubated for 1 hour. Finally streptavidin donor beads are added and after 1 hour incubator the plate is read on the Envision microplate reader.

ii) Temperature Stability Tests

Temperature stability tests are performed for humanized clones. Samples are diluted at 200 μg/ml and divided in 5*2 aliquots containing 60 μl. The different vials are incubated each at a given temperature ranging from 37° C. to 90° C. (37, 50, 70 and 90° C.) for a period of 1 hr. (lid temperature: 105° C.) (control was stored at 4° C.). Thereafter, the samples are hold at 25° C. for 2 hrs (ramping rate: 0.05) and stored over night at 4° C. Precipitates are removed by centrifugation for 30 min at 14.000 rpm. Supernatant is carefully removed and further analysed. OD at 280 nm is measured and the concentration is calculated based on the extinction coefficients.

TABLE B-3
List of sequences

< FR1, SEQ ID NO: 126; PRT;->
QVQLQESGGGXVQAGGSLRLSCAASG
< FR2, SEQ ID NO: 127; PRT;->
WXRQAPGKXXEXVA
< FR3, SEQ ID NO: 128; PRT;->
RFTISRDNAKNTVYLQMNSLXXEDTAVYYCAA
< FR4, SEQ ID NO: 129; PRT;->
XXQGTXVTVSS
< FR1, SEQ ID NO: 130; PRT;->
QVQLQESGGGLVQAGGSLRLSCAASG
< FR2, SEQ ID NO: 131; PRT;->
WFRQAPGKERELVA
< FR2, SEQ ID NO: 132; PRT;->
WFRQAPGKEREFVA
< FR2, SEQ ID NO: 133; PRT;->
WFRQAPGKEREGA
< FR2, SEQ ID NO: 134; PRT;->
WFRQAPGKQRELVA
< FR2, SEQ ID NO: 135; PRT;->
WFRQAPGKQREFVA
< FR2, SEQ ID NO: 136; PRT;->
WYRQAPGKGLEWA
< FR3, SEQ ID NO: 137; PRT;->
RFTISRDNAKNTVYLQMNSLKPEDTAVYYCAA
< FR4, SEQ ID NO: 138; PRT;->
WGQGTQVTVSS
< FR4, SEQ ID NO: 139; PRT;->
WGQGTLVTVSS
< CDR1, SEQ ID NO: 140; PRT;->
SFGMS
< CDR1, SEQ ID NO: 141; PRT;->
LNLMG
< CDR1, SEQ ID NO: 142; PRT;->
INLLG
< CDR1, SEQ ID NO: 143; PRT;->
NYWMY
< CDR2, SEQ ID NO: 144; PRT;->
SISGSGSDTLYADSVKG
< CDR2, SEQ ID NO: 145; PRT;->
TTTVGDSTNYADSVKG
< CDR2, SEQ ID NO: 146; PRT;->
TTTVGDSTSYADSVKG
< CDR2, SEQ ID NO: 147; PRT;->
SINGRGDDTRYADSVKG
< CDR2, SEQ ID NO: 148; PRT;->
AISADSSTKNYADSVKG
< CDR2, SEQ ID NO: 149; PRT;->
AISADSSDKRYADSVKG
< CDR3, SEQ ID NO: 150; PRT;->
RISTGGGYSYYADSVKG
< CDR3, SEQ ID NO: 151; PRT;->
DREAQVDTLDFDY
< CDR3, SEQ ID NO: 152; PRT;->
GGSLSR
< CDR3, SEQ ID NO: 153; PRT;->
RRTWHSEL
< CDR3, SEQ ID NO: 154; PRT;->
GRSVSRS
< CDR3, SEQ ID NO: 155; PRT;->
GRGSP
< MYC-TAG, SEQ ID NO: 156; PRT;->
AAAEQKLISEEDLNGAA
< PMP 6A6(ALB-1), SEQ ID NO: 157; PRT;->
AVQLVESGGGLVQPGNSLRLSCAASGFTFRSFGMSWVRQAPGKEPEWVSSISGSGSDTLYADSVKGRFTISRDNAKTTLYLQ
MNSLKPEDTAVYYCTIGGSLSRSSQGTQVTVSS
< ALB-8, SEQ ID NO: 158; PRT;->
EVQLVESGGGLVQPGNSLRLSCAASGFTFSSFGMSWVRQAPGKGLEWVSSISGSGSDTLYADSVKGRFTISRDNAKTTLYLQ
MNSLRPEDTAVYYCTIGGSLSRSSQGTLVTVSS
< PMP 6A8(ALB-2), SEQ ID NO: 159; PRT; ->
AVQLVESGGGLVQGGGSLRLACAASERIFDLNLMGWYRQGPGNERELVATCITVGDSTNYADSVKGRFTISMDYTKQTVYLH
MNSLRPEDTGLYYCKIRRTWHSELWGQGTQVTVSS
< FC44, SEQ ID NO: 160; PRT;->
EVQLQASGGGLVQAGGSLRLSCSASVRTFSIYAMGWFRQAPGKEREFVAGINRSGDVTKYADFVKGRFSISRDNAKNMVYLQ
MNSLKPEDTALYYCAATWAYDTVGALTSGYNFWGQGTQVTVSS
< FC5, SEQ ID NO: 161; PRT;->
EVQLQASGGGLVQAGGSLRLSCAASGFKITHYTMGWFRQAPGKEREFVSRITWGGDNTFYSNSVKGRFTISRDNAKNTVYL
QMNSLKPEDTADYYCAAGSTSTATPLRVDYWGKGTQVTVSS
< GS30, SEQ ID NO: 162; PRT;->
GGGGSGGGGSGGGGSGGGGSGGGGSGGGGS
< GS15, SEQ ID NO: 163; PRT;->
GGGGSGGGGSGGGGS
< GS9, SEQ ID NO: 164; PRT;->
GGGGSGGGS
< GS7, SEQ ID NO: 165; PRT;->
SGGSGGS
< LLAMA UPPER LONG HINGE REGION, SEQ ID NO: 166; PRT;->
EPKTPKPQPAAA
< CDR1, SEQ ID NO: 167; PRT;->
PYTMG
< CDR1, SEQ ID NO: 168; PRT;->
DYAMS
< CDR1, SEQ ID NO: 169; PRT;->
YYAIG
< CDR1, SEQ ID NO: 170; PRT;->
INAMG
< CDR1, SEQ ID NO: 171; PRT;->
IYTMG
< CDR1, SEQ ID NO: 172; PRT;->
RLAMD
< CDR1, SEQ ID NO: 173; PRT;->
RLAMD
< CDR1, SEQ ID NO: 174; PRT;->
FNIMG
< CDR1, SEQ ID NO: 175; PRT;->
FNIMG
< CDR1, SEQ ID NO: 176; PRT;->
YYGVG
< CDR1, SEQ ID NO: 177; PRT;->
YYGVG
< CDR1, SEQ ID NO: 178; PRT;->
YYGVG
< CDR1, SEQ ID NO: 179; PRT;->
DSAIG
< CDR1, SEQ ID NO: 180; PRT;->
PYTIA
< CDR1, SEQ ID NO: 181; PRT;->
PYTIG
< CDR1, SEQ ID NO: 182; PRT;->
INVMN
< CDR1, SEQ ID NO: 183; PRT;->
SYAMG
< CDR1, SEQ ID NO: 184; PRT;->
PYTMG
< CDR1, SEQ ID NO: 185; PRT;->
PYTVG
< CDR1, SEQ ID NO: 186; PRT;->
PYTMG
< CDR1, SEQ ID NO: 187; PRT;->
PYTMG
< CDR1, SEQ ID NO: 188; PRT;->
PYTMG
< CDR1, SEQ ID NO: 189; PRT;->
INPMG
< CDR1, SEQ ID NO: 190; PRT;->
INPMG
< CDR1, SEQ ID NO: 191; PRT;->
INPMA
< CDR1, SEQ ID NO: 192; PRT;->
SYPMG
< CDR1, SEQ ID NO: 193; PRT;->
SYPMG
< CDR1, SEQ ID NO: 194; PRT;->
SYPMG
< CDR1, SEQ ID NO: 195; PRT;->
SYPMG
< CDR1, SEQ ID NO: 196; PRT;->
SYPMG
< CDR1, SEQ ID NO: 197; PRT;->
SYPMG
< CDR1, SEQ ID NO: 198; PRT;->
SFPMG
< CDR1, SEQ ID NO: 199; PRT;->
SFPMG
< CDR1, SEQ ID NO: 200; PRT;->
SFPMG
< CDR1, SEQ ID NO: 201; PRT;->
AFPMG
< CDR1, SEQ ID NO: 202; PRT;->
AFPMG
< CDR1, SEQ ID NO: 203; PRT;->
AFPMG
< CDR1, SEQ ID NO: 204; PRT;->
AFPMG
< CDR1, SEQ ID NO: 205; PRT;->
AFPMG
< CDR1, SEQ ID NO: 206; PRT;->
TYAMG
< CDR1, SEQ ID NO: 207; PRT;->
NYHMV
< CDR1, SEQ ID NO: 208; PRT;->
NYAMA
< CDR1, SEQ ID NO: 209; PRT;->
IDAMA
< CDR1, SEQ ID NO: 210; PRT;->
KHHATG
< CDR1, SEQ ID NO: 211; PRT;->
SYVMG
< CDR1, SEQ ID NO: 212; PRT;->
SYVMG
< CDR1, SEQ ID NO: 213; PRT;->
SSPMG
< CDR1, SEQ ID NO: 214; PRT;->
SSPMG
< CDR1, SEQ ID NO: 215; PRT;->
SSPMG
< CDR1, SEQ ID NO: 216; PRT;->
NGPMA
< CDR1, SEQ ID NO: 217; PRT;->
SYPIA
< CDR2, SEQ ID NO: 218; PRT:->
RINWSGIRNYADSVKG
< CDR2, SEQ ID NO: 219; PRT;->
AITGNGASKYYAESMKG
< CDR2, SEQ ID NO: 220; PRT;->
CISSSVGTTYYSDSVKG
< CDR2, SEQ ID NO: 221; PRT;->
DIMPYGSTEYADSVKG
< CDR2, SEQ ID NO: 222; PRT;->
AAHWTVFRGNTYYVDSVKG
< CDR2, SEQ ID NO: 223; PRT;->
SIAVSGTTMLDDSVKG
< CDR2, SEQ ID NO: 224; PRT;->
SISRSGTTMAADSVKG
< CDR2, SEQ ID NO: 225; PRT;->
DITNRGTTNYADSVKG
< CDR2, SEQ ID NO: 226; PRT;->
DITNGGTTMYADSVKG
< CDR2, SEQ ID NO: 227; PRT;->
CISSSDGDTYYADSVKG
< CDR2, SEQ ID NO: 228; PRT;->
CISSSDGDTYYADSVKG
< CDR2, SEQ ID NO: 229; PRT;->
CTSSSDGDTYYADSVKG
< CDR2, SEQ ID NO: 230; PRT;->
CISSSDGDTYYDDSVKG
< CDR2, SEQ ID NO: 231; PRT;->
TIIGSDRSTDLDGDTYYADSVRG
< CDR2, SEQ ID NO: 232; PRT;->
TIIGSDRSTDLDGDTYYADSVRG
< CDR2, SEQ ID NO: 233; PRT;->
AITSGGRKNYADSVKG
< CDR2, SEQ ID NO: 234; PRT;->
AISSNGGSTRYADSVKG
< CDR2, SEQ ID NO: 235; PRT;->
RINWSGIRNYADSVKG
< CDR2, SEQ ID NO: 236; PRT;->
RINWSGIRNYADSVKG
< CDR2, SEQ ID NO: 237; PRT;->
RINWSGIRNYADSVKG
< CDR2, SEQ ID NO: 238; PRT;->
RINWSGITNYADSVKG
< CDR2, SEQ ID NO: 239; PRT;->
RINWSGITNYADSVKG
< CDR2, SEQ ID NO: 240; PRT;->
RIHGSTTNYADSVKG
< CDR2, SEQ ID NO: 241; PRT;->
RIHGSTTNYADSVKG
< CDR2, SEQ ID NO: 242; PRT;->
RIFGGGSTNYADSVKG
< CDR2, SEQ ID NO: 243; PRT;->
GISQSGVGTAYSDSVKG
< CDR2, SEQ ID NO: 244; PRT;->
GISQSGGSTAYSDSVKG
< CDR2, SEQ ID NO: 245; PRT;->
GISQSSSSTAYSDSVKG
< CDR2, SEQ ID NO: 246; PRT;->
GISQSGGSTAYSDSVKG
< CDR2, SEQ ID NO: 247; PRT;->
GISQSGGSTAYSDSVKG
< CDR2, SEQ ID NO: 248; PRT;->
GISQSGGSTAYSDSVKG
< CDR2, SEQ ID NO: 249; PRT;->
GISQSGGSTHYSDSVKG
< CDR2, SEQ ID NO: 250; PRT;->
GISQSGGSTHYSDSVKG
< CDR2, SEQ ID NO: 251; PRT;->
GISQSGGSTHYSDSVKG
< CDR2, SEQ ID NO: 252; PRT;->
GISQSGGSTHYSDSVKG
< CDR2, SEQ ID NO: 253; PRT;->
GISQSGGSTHYSDSVKG
< CDR2, SEQ ID NO: 254; PRT;->
GISQSGGSTHYSDSVKG
< CDR2, SEQ ID NO: 255; PRT;->
GISQSGGSTHYSDSVKG
< CDR2, SEQ ID NO: 256; PRT;->
GISQSGGSTHYSDSVKG
< CDR2, SEQ ID NO: 257; PRT;->
AISWSGANTTYADSVKG
< CDR2, SEQ ID NO: 258, PRT;->
AASGSTSSTYYADSVKG
< CDR2, SEQ ID NO: 259; PRT;->
VISYAGGRTYYADSVKG
< CDR2, SEQ ID NO: 260; PRT;->
TMNWSTGATYYADSVKG
< CDR2, SEQ ID NO: 261; PRT;->
ALNWSGGNTVYTDSVKG
< CDR2, SEQ ID NO: 262; PRT;->
TINWSGSNGYYADSVKG
< CDR2, SEQ ID NO: 263; PRT;->
TINWSGSNKYYADSVKG
< CDR2, SEQ ID NO: 264; PRT;->
AISGRSGNTYYADSVKG
< CDR2, SEQ ID NO: 265; PRT;->
AISGRSGNTYYADSVKG
< CDR2, SEQ ID NO: 266; PRT;->
AISGRSGNTYYADSVKG
< CDR2, SEQ ID NO: 267; PRT;->
AISWRTGTTYYADSVKG
< CDR2, SEQ ID NO: 268; PRT;->
AISWRGGNTYYADSVKG
< CDR3, SEQ ID NO: 269; PRT;->
ASQSGSGYDS
< CDR3, SEQ ID NO: 270; PRT;->
VAKDTGSFYYPAYEHDV
< CDR3, SEQ ID NO: 271; PRT;->
SSWFDCGVQGRDLGNEYDY
< CDR3, SEQ ID NO: 272; PRT;->
YDPRGDDY
< CDR3, SEQ ID NO: 273; PRT;->
TRSTAWNSPQRYDY
< CDR3, SEQ ID NO: 274; PRT;->
FDGYTGSDY
< CDR3, SEQ ID NO: 275; PRT;->
FDGYSGSDY
< CDR3, SEQ ID NO: 276; PRT;->
YYPTTGFDD
< CDR3, SEQ ID NO: 277; PRT;->
YYPTTGFDD
< CDR3, SEQ ID NO: 278; PRT;->
DLSDYGVCSRWPSPYDY
< CDR3, SEQ ID NO: 279; PRT;->
DLSDYGVCSRWPSPYDY
< CDR3, SEQ ID NO: 280; PRT;->
DLSDYGVCSRWPSPYDY
< CDR3, SEQ ID NO: 281; PRT;->
DLSDYGVCSKWPSPYDY
< CDR3, SEQ ID NO: 282; PRT;->
TGKGYVFTPNEYDY
< CDR3, SEQ ID NO: 283; PRT;->
TAKGYVFTDNEYDY
< CDR3, SEQ ID NO: 284; PRT;->
DAPLASDDDVAPADY
< CDR3, SEQ ID NO: 285; PRT;->
DETTGWVQLADFRS
< CDR3, SEQ ID NO: 286; PRT;->
ASQSGSGYDS
< CDR3, SEQ ID NO: 287; PRT;->
ASQSGSGYDS
< CDR3, SEQ ID NO: 288; PRT;->
ASRSGSGYDS
< CDR3, SEQ ID NO: 289; PRT;->
ASRSGSGYDS
< CDR3, SEQ ID NO: 290; PRT;->
ASQVGSGYDS
< CDR3, SEQ ID NO: 291; PRT;->
RRWGYDY
< CDR3, SEQ ID NO: 292; PRT;->
RRWGYDY
< CDR3, SEQ ID NO: 293; PRT;->
RRWGYDY
< CDR3, SEQ ID NO: 294; PRT;->
RDKTLALRDYAYTTDVGYDD
< CDR3, SEQ ID NO: 295; PRT;->
RDKTLALRDYAYTTDVGYOD
< CDR3, SEQ ID NO: 296; PRT;->
RGRTLALRDYAYTTEVGYDD
< CDR3, SEQ ID NO: 297; PRT;->
RGRTLFLRDYAYTTEVGYDD
< CDR3, SEQ ID NO: 298; PRT;->
RGRTLFLRGYAYTTEVGYDD
< CDR3, SEQ ID NO: 299; PRT;->
RGRTIALRNYAYTTEVGYDD
< CDR3, SEQ ID NO: 300; PRT;->
RGRTLALRNYAYTTEVGYDD
< CDR3, SEQ ID NO: 301; PRT;->
RGRTLALRNYAYTTEVGYDD
< CDR3, SEQ ID NO: 302; PRT;->
RGRTLALRNYAYTTEVGYDD
< CDR3, SEQ ID NO: 303; PRT;->
RGRTLALRNYAYTTEVGYDD
< CDR3, SEQ ID NO: 304; PRT;->
RGRTLALRNYAYTTEVGYDD
< CDR3, SEQ ID NO: 305; PRT;->
RGRTLALRNYAYTTEVGYDD
< CDR3, SEQ ID NO: 306; PRT;->
RGRTLALRNYAYTTEVGYDD
< CDR3, SEQ ID NO: 307; PRT;->
RGGTLALRNYAYTTEVGYDD
< CDR3, SEQ ID NO: 308; PRT;->
SAIIEGFQDSIVIFSEAGYDY
< CDR3, SEQ ID NO: 309; PRT;->
VAGLLLPRVAEGMDY
< CDR3, SEQ ID NO: 310; PRT;->
VDSPLIATHPRGYDY
< CDR3, SEQ ID NO: 311; PRT;->
ARGLLIATDARGYDY
< CDR3, SEQ ID NO: 312; PRT;->
GSYVFYFTVRDQYDY
< CDR3, SEQ ID NO: 313; PRT;->
SAGGFLVPRVGQGYDY
< CDR3, SEQ ID NO: 314; PRT;->
SAGGFLVPRVGQGYDY
< CDR3, SEQ ID NO: 315; PRT;->
ERVGLLLTVVAEGYDY
< CDR3, SEQ ID NO: 316; PRT;->
ERVGLLLTVVAEGYDY
< CDR3, SEQ ID NO: 317; PRT;->
ERVGLLLTVVAEGYDY
< CDR3, SEQ ID NO: 318; PRT;->
ERVGLLLAVVAEGYDY
< CDR3, SEQ ID NO: 319; PRT;->
ERAGVLLTKVPEGYDY
< PMP6D5, SEQ ID NO: 320; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASQSGSGYDSWGQGTQVTVSS
< PMP8F2, SEQ ID NO: 321; PRT;->
DVQLVESGGDLVQPGGSLRLSCAASGFSFDDYAMSWLRQTPGKGLEWVGAITGNGASKYYAESMKGRFTISRDNAKNMLYL
HLNNLKSEDTAVYYCRRVAKDTGSFYYPAYEHDVLGQGTQVTVSS
< PMP6B12, SEQ ID NO: 322; PRT;->
AVQLVESGGGLVQPGGSLRLSCAASGFTLAYYAIGWFRQAPGKEREGVSCISSSVGTTYYSDSVKGRFTISRDNAKNTVYLQM
NSLKPEDTAVYYCVRSSWFDCGVQGRDLGNEYDYRGQGTQVTVSS
< PMP6B6, SEQ ID NO: 323; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSS
< PMP11C1, SEQ ID NO: 324; PRT;->
EVQLVESGGGLVQTGGSLRLSCA3GLAFSIYTMGWFRQAPGKEREFVAAAHWTVFRGNTYYVDSVKGRFTISRDNAKNTVY
LQMNSLKPEDSAVYYCAATRSTAWNSPQRYDYWGQGTQVTVSS
< PMP23H2, SEQ ID NO: 325; PRT;->
AVQLVDSGGGLVQPGGSLRLSCMSGSIFSRLAMDWYRQAPGKQRELVASIAVSGTTMLDDSVKGRFTISRDNAENTVYLQM
NSLKPEDTAVYYCMAFDGYTGSDYWGRGTQVTVSS
< PMP7G4, SEQ ID NO: 326; PRT;->
AVQLVESGGGLVQPGGSLRLSCAASGSIFSRLAMDWYRQAPGKQRELVASISRSGTTMAADSVKGRFTISRDNAENMVYLQM
NSLKPEDTAVYVCMAFDGYSGSDYWGRGTQVTVSS
< PMP20D2, SEQ ID NO: 327; PRT;->
AVQLVESGGGLVQPGGSLRLSCAASGSISRFNIMGWYRQAPGKQRELVADITNRGTTNYADSVKGRFTISRDNTKNTVYLQM
NSLKPDDTAVYYCHTYYPTTGFDDWGQGTQVTVSS
< PMP7G5, SEQ ID NO: 328; PRT;->
QVKLEESGGGLVQPGGSLRLSCAASGSISRFNIMGWYRQAPGKQRELVADTTNGGTTMYADSVKGRFTISRDNTKNTVYLQM
NSLKPEDTAVYYCHTYYPTTGFDDWGQGAQVTVSS
< PMP7H3, SEQ ID NO: 329; PRT;->
DVQLVESGGGLVQPGGSLRLSCAASGFTLDYYGVGWFRQAPGKEREGVSCISSSDGDTYYADSVKGRFTISRDNAKNTVYLQ
MNSLKPEDTAVYYCATDLSDYGVCSRWPSPYDYWGQGTQVTVSS
< PMP7G9, SEQ ID NO: 330; PRT;->
QVQLVESGGGLVQPGGSLRLSCAASGFSLDYYGVGWFRQAPGKEREGVSCISSSDGDTYYADSVKGRFTISRDNAKNTVYLQ
MNSLKPEDTAVYYCATDLSDYGVCSRWPSPYDYWGQGTQVTVSS
< PMP9A9, SEQ ID NO: 331; PRT;->
QVQLVESGGGLVQPGGSLRLSCAASGFSLDYVGVGWFRQAPGKEREGVSCTSSSDGDTYYADSVKGRFTISRDNAKNTVYL
QMNSLKPEDTAVYYCATDLSDYGVCSRWPSPYDYWGQGTQVTVSS
< PMP22E3, SEQ ID NO: 332; PRT;->
QVQLVESGGGLVQPGGSLRLSCAASGFTLDDSAIGWFRQAPGKEREGVSCISSSDGDTYYDDSVKGRFTISRDNVKNMVYLQ
MNSLKPEDTAVYFCAIDLSDYGVCSKWPSPYDYWGQGTQVTVSS
< PMP6E10, SEQ ID NO: 333; PRT;->
QVKLEESGGGLVQAGGSLRLSCVVSGRTFSPYTIAWFRQAPGKEREFVTTIIGSDRSTDLDGDTYYADSVRGRFTISRNDAKN
TVFLQMSSLKPEDTAVYYCALTGKGYVFTPNEYDYWGQGTQVTVSS
< PMP6G10, SEQ ID NO: 334; PRT;->
QVQLVESGGGLAQAGGSLRLSCVVSGRTFSPYTIGWFSQRPGKEREWVATIIGSDRSTDLDGDTYYADSVRGRFTISRNDAK
NTVSLQMNSLKPEDSAVYYCALTAKGYVFTDNEYDYWGQGTQVTVSS
< NC3, SEQ ID NO: 335; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSS
< NC6, SEQ ID NO: 336; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVTVSS
< PMP13A1, SEQ ID NO: 337; PRT;->
AVQLVDSGGGLVQAGGSLRLSCAASGRTFSPYTMGWFRQPPGKVREFVGRINWSGIRNYADSVKGRFTISRDNNNNTVYLQ
MNRLKPEDTAVYYCAAASQSGSGYDSWGQGTQVTVSS
< PMP20G9, SEQ ID NO: 338; PRT;->
QVQLVESGGGLVQAGGSLRLSCAASGRTFSPYIVGWFRQPPGKVREFVGRINWSGIRNYADSVKGRFTISRDNNNNTVYLQ
MNRLKPEDTAVYYCAAASQSGSGYDSWGQGTQVTVSS
< PMP20F4, SEQ ID NO: 339; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSS
< PMP21A7, SEQ ID NO: 340; PRT;->
AVQLVESGGGLVQAGSSLRLSCAASGRTFSPYTMGWFRQPPGKVREFVGRINWSGITNYADSVKGRFTISRDNNKNTVYLQ
MNRLKPEDTAVYYCAAASRSGSGYDSWGQGTQVTVSS
< PMP13D8, SEQ ID NO: 341; PRT;->
QVKLEESGGGLVQAGSSLRLSCAASGRTSSPYTMGWFRQPPGKVREFVGRINWSGITNYADSVKGRFTISRDNNKNTVYLQ
MNRLKPEDTAVYYCASASQVGSGYDSWGQGTQVTVSS
< PMP21E12, SEQ ID NO: 342; PRT;->
AVQLVESGGGLVQPGGSLRLSCAASGSITSINPMGWYRQAPGKQRELVARIHGSITNYADSVKGRFTISRDIAKNTVYLQMNS
LKPEDTAVYYCNARRWGYDYWGQGAQVTVSS
< PMP21C12, SEQ ID NO: 343; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVTVSS
< PMP21C2, SEQ ID NO: 344; PRT;->
QVQLVESGGGLVQPGGSLRLSCAASEYTTSINPMAWYRQAPGKQRDLVARIFGGGSTNYADSVKGRFTISRDIAKNTVSLQM
NSLKPEDTAVYYCNARRWGYDYWGQGTQVTVSS
< PMP14G4, SEQ ID NO: 345; PRT;->
AVQLVDSGGGLVQAGGSLRLSCAASGRTFSSYPMGWFRQGPGKERKFVAGISQSGVGTAYSDSVKGRFTISRENAKNTVYLQ
MNSLKPEDTAVYYCAARDKTLALRDYAYTTDVGYDDWGQGTQVTVSS
< PMP14E1, SEQ ID NO: 346; PRT;->
QVQLVESGGGLVQAGGSLRLSCAASGRTFSSYPMGWFRQAPGKERKFVAGISQSGGSTAYSDSVKGRFTISRENAKSTVYLQ
MNSLKPEDTAVYYCAARDKTLALRDYAYTTDVGYDDWGQGTQVTVSS
< PMP6E9, SEQ ID NO: 347; PRT;->
EVQLVESGGGLVQAGGSLRLSCAASGRTFSSYPMGWFRQAPGKERKFVAGISQSSSSTAYSDSVKGRFTISRENAKNTVYLQ
MNSLKPEDTAVYVCAARGRTLALRDYAYTTEVGYDDWGQGTQVTVSS
< PMP12H3, SEQ ID NO: 348; PRT;->
AVQLVDSGGGLVQAGGSLRLSCAASGGTFTSYPMGWFRQAPGKERKFVAGISQSGGSTAYSDSVKGRFTISRENAKTTVYLQ
MNSLKPEDTAVYYCAARGRTLFLRDYAYTTEVGYDDWGQGTQVTVSS
< PMP12C5, SEQ ID NO: 349; PRT;->
DVQLVESGGGLVQAGGSLRLSCAASGGTFTSYPMGWFRQAPGKERKFVAGISQSGGSTAYSDSVKGRFTISRENAKTTVYLQ
MNSLKPEDTAVYYCAARGRTLFLRGYAYTTEVGYDDWGQGTQVTVSS
< PMP17G7, SEQ ID NO: 350; PRT;->
QVKLEESGGGLVQAGGSLRISCAASGGTFSSYPMGWFRQAPGKEREFVTGISQSGGSTAYSDSVKGRFTISRENAKNTVYLQ
MNSLKPEDTAVYYCAARGRTIALRNYAYTTEVGYDDWGQGTQVTVSS
< PMP14G11, SEQ ID NO: 351; PRT;->
QVQLVESGGGLVQAGGSLRLSCAASGGTFSSFPMGWFRQAPGKGREFVAGISQSGGSTHYSDSVKGRFTISRENAKNTVYLQ
MNSLKPEDTAVYCAARGRTLALRNYAYTTEVGYDDWGQGTQVTVSS
< PMP9F9, SEQ ID NO: 352; PRT;->
AVQLVESGGGLVQAGGSLRLSCAASGGTFSSFPMGWFRQAPGEKREFVAGISQSGGSTHYSDSVKGRFTISRENARNTVYLQ
MNSLKPEDTAVYYCAARGRTLALRNYAYTTEVGYDDWGQGTQVTVSS
< PMP14A8, SEQ ID NO: 353 ; PRT;->
EVQLVESGGGLVQAGGSLRLSCAASGGTFSSFPMGWFRQAPGKERKFVAGISQSGGSTHYSDSVKGRFTISRENAKNTVYLQ
MNNLKPEDTAVYYCAARGRTLALRNYAYTTEVGYDDWGQGTQVTVSS
< PMP17B5, SEQ ID NO: 354; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSS
< PMP6B7, SEQ ID NO: 355; PRT;->
QVQLVESGGGLVQAGGSLRLSCAASGGTFSAFPMGWFRQAPGKERKFVAGISQSGGSTHYSDSVKGRFTISRENAKNTVYLQ
MNSLKPEDTAVYYCAARGRTLALRNYAYTTEVGYDDWGQGTQVTVSS
< PMP14E9, SEQ ID NO: 356; PRT;->
AVQLVESGGGLVQAGGSLRLSCAASGGTFSAFPMGWFRQAPGKEREFVAGISQSGGSTHYSDSVKGRFTISKENAKSTVYLQ
MNSLKPEDTAVYYCAARGRTLALRNYAYTTEVGYDDWGQGTQVTVSS
< PMP17D7, SEQ ID NO: 357; PRT;->
AVQLVDSGGGLVQAGGSLRLSCAASGGTFSAFPMGWFRQAPGKEREFVAGISQSGGSTHYSDSVKGRfTISKENAKNTVYLQ
MNSLKPEDTAVYYCAARGRTLALRNYAYTTEVGYDDWGQGTQVTVSS
< PMP14G1, SEQ ID NO: 358; PRT;->
QVKLEESGGGLVQAGGSLRLSCAASGGTFSAFPMGWFRQAPGKEREFVAGISQSGGSTHYSDSVKGRFTISKENAKNTVYLQ
MNSLKPEDTAVYYCAARGGTLALRNYAYTTEVGYDDWGQGTQVTVSS
< PMP17B11, SEQ ID NO: 359; PRT;->
QVQLVESGGGLVQAGGSLRLSCAASGPTFSTYAMGWFRQAPGKEREFVAAISWSGANTYYADSVKGRFTISRDNAKNTVYLR
MNSLKPEDTAAYYCAASAIIEGFQDSIVIFSEAGYDYWGQGTQVTVSS
< PMP10C4, SEQ ID NO: 360; PRT;->
AVQLVDSGGGLVQAGGSLRLSCAASGRSFSNYHMVWFRQAPGKEREFVAAASGSTSSTYYADSVKGRFTISRDNAKNTVYLQ
MNSLKPEDTAVYYCAAVAGLLLPRVAEGMDYWGKGTLVTVSS
< PMP17C4, SEQ ID NO: 361; PRT;->
AVQLVDSGGGLVQAGDSLRLSCAASGRTFSNYAMAWFRQAPGKEREFVAVISYAGGRTYYADSVKGRFTISRDNAKNTVYLQ
MNSLKPEDTAVYYCAAVDSPLIATHPRGYDYWGQGTQVTVSS
< PMP21B4, SEQ ID NO: 362; PRT;->
QVQLVESGGGLVQAGDSLRVACAASGRTFSIDAMAWFRQAPGKEREFVSTMNWSTGATYYADSVKGRFTSSRDNAKSTSYL
QMNSLKPEDTAWYYCAAARGLLIATDARGYDYWGQGTQVTVSS
< PMP21H1, SEQ ID NO: 363; PRT;->
QVQLVESGGGLVQTGGSLRLSCAASGSTFSKHHATGWFRQAPGKEREFVAALNWSGGNTYYTDSVKGRFTISRDNAQNTVY
LQMNSLKPEDTAVYYCAAGSYVFYFTVRDQYDYWGQGTQVTVSS
< PMP10A6, SEQ ID NO: 364; PRT;->
QVQLVESGGGLVQAGGSLRLSCASSGRTFSSYVMGWFRQTPGKEREFVSTINWSGSNGYYADSVKGRFTISRDNAKNTVYL
QMNNLKPEDTAVYYCAASAGGFLVPRVGQGYDYWGQGTQVTVSS
< PMP13H6, SEQ ID NO: 365; PRT;->
QVKLEESGGGLVQAGGSLRLSCASSGRTFSSYVMGWFRQTPGKEREFVSTINWSGSNKYYADSVKGRFTISRDNAKNTVYLQ
MNSLKPEDTAVYYCAASAGGFLVPRVGQGYDYWGTGTQVTVSS
< PMP13F12, SEQ ID NO: 366; PRT;->
AVQLVDSGGGLVQAGGSLRLSCAASGRFTSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSS
< PMP21A2, SEQ ID NO: 367; PRT;->
DVQLVESGGGLVQAGGSLRLSCAASGRTFSSSPMGWFRQAPGKEREFVAAISGRSGNTYYADSVKGRFTISRDNAKNTVYLQ
MNSLKPEDTAVYYCAGERVGLLLTVVAEGVDYWGQGTQVTVSS
< PMP21F7, SEQ ID NO: 368; PRT;->
EVQLVESGGGLVQAGGSLRLSCAASGRTFSSSPMGWFRQAPGKEREFVAAISGRSGNTYYADSVKGRFTISRDNAKNTVYLQ
MNSLKPEDTAVYYCAGERVGLLLTVVAEGYDYWGRGTQVTVSS
< PMP21H3, SEQ ID NO: 369; PRT;->
QVQLVESGGGLVQAGGSLRLSCAASGRTFSNGPMAWFRQAPGKEREFVSAISWRTGTTYYADSVKGRFTISRDNAKNTVYL
QMNSLKPEDTAVYYCAAERVGLLLAVVAEGYDYWGQGTQVTVSS
< PMP21E7, SEQ ID NO: 370; PRT;->
AVQLVESGGGLVQAGGSLRLSSVVSGGTFSSYPIAWFRQPPGKEREFVAAISWRGGNTYYADSVKGRFTISRDNAKNTVYLQ
MNSLKPEDTAVYYSAAERAGVLLTKVPEGYDYWGQGTQVTVSS
< NC3-25GS-6B6, SEQ ID NO: 371; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGGSGGGGSGGGGSGGGGSQVQLVESAGGLVQPGGSLR
LSCAASGIIFSINAMGWYRQAPGKRRELVADIMPYGSTEYADSVKGRFTISRDNAKNTVYLQMNSLKPEDTAVYYCHSYDPRGDDYWGQ
GTQVTVSS
< NC6-25GS-6B6, SEQ ID NO: 372; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVTVSSGGGGSGGGGSGGGGSGGGGSGGGGSQVQLVESAGGLVQPGGSLRL
SCAASGIIFSINAMGWYRQAPGKRRELVADIMPYGSTEYADSVKGRFTISRDNAKNTVYLQMNSLKPEDTAVYYCHSYDPRGDDYWGQG
TQVTVSS
< 20F4-25G5-6B6, SEQ ID NO: 373; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGGSGGGGSGGGGSGGGGSQVQLVESAGGLVQPGGSLRLSCAA
SGIIFSINAMGWYRQAPGKRRELVADIMPYGSTEYADSVKGRFTISRDNAKNTVYLQMNSLKPEDTAVYYCHSYDPRGDDYWGQGTQV
TVSS
< 21C12-25GS-6B6, SEQ ID NO: 374; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVTVSSGGGGSGGGGSGGGGSGGGGSGGGGSQVQLVESAGGLVQPGGSLRLSCAASGIIFS
INAMGWYRQAPGKRRELVADIMPYGSTEYADSVKGRFTISRDNAKNTVYLQMNSLKPEDTAVYYCHSYDPRGDDYWGQGTQVTVSS
< 17B5-25GS-6B6, SEQ ID NO: 375; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGGSGGGGSGGGGSGGGGSQVQLVESAGGLVQP
GGSLRLSCAASGIIFSINAMGWYRQAPGKRRELVADIMPYGSTEYADSVKGRFTISRDNAKNTVYLQMNSLKPEDTAVYYCHSYDPRGDD
YWGQGTQVTVSS
< NC3-25GS-7G5, SEQ ID NO: 376; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGGSGGGGSGGGGSGGGGSQVKLEESGGGLVQPGGSLR
LSCAASGSISRFNIMGWYRQAPGKQRELVADITNGGTTMYADSVKGRFTISRDNTKNTVYLQMNSLKPEDTAVYYCHTYYPTTGFDDW
GQGAQVTVSS
< NC6-25GS-7G5, SEQ ID NO: 377; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVTVSSGGGGSGGGGSGGGGSGGGGSGGGGSQVKLEESGGGLVQPGGSLRL
SCAASGSISRFNIMGWYRQAPGKQRELVADITNGGTTMYADSVKGRFTISRDNTKNTVYLQMNSLKPEDTAVYYCHTYYPTTGFDDWG
QGAQVTVSS
< 20F4-25GS-7G5, SEQ ID NO: 378; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGGSGGGGSGGGGSGGGGSQVKLEESGGGLVQPGGSLRLSCAAS
GSISRFNIMGWYRQAPGKQRELVADITNGGTTMYADSVKGRFTISRDNTKNTVYLQMNSLKPEDTAVYYCHTYYPTTGFDDWGQGAQV
TVSS
< 21C12-25GS-7G5, SEQ ID NO: 379; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVWSSGGGGSGGGGSGGGGSGGGGSGGGGSQVKLEESGGGLVQPGGSLRLSCAASGSISR
FNIMGWYRQAPGKQRELVADITNGGTTMYADSVKGRFTISRDNTKNTVYLQMNSLKPEDTAVYYCHTYYPTTGFDDWGQGAQVTVSS
< 17B5-25GS-7G5, SEQ ID NO: 380; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGGSGGGGSGGGGSGGGGSQVKLEESGGGLVQP
GGSLRLSCAASGSISRFNIMGWYRQAPGKQRELVADITNGGTTMYADSVKGRFTISRDNTKNTVYLQMNSLKPEDTAVYYCHTYYPTTG
FDDWGQGAQVTVSS
< 6B6-25GS-NC3, SEQ ID NO: 381; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGGSGGGGSGGGGSGGGGSEVQLVESGGGLVQPGGSLRLSCAASGNI
AAINVMNWYRQAPGTQREFVAAITSGGRKNYADSVKGRFTISRDNAKNTVHLQMNSLKPEDTAVYYCNADAPLASDDDVAPADYWGQ
GTQVTVSS
< 6B6-25GS-NC6, SEQ ID NO: 382; PRT,->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGGSGGGGSGGGGSGGGGSEVQLVESGGGLVQAGGSLRLSCAASGPT
FSSYAMGWFRQAPGKDREFVAAISSNGGSTRYADSVKGRFTISRDSAKNTAYLQMNSLKLEDTAVYYCAADETTGWVQLADFRSWGQG
TQVTVSS
< 6B6-25GS-20F4, SEQ ID NO:383; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGGSGGGGSGGGGSGGGGSQVQLVESGGGLVQAGGSLRLSCAASGR
TFSPYTMGWFRQPPGKVREFVGRINWSGIRNYADSVKGRFTISRDNNNNTVYLQMNRLKPEDTAVYYCAAASRSGSGYDSWGQGTQV
TVSS
< 6B6-25GS-21C12, SEQ ID NO: 384; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSVDPRGDDYWGQGTQVTVSSGGGGSGGGGSGGGGSGGGGSGGGGSAVQLVESGGGLVQPGGSLRLSCAASGSI
TGINPMGWYRQAPGKQRELVARIHGSITNYADSVKGRFTISRDIAKNTAYLQMNSLKPEDTAVYYCNARRWGYDYWGQGAQVTVSS
< 6B6-25GS-17B5, SEQ ID NO: 385; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSVDPRGDDYWGQGTQVTVSSGGGGSGGGGSGGGGSGGGGSGGGGSQVQLVESGGGLVQAGGSLRLSCAASGG
TFSAFPMGWFRQAPGKERKFVAGISQSGGSTHYSDSVKGRFTISRENAKNTIYLQMNSLKPEDTAVYYCAARGRTLALRNYAYTTEVGYD
DWGQGTQVTVSS
< 7G5-25GS-NC3, SEQ ID NO: 386; PRT;->
QVKLEESGGGLVQPGGSLRLSCAASGSISRFNIMGWYRQAPGKQRELVADITNGGTTMYADSVKGRFTISRDNTKNTVYLQM
NSLKPEDTAVYYCHYPTTGFDDWGQGAQVTVSSGGGGSGGGGSGGGGSGGGGSGGGGSEVQLVESGGGLVQPGGSLRLSCAASGN
IAAINVHNWYRQAPGTQREFVAAITSGGRKNYADSVKGRFTISRDNAKNTVHLQMNSLKPEDTAVYYCNADAPLASDDDVAPADYWGQ
GTQVTVSS
< 7G5-25GS-NC6, SEQ ID NO: 387; PRT;->
QVKLEESGGGLVQPGGSLRLSCAASGSISRFNIMGWYRQAPGKQRELVADITNGGTTMYADSVKGRFTISRDNTKNTVYLQM
NSLKPEDTAVYYCHTYYPTTGFDDWGQGAQVTVSSGGGGSGGGGSGGGGSGGGGSGGGGSEVQLVESGGGLVQAGGSLRLSCAASGP
TFSSYAMGWFRQAPGKDREFVAAISSNGGSTRYADSVKGRFTISRDSAKNTAYLQMNSLKLEDTAVYYCAADETTGWVQLADFRSWGQ
GTQVTVSS
< 7G5-25GS-20F4, SEQ ID NO: 388; PRT;->
QVKLEESGGGLVQPGGSLRLSCAASGSISRFNIMGWYRQAPGKQRELVADITNGGTTMYADSVKGRFTISRDNTKNTVYLQM
NSLKPEDTAVYYCHTYYPTTGFDDWGQGAQVTVSSGGGGSGGGGSGGGGSGGGGSGGGGSQVQLVESGGGLVQAGGSLRLSCAASG
RTFSPYTMGWFRQPPGKVREFVGRINWSGIRNYADSVKGRFTISRDNNNNTVYLQMNRLKPEDTAVYYCAAASRSGSGYDSWGQGTQ
VTVSS
< 7G5-25GS-21C12, SEQ ID NO: 389; PRT;->
QVKLEESGGGLVQPGGSLRLSCAASGSISRFNIMGWYRQAPGKQRELVADITNGGTTMYADSVKGRFTISRDNTKNTVYLQM
NSLKPEDTAVYYCHYPTTGFDDWGQGAQVTVSSGGGGSGGGGSGGGGSGGGGSGGGGSAVQLVESGGGLVQPGGSLRLSCAASGS
ITGINPMGWYRQAPDKQRELVARIHGSITNYADSVKGRFTISRDIAKNTAYLQMNSLKPEDTAVYYCNARRWGYDYWGQGAQVTVSS
< 7G5-25GS-17B5, SEQ ID NO: 390; PRT;->
QVKLEESGGGLVQPGGSLRLSCAASGSISRFNIMGWYRQAPGKQRELVADITNGGTTMYADSVKGRFTISRDNTKNTVYLQM
NSLKPEDTAVYYCHTYYPTTGFDDWGQGAQVTVSSGGGGSGGGGSGGGGSGGGGSGGGGSQVQLVESGGGLVQAGGSLRLSCAASG
GTFSAFPMGWFRQAPGKERKFVAGISQSGGSTHYSDSVKGRFTISRENAKNTIYLQMNSLKPEDTAVYTCAARGRTLALRNYAYTTEVGY
DDWGQGTQVTVSS
< 6B12-9GS-ALB8, SEQ ID NO: 391; PRT;->
AVQLVESGGGLVQPGGSLRLSCAASGFTLAYYAIGWFRQAPGKEREGVSCISSSVGTTYYSDSVKGRFTISRDNAKNTVYLQM
NSLKPEDTAVYYCVRSSWFDCGVQGRDLGNEYDYRGQGTQVTVSSGGGGSGGGSEVQLVESGGGLVQPGNSLRLSCAASGFTFSSFGM
SWVRQAPGKGLEWVSSISGSGSDTLYADSVKGRFTISRDNAKTTLYLQMNSLRPEDTAVYYCTIGGSLSRSSQGTLVTVSS
< 6B6-9GS-ALB8, SEQ ID NO: 392; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYOPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSS
< 7G4-9GS-ALB8, SEQ ID NO: 393; PRT;->
AVQLVESGGGLVQPGGSLRLSCAASGSIFSRLAMDWYRQAPGKQRELVASISRSGTTMAADSVKGRFTISRDNAENMVYLQM
NSLKPEDTAVYVCMAFDGYSGSDYWGRGTQVTVSSGGGGSGGGSEVQLVESGGGLVQPGNSLRLSCAASGFTFSSFGMSWVRQAPGK
GLEWVSSISGSGSDTLYADSVKGRFTISRDNAKTTLYLQMNSLRPEDTAVYYCTIGGSLSRSSQGTLVTVSS
< NC3-9GS-ALB8, SEQ ID NO: 394; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSS
< NC6-9G5-ALB8, SEQ ID NO: 395; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVNSSGGGGGGGSEVQLVESGGGLVQPGNSLRLSCAASGFTFSSFGMSWV
RQAPGKGLEWVSSISGSGSDTLYADSVKGRFTISRDNAKTTLYLQMNSLRPEDTAVYYCTIGGSLSRSSQGTLVTVSS
< 20F4-9GS-ALB8, SEQ ID NO: 396; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGSEVQLVESGGGLVQPGNSLRLSCAASGFTFSSFGMSWVRQAP
GKGLEWVSSISGSGSDTLYADSVKGRFTISRDNAKTTLYLQMNSLRPEDTAVYYCTIGGSLSRSSQGTLVTVSS
< 21C12-9GS-ALB8, SEQ ID NO: 397; PRT;->
AVQLVESGGGLVQPGGSLRLSCAASGSITGINPMGWVRQAPGKQRELVARIHGSITNYADSVKGRFTISRDIAKNTAYLQMN
SLKPEDTAVYYCNARRWGYDYWGQGAQVTVSSGGGGSGGGSEVQLVESGGGLVQPGNSLRLSCAASGFGTSSFGMSWVRQAPGKGLE
WVSSISGSGSDTLYADSVKGRFTISRDNAKTTLYLQMNSLRPEDTAVYYCTIGGSLSRSSQGTLVTVSS
< 17B5-9GS-ALB8, SEQ ID NO: 398; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGSEVQLVESGGGLVQPGNSLRLSCAASGFTFSSF
GMSWVRQAPGKGLEWVSSISGSGSDTLYADSVKGRFTISRDNAKTTLYLQMNSLRPEDTAVYYCTIGGSLSRSSQGTLVTVSS
< 17C4-9GS-ALB8, SEQ ID NO: 399; PRT;->
AVQLVDSGGGLVQAGDSLRLSCAASGRTFSNYAMAWFRQAPGKEREFVAVISYAGGRTYYADSVKGRFTISRDNAKNTVYLQ
MNSLKPEDTAVYYCAAVDSPLIATHPRGYDYWGQGTQVTVSSGGGGSGGGSEVQLVESGGGLVQPGNSLRLSCAASGFTFSSFGMSWV
RQAPGKGLEWVSSISGSGSDTLYADSVKGRFTISRDNAKTTLYLQMNSLRPEDTAVYYCTIGGSLSRSSQGTLVTVSS
< 13F12-9GS-ALB8, SEQ ID NO: 400; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSS
< 6B6-9GS-ALB8-9GS-13F12, SEQ ID NO: 401; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSAVQLVDSGGG
LVQAGGSLRLSCAASGRTFSSSPMGWFRQAPGKEREFVAAISGRSGNTYYADSVKGRFTISRDNAKNTVYLQMNSLKPEDTAVYYCAAER
VGLLLTVVAEGYDYWGQGTQVTVSS
< 7G4-9GS-ALB8-9GS-13F12, SEQ ID NO: 402; PRT;->
AVQLVESGGGLVQPGGSLRLSCAASGSIFSRLAMDWYRQAPGKQRELVASISRSGTTMAADSVKGRFTISRDNAENMVYLQM
NSLKPEDTAVYYCMAFDGYSGSDYWGRGTQVTVSSGGGGSGGGSEVQLVESGGGLVQPGNSLRLSCAASGFTFSSFGMSWVRQAPGK
GLEWVSSISGSGSDTLYADSVKGRFTISRDNAKTTLYLQMNSLRPEDTAVYYCTIGGSLSRSSQGTLVTVSSGGGGSGGGSAVQLVDSGG
GLVQAGGSLRLSCAASGRTFSSSPMGWFRQAPGKEREFVAAISGRSGNTYYADSVKGRFTISRDNAKNTVYLQMNSLKPEDTAVYYCAA
ERVGLLLTVVAEGYDYWGQGTQVTVSS
< NC3-9GS-ALB8-9GS-6B6, SEQ ID NO: 403; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVIVSSGGGGSGGGSQV
QLVESAGGLVQPGGSLRLSCAASGIIFSINAMGWYRQAPGKRRELVADIMPYGSTEYADSVKGRFTISRDNAKNTVYLQMNSLKPEDTAV
YYCHSYDPRGDDYWGQGTQVTVSS
< NC6-9GS-ALB8-9GS-6B6, SEQ ID NO: 404; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVTVSSGGGGSGGGSEVQLVESGGGLVQPGNSLRLSCAASGFTFSSFGMSWV
RQAPGLGLEWVSSISGSGSDTLYADSVKGRFTISRDNAKTTLYLQMNSLRPEDTAVYYCTIGGSLSRSSQGTLVTVSSGGGGSGGGSQV
QLVESAGGLVQPGGSLRLSCAASGIIFSINAMGWYRQAPGKRRELVADIMPYGSTEYADSVKGRFTISRDNAKNTVYLQMNSLKPEDTAV
YYCHSYDPRGDDYWGQGTQVTVSS
< 20F4-9GS-ALB8-9GS-6B6, SEQ ID NO: 405; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGSEVQLVESGGGLVQPGNSLRLSCAASGFTFSSFGMSWVRQAP
GKGLEWVSSISGSGSDTLYADSVKGRFTISRDNAKTTLYLQMNSLRPEDTAVYYCTIGGSLSRSSQGTLVTVSSGGGGSGGGSQVQLVES
AGGLVQPGGSLRLSCAASGIIFSINAMGWYRQAPGKRRELVADIMPYGSTEYADSVKGRFTISRDNAKNTVYLQMNSLKPEDTAVYYCHS
YDPRGDDYWGQGTQVTVSS
< 21C12-9GS-ALB8-9GS-686, SEQ ID NO: 406; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVTVSSGGGGSGGGSEVQLVESGGGLVQPGNSLRLSCAASGFTFSSFGMSWVRQAPGKGLE
WVSSISGSGSDTLYADSVKGRFTISRDNAKTTLYLQMNSLRPEDTAVYYCTIGGSLSRSSQGTLVTVSSGGGGSGGGSQVQLVESAGGLV
QPGGSLRLSCAASGIIFSINAMGWYRQAPGKRRELVADIMPYGSTEYADSVKGRFTISRDNAKNTVYLQMNSLKPEDTAVYYCHSVDPRG
DDYWGQGTQVTVSS
< 17B5-9GS-ALB8-9GS-6B6, SEQ ID NO: 407; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGSEVQLVESGGGLVQPGNSLRLSCAASGFTFSSF
GMSWVRQAPGKGLEWVSSISGSGSDTLYADSVKGRFTISRDNAKTTLYLQMNSLRPEDTAVYYCTIGGSLSRSSQGTLVTVSSGGGGSG
GGSQVQLGESAGGLVQPGGSLRLSCAASGIIFSINAMGWYRQAPGKRRELVADIMPYGSTEYADSVKGRFTISRDNAKNTVYLQMNSLKP
EDTAVYYCHSYDPRGDDYWGQGTQVTVSS
< 17C4-9GS-ALB8-9GS-6B6, SEQ ID NO: 408; PRT;->
AVQLVDSGGGLVQAGDSLRLSCAASGRTFSNYAMAWFRQAPGKEREFVAVISYAGGRTYYADSVKGRFTISRDNAKNTVYLQ
MNSLKPEDTAVYYCAAVDSPLIATHPRGYDYWGQGTQVTVSSGGGGSGGGSEVQLVESGGGLVQPGNSLRLSCAASGFTFSSFGMSWV
RQAPGKGLEWVSSISGSGSDTLYADSVKGRFTISRDNAKTTLYLQMNSLRPEDTAVYYCTIGGSLSRSSQGTLVTVSSGGGGSGGGSQV
QLVESAGGLVQPGGSLRLSCAASGIIFSINAMGWYRQAPGKRRELVADIMPYGSTEYADSVKGRFTISRDNAKNTVYLQMNSLKPEDTAV
YYCHSYDPRGDDYWGQGTQVTVSS
< 13F12-9GS-ALB8-9GS-6B6, SEQ ID NO: 409; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSSGGGGSGGGSQV
QLVESAGGLVQPGGSLRLSCAASGIIFSINAMGWYRQAPGKRRELVADIMPYGSTEYADSVKGRFTISRDNAKNTVYLQMNSLKPEDTAV
YYCHSYDPRGDDYWGQGTQVTVSS
< 13F12-9GS-ALB8-9GS-6B6, SEQ ID NO: 410; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSSGGGGSGGGSQV
QLVESAGGLVQPGGSLRLSCAASGIIFSINAMGWYRQAPGKRRELVADIMPYGSTEYADSVKGRFTISRDNAKNTVYLQMNSLKPEDTAV
YYCHSYDPRGDDVWGQGTQVTVSS
< 13F12-9GS-ALB8-9GS-7G4, SEQ ID NO: 411; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSSGGGGSGGGSAV
QLVESGGGLVQPGGSLRLSCAASGSIFSRLAMDWYRQAPGKQRELVASISRSGTTMAADSVKGRFTISRDNAENMVYLQMNSLKPEDTA
VYVCMAFDGYSGSDYWGRGTQVTVSS
< 6B6-9GS-ALB8-9GS-NC3, SEQ ID NO: 412; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSEVQLVESGGG
LVQPGGSLRLSCAASGNIAAINVMNWYRQAPGTQREFVAAITSGGRKNYADSVKGRFTISRDNAKNTVHLQMNSLKPEDTAVYYCNADA
PLASDDDVAPADYWGQGTQVTVSS
< 6B6-9GS-ALB8-9GS-NC6, SEQ ID NO: 413; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTYLLQMNSLRPEDTAVYYCTIGGSLSRSSQGTLVTVSSGGGGSGGGSEVQLVESGGG
LVQAGGSLRLSCAASGPTFSSYAMGWFRQAPGKDREFVAAISSNGGSTRYADSVKGRFTISRDSAKNTAYLQMNSLKLEDTAVYYCAADE
TTGWVQLADFRSWGQGTQVTVSS
< 6B6-9GS-ALB8-9GS-20F4, SEQ ID NO: 414; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSQVQLVESGGG
LVQAGGSLRLSCAASGRTFSPYTMGWFRQPPGKVREFVGRINWSGIRNYADSVKGRFTISRDNNNNTVYLQMNRLKPEDTAVYYCAAAS
RSGSGYDSWGQGTQVTVSS
< 6B6-9GS-ALB8-9GS-21C12, SEQ ID NO: 415; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSAVQLVESGGG
LVQPGGSLRLSCAASGSITGINPMGWYRQAPGKQRELVARIHGSITNYADSVKGRFTISRDIAKNTAYLQMNSLKPEDTAVYYCNARRWG
YDYWGQGAQVTVSS
< 6B6-9GS-ALB8-9GS-17B5, SEQ ID NO: 416; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSQVQLVESGGG
LVQAGGSLRLSCAASGGTFSAFPMGWFRQAPGKERKFVAGISQSGGSTHYSDSVKGRFTISRENAKNTIYLQMNSLKPEDTAVYYCAARG
RTLALRNYAYTTEVGYDDWGQGTQVTVSS
< 6B6-9GS-ALB8-9GS-17C4, SEQ ID NO: 417; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSAVQLVDSGGG
LVQAGDSLRLSCAASGRTFSNYAMAWFRQAPGKEREFVAVISYAGGRTYYADSVKGRFTISRDNAKNTVYLQMNSLKPEDTAVYYCAAV
DSPLIATHPRGYDYWGQGTQVTVSS
< 6B6-9GS-ALB8-9GS-13F12, SEQ ID NO: 418; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSAVQLVDSGGG
LVQAGGSLRLSCAASGRTFSSSPMGWFRQAPGKEREFVAAISGRSGNTYYADSVKGRFTISRDNAKNTVYLQMNSLKPEDTAVYYCAAER
VGLLLTVVAEGYDYWGQGTQVTVSS
< 6B12-9GS-TNF30, SEQ ID NO: 419; PRT;->
AVQLVESGGGLVQPGGSLRLSCAASGFTLAYYAIGWFRQAPGKEREGVSCISSSVGTTYYSDSVKGRFTISRDNAKNTVYLQM
NSLKPEDTAVYYCVRSSWFDCGVQGRDLGNEYDYRGQGTQVTVSSGGGGSGGGSEVQLVESGGGLVQPGGSLRLSCAASGFTFSDYW
MYWVRQAPGKGLEWVSEINTNGLITKYPDSVKGRFTISRDNAKNTLYLQMNSLRPEDTAVYYCARSPSGFNRGQGTLVTVSS
< 6B6-9GS-TNF30, SEQ ID NO: 420; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGGSLRLSCAASGFTFSDYWMYWVRQAPGK
GLEWVSEINTNGLITKYPDSVKGRFTISRDNAKNTLYLQMNSLRPEDTAVYYCARSPSGFNRGQGTLVTVSS
< 7G4-9GS-TNF30, SEQ ID NO: 421; PRT;->
AVQLVESGGGLVQPGGSLRLSCAASGSIFSRLAMDWYRQAPGKQRELVASISRSGTTMAADSVKGRFTISRDNAENMVYLQM
NSLKPEDTAVYVCMAFDGYSGSDYWGRGTQVTVSSGGGGSGGGSEVQLVESGGGLVQPGGSLRLSCAASGFTFSDYWMYWVRQAPGK
GLEWVSEINTNGLITKYPDSVKGRFTISRDNAKNTLYLQMNSLRPEDTAVYYCARSPSGFNRGQGTLVTVSS
< NC3-9GS-TNF30, SEQ ID NO: 422; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGSEVQLVESGGGLVQPGGSLRLSCAASGFTFSDYWMYW
VRQAPGKGLEWVSEINTNGLITKYPDSVKGRFTISRDNAKNTLYLQMNSLRPEDTAVYYCARSPSGFNRGQGTLVTVSS
< NC6-9GS-TNF30, SEQ ID NO: 423; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADETTGWVQLADFRSWGQGTQVTVSSGGGGSGGGSEVQLVESGGGLVQPGGSLRLSCAASGFTFSDYWMYW
VRQAPGKGLEWVSEINTNGLITKYPDSVKGRFTISRDNAKNTLYLQMNSLRPEDTAVYYCARSPSGFNRGQGTLVTVSS
< 20F4-9GS-TNF30, SEQ ID NO: 424; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGSEVQLVESGGGLVQPGGSLRLSCAASGFTFSDYWMYWVRQAP
GKGLEWVSEINTNGLITKYPDSVKGRFTISRDNAKNTLYLQMNSLRPEDTAVYYCARSPSGFNRGQGTLVTVSS
< 21C12-9GS-TNF30, SEQ ID NO: 425; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVTVSSGGGGSGGGSEVQLVESGGGLVQPGGSLRLSCAASGFTFSDYWMYWVRQAPGKGL
EWVSEINTNGLITKYPDSVKGRFTISRDNAKNTLYLQMNSLRPEDTAVYYCARSPSGFNRGQGTLVTVSS
< 17B5-9GS-TNF30, SEQ ID NO: 426; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGSEVQLVESGGGLVQPGGSLRLSCAASGFTFSDY
WMYWVRQAPGKGLEWVSEINTNGLITKYPDSVKGRFTISRDNAKNTLYLQMNSLRPEDTAVYYCARSPSGFNRGQGTLVTVSS
< 17C4-9GS-TNF30, SEQ ID NO: 427; PRT;->
AVQLVDSGGGLVQAGDSLRLSCAASGRTFSNYAMAWFRQAPGKEREFVAVISYAGGRTYYADSVKGRFTISRDNAKNTVYLQ
MNSLKPEDTAVYYCAAVDSPLIATHPRGYDYWGQGTQVTVSSGGGGSGGGSEVQLVESGGGLVQPGGSLRLSCAASGFTFSDYWMYW
VRQAPGKGLEWVSEINTNGLITKYPDSVKGRFTISRDNAKNTLYLQMNSLRPEDTAVYYCARSPSGFNRGQGTLVTVSS
< 13F12-9GS-TNF30, SEQ ID NO: 428; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGVDYWGQGTQVTVSSGGGGSGGGSEVQLVESGGGLVQPGGSLRLSCAASGFTFSDYWMY
WVRQAPGKGLEWVSEINTNGLITKYPDSVKGRFTISRDNAKNTLYLQMNSLRPEDTAVYYCARSPSGFNRGQGTLVTVSS
< TNF30-9GS-6B12, SEQ ID NO: 429; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSAVQLVESGGGLVQPGGSLRLSCAASGFTLAYYAIGWFRQAPGKEREG
VSCISSSVGTTYYSDSVKGRFTISRDNAKNTVYLQMNSLKPEDTAVYYCVRSSWFDCGVQGRDLGNEYDYRGQGTQVTVSS
< TNF30-9GS-6B6, SEQ ID NO: 430; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSQVQLVESAGGLVQPGGSLRLSCAASGIIFSINAMGWYRQAPGKRREL
VADIMPYGSTEYADSVKGRFTISRDNAKNTVYLQMNSLKPEDTAVYYCHSYDPRGDDYWGQGTQVTVSS
< TNF30-9GS-7G4, SEQ ID NO: 431; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSAVQLVESGGGLVQPGGSLRLSCAASGSIFSRLAMDWYRQAPGKQRE
LVASISRSGTTMAADSVKGRFTISRDNAENMVYLQMNSLKPEDTAVYVCMAFDGYSGSDYWGRGTQVTVSS
< TNF30-9GS-NC3, SEQ ID NO: 432; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSEVQLVESGGGLVQPGGSLRLSCAASGNIAAINVMNWYRQAPGTQRE
FVAATSGGRKNYADSVKGRFTISRDNAKNTVHLQMNSLKPEDTAVYYCNADAPLASDDDVAPADYWGQGTQVTVSS
< TNF30-9GS-NC6, SEQ ID NO: 433; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSEVQLVESGGGLVQAGGSLRLSCAASGPTFSSYAMGWFRQAPGKDRE
FVAAISSNGGSTRYADSVKGRFTISRDSAKNTAYLQMNSLKLEDTAVYCAADETTGWVQLADFRSWGQGTQVTVSS
< TNF30-9GS-20F4, SEQ ID NO: 434; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSQVQLVESGGGLVQAGGSLRLSCAASGRTFSPYTMGWFRQPPGKVRE
FVGRINWSGIRNYADSVKGRFTISRDNNNNTVYLQMNRLKPEDTAVYYCAAASRSGSGYDSWGQGTQVTVSS
< TNF30-9GS-21C12, SEQ ID NO: 435; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSAVQLVESGGGLVQPGGSLRLSCAASGSITGINPMGWYRQAPGKQRE
LVARIHGSITNYADSVKGRFTISRDIAKNTAYLQMNSLKPEDTAVYYCNARRWGYDYWGQGAQVTVSS
< TNF30-9GS-17B5, SEQ ID NO: 436; PRT;->
EVQLVESGGGLVQPGGSLRLSCAASGFTFSDYWMYWVRQAPGLGLEWVSEINTNGLITKYPDSVKGRFTISRDNAKNTLYLQ
MNSLRPEDTAVYYCARSPSGFNRGQGTLVTVSSGGGGSGGGSQVQLVESGGGLVQAGGSLRLSCAASGGTFSAFPMGWFRQAPGKERK
FVAGISQSGGSTHYSDSVKGRFTISRENAKNTIYLQMNSLKPEDTAVYYCAARGRTLALRNYAYTTEVGYDDWGQGTQVTVSS
< TNF30-9GS-17C4, SEQ ID NO: 437, PRT;->
EVQLVESGGGLVQPGG5LRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSAVQLVDSGGGLVQAGDSLRLSCAASGRTFSNYAMAWFRQAPGKERE
FVAVISYAGGRTYYADSVKGRFTISRDNAKNTVYLQMNSLKPEDTAVYYCAAVDSPLIATHPRGYDYWGQGTQVTVSS
< TNF30-9GS-13F12, SEQ ID NO: 438; PRT;->
EVQLVESGGGLVQPGGSLRLSCAASGFTFSDYWMYWVRQAPGKGLEWVSEINTNGLITKYPDSVKGRFTISRDNAKNTLYLQ
MNSLRPEDTAVYYCARSPSGFNRGQGTLVTVSSGGGGSGGGSAVQLVDSGGGLVQAGGSLRLSCAASGRTFSSSPMGWFRQAPGKERE
FVAAISGRSGNTYYADSVKGRFTISRDNAKNTVYLQMNSLKPEDTAVYYCAAERVGLLLTVVAEGYDYWGQGTQVTVSS
< 6B6-9GS-ALB8-9GS-TNF30, SEQ ID NO: 439; PRT;->
QVQLVESAGGLVQPGGSLRLSCAASGIIFSINAMGWYRQAPGKRRELVADIMPYGSTEYADSVKGRFTISRDNAKNTVYLQM
NSLKPEDTAVYYCHSYDPRGDDYWGQGTQVTVSSGGGGSGGGSEVQLVESGGGLVQPGNSLRLSCAASGFTFSSFGMSWVRQAPGKG
LEWVSSISGSGSDTLYADSVKGRFTISRDNAKTTLYLQMNSLRPEDTAVYYCTIGGSLSRSSQGTLVTVSSGGGGSGGGSEVQLVESGGG
LVQPGGSLRLSCAASGFTFSDYWMYWVRQAPGKGLEWVSEINTNGLITKYPDSVKGRFTISRDNAKNTLYLQMNSLRPEDTAVYYCARS
PSGFNRGQGTLVTVSS
< 7G4-9GS-ALB8-9GS-TNF30, SEQ ID NO: 440; PRT;->
AVQLVESGGGLVQPGGSLRLSCAASGSIFSRLAMDWYRQAPGKQRELVASISRSGTTMAADSVKGRFTISRDNAENMVYLQM
NSLKPEDTAVYVCMAFDGYSGSDYWGRGTQVTVSSGGGGSGGGSEVQLVESGGGLVQPGNSLRLSCAASGFTFSSFGMSWVRQAPGK
GLEWVSSISGSGSDTLYADSVKGRFTISRDNAKTTLYLQMNSLRPEDTAVYYCTIGGSLSRSSQGTLVTVSSGGGGSGGGSEVQLVESGG
GLVQPGGSLRLSCAASGFTFSDYWMYWVRQAPGKGLEWVSEINTNGLITKYPDSVKGRFTISRDNAKNTLYLQMNSLRPEDTAVYYCAR
SPSGFNRGQGTLVTVSS
< NC3-9GS-ALB8-9GS-TNF30, SEQ ID NO: 441; PRT;->
EVQLVESGGGLVQPGGSLRLSCAASGNIAAINVMNWYRQAPGTQREFVAAITSGGRKNYADSVKGRFTISRDNAKNTVHLQ
MNSLKPEDTAVYYCNADAPLASDDDVAPADYWGQGTQVTVSSGGGGSGGGSEVQLVESGGGLVQPGNSLRLSCAASGFTFSSFGMSW
VRQAPGKGLEWVSSISGSGSDTLYADSVKGRFTISRDNAKTTLYLQMNSLRPEDTAVYYCTIGGSLSRSSQGTLVTVSSGGGGSGGGSEV
QLVESGGGLVQPGGSLRLSCAASGFTFSDYWMYWVRQAPGKGLEWVSEINTNGLITKYPDSVKGRFTISRDNAKNTLYLQMNSLRPEDT
AVYYCARSPSGFNRGQGTLVTVSS
< NC6-9GS-ALB8-9GS-TNF30, SEQ ID NO: 442; PRT;->
EVQLVESGGGLVQAGGSLRLSCAASGPTFSSYAMGWFRQAPGKDREFVAAISSNGGSTRYADSVKGRFTISRDSAKNTAYLQ
MNSLKLEDTAVYYCAADEITGWVQLADFRSWGQGTQVTVSSGGGGSGGGSEVQLVESGGGLVQPGNSLRLSCAASGFTFSSFGMSWV
RQAPGKGLEWVSSISGSGSDTLYADSVKGRFTISRDNAKTTLYLQMNSLRPEDTAVYYCTIGGSLSRSSQGTLVTVSSGGGGSGGGSEVQ
LVESGGGLVQPGGSLRLSCAASGFTFSDYWMYWVRQAPGKGLEWVSEINTNGLITKYPDSVKGRFTISRDNAKNTLYLQMNSLRPEDTA
VYYCARSPSGFNRGQGTLVTVSS
< 20F4-9GS-ALB8-9GS-TNF30, SEQ ID NO: 443; PRT;->
QVQLVESGGGLVQAGGSLRLSCAASGRTFSPYTMGWFRQPPGKVREFVGRINWSGIRNYADSVKGRFTISRDNNNNTVYLQ
MNRLKPEDTAVYYCAAASRSGSGYDSWGQGTQVTVSSGGGGSGGGSEVQLVESGGGLVQPGNSLRLSCAASGFTFSSFGMSWVRQAP
GKGLEWVSSISGSGSDTLYADSVKGRFTISRDNAKTTLYLQMNSLRPEDTAVYYCTIGGSLSRSSQGTLVTVSSGGGGSGGGSEVQLVES
GGGLVQPGGSLRLSCAASGFTFSDYWMYWVRQAPGKGLEWVSEINTNGLITKYPDSVKGRFTISRDNAKNTLYLQMNSLRPEDTAVYYC
ARSPSGFNRGQGTLVTVSS
< 21C12-9GS-ALB8-9GS-TNF30, SEQ ID NO: 444; PRT;->
AVQLVESGGGLVQPGGSLRLSCAASGSITGINPMGWYRQAPGKQRELVARIHGSITNYADSVKGRFTISRDIAKNTAYLQMN
SLKPEDTAVYYCNARRWGYDYWGQGAQVTVSSGGGGSGGGSEVQLVESGGGLVQPGNSLRLSCAASGFTFSSFGMSWVRQAPGKGLE
WVSSISGSGSDTLYADSVKGRFTISRDNAKTTLYLQMNSLRPEDTAVYYCTIGGSLSRSSQGTLVTVSSGGGGSGGGSEVQLVESGGGLV
QPGGSLRLSCAASGTFTSDYWMYWVRQAPGKGLEWVSEINTNGLITKYPDSVKGRFTISRDNAKNTLYLQMNSLRPEDTAVYYCARSPS
GFNRGQGTLVTVSS
< 17B5-9GS-ALB8-9GS-TNF30, SEQ ID NO: 445; PRT;->
QVQLVESGGGLVQAGGSLRLSCAASGGTFSAFPMGWFRQAPGKERKFVAGISQSGGSTHYSDSVKGRFTISRENAKNTIYLQ
MNSLKPEDTAVYYCAARGRTLALRNYAYTTEVGYDDWGQGTQVTVSSGGGGSGGGSEVQLVESGGGLVQPGNSLRLSCAASGFTFSSF
GMSWVRQAPGKGLEWVSSISGSGSDTLYADSVKGRFTISRDNAKTTLYLQMNSLRPEDTAVYYCTIGGSLSRSSQGTLVTVSSGGGGSG
GGSEVQLVESGGGLVQPGGSLRLSCAASGFTFSDYWMYWVRQAPGKGLEWVSEINTNGLITKYPDSVKGRFTISRDNAKNTLYLQMNSL
RPEDTAVYYCARSPSGFNRGQGTLVTVSS
< 17C4-9GS-ALB8-9GS-TNF30, SEQ ID NO: 446; PRT;->
AVQLVDSGGGLVQAGDSLRLSCAASGRTFSNYAMAWFRQAPGKEREFVAVISYAGGRTYYADSVKGRFTISRDNAKNTVYLQ
MNSLKPEDTAVYYCAAVDSPLIATHPRGYDVWGQGTQVTVSSGGGGSGGGSEVQLVESGGGLVQPGNSLRLSCAASGFTFSSFGMSWV
RQAPGLGLEWVSSISGSGSDTLYADSVKGRFTISRDNAKTTLYLQMNSLRPEDTAVYYCTIGGSLSRSSQGTLVTVSSGGGGSGGGSEVQ
LVESGGGLVQPGGSLRLSCAASGFTFSDYWMYWVRQAPGKGLEWVSEINTNGLITKYPDSVKGRFTISRDNAKNTLYLQMNSLRPEDTA
VYYCARSPSGFNRGQGTLVTVSS
< 13F12-9GS-ALB8-9GS-TNF30, SEQ ID NO: 447; PRT;->
AVQLVDSGGGLVQAGGSLRLSCAASGRTFSSSPMGWFRQAPGKEREFVAAISGRSGNTYYADSVKGRFTISRDNAKNTVYLQ
MNSLKPEDTAVYYCAAERVGLLLTVVAEGYDYWGQGTQVTVSSGGGGSGGGSEVQLVESGGGLVQPGNSLRLSCAASGFTFSSFGMSW
VRQAPGLGLEWVSSISGSGSDTLYADSVKGRFTISRDNAKTTLYLQMNSLRPEDTAVYYCTIGGSLSRSSQGTLVTVSSGGGGSGGGSEV
QLVESGGGLVQPGGSLRLSCAASGFTFSDYWMYWVRQAPGKGLEWVSEINTNGLITKYPDSVKGRFTISRDNAKNTLYLQMNSLRPEDT
AVYYCARSPSGFNRGQGTLVTVSS

1. Amino acid sequence comprising or essentially consisting of an immunoglobulin variable domain or an antigen binding fragment thereof directed against IL-6, which modulates the interaction between IL-6 and IL-6R.

2. Amino acid sequence comprising or essentially consisting of an immunoglobulin variable domain or an antigen binding fragment thereof directed against IL-6, which competes with IL-6R for binding to IL-6.

3. Amino acid sequence according to claim 1, wherein said immunoglobulin variable domain or an antigen binding fragment thereof binds to an epitope of IL-6 which lies in, comprises, or fully or partially overlaps with the IL-6R interaction site of IL-6.

4. Amino acid sequence comprising or essentially consisting of an immunoglobulin variable domain or an antigen binding fragment thereof directed against IL-6, which modulates the interaction between IL-6/IL-6R complex and gp130.

5. Amino acid sequence comprising or essentially consisting of an immunoglobulin variable domain or an antigen binding fragment thereof directed against IL-6, which competes with gp130 for binding to the gp130 interaction site II of IL-6.

6. Amino acid sequence comprising or essentially consisting of an immunoglobulin variable domain or an antigen binding fragment thereof directed against IL-6, which competes with gp130 for binding to the gp130 interaction site III of IL-6.

7. Amino acid sequence according to claim 1, wherein said immunoglobulin variable domain or an antigen binding fragment thereof binds to IL-6 with a dissociation constant (Kd) of 10⁻⁵ to 10⁻¹² moles/liter or less, and preferably 10⁻⁷ to 10⁻¹² moles/liter or less and more preferably 10⁻⁸ to 10⁻¹² moles/liter.

8. Amino acid sequence according to claim 1, wherein said immunoglobulin variable domain is chosen from the group consisting of a light chain variable domain, a heavy chain variable domain, a (single) domain antibody, and a Nanobody®.

9. Amino acid sequence according to claim 1, wherein said immunoglobulin variable domain is a Nanobody®.

10. Amino acid sequence according to claim 1, wherein said immunoglobulin variable domain is a humanized Nanobody®.

11. Amino acid sequence according to claim 8, wherein said Nanobody® comprises or consists of 4 framework regions (FR1 to FR4 respectively) and 3 complementarity determining regions (CDR1 to CDR3 respectively), in which:
CDR1 is an amino acid sequence chosen from the group consisting of:

| | |
|---|---|
| SEQ ID NO: 167 | PYTMG |
| SEQ ID NO: 168 | DYAMS |
| SEQ ID NO: 169 | YYAIG |
| SEQ ID NO: 170 | INAMG |
| SEQ ID NO: 171 | IYTMG |
| SEQ ID NO: 172 | RLAMD |
| SEQ ID NO: 173 | RLAMD |
| SEQ ID NO: 174 | FNIMG |
| SEQ ID NO: 175 | FNIMG |
| SEQ ID NO: 176 | YYGVG |
| SEQ ID NO: 177 | YYGVG |
| SEQ ID NO: 178 | YYGVG |
| SEQ ID NO: 179 | DSAIG |
| SEQ ID NO: 180 | PYTIA |
| SEQ ID NO: 181 | PYTIG |
| SEQ ID NO: 182 | INVMN |
| SEQ ID NO: 183 | SYAMG |
| SEQ ID NO: 184 | PYTMG |
| SEQ ID NO: 185 | PYTVG |
| SEQ ID NO: 186 | PYTMG |
| SEQ ID NO: 187 | PYTMG |
| SEQ ID NO: 188 | PYTMG |
| SEQ ID NO: 189 | INPMG |
| SEQ ID NO: 190 | INPMG |
| SEQ ID NO: 191 | INPMA |
| SEQ ID NO: 192 | SYPMG |
| SEQ ID NO: 193 | SYPMG |
| SEQ ID NO: 194 | SYPMG |
| SEQ ID NO: 195 | SYPMG |
| SEQ ID NO: 196 | SYPMG |
| SEQ ID NO: 197 | SYPMG |
| SEQ ID NO: 198 | SFPMG |
| SEQ ID NO: 199 | SFPMG |
| SEQ ID NO: 200 | SFPMG |
| SEQ ID NO: 201 | AFPMG |
| SEQ ID NO: 202 | AFPMG |
| SEQ ID NO: 203 | AFPMG |
| SEQ ID NO: 204 | AFPMG |
| SEQ ID NO: 205 | AFPMG |
| SEQ ID NO: 206 | TYAMG |
| SEQ ID NO: 207 | NYHMV |
| SEQ ID NO: 208 | NYAMA |
| SEQ ID NO: 209 | IDAMA |
| SEQ ID NO: 210 | KHHATG |
| SEQ ID NO: 211 | SYVMG |
| SEQ ID NO: 212 | SYVMG |
| SEQ ID NO: 213 | SSPMG |
| SEQ ID NO: 214 | SSPMG |
| SEQ ID NO: 215 | SSPMG |
| SEQ ID NO: 216 | NGPMA |
| SEQ ID NO: 217 | SYPIA |

or from the group consisting of amino acid sequences that have at least 80%, preferably at least 90%, more preferably at least 95%, even more preferably at least 99% sequence identity (as defined herein) with one of the above amino acid sequences; in which
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s);
and/or in which:
CDR2 is an amino acid sequence chosen from the group consisting of:

| | |
|---|---|
| SEQ ID NO: 218 | RINWSGIRNYADSVKG |
| SEQ ID NO: 219 | AITGNGASKYYAESMKG |
| SEQ ID NO: 220 | CISSSVGTTYYSDSVKG |
| SEQ ID NO: 221 | DIMPYGSTEYADSVKG |
| SEQ ID NO: 222 | AAHWTVFRGNTYYVDSVKG |
| SEQ ID NO: 223 | SIAVSGTTMLDDSVKG |
| SEQ ID NO: 224 | SISRSGTTMAADSVKG |
| SEQ ID NO: 225 | DITNRGTTNYADSVKG |
| SEQ ID NO: 226 | DITNGGTTMYADSVKG |
| SEQ ID NO: 227 | CISSSDGDTYYADSVKG |
| SEQ ID NO: 228 | CISSSDGDTYYADSVKG |
| SEQ ID NO: 229 | CTSSSDGDTYYADSVKG |
| SEQ ID NO: 230 | CISSSDGDTYYDDSVKG |
| SEQ ID NO: 231 | TIIGSDRSTDLDGDTYYADSVRG |
| SEQ ID NO: 232 | TIIGSDRSTDLDGDTYYADSVRG |
| SEQ ID NO: 233 | AITSGGRKNYADSVKG |
| SEQ ID NO: 234 | AISSNGGSTRYADSVKG |
| SEQ ID NO: 235 | RINWSGIRNYADSVKG |
| SEQ ID NO: 236 | RINWSGIRNYADSVKG |
| SEQ ID NO: 237 | RINWSGIRNYADSVKG |
| SEQ ID NO: 238 | RINWSGITNYADSVKG |
| SEQ ID NO: 239 | RINWSGITNYADSVKG |
| SEQ ID NO: 240 | RIHGSITNYADSVKG |
| SEQ ID NO: 241 | RIHGSITNYADSVKG |
| SEQ ID NO: 242 | RIFGGGSTNYADSVKG |
| SEQ ID NO: 243 | GISQSGVGTAYSDSVKG |
| SEQ ID NO: 244 | GISQSGGSTAYSDSVKG |
| SEQ ID NO: 245 | GISQSSSSTAYSDSVKG |
| SEQ ID NO: 246 | GISQSGGSTAYSDSVKG |
| SEQ ID NO: 247 | GISQSGGSTAYSDSVKG |
| SEQ ID NO: 248 | GISQSGGSTAYSDSVKG |
| SEQ ID NO: 249 | GISQSGGSTHYSDSVKG |
| SEQ ID NO: 250 | GISQSGGSTHYSDSVKG |
| SEQ ID NO: 251 | GISQSGGSTHYSDSVKG |
| SEQ ID NO: 252 | GISQSGGSTHYSDSVKG |
| SEQ ID NO: 253 | GISQSGGSTHYSDSVKG |
| SEQ ID NO: 254 | GISQSGGSTHYSDSVKG |
| SEQ ID NO: 255 | GISQSGGSTHYSDSVKG |
| SEQ ID NO: 256 | GISQSGGSTHYSDSVKG |
| SEQ ID NO: 257 | AISWSGANTYYADSVKG |
| SEQ ID NO: 258 | AASGSTSSTYYADSVKG |
| SEQ ID NO: 259 | VISYAGGRTYYADSVKG |
| SEQ ID NO: 260 | TMNWSTGATYYADSVKG |
| SEQ ID NO: 261 | ALNWSGGNTYYTDSVKG |
| SEQ ID NO: 262 | TINWSGSNGYYADSVKG |
| SEQ ID NO: 263 | TINWSGSNKYYADSVKG |
| SEQ ID NO: 264 | AISGRSGNTYYADSVKG |
| SEQ ID NO: 265 | AISGRSGNTYYADSVKG |
| SEQ ID NO: 266 | AISGRSGNTYYADSVKG |
| SEQ ID NO: 267 | AISWRTGTTYYADSVKG |
| SEQ ID NO: 268 | AISWRGGNTYYADSVKG |

or from the group consisting of amino acid sequences that have at least 80%, preferably at least 90%, more preferably at least 95%, even more preferably at least 99% sequence identity (as defined herein) with one of the above amino acid sequences; in which
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s);
and/or in which:
CDR3 is an amino acid sequence chosen from the group consisting of:

| | |
|---|---|
| SEQ ID NO: 269 | ASQSGSGYDS |
| SEQ ID NO: 270 | VAKDTGSFYYPAYEHDV |
| SEQ ID NO: 271 | SSWFDCGVQGRDLGNEYDY |
| SEQ ID NO: 272 | YDPRGDDY |
| SEQ ID NO: 273 | TRSTAWNSPQRYDY |
| SEQ ID NO: 274 | FDGYTGSDY |
| SEQ ID NO: 275 | FDGYSGSDY |
| SEQ ID NO: 276 | YYPTTGFDD |
| SEQ ID NO: 277 | YYPTTGFDD |
| SEQ ID NO: 278 | DLSDYGVCSRWPSPYDY |
| SEQ ID NO: 279 | DLSDYGVCSRWPSPYDY |
| SEQ ID NO: 280 | DLSDYGVCSRWPSPYDY |
| SEQ ID NO: 281 | DLSDYGVCSKWPSPYDY |
| SEQ ID NO: 282 | TGKGYVFTPNEYDY |
| SEQ ID NO: 283 | TAKGYVFTDNEYDY |
| SEQ ID NO: 284 | DAPLASDDDVAPADY |
| SEQ ID NO: 285 | DETTGWVQLADFRS |
| SEQ ID NO: 286 | ASQSGSGYDS |
| SEQ ID NO: 287 | ASQSGSGYDS |
| SEQ ID NO: 288 | ASRSGSGYDS |
| SEQ ID NO: 289 | ASRSGSGYDS |
| SEQ ID NO: 290 | ASQVGSGYDS |
| SEQ ID NO: 291 | RRWGYDY |
| SEQ ID NO: 292 | RRWGYDY |
| SEQ ID NO: 293 | RRWGYDY |
| SEQ ID NO: 294 | RDKTLALRDYAYTTDVGYDD |
| SEQ ID NO: 295 | RDKTLALRDYAYTTDVGYDD |
| SEQ ID NO: 296 | RGRTLALRDYAYTTEVGYDD |
| SEQ ID NO: 297 | RGRTLFLRDYAYTTEVGYDD |
| SEQ ID NO: 298 | RGRTLFLRGYAYTTEVGYDD |
| SEQ ID NO: 299 | RGRTIALRNYAYTTEVGYDD |
| SEQ ID NO: 300 | RGRTLALRNYAYTTEVGYDD |
| SEQ ID NO: 301 | RGRTLALRNYAYTTEVGYDD |
| SEQ ID NO: 302 | RGRTLALRNYAYTTEVGYDD |
| SEQ ID NO: 303 | RGRTLALRNYAYTTEVGYDD |
| SEQ ID NO: 304 | RGRTLALRNYAYTTEVGYDD |
| SEQ ID NO: 305 | RGRTLALRNYAYTTEVGYDD |
| SEQ ID NO: 306 | RGRTLALRNYAYTTEVGYDD |
| SEQ ID NO: 307 | RGGTLALRNYAYTTEVGYDD |
| SEQ ID NO: 308 | SAIIEGFQDSIVIFSEAGYDY |
| SEQ ID NO: 309 | VAGLLLPRVAEGMDY |
| SEQ ID NO: 310 | VDSPLIATHPRGYDY |
| SEQ ID NO: 311 | ARGLLIATDARGYDY |
| SEQ ID NO: 312 | GSYVFYFTVRDQYDY |
| SEQ ID NO: 313 | SAGGFLVPRVGQGYDY |
| SEQ ID NO: 314 | SAGGFLVPRVGQGYDY |
| SEQ ID NO: 315 | ERVGLLLTVVAEGYDY |
| SEQ ID NO: 316 | ERVGLLLTVVAEGYDY |
| SEQ ID NO: 317 | ERVGLLLTVVAEGYDY |
| SEQ ID NO: 318 | ERVGLLLAVVAEGYDY |
| SEQ ID NO: 319 | ERAGVLLTKVPEGYDY |

or from the group consisting of amino acid sequences that have at least 80%, preferably at least 90%, more preferably at least 95%, even more preferably at least 99% sequence identity (as defined herein) with one of the above amino acid sequences; in which
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
a) any amino acid substitution is preferably a conservative amino acid substitution (as defined herein); and/or
b) said amino acid sequence preferably only contains amino acid substitutions, and no amino acid deletions or insertions, compared to the above amino acid sequence(s).

12. Nanobody® that is directed against and/or that can specifically bind to IL-6.

13. Nanobody® according to claim 12, that is in essentially isolated form.

14. Nanobody® according to claim 11, that can specifically bind to IL-6 with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter.

15. Nanobody® according to claim 12, that can specifically bind to IL-6 with a rate of association (kₒₙ-rate) of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.

16. Nanobody® according to claim 12, that can specifically bind to IL-6 with a rate of dissociation (k_{off} rate) between 1 s⁻¹ and 10⁻⁶ s⁻¹ preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

17. Nanobody® according to claim 12, that can specifically bind to IL-6 with an affinity less than 500 nM, preferably less than 200 nM, more preferably less than 10 nM, such as less than 500 pM.

18. Nanobody® according to claim 12, that is a naturally occurring Nanobody® (from any suitable species) or a synthetic or semi-synthetic Nanobody®.

19. Nanobody® according to claim 12 that is a V_{HH} sequence, a partially humanized V_{HH} sequence, a fully humanized V_{HH} sequence, a camelized heavy chain variable domain or a Nanobody® that has been obtained by techniques such as affinity maturation.

20. Nanobody® according to claim 12, that
i) has 80% amino acid identity with at least one of the amino acid sequences of SEQ ID NO's: 320 to 447, in which for the purposes of determining the degree of amino acid identity, the amino acid residues that form the CDR sequences are disregarded;
and in which:
ii) preferably one or more of the amino acid residues at positions 11, 37, 44, 45, 47, 83, 84, 103, 104 and 108 according to the Kabat numbering are chosen from the Hallmark residues mentioned in Table A-3.

21. Nanobody® according to claim 12, in which:
CDR1 is chosen from the group consisting of:
a) the amino acid sequences of SEQ ID NO's: 167 to 217
b) amino acid sequences that have at least 80% amino acid identity with at least one of the amino acid sequences of SEQ ID NO's: 167 to 217
c) amino acid sequences that have 3, 2, or 1 amino acid difference with at least one of the amino acid sequences of SEQ ID NO's: 167 to 217;
and/or
CDR2 is chosen from the group consisting of:
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
and/or
CDR3 is chosen from the group consisting of:
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319.

22. Nanobody® according to claim 12, in which:
CDR1 is chosen from the group consisting of:
a) the amino acid sequences of SEQ ID NO's: 167 to 217
b) amino acid sequences that have at least 80% amino acid identity with at least one of the amino acid sequences of SEQ ID NO's: 167 to 217;
c) amino acid sequences that have 3, 2, or 1 amino acid difference with at least one of the amino acid sequences of SEQ ID NO's: 167 to 217;
and
CDR2 is chosen from the group consisting of:
d) the amino acid sequences of SEQ ID NO's: 218 to 268;
e) amino acid sequences that have at least 80% amino acid identity with at least one of the amino acid sequences of SEQ ID NO's: 218 to 268;
f) amino acid sequences that have 3, 2, or 1 amino acid difference with at least one of the amino acid sequences of SEQ ID NO's: 218 to 268;
and
CDR3 is chosen from the group consisting of:
g) the amino acid sequences of SEQ ID NO's: 269 to 319;
h) amino acid sequences that have at least 80% amino acid identity with at least one of the amino acid sequences of SEQ ID NO's: 269 to 319;
i) amino acid sequences that have 3, 2, or 1 amino acid difference with at least one of the amino acid sequences of SEQ ID NO's: 269 to 319.

23. Nanobody® according to claim 12, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 320 to 447.

24. Nanobody® according to claim 12, which is a partially humanized Nanobody®.

25. Nanobody® according to claim 12, which is a fully humanized Nanobody®.

26. Nanobody® according to claim 12 that is chosen from the group consisting of SEQ ID NO's: 320 to 447 or from the group consisting of from amino acid sequences that have more than 80%, preferably more than 90%, more preferably more than 95%, such as 99% or more sequence identity (as defined herein) with at least one of the amino acid sequences of SEQ ID NO's: 320 to 447.

27. Nanobody® according to claim 12, which is a humanized Nanobody®.

28. Nanobody® according to claim 12, that is chosen from the group consisting of SEQ ID NO's: 320 to 447.

29. Compound or construct, that comprises or essentially consists of one or more amino acid sequences according to claim 1, and optionally further comprises one or more other groups, residues, moieties or binding units, optionally linked via one or more linkers.

30. Compound or construct according to claim 29, in which said one or more other groups, residues, moieties or binding units are amino acid sequences.

31. Compound or construct according to claim 29, in which said one or more linkers, if present, are one or more amino acid sequences.

32. Compound or construct according to any of claim 29, in which said one or more other groups, residues, moieties or binding units are immunoglobulin sequences.

33. Compound or construct according to claim 29, in which said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a “dAb”, or Nanobodies®.

34. Compound or construct according to claim 29, in which said one or more amino acid sequences of the invention are immunoglobulin sequences.

35. Compound or construct according to claim 29, in which said one or more amino acid sequences of the invention are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a “dAb”, or Nanobodies®.

36. Compound or construct, that comprises or essentially consists of one or more Nanobodies® according to claim 12 and in which said one or more other groups, residues, moieties or binding units are Nanobodies®.

37. Compound or construct according to claim 29, which is a multivalent construct.

38. Compound or construct according to claim 29, which is a multispecific construct.

39. Compound or construct according to claim 29, in which said one or more other groups, residues, moieties or binding units bind to a therapeutically relevant target.

40. Compound or construct according to claim 39, in which said therapeutically relevant target is TNF-α.

41. Compound or construct that comprises or essentially consists of one or more amino acid sequences according to claim 1 and optionally further comprises one or more other groups residues, moieties or binding units, optionally linked via one or more linkers, which has an increased half-life, compared to the corresponding amino acid sequence according to claim 1.

42. Compound or construct according to claim 41, in which said one or more other groups, residues, moieties or binding units provide the compound or construct with increased half-life, compared to the corresponding amino acid sequence according to claim 1.

43. Compound or construct according to claim 41, in which said one or more other groups, residues, moieties or binding units that provide the compound or construct with increased half-life is chosen from the group consisting of serum proteins or fragments thereof, binding units that can bind to serum proteins, an Fc portion, and small proteins or peptides that can bind to serum proteins.

44. Compound or construct according to claim 41, in which said one or more other groups, residues, moieties or binding units that provide the compound or construct with increased half-life is chosen from the group consisting of human serum albumin or fragments thereof.

45. Compound or construct according to claim 41, in which said one or more other groups, residues, moieties or binding units that provide the compound or construct with increased half-life are chosen from the group consisting of binding units that can bind to serum albumin (such as human serum albumin) or a serum immunoglobulin (such as IgG).

46. Compound or construct according to claim 41, in which said one or more other groups, residues, moieties or binding units that provides the compound or construct with increased half-life are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a “dAb”, or Nanobodies® that can bind to serum albumin (such as human serum albumin) or a serum immunoglobulin (such as IgG).

47. Compound or construct according to claim 41, in which said one or more other groups, residues, moieties or binding units that provide the compound or construct with increased half-life is a Nanobody® that can bind to serum albumin (such as human serum albumin) or a serum immunoglobulin (such as IgG).

48. Compound or construct according to claim 41, that has a serum half-life that is at least 1.5 times, preferably at least 2 times, such as at least 5 times, for example at least 10 times or more than 20 times, greater than the half-life of the corresponding amino acid sequence according to claim 1.

49. Compound or construct according to claim 41, that has a serum half-life that is increased with more than 1 hours, preferably more than 2 hours, more preferably more than 6 hours, such as more than 12 hours, or even more than 24, 48 or 72 hours, compared to the corresponding amino acid sequence according to claim 1.

50. Compound or construct according to claim 41, that has a serum half-life in human of at least about 12 hours, preferably at least 24 hours, more preferably at least 48 hours, even more preferably at least 72 hours or more, preferably at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

51. Monovalent construct, comprising or essentially consisting of one amino acid sequence according to claim 1.

52. Monovalent construct according to claim 51, in which said amino acid sequence of the invention is chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a “dAb”, or Nanobodies®.

53. Monovalent construct, comprising or essentially consisting of one Nanobody® according to claim 12.

54. Nucleic acid or nucleotide sequence, that encodes an amino acid sequence according to claim 1.

55. Nucleic acid or nucleotide sequence according to claim 54, that is in the form of a genetic construct.

56. Host or host cell that expresses, or that under suitable circumstances is capable of expressing, an amino acid sequence according to claim 1.

57. Method for producing an amino acid sequence, said method at least comprising the steps of:
a) expressing, in a suitable host cell or host organism or in another suitable expression system, a nucleic acid or nucleotide sequence that encodes an amino acid sequence according to claim 1,
optionally followed by:
b) isolating and/or purifying the amino acid sequence according to claim 1 thus obtained.

58. Method for producing an amino acid sequence, said method at least comprising the steps of:
a) cultivating and/or maintaining a host or host cell according to claim 56 under conditions that are such that said host or host cell expresses and/or produces at least one amino acid sequence,
optionally followed by:
b) isolating and/or purifying the amino acid sequence thus obtained.

59. Composition, comprising at least one amino acid sequence according to claim 1.

60. Composition according to claim 59, which is a pharmaceutical composition.

61. Composition according to claim 60, which is a pharmaceutical composition, that further comprises at least one pharmaceutically acceptable carrier, diluent or excipient and/or adjuvant, and that optionally comprises one or more further pharmaceutically active polypeptides and/or compounds.

62. Method for the prevention and/or treatment of at least one disease and/or disorder associated with IL-6 and/or with the IL-6/IL-6-R complex and/or with the signalling pathways and/or the biological functions and responses in which IL-6 and/or the IL-6/IL-6-R complex are involved, said method comprising administering, to a subject in need thereof, a pharmaceutically active amount of at least one amino acid sequence according to claim 1.

63. Method according to claim 62, wherein said disease and/or disorder associated with IL-6 and/or with the IL-6/IL-6-R complex and/or with the signalling pathways and/or the biological functions and responses in which IL-6 and/or the IL-6/IL-6-R complex are involved, is chosen from the group consisting of sepsis, various forms of cancer, bone resorption, osteoporosis, cachexia, psoriasis, mesangial proliferative glomerulonephritis, Kaposi's sarcoma, AIDS-related lymphoma, and inflammatory diseases.

64. Method according to claim 63, wherein said various forms of cancer are chosen from the group consisting of multiple myeloma disease (MM), renal cell carcinoma (RCC), plasma cell leukaemia, lymphoma, B-lymphoproliferative disorder (BLPD), and prostate cancer.

65. Method according to claim 63, wherein said inflammatory diseases are chosen from the group consisting of rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma, allergic asthma and autoimmune insulin-dependent diabetes mellitus.

66. Method for the prevention and/or treatment of at least one disease and/or disorder associated with IL-6 and/or with the IL-6/IL-6-R complex and/or with the signalling pathways and/or the biological functions, pharmacological activities and responses in which IL-6 and/or the IL-6/IL-6-R complex are involved, said method comprising administering, to a subject in need thereof, a pharmaceutically active amount of at least one amino acid sequence according to any of claim 1.

67. Method for the prevention and/or treatment of at least one disease or disorder that can be prevented and/or treated by administering, to a subject in need thereof, an amino acid sequence according to claim 1, said method comprising administering, to a subject in need thereof, a pharmaceutically active amount of at least one amino acid sequence according to claim 1.

68. Method for immunotherapy, said method comprising administering, to a subject in need thereof, a pharmaceutically active amount of at least one amino acid sequence according to claim 1.

69. (canceled)

70. (canceled)
